SEQUENCE LISTING (37 sequences)

SEQ ID NO: 1 - DNA, 22 bp, Artificial Sequence
  Synthetic - Forward Primer
gcagcctctgttccacatacac

SEQ ID NO: 2 - DNA, 20 bp, Artificial Sequence
  Synthetic - Reverse Primer
taagcctgcccagaagactc

SEQ ID NO: 3 - DNA, 21 bp, Artificial Sequence
  Synthetic - Second Forward Primer
aaagtcgctctgagttgttat

SEQ ID NO: 4 - DNA, 21 bp, Artificial Sequence
  Synthetic - KRAB Forward Primer
ggcgcgcctgcagccttcaag

SEQ ID NO: 5 - DNA, 21 bp, Artificial Sequence
  KRAB Reverse Primer
gaatcaggatgggtctcttgg

SEQ ID NO: 6 - DNA, 21 bp, Artificial Sequence
  Synthetic - lacZ ForwardPrimer
tttaacgccgtgcgctgttcg

SEQ ID NO: 7 - DNA, 21 bp, Artificial Sequence
  Synthetic - lacZ Reverse Primer
atccagcgatacagcgcgtcg

SEQ ID NO: 8 - DNA, 20 bp, Artificial Sequence
  Synthetic - Agrn gRNA
gactgcggcgcccgccgagc

SEQ ID NO: 9 - DNA, 20 bp, Artificial Sequence
  Synthetic - sav1 gRNA
agtttaccggacgtaggcgg

SEQ ID NO: 10 - DNA, 20 bp, Artificial Sequence
  Synthetic - Agrn Forward
ttcgatggtccttgtgaccc

SEQ ID NO: 11 - DNA, 20 bp, Artificial Sequence
  Synthetic - Agrn Reverse
agataggtgtgtgttgggcg

SEQ ID NO: 12 - DNA, 20 bp, Artificial Sequence
  Synthetic - sal1 Forward
gggaggcacacttcaggtat

SEQ ID NO: 13 - DNA, 20 bp, Artificial Sequence
  Synthetic - sal1 Reverse
cagcattccctggtacgtgt

SEQ ID NO: 14 - DNA, 20 bp, Artificial Sequence
  Synthetic - Beta-Actin Forward
aaggccaaccgtgaaaagat

SEQ ID NO: 15 - DNA, 21 bp, Artificial Sequence
  Synthetic - Beta-Actin Reverse
gtggtacgaccagagggatac

SEQ ID NO: 16 - DNA, 163634 bp, Mus musculus
gatccttctgactccacctccaagtgctaggttgcaggtacacctaacccatgagagcttatggcttgctaagcaggcaccaggagtctctggccaagttcattcaccaggcaagcacacctggagtctactggtggggtctccagaaggctggggtgcttgatgctgtcctttaccctctgcagtgggagcttaggggaggggaggccgacttgttcctagcaggtagatctgaccgattcagatgctaatctgctaaaactatggtcagccttacaggtctgtatagaattttcatctgtacttccagaacagatgctgttaagggaccaggaaccagacacatgaaatgactagctccttcctgggcatttgatgtaggcagtcaggtggtgacacctgtgtggtaaggttaaaaccttattcccatatatcttctcacacctccccacaaagtgacacagaaaaagtctatttaagtacttttaaaagtattttattgagatcgttcaggaaactatgcatttccaagccattatatagtctgggcaagataagttcttatttcatttgtctaatactcatgttcaagggaggccctggttcagtctgggcgcagggctcgcagattacaccttacagcctctcatgttcagataactggcaacaaagcaataaaaagccgtccaacttgtcagtgcgtagcagcaaagccctgaaaattcagaagaacattttcagcaggggaacagcaaacacaaagctgatttatgtgtttctaaaaccaggatattccaacaaggtcagctttacaatctgcacagcgggttcattgtgctaactttacaggccctagtgacatcatacaaagccttcaacaacccctccaccccgcctttactgctttattaccctgaaaagtgagctaaccacccaccttcatgtgggcaggacaaagggctggctctcattagatgattagctcattcaggtcacatctaggtcacttccacctttgtctggattccaaggttagccctcatctaggtgaggggatggggcccctgtgaagtcctcagagctcaccctggagagttaagatgggcacaatgagaaacaggagagcagggtatgttcctcaccagagccagtgttggcacactgggctcaatctcaagaggttccccaaatgagtcagatttatagctgacatcaaggacagcgtcagagactctagtctgtgaaatcatcactctcaattgagggagaccagaacctagggtaccacccagggaatgtcaatccgatagacacaggatgcggtagccagtgtgtgtagttaggcttcggactgttgcagagctatataaagccaaaactcctggggactgaaatttaactgctaaaacctttaacgaaaaaaggtggcatgggccggagagatggctcataaccatgtgtaatgggatctgacgccctcttctggtgtgtctgaagacagcaatagtgtactcacaaacatctaaatcttaaaaaaaaaaaaaaaaaggtggttggagccaggactaaggccatcctgaattgagagcccgtctaaatacccaaaagcatagtctaaaacagtggctctcaacctgtgtcaccaaggaacccttacataggggtcccagacaggacagcctgtgttatcacaatagcaaactttagttatgaagatacaaggaaaataaaaccgatggttaggaacatcacagcccgaggaactgcattaaaggccctcaggaaggctgagacccactgccgtggactagcatacagaacttaaaggcacaagaaataatcaaggcagctcctggatccaccatagatttagagttagggagattagttaagatctaaaaacaaaaagatggctgggtggtagcttgcttggagactggctaatgcaggcaggatattaagaacaaaacaagggccagtcggtggtgctgtatgcatttaattccagagagtagaggcaggcggatctgagatcagggctacatggagaaaccctgtctcgaatccaccccttccaaaaagtcaagccaagagacaagcatgccagtgaaacctaagactaacctttcttcaggtgtaggcagctggcgtgtgtcctcaaatccctaccagaaagcaagacacagaggtaaattactcagaactctaggccactcaaaacccacgccactgccgattttggacccggggacggtcagcgcgctgcggtgatggcgtcagcgagacactcagaatcaccagaaatcatcattactgtcctctccccgaaaccagcccctcctcacaccgcctacctctcgccagcatgcgcttaccattgtgctactgggtacggctggcgtgggccaggaacctggaagagatcattgaatgagcacaggtcgtagagaccccggcctgcagcccccaccccggcctgggtcagttacaaggagtggcgtcacgtggacatcagaaactctagtctgtaaatgtgggtcatcatacccggccccacaccgcagagccctccccacaggaccccaggatacctgcggcacgaggcggacccatgccttgctctccggagtcctcagaccctgcccagaccggtggagaacaaagaagggtgaaaacacggcaaaccgtgtgggcgggtgcacaaagagacccgtgtgccgcccgcctcggccatttatacccacgcctggcccgggggtcctgacggacatagtcgtcttctcatatgcctgacttgatcgcgctgacaaatgcgaagggaacaaagaggaatcatgtttcttactgtcctaagtagagaatcatgtatatgacgacaatttgataaactaccaatccaaaggaaagaaaaaaaaaaaagaaagaaagaaacaggcagcctaatcggccaagattgggtaaagaccctggagctcctacaggataggtcagctctttggtctcctttaggcttatggctttgcggattctgtactcatgtttggatccagtctacccaggaggactcatattgcctacacctagcagaaaaatctgaatttgcacctcagcttctgctctccgttaaaaagaaaaaaaaaagaaaaaaaaaagattactgagtgctctgctgcatgtacacgtgcgtgccagaagagggcatcagattcccttatacatggtcacgagcatttggttgctgggaattgaactcaggacttcaggaaaagcaaccagtgctcttaactggtgagccatctcaccagccccaacatttttatttaaagggggataaaagccacacacacacaaacacacatctggctcatagtctaagatgggttccaaccatgcctacaattacaggcgtgacacactactatgttgagagagagagagagagagagagagagagagggggggggggtcggtttcaagaccgggtttctctgtgtaaccctgactgtcctggaacttgctctgtagactaggctggcctcgaactcacagatatctacttgcctctgccctgagtgctgggattaaaggcatacaacaccactggccagcttttaagaaatatttttagattgattgtatttgtgtctatgtgtgttagaggaagttgtaagccacctggtgataggtgctgggaactgaagccaggtccttagaaagatcagtaagtgctcctgaccactgagccactgctccagcccctttgtctttttttttttttttgagacagcaattctgtaccatgacggtcaacattcctacttccttttcctaaatgctgaaattacatgtgcaaggcactatacgcttctctatcttcattttaaaatacattttagccagaagcagtggtgcacaactttaatcctagcacttgagaggcaggggcaggtagatctctttgagttcaaggccagcctgggctacgcagtgatgagatagtgcctcagaacataactaaaatttaaaatctaagaatcctatagtagccgggcagtggtggcacatgcctttaatcccagtacttgggaggcagaggcaggcagatttctgagttcaaggccagcctggtctacagagttagttccaggacagccagggctacacagagaaaccctgtctcaaaaaacaaaacaaaacaaaaaacaaacaaaaacaaaaacaaataaaagaatcctatagtaattatgtgttcgataagtctctcagttcctaggcccaagttccacctgggagggttataggaagagactcttcagttccaaactgagtgcacgttagccagaaatgacctctaacagtgtaaaatggcttacgaattaactgcttctctaatgagcataattgccttggcctctattaaactattactgaagacttcttttttttttttttaaacgcagatgtagtacaatataatagtaactggttgaaagtagtacatgctaacagacaatatgatacacaaacagggtggcagtagacaggaaagggcagaggccacggcctacaccaagagcctttcaatagactgctgaatggactggatctgctgctttagttgagagccttctttgatggtaacagaacaggcgatgactggcctggagatgccacacgccccgtcctaaagcctgcttggagcgcacaaatacataggggacattcttgtcttctcacagcagagggaggtgcaggatgatccccaagggctcagcgtctgctgccatcacagtgaactcagagatgcctctgttgggggttttggtggcttcattggctcctttccgaagctgcttgtagttacatgactgttgaacaaggcccagcagcttcttggtgaggtgggcatctgcgaggggataggccttcggagtcacatcagtctctgtcatggctgcggtgtgccggcctcgccgcgactcgacgacagcccacctcaagagggactgaaaaactactgaagacatctttaccctagggtggccttaaagtttctgtttggacatgttagaattgcatgcagtttacaccttgcccagcaaatcgattgattctccatggagctggagattgaattgagtgcctcatgcatatactaggcaagcagtctcccctgacctaccctccccagctctcaaaggctggcattgccccactaaaaccctggaagagccaggggtcaaaaggtttctgaggaaagcagctctgtgccctaggttctgcctgcttaaaaagccttggacaaggtaagggctctgctacgtcaccaggcctggaaagagagatggggatggtggggataaagtggatgggtcaggtagcacagtgcccataatcccaccattcaagaggcagagacaggagtcggagagcagcgtggtctagataatcacttccagcccagctaaggctacactgaaagactccgtctctctgactctctgtctctgtctctgtctctcacacacacacacacacatacgcacacaaaccctgcccctccccccaaaagaaaaaagatagccagacatggtgcctggtcacaactttataatttcagctagcaggttcaggggtatggtctggaaagggcaagttcaaagccaggctgagctacagagaaagtgtaaatatagtttgggcaaccctgtaagaggtctcaaaaaagtaataaagaagccaggttgggaaaggcatatgggtcagaggttgaggccagccaggactgcatggggagatgctgtctcagaaaataaagctaaaaaggagggctcagtggctgggggatgaagctcactgtctagagcagtggttctcaaacttcctaatgttgctattcttaagttccacatgttctggtgacacccaaccatgagaaatagttggtgttgatactttataactataattttagcctggtagtagtggcacaggcctttaatcccagcacttgggagccagaggaaggcaaatttcttagtttataggccagcctggtctataaggtcgttccaaagcagccagggctacagagaaatctgtcttgaaaaacaaaaaacaaaacaaaaaccaaaaacaaacaacccctgtaattttcaatgtcttatgcaacccttgtgaaagagtcacttgactccaataggggttcaaacactgtttaggattcaggccctggagggtaagggagagctgagctctcatagccccttgagtccctcttccactgaccccaaacaacagcaccaccctactttttcccatgaatgagcccgaccctcaaagtaaatacctgtactccctgcataactgcactgaaaatccaccaaaaaaaaatcccttttccagttatcacaaaactaagaaatccgggggctggagagacagctcagcagttaagagcactgactactcttccgaaggtcctgagttcaaatcccaggaaccacatggtggctcacacccatctgtaatgagatctgatgcccttttctggtgtgtctgaagatagctacaatgtgttcacatataataataaatcttaaaaaaaacttaagaaatccacagaagcccagaatccattagcccttccaacatacaaacagacaaaatatacttgtaaccttttattttgaatatttaagtcataaaagtttttttcattagtttgtagtgatgcaaagaaaaaaatgtacacagggtggcagtgtagacatgtatgcaggaaggatacaccatctttaacctacttagattaaatgcaaagaacatactggttataccacaaaaccttaacatggacaagcagataaagttttaagtacaaaagagtaaagttatgtagtacacagtgccaagacgtttgtttaagacactttgtcttagagatgaccatctccagggaactgggcagctagactatatgtcttcccaggctactggaacactgatataaaaattacaaaaaaatctttagactttacacatcattagctataaaacataatcccctggtacagtgcacagtgctgctcccaacattactttgaatatatttgaatatatatatatatatatatatatatatatatatatatgcatacaaaatttaacagtcaacgcaattaccttaagaaagactgtaactttaaactgaaccatgaagaagactttaaataaccttgacagtaatcggtgcccaagcagctatcaagtgcaaagagtctgttttctagaccacaccgggagagccagcagaggcctctaatccacctcctcgatggtgggtcctgagccagaggctcctttcggcggcgcctgggccccgaagcccccagccccaggagcacccgcaccctggtacagcccactgatgatggggctgcacacccgctccagctcctcccgcttgtgcacgaactcctccttgtcggccagcgtgttggagtccagccaggagatgacctcctggcacttgtccagcaccttcttcttgtcagcctcgctgagcttgcccttgagaccctcgtcctccacggcgctcttcatgttgaaggcataggactcgagcgcgttcttggcggccaccctgtcgcgctgcacctcgtcctcggccttgtagcgctcggcctcctgcaccatgcgctcgatctcctccttgctcaggcggcccttgtcgttggtgatggtgatcttgttggccttgccggtgctcttgtcggtggccgtgacgttcaggatgccgttggcgtcgatgtcgaaggtcacctcgatctgcggcacgcccctgggcgccggcgggatgccgctcagctcgaagcgccccagcaggttgttgtcgcgcgtcatggccctctcgccctcgtacacctggatcagcaccccgggctggttgtccgagtaggtggtgaaggtctgcgtctgcttggtggggatggtggagttgcgcttgatgagcgccgtcatcacgccgcccgcagtctccaggcccagcgacagcggcgccacgtccagcagcagcaggtcctgcacgttctccgacttgtcccccatcaggatggccgcctgcaccgccgccccgtaggccaccgcctcgtccgggttgatgctcttgttcaggtcgcgcccgttgaagaagtcctgcagcagcttctgcaccttggggatgcgcgtcgagccgcccaccagcaccaggtcgtggatctgcgccttgtccatcttggcgtcgcgcagggccttctccacgggctccagcgtgccgcggaacaggtccgagcacagctcttcgaaccgcgcccgcgtgatggatgtgtagaagtcgatgccctcgaacagagagtcgatctccaggctggcctgggtgctggacgacagcgtcctcttggccctctcacacgccgtgcgcagccgccgcaccgcgcgcttgttctggctgatgtccttcttgtgcttcctcttgaactcctccacgaagtggctcaccagccggttgtcgaagtcctcccctcccaggtgcgtgtcgcccgccgtggccttcacctcgaagatgccgtcgtcgatcgtcaggatggacacgtcgaacgtgccgccccccaggtcgaagatgagcacgttgcgctcgcccttgccggtccggtccagcccgtaggcgatggcggccgccgtgggctcgttgatgatccgcagcacgtttagaccggcgatcacgcccgcgtccttggtggcctgccgctgagagtcgttgaagtaggcgggcaccgtgatcaccgcgttggtcaccgggtggcccaggtacgcctcagcgatctccttcatcttcgtcagcaccatggacgagatctcctccgggaagaacgaccggctctcgcccttgtagttcacctgcaccttgggcttgtcgccgtcgttcaccacctggaagggccagtgcttcatgtcggactgcaccaccgcatcgccgaacttgcggccgatcagccgcttcgcgtcgaacacggtgttctgcgggttcagcgccacctggttcttggcggcgtccccgatgaggcgctcggtgtcggtgaaggccacgtagctgggggtcgtgcggttgccctggtcgttggcgatgatctccaccttgccgtgctggaacacgcccacgcacgagtaggtggtgcccaggtcgatgccgatcgccgtgttcttggccatggcgccgcgctctgcttctggaaggctgcgctccgcggcgtggatgctccggggaaagttgcgctccgcggcagggatgctcctgggaaggttgcgctccgcggcgtggatgctctggggaaggctggtcctggccgaggatcgggaacgcgccgctcgctctgcttctcttgtcttcgcttgtctctggatggaaccagatttgattctgagtagctgtcagcgtctggtgccctgctcgccgccctgcgcctttaaggagtcttcaccggccccgcccactctccgctgggccaatcagcgagccggaggaggccttggggccaggaatcttccagcagtttcgcgtctggtggagcttccccgcctcccttgagtaatcggagttgtgggttccgcccttgtccagaactctccagaggtttctggggttcgctagagagtacggattcctgagggggagggtgtggggaagtgctggtgctgctattgacactgttgcggaggcgacgcaacttcggggtccctgtgggtgaagctgttaagggtcagctcctgaaggtgctgggtggtgagagtgtgggactcagaccagggagggagtttaaagtgtacaaatccttcctacaggatggcggacgcagaaagtttctcaagagaggcgggataaggcttggctaaaaagtctgtgattgggtcctggtttgagtgacagcgttgggcaggggcgcctgtggaatgcttgcttgtccatgtctccccctccctcttcattttaggtttgaatagggaaatagaagagatcttgcagtatagttcatttgttttgctgtctttacatcacggttaaggtaggagtaggcggtacatcgcctatggagttctcacaattttataaggaaatcagcgtgaaaatacttgattctcatgttaggaggacctgtgactcaaaagaggaaaaaggaaagtgtcccaggacccggagagcagggcttaatcgaccgtgccaggctgtagccactccctttgcctcaggctgcagcctgacattagtaagaaaaaactttccatagacggtgaggcagaaggactgtagagagtccaagaccagcctccgttatagaggaaccctatctcaaaagattaaataaaaattgctctttgccaagcaccactgtagggtctgtgctctgctgtagtaaggtcaggttaatcaacagttcgttggttcttctgtctgtgtctgccgtctctgtctgttcacctcttttagaagagatggcgtgccctatcctctgatagcatcttgcctcacagtgccctgcgaactttccggtgagatcctaagcctgggtgttggggcccgcctactaattcaccctattagctttttgtcctaaacatcactttctatgcctggtctaccctctcccttttaattataagtacatttagctgtcagtgatgtccccttttctccttctctgcccctcctttctcctcctgtgtaggaattaaagaccccctagtcagatgccttgaaaccacagtatgaggttactgggtatggtggcccacctctgtaatcctcatgctttggaggggcgggcaggaggatgaagagtttgaggctagccttagctacattagagcctctctttcagaaaaaaaaaaaaaaaaaaaaaaaacaacgtagataacacccatagcagagagagactgttaattgtcacatacacacatctttttcatattaaacttttttttcttttattttgaaatagggtcttacatagcccaggctggccttgaacttactgtgtagccaaagctgatcttaaactcttgctcctctagccttgactcctaagtgctggaattccagacaagcactacaaactacacacacaggtctctgagtgtgaattggaggaggcagtttctggagggagccctgcttttaaatacaactccattggcctgcctctctctctctctctctctctccctctctctctctctctctctctctttctctctctctctctctctgtgtgtgtgtgtgtgtgtgtgtgtgtgtgtgtgtgtgtgtgtacataagtccatatcagaatatctcttctgggaatccaagggcatttagttgtctgtgctgtagtttgctgatctcagccagtgtttggaagatgctgcttcatagatcctctgtttgtgtgggaagaattggccagcagcaacttgttccatagttagacctgatgaattcagttctaatgagtcaccaccctgatctagtcaaactggttaaacttcaaaaaattttccattactctgtgcacctctggctggggattcctccaggagtccatgttagttagctacctagtcacttctggagacttgcagactcccttcccttagaggcctctcaccaccacctgtgaccacttgggtttactcttaccctgtcaatcagcaatacatagaactctcttttaaaagaaaatttgagtcagggtctcattctgtagcacaggatatctgggactgtgtgcctggaagctggccttgaactcagctccatcctcttgtctccccacacaagtgctaggattaacggaaacagctgctgtactcagcccatgtccatttctccacaagaaggtatttgaaattttgtctgttctcttagcctgggtacttctgaggcagaagcacagtggtaggttttgttgttgttgttattattttaagatttatttattatatgtaagtacactgtagctgacttcagacactccagaaaagggcatcagatctcgttacggatggttgtgagccaccacatggttgctgggatttgaactcatgaccttcggaagggcagtcagtgctcttaccccctgagccatctcaccagcccccacagtggtagatttaactgctgaagactggagggtctgaaaaatcccccaaactgccagggctgttgctcatttgtagaaaacatgctaagcatccaagaggccctgaatttaattcccagtaccagtcctctcactcccaacacacacacacacacatacacacacacaaaagagaaactttgtttcattcgggaattaggcttttattgagttccatatttggagacaacagagttcagggttctgaggctgggaggactgtggaggacttttaaggggtgggaagcatcaggggcagaagaggaagccaagaacacttgaggagagagagagcctcagccagaagctccagcatctcctcccccatcccgcccccaccttataatgcttctctaaacagccgttttctcctcagtcttcttggttttttaaagatgacataattttaagcatacccaggggaaaccaaaggaaatgcgttgtgacccctggaggccagctttctggttgtgtgtgtgtgtgtgtgtgtgtgtgtgtgtgtgtgtgtgtgtgtgtgagagagagagagagagagagagagagagagagagagagagagagagagggagagagagagggagagagagagagagagacttatagttcaagctggctcttggttctgcctcagctcctcaaatgcaggtttaactggtgtaagcaccacaccgtgctgagacaggcattcttaagcaaatttcaaaggtttcggtgacaagagaactctggagatttccagtgatttggaaaaacacccttggttggggaagacctgtatttgtgtctggaatctaccccttggtcaaggatgggttgttttggtttttactttttaacaatgaagtacatgtgtgctcctaaactgcctaagtttctatctgatcatctctaaaatagaattaaggataactacttcaggcaggtgtgatggtgtgctcttgtaattctagaatgggagagggagaggttgaggctcgaggatgctgtgagactttgaacaacctgggccataagaagaaaccatgcctcaaatccactggtgtagctgagctggtgccttgcctgcctagtatgcacaatgcccagggtcaggtccctggcaccacagaaaccaggtctggtatcacacacttgtaataccatcaatggggtagaggcaggggatcagaagtctaaggttttccttggctacccaatgagcttgtagccagcctggaatgcataagatcatgtctcaggaaacaaacacctccccaaactcagacaaaacaaaccaaaaaacaatttcatcgcatggtggcacaagtgattaactgactggcacttcagtacctgttagtgtgtgctcaggcagtttcctctcctctcatggctgtcccacacactcaggtctggctgatgaggtgcttacaagctgggaacattcacagacctatggaattactttgacactaacacaaaaatggtggtaaagtcagggtgctggtttctatctgtaaccccagcccttgagaggaagcaacagagggattgtcacaagtttgaaacagcctggtctctacagccagtttaggccagccagagctatattgtaagaccctgctccaaaaccaaaccaaaccagcagaccttagctctatgttgtaccagtctaccaggagagaggcttcagtattagcttccttataccacaaaacagtgacagaagacaggtgtcgcttttatatgcaaagctggagaggtgtggctgtcttcagacacaccagaagagggcatcagatctcatttcagatggttgtgagccaccatgtggttgttgggatttgaactcaggacctttggaagagcagtcagtgctcttaaccgctgaaccatctcaccggccctggagagttttgtgggtttttttgttgttattttaaagatttatttttaataatgtgtatgtgtacagaagtgcaggtgcctacagaggacaggaaagggcattgagtccactgggactggaatgacagatgtttgtaagttccaaaccatggcgcctgggaaccaaatgcaaatcctctagaagagtaggagaaaaaaatatataaacaatacataaaaatatataaattatatatatatatatatacataaaagtaggagcagctcttcaccatggacccatcatctctccaaccctgagttagaggttatttatttattcattttggtttttccagacagggtttctctgtgtacacctggcccggctgttctgggaaccatagaccaggctgaccttgaactcaggatccatctgcctcagcctcccgagtgtgccaacagtgctgggctgagttggggtttattaagacattttaaagcatagatcttgagtacaagagttaagacagagaggcacctgagagcaggggtgtgggcttttgaaagaagaacggcagacacaaatagatttagtccataacacaaacccggacaagccatggtccctgacctgcacaacaaagagcagacacatacggcttacattgcacgagagcttcatagtactgggtaagccagcaccagcctgtctgggacccacagagcttcccactttttcttagaaccccatgaaagatcggggtgagggagaaggcaagaaggcagtagctgaaaggccagagcacagatcgtccatcctctgccttggagccttttcaaagctttgccttgagtctgcctctcctttcactgacagtatagactcagaagggcagcagagtgaaggtcagggctctgtaagctttaaatccaggtggattaagtgaccacccagcaattcccctcctcctgcccctccctctcaaggccctgcccctaagctcctgtccaaccaagcttccagcttctccttgtctgccagaaactgggaaaggaaggcaggccccaaggcatggctggcacacagatcataggatcttgggatgttaaattcagagcctgactaaacttgctgttcaagagcagagtggaggaaaggggagagagccaggataatagtttgtttgttaagcagtgttcctgagaataatgacagggcaattccttccaggacaaacaattcctccagggcaaagccagcaaaaaatgcaaatatgcagaagtctgacagactcagagaggcaggaaagagtggaccacatccccaactcccaactccccctgtcctccccccctcgtgatttttcaagacagggtttcctctgtgtagccctggctgtcctgagacttgctttgtaaaccaagctccactcgaactcagagatgcacctgcctctgcctcccaagtgctgggattaaaggcgtgtgcaacccccagcccctggcccttgatgtttttttttttttttaattttctttaaaatttttgtgtgtttgagtgctttgcatgtgtgtctttgcacaacctgtgtgcctggtacctgcagaggccagatggtagtgagccagcatttgggtgctgggaattgaacttgagtcctctggggaagcgcccagtgctgagccatctcttcctagacccttcccacgatgtttaaagttgcttctggttttgtttgtttgcttggtagacacggtcttaatctgcaaaccacacgagccttaaactcccagcaatcctcctgccttagccctgcaagagctgggattacaagtttgtgtcacctctgtggtccccctgaagcttcctctcctcctctccttcctcttcctcctcctccctccatcatcgaggaaggcagtatatctattagcccagacttccactctaaaagtgcaagttcaggcgtgtaccacacccagctctgattcaccagatggaaacaatggaaattttcatgactcttacgatactgagataaacaaattggactgcaggaggtgggaaaacagtcctcagaaatgaaaatagccaagcttctgggtagctgcccctcccacctgaccccagctctgcacccagctcacgttctccatcagagaaaatgcaggcgtgcagagatggtagctgtcctgctgtaatgtaactataatgaaataccatgataaaaaaacaggccggtagtggtggcgcacgctggtaggatttgctgaaggaggcagaggcaggaggatcacgagttcggggccagcttgggctacacatttgccaggcgtggtggcgcacgcctttgatcccagctctagggaggcagaggcaggcagatttctgagttcaaggccagcctgttctacaaagtgagttccaggacagccagagctataccgagaaaccctgtctcgaaaaacaaaacacacaaacaaacaaaaaacaacaggtgagaggagagaattcatttggcttacacttccacattgtagtccatcatcgaaggaagtcaggacaggaactcaggggaggaaacctagaggtagaagccaatgcagaggccatggaaggatgctgttactggcttgcttggcctactttcttatagaacccaccaccagcccaagaatagtctcatccacaatgggctggtcccttccctatcaatctaattaagaaaatgctttacagcaaatcccatggaagaactcactcagttgaggttccctcctttcagatcactctagcttgtgtcaaagttgccataaaattatccggctcaagccggacggtggtggtgcacacctttaatcccagcacttgggaggcagaggcaggcggatttttgagttcgaggccagcctggtctacagagtgagttccaggacagccagggctatacagagaaaccctgtctcgaaaaaaccaacaaaacaaaacaaaacaaaaaaaacaaaaaaaccctatccagctcagtaaggattaataaagcaacatacaagaggtcccgaaaaagtgtctactctgttacaccacaccgatgttgagatcagaggagtgttcactggaatacttaaaacatgggcaacaaaaactgtgaagtggcccagcaagatgactcagtaggtacaagtgtttgtacacaaacctgacttcctgagttcaatactccagagcccagtgaaagaaaactgactccccaagttatcctctaacctgtacatgtaccaaacccaccctccccccccactctctctctctctctctctctctctctctctctctctctctcacacacacacacacacacacacacacacacacacacacacacacacacacacacactgtcttagtctgggtttctattcctgaacaagcatcatgaggactgggactcaagcagggcaggaagcaggagctgatgcagcggccatggagggatgttacttgctggcttgcttcccctgccttgctcaacttgctctcttatagaacccaagactatcagcccagggatggcaccacccaaaatagactcccccacccccacccccttgatctaaatgagaaaatgccttacagctggatctcatggcggcatttcctcaagggggctcctttctctgataactccagcttgtgtcaagttgacacacaaaaccagccagtacgcatatacacacacagttttaaaaaccaaaagtatgatttctgttccagcctcattccagaaataactgttaactgaagcattagcccaaccgttgcattcttccccatctggagctaaggactgaacccagagctagttggtgacgacatagctttcaccacagggcccggttggtgagcatgaagctctcaccacagagctatatctccttaaactttaaaggaattcagaggaggggcagtgaggtggcccagcctgggatgtgaaagaacccatctcaaaaagctcagcaagccccccggaacctgggcttagggtggacatcagtaaaacagtcacctagcatctgcaagctctaagtccgattcctagccaataagtgaagtaaaataaaattgctgggctcagtggtagagctggcctcaagctcaacctcaaaccctcacaagtagatcctaagggccagtggcatgcttcagtaggtaaaggtgcttgctggcaagccttcagatttcagtgtctcccctgggaccaacccagagggtgaggagagaaaacttacttttatagaggtcctctgacctctgagtgtgtgctctggcatgtacaggctccacgaaatggtcctgatcctgcatgtgtgccctgatgtgtgcctgttccacttcaccagtggctctttgtcccgagtgtgtgcccacccacaatggtcctgtcctctgaatgtgtgccctgagaagggcatgcccataaaagattctctgccccctgtgtgtgccttggaatgtgcacaccccacacagtggtcccctgatccctgcatgcatgccctggcatgtgcacaccccacacaatggccccaatcctgcatgtgtgccttgctatgtgcatgctccacaccaccagtggtcctttgtcccctgagtgtgtgccttggtatgtatgtgccccacatagtggtcctctgactgctctgtgtgtgccttggcatgtaaacaccccacacagtggtcctgtgatcactgaatgtgtgtcttggcatgtgtgtgcagcacacctttgattgctctgtgtgccaaggcatgtaaacaccccacacagtggtcccctgatccctgcatgtgtgtcctggcatgtgcatgccccatacataactgcagtaacgatgacaagtagggtggtacccttaatctgtgttgtcagttctcaccttgtaaagaacatcacctgcagcctcctttctgactttatttcactgacgccacctggttttgctacagtagtgtgtacactaagtgagctctctacctttttgtttgtttgtttgttttttgtttttcgagacagggtttctctgtatagccctggctgtcctggaactcactttgtagaacaggctgaccttgaactcagaaatctgcctgcctctgcctcccgagtgctgggatcaaaggcatgcgccaccatgcctggctccctaccgccttttctgaagtcagttttaagtcacttgcatccctgccaccctgccaggggcctgattctctggacccccatgctcaagctctcccccacttccacgagaatttactccagaccctcttcactaagctggacctcagccccgggcagggtttttatttgctctgactttaatttttctcacacaagcagatcacaatgcaatgtccctgtgagtgctccacacccgtgtctgtgtcttcagataaaatgttagtcaagcgtagccgaaactgtcatacacagcaactcctgacctccaaacacaacaggcacaagacctggcaagtgattttgaagtttattttcaaatgaccggtaaaaattgacccgagttcaggatggttgtgtaaaaactgaacacatgctggtgctgtcacttcacctccaagttcaccaacctggaaacaagtcctacagtgcaaccaccatgcaagattataaacgcaaggagaagcagcagagtctccgctgtcagtaatccagccctggcctctgaaggacccgacacaagcatgtatcaacaatgtggcccaggggagagtccaaacacaaagaactcaacagtctcctgatgttttcataacatatctctgtctctttgtgtattctgatacagaatatctctctacatagctttggctgtcctgcaaaacaaatcacatcagcggggcagtgctgaattgaagaatatatatacttatttatttatatttacaaagacacacttgcagtgttaattaacagggaagataaagcccacgtgcaatacacaaagtaactgaaagacagtttagtttccctaagtctaggacttgattgcaggacaaactaaggagtgaagtactaaacttgaaataatgacagtcctcaaggccacatatctgtctcctagccagcaccgggagagccagcagaggcctctaatccacctcctcgatggtgggtcctgagccagaggctcccttgggcgcctgggccccgaagcccccagccccaggagcacccgcaccctggtacagcccactgatgatggggctgcacacccgctccagctcctcccgcttgtgcacgaactcctccttgtcggccagcgtgttggagtccagccaggagatgacctcctggcacttgtccagcaccttcttcttgtcagcctcgctgagcttgcccttgagaccctcgtcctccacggcgctcttcatgttgaaggcataggattcgagcgcgttcttggcggccaccctgtcgcgctgcacctcgtcctcggccttgtagcgctcggcctcctgcaccatgcgctcgatctcctccttgctcaggcggcccttgtcgttggtgatggtgatcttgttggccttgccggtgctcttgtcggtggccgtgacgttcaggatgccgttggcgtcgatgtcgaaggtcacctcgatctgtggcacgcccctgggcgccggcgggatgccgctcagttcgaagcgccccagcaggttgttgtcgcgcgtcatggccctctcgccctcgtacacctggatcagcaccccgggctggttgtccgagtaggtggtgaaggtctgcgtctgcttggtggggatggtggagttgcgcttgatgagcgccgtcatcacgccgcccgcagtctccaggcccagcgacagcggcgccacgtccagcagcagcaggtcctgcacgttctccgacttgtcccccatcaggatggccgcctgcaccgccgccccgtaggccaccgcctcgtccgggttgatgctcttgttcaggtcgcgcccgttgaagaagtcctgcagcagcttctgcaccttggggatgcgcgtcgagccgcccaccagcaccaggtcgtggatctgcgccttgtccatcttggcgtcgcgcagggccttctccacgggctccagcgtgccgcggaacaggtctgagcacagctcttcgaaccgcgcccgcgtgatggatgtgtagaagtcgatgccctcgaacagagagtcgatctccaggctggcctgggtgctggacgacagcgtcctcttggccctctcacacgcagtgcgcagccgccgcaccgcgcgcttgttctggctgatgtccttcttgtgcttcctcttgaactcctccacgaagtggctcaccagccggttgtcgaagtcctcccctcccaggtgcgtgtcgcccgccgtggccttcacctcgaagatgccgtcgtcgatcgtcaggatggacacgtcgaacgtgccgccccccaggtcgaagatgagcacgttgcgctcgcccttgccggtccggtccagcccgtaggcgatggcggccgccgtgggctcgttgatgatccgcagcacgtttagaccggcgatcacgcccgcgtccttggtggcctgccgctgagagtcgttgaagtaggcgggcaccgtgatcaccgcgttggtcaccgggtggcccaggtacgcctcagcgatctccttcatcttcgtcagcaccatggacgagatctcctccgggaagaacgaccggctctcgcccttgtagttcacctgcaccttgggcttgtcgccgtcgttcaccacctggaagggccagtgcttcatgtcggactgcaccaccgcatcgccgaacttgcggccgatcagccgcttcgcgtcgaacacggtgttctgcgggttcagcgccacctggttcttggcggcgtctccgatgaggcgctcggtgtcggtgaaggccacgtagctgggggtcgtgcggttgccctggtcgttggcgatgatctccaccttgccgtgctggaacacgcccacgcacgagtaggtggtgcccaggtcgatgccgatcgccgtgttcttggccatggcgccgcgctctgcttctggaaggctgcgctccgcggcgtggatgctccggggaaagttgcgctccgcggcagggatgctcctgggaaggttgcgctccgcggcagggatgctctggggaaggctggtcctggccgaggatcgggaacgcgccgctcgctctgcttctcttgtcttcgcttgtctctggatggaaccagatttggttctgagtagctgtcagcgtctggtgacctgctcgccgccctgcgcctttaaggagtcttcaccggccccgcccactctccgctgggccaatcagcgagccggaggaggccttggggccaggaatcttccagcagtttcgcgtctggtggagcttccccgcctcccttgagtaatcggagttgtgggttccgcccttgtccagaactctccagaggtttctggggttcactggagagtacggattcctgagggggagggtgtggggaagtgctggtgctactagtgacactgttgctatggcgacgcattactaaggcctgtgtggaatggacaagaaagatcacctctagctcggtgttgtgtacagtttgttgtgatttgtggggtttcgccaactcgcacagttctgaatatgggggttaaaggctaaaacttaagggctaaaacttctccccgccaagtttaggagacccagggagatgcctgggggcgtgtccggtgacgtgatcctctccaatcgcgttacaatggcagtgctgcctctgacctcatggactaatttaggaactagaggctctgtcccagcacaggctcaaagttgccgggaggggcggggtggggggtgggggggaccccggctgctcagtttggatgttcctggagctccctgctcgtaggggctcagcgacttcaaattttccaaactggatcgaaggcgtagagatcccaggtgagctgggaaggctctgattggagtggggcagaagcctctgtgtcccagttggtcctcgtttgatgggttgagaattttcttaggtttgactgatttacagtcatggtctattggaagccaccatccttaggcctcatcgtctttaagtgttgggattactatgtggctcttcacacacagatccccagttgtattaattttggggttggtggttggcaatcaagaagaaatggggctggacgcttgagggacacaagtggctggaattagtctgagctcaaatcggaggcagacatcttaagacctttttgaatatggtattgaccctgaagacaggagttacaatcaagagagggaccgaagcctaggattccagcactccggattttcagacaggatggctgcggagatggggctggtgtgggaacagggtcgaagatgagactttgagccctacttctatgaaaagctagaaaagttgctgggagccgcctggaggctcaagcctttaatcccaactgttaggaatggaggcagagtattgcctgagctacatagttggaccctgactcttcttttctttttttttttttttaagcttggagggtgcggggatcctaacagccatccttagagtgagctaaattactccaccataggttgctaaggaaggcaacctgatctgaggaaagcctagtgcatgtctttaatggtttgtctgtctctgtgtgtgtgtgtgtgtctgtgtatctgtctgtctgtctgtctgtgtgtgagaagaggctgtctcaccccctgaatgagacagttatgagctgcccgctatgtgtgtgtctgtgtgtgagaagaggctgttttatccctggatgagacaattatgagctgcctgctgtctctctctctctctctctctctctctctctctctctctctctctctctctgagagagagagagagagagagagaagaggctgtgtcaccccctggggtaggccagttatgagctgcccgctgagggtttgggaactgggctcaggttctctagaagagcaggaggcccctgtcactgccacctcttaggtgcccccaatatgtcttaaggagttcagaacacagtcttaggaggcaggtagacaagtgtggtcccacagtaaaatatatcactgacataggaagagtagagttgatttcggtcctgtaggaaatggaggatacagacccacccttggggtagctgagaacactgttgtctaccatctatgtaagtcctttcccatgaaagccaggtctggatgtgggtgtgtgaacctatgctaacagcagcatcattcacaatagccaagaagtggaagcaacctggtgtctatggagagatgaacatgtaaggaaagcaagggagatgtgtacatgaacagtgtttagttttggaattaccaaaatagacaagctcacgcgtaagaattaaggaccattatcttgagtgaaataaggccagccacaaatagacaaacacagtataattccaattgtatgagttactaagcgtagacaaatccagagacaagaatccagcaaacaggcaactcatggtgaagaagcccagccatggttagcacaggctgtactcccagctacttggcagactgtgatgagagactgccagggcccatggcctagaaaatatgatgagacccattttgaaaagaattcctattacaatgagtctctttcaaaatgaatagaggtgcctggcagtgatggtacacaccattaattccagcacttgggaggcagaggcaggtgaatttctgagttcgaggccagcctggtctacagagtgagttccaggacagccagggctacacagagaaaccttgtcttgaaactctcccccacccccaaagaaaaaatgaaaaccaccaaaggaaaaataaaacaaaatgaatagaggcaaagttcattattgacttaaagattttaatcaagtctattgggatgaggttcagactaaataggaagtgagtttggcagtggaaagaggcctatggatgccccctatgccatgttgatgtttgaccagcctgtctgttttttggtttttcaaaacagggtttctctgtgtggccctggctgtcctggaactcactctgtaaaccaggctggcctgtcttaatgtatgacatctggtagacaagaaacactgtagaagccagagatgatcccaaaacagatacttgggagactcaggggctgctggagggtctgtgctctgagtcaagagtccagtgaaagcacaaccatgctgtggtaatgtgagctcagtggtacagccctggcttagaatccccaaagtcctagcttctgccccagcacacaaagacacagattccagagccaatcccaaagaataaagacagtgagaggtgaacagagagggatttggacaaggtttgatattcttcagagactccaactcaaaacgtatacagttgttagccctggacaactcaaaacgtatacactgttgttagccctggacaactcaaaacctatacactgttgttagccctggacacctctggggttgagactttgtagcacttttccctcgttggtttcatgtttctcacaaaagaagagaaagtgtggaattgtagggtgtatcagacagctcagacagttgttaagaaaaacagcctggctatatagagaaaccctgtcttgaaaaaaacaaaaacaaacaaacaaaaaacaaaaacaaaaagaaaaaaaaaggaaaagaaaaagaaaaagaaaaaggaaaaacagcctggtttggtgactcacgcctgtgaccttgccagaccttgggaaggctaagaaaggagaatagcagcacttttgaagctagcctggtatacatggtgagttacaggccgagttgcaggccaacctccaggcatacagagagacactgtctaaaaaaaaaaaagcaacccaaaacaacctttgagaagccatggagattgctcagtgggtacagtgtttgttgtgcaagcatgcagatctgagttcagaacaacaatctgatcatgcttgcattcagctgtgatcacaggccaagctggatggatcactgggttcactggcaagccagtataacctacttgaccagggtttcaggccagtgagtggccctatttcaacaaggtgattggcaggagaatgagacccagggttgacccctgaaggccacatgttcatgcacacacacacacacacacacacacacacacacacacacacactctgacacacacgaaaacagccacagagacattttcaagctctctgtgtaggacttccttctgtccagaagaaggtagagagtctgggctgtgtttatctttacatttgtggctttaagactgatggatggaactggagagctgactcagcaattaaaagcactttctggcacctatatggtgacttatgactgtaaccccagtctcaggggggtctgacaacctcccctgacctcctagggcacatatatatatgaagacaaaatgttcagacacaaaatactttttaaagtttttttttttttttttaataagaaggctagagaggggctggtgagatggctcagtgggtaagagcacccgactgctcttccgaaggtccagagttcaaatcccagcaaccacatggtggctcacaaccatccgtaagaagatctgactccctcttctggtgtgtctgaagacagctacagtgtacttacatataataaataaataaatcttaaaaaaaaaaaaaaaaaagaaggctagagagggttcagccattaagaacaaagataggaagcccggattttgttcctggccccgacccatgttgtgcggttcccaagtgtaactccagctccaagggcatcccataactctgacctccacaggtacctgcactcgtgtgcatatacccattcagacatacatgcatacacacatgaaatattaaaaatgcatcttgaagagaaaaactgatcaataaatagcttaagttttatttaaaaagaaaacactagcctaaatatgattttcaagtctcctttctccataggcctcggaaaaccatggctgctaataaaggaatggcgatcggcatcgacttgggcaccacctactcgtgcgtgggcgtgttccagcacggcaaggtggagatcatcgccaacgaccagggcaaccgcacgacccctagctacgtggccttcaccgacaccgagcgcctcatcggagacgctgccaagaaccaggtggccatgaatccccagaacactgtttttgatgccaaacgtctaattggcaggaagtttaatgatcctgttgtgcagtcagatatgaagctttggccatttcaagtgatcaatgaagccggcaaacccaaggtgatggtgtcctataaaggagagaagaaagccttctacccagaggagatctcatccatggtactgacgaagatgaaggagactgcagaggcttttttgggccacaatgtcaccaacgctgtgatcacggtgccagcctatttcaatgactctcagcggcaagccaccaaggatgcaggtgtcatcgcaggactcaatgtgctgagaataatcaatgagcccacggcggccgccatcgcctacggcttggataaaggaagtcacggagagcggcacgtgctcatcttcgacctggggggtggcacgttcgacgtgtccatcctgacgatcgacgacggcatcttcgaggtgaaggccacggcgggcgacacgcacctgggaggggaggacttcgacaaccggctggtgagccacttcgtggaggagttcaagaggaagcacaagaaggacatcagccagaacaagcgcgcggtgcggcggctgcgcactgcgtgtgagagggccaagaggacgctgtcgtccagcacccaggcaaacctggagatcgactctttatatgagggcatcgacttctacacgtccatcactagagcacggtttgaagagctgtgtgcagacctatttagaggcacacttgagcccgtggaaaagtctcttcgggatgccaagatggataaggctaaaatccatgacattgttctagtagggggctccacccgcatcccaaaggtgcaaaaactgcttcaggactactttaatggacgggatctcaacaagagtatcaatcccgatgaggcagtcgcctacggagctgcagtccaggcagctattttaatgggcgacaaatctgaaaaagtacaggatttgcttttgttggacgtagctcccctgtctctaggattggagacagctgggggtgtgatgactgtactgatcaagcgcaactccaccatccccaccaagcagacgcagatcttcaccacctactcggacaaccagcccggggtgctgatccaggtgtacgagggcgagagagccatgacgcgcgacaacaacctgctggggcgctttgacttgactggaatacctcctgcacctaggggcgtgccacagatcgaggtgaccttcgacatcgacgccaacggtatcctgaacgtcacggccatggacaagagcaccggcaaggccaacaagatcaccatcaccaacgacaagggtcgcctgagcaaggaggagattgagcgcatggtgcaggaggccgagcgctacaaagcggaggatgagggccagagggagaaaatcgctgccaaaaatgccttagaatcgtacgcctttaatatgaagagcgctgtgggtgatgagggtctgaaggacaagatcagcgagtccgataaaaagaaaatactggataaatgcaatgaggtcctttcctggctggaggccaaccagctggctgagaaagatgagtttgatcataaaagaaaagaactggaaaatatgtgtaatccgatcatcacaaaactgtaccagagcggatgcaccgggcccacctgtacgccagggtatactcccggcagggctgccacaggccctaccatcgaggaagtagattagccttttccagaattgcagggtgctagggtgcctctaggcgaattttattcatcttcaaacatcaatatgattcttgaactgactggactcaagcctacgtcaccatcctttgggatcccgatggagaagcctcgaactcgaccttttcacacccccaccctctcatctatgatcctgaactggacctttaggaaaaccaggcccctctttgagccatttgaagaatttggatgtctgttatttattatccacaccccacctttctccttcctgtgtggatggttatttgtctctcagtaaatttgttcccaaaggaaacaacttctgtcatcttttattttttttagtcagggccttactgtatatgtctagctgacttgacacttactatgtaggccaacttggtccctaacttgcagcaattcccctgttctgcacctgagtcccgagatcacaggtatgcaccatcatgaccagcttttgatttttcagatggatgcagagtttctttttcttttttttttgggggggggggttcgagacaggagttctctgtgtagccctggctgtcctggaactcagagatccccctgcctctggctcccaagtgctgggattaaaggcgtgtgccaccactgccccacacggcagagtttcttggagcccaggctagcctaaaacattctatacaccattttatttcctggtatctgtaagctgtcaatggtccttgaaaattaagaaaggaagatatgcatttttccattcgtgggtcccttgtgaatgtttaacttgataagggcaaagtacttgaatttgtgtaagttcatttccaacaaaaccattttgctttttgttttgttttgtggttttgagacagggtctaatatagcctaggtctgccttgaactctgatcacgatacctccacctgccaagacaaacatttcctaattctcaggcaccctgggcccaaaggacgcagttgctaaatgtactgactgcttctatccagtgacagcttgttttcgattcaaatcttaaggattctgctgaacttgggaacaaatcctgcttggtaccatgctggatccagctctgaacacagtgatgtgcaccggtaatgccagcactgggggagtgggtaggtagggcagacagaagaagcatcttagggtcttgctggcaagtcagtctagctgaatgaacctgtcagctccaggatctatggtagatcctatctcagaaaatgagagaaatgactaagtggttaagaacacgtgttgctcttccacaggaccagggtttgattcccagcacctatgtggtggctaacaactgtaactctagaggatccaacactctcttctagtgtctgggagcactgcccatgtgtacacagatggacatgcaaggaaaaacgctcacacatatacaattttaaaatattaaataaggtggaaagaacaggcagtatctatctctggcctacacagtcatgcatgctccagccctcactcccacccccaggaaaccctggctacaatctatttagattattacactagagaattccaccctgcaatcagcaggtttagactctcagaattttcacctgtgtcccgtgactgccttgcacctacagccagcctcaagaagagaagacagaatagaaagtgtttccctatttatccagacatgatatccacagaagatacaacctagcaacttcccacttaatgtaattatttaaaaaaaaaaaaacaatagggctggagagatggctcagcagttaagagcactgactgctcttccagaggtcctaagttcaatttccagcaagcacagagtggctcacaatcatctgtagtgggatctgatgccctcttctggatgtctgaagaaagccacagcatacttacataaaacaagtcaatcgaaataacaacaaagccctttcatctcagaccatctctttcttctctggaacttgcctactgaggcttttctagttttttgttttttttttaaatcagtcagctcttgtgaactccttcacaaccagcatgttacaagcctggtaatgctgttcccagaacatccaaagtagtatctgtcggtgatctagttgaaaatatttcagtaatgcagggatggatcagcagttacaagcatctgttgctcttccagaacacccagatttgattgccagcacccacaaggaggctcaccattgtaactccaggcccagggcatttgaagccctctcttctaccaggaactcatgtagtacagacataacatacaggcaaaaactgtcaaacacacacaaaaaaagctaagataaagtaaaatatttctgtcactgagttccagtcagtttagtcctttacagacgagtcctatgccaaattatatatatatatataacattagagctgggatgttgttcatttgctttgcttagaatgtgtccgctctgggtcaactttgcaccccaaaagactgaaattgagagaaacttacttactgggaacctttgataagaatcaggatttgtgttccggtttctctctcacactcacacacaccataaccagtgtgtattggtgtatattttattctctgacacagggtcttgctataatagctttagttgacctctaaaccagaatcctcccgtctcaactccaggattccagttgggcacgaacgctgtccagctcttagcaacattttaaattttactaaagaatacgcaagtcataattttctcaatcttcccaataattccccatagaaaacgtagactacgagctaatcacgcggcagagagaaaagtctcaacaaccatcagaaaaagagacgaactacgtccaacgcgcagctgccacggcccacgcatgcgcttgtgagaaagcggctgctcgggaggaagtgggggggacaggaagttccgcccctctaccggtttggcggtcctctcggctctgccctaccccgctccctttgagcctgaattcagccgaggttttcgcggctccccgtgcgcctgcgtgatccagtcttgcttcacgcgcgggctcgcgctccggctcccgccctgcctccggctcgcagtccccggcgccttccttcgcggtccaggccgccccgcgtggcgccagcaccatggtgggtaccgtgacgtcaaaagggagcgccgccgctcgcggcgggcggttcgaaccgtgcgggcgtgggtgcgggcactcgtgtcggtcgcttggggcggcagccaggccccagggttttccgtacagccttactgcccacaccccagtgtgaggaggctgctcggagccgatggtgttttgggggaaacctttagcaagtccaagagaaagcccacgggcgccgggggagaacgggaagcgagccttgcacttctgtgctgttgacgaggactttgtgagggtgcggttgggaagcggtctcagctgggaggtggtcgactcagagagtgggttctagcgtcgaggggtgcaacgcaggtcctgtcactccgtggctccgtgaactgcagagtcccgtttaacggattgttgttggttatgaagttttgagaactactaggaggggctacaaagactagtatttcaaaatactgtcacgggctcctcctccccgtcccttttcatgtgatagttcctccctcttttccaatagctcttctactcctttttcaagtctcttgtgggcaaggatgtagtcgtggaactcaagaatgacctgaggtaggtccttattttagcgagttggtggaaggtgggggtgccgggtgtcctgatcctttttgtcgttgttgttgacaaagttacttcagagtaggtaggcaaaacacagaagaaatacctttcgtcttaagtgtaaaagattagtttaagagtgagcctggtattgtgacacatgcttgtaatcccaacactgaacgcagaggtgtgagttcaaggacagcttggtctacatatgtccagggccatccggagttacacagtgagatacagttaagtaacatgtatgtgtagtcctggctgtcctggaacacacagatccacatgcctctgtctcctgagtgctaggatttgcctggctgtatcatttgctcttaacgattcaggagttggggtaaatgcttaaatgctgtaccactggaaatatgtttatgggttcttgtcacatccgttaaggtctacttggttcctaatagtgcttcgaaaattatagtttcattgaagccgggttttgtttgcttttgttttctttcatgtactgggcatgctcaacaagtgaacttcatatgcagccctcatgtttctctttaaaataacttttttttttttttccagacagggtttttctgtatagccctggctgtcttggaactcactctgtagaccaggctggcctcgaactcagaaatccgcctgcctctgtctcccgagtgctgggattaaaggcgtgtgccaccaccgcccagcaaaataactttatttgtagactcttgaaacctttgaacacacatcagtggttgaccagcaggatggttctgaccagtgtggccaagttagaagctggcccagactgaggtgcagactaagacctttcatagtctaaaagtcaaatcacacgaatggtctacagagctgtggggattcataggatgtagagagtgaggaagtaaaactcaggatctgaggtggttaaagttggttacacagagggtaaatataatataaggtggggcatgtaaggtggtatgggagcactggccttgataaccttgcttaggggctagagggctggagaactgagaagacttgacttgtatcttcattattaaagataagttgggggggataacatttgaaatgtaaataaagaaaatatccaattttttaaaaaagaagaagaagccgggtgtggtggcgcaggccattaatcccagcactcgggctacacagagaaaccctgtctcgaaaaaaaacaaaaacaaaaaaaaaccaaacaaacaaacaaaaaactcatacgagaaaaaaaagagtgtagcaaaaataataaagagtaattccagctaccagtatgtgtacataggtctgtgactccaaagcagtaaatgtttacaaataacagttagggaagctgcagagattgctcagtgctttaaaaagagcttgctgggggctggagaaatggctcagagattttaagagcactgactgctcttccgaaggtcctgagttcaaatcccagcaaccacatggtagctcacaaccatctgtaaagagagcggacatcagacgccctcttctgtagtgtctgaagacagctacagtgtacttagatataataataaataaatctttaaaacaaaacaaaacttgttgctctccctgaaaaataagagttcagtttccagcaactacattaggtgattcacacctacctagaactctggctgcaagtgatctgatgccctcttctggtctctgtgggtaactacatgcacatacacacaaataataaaaatcaatattttaaaaataatagttgatgcttctgggagtcatgcagatctgggctgaccttcagtttattggttgtaagttttgttaccttccatgacctctctcatatcctggaggatgataatactttgaaagatgattagctaagtggtgttgatagcagtgcatcccttaaacctttaatccagcactcaggaggtagaggcagaaacaggtgtgagtttgaggccagccttatctacatagctagttctaggtcctcacagagaaacccagtggggaatgatcatgaatagtgggtcccagctgtagttccacaagtggaggtaggaggattgccatagtttgaggccagcctgggcgcagtgagatcctgtctttaaagggaaatacaagtagtaaatggaatgcaagcctggcctgcagtagttaaatattagcttctgatgcattggtgtgagcaaagactgagtctgggatgcattttcgtttctacctcagaactcacagatgctgcctgcaggcctcaggtccttaagttcctccccgacttgtcgttctagttttgatttttccctccacgctgatttcagcataaaccaatgcttcttccctctttgcccttgacagcatctgtggaaccctccactctgtggaccaggtgagatatacaataagggtcagtttgttcttggggaaggaaagcagccagtgagggtgggttgttgtatcttgtctctagggttaatgacgatgttcctctttcttagtacctcaatatcaaactaaccgacatcagtgtcacagaccctgagaaataccctcacatggtaagttggggctggggagggggaggaggaggaaacctttaaagcaaaaggcccctgggtggccggaggacacacttttaatggaggtgctttcatatttgtctccagttatcagtcaagaactgcttcatcaggggctcagtcgtccgctatgtgcagctgccagcagatgaggtggacacacaactgctacaggatgcagcaaggaaggaagcccttcagcagaagcagtgatggcacctcccccctccccctcacttccccattggtgacccagaacttgacgtcctactgaggacaagaacacccctgcccaatacttaaggtgtgttttgttttgttaatgattttttttttttctttttaactgaatggatgagagataattcctctgctgagaagggagggtaagagagccaggaactgcagaatggtcctggactggctcccagtctgtacctttttctcatctttcctggagatgggagcagcatcctaagcccttccgtggcgacatgtaatacatttgagggatggaaagatctgtcccgcatcgggaataaaacttatgatgcaagctgaattcctggtttgtcgtctgctttgggcagggagttgagaaaggatgttgaattcactgacactccagcaccctacattcctcctcctcccatctccaaccaagattccaacctgatttaccagcatgatgtacttctgccctaaagcttgtagtcgggtaaattaaggtgccagtgccaactcttaaacatccagcctttcctccagcccaccacccaccaaaggtaatggaagagaaagctcttaggatgtgggggaagtagattggtttagcaataggtgagctcaatctttatcagcagttcagtcctgtagcaaataccaaatatgaaactgcagctgcagatggttctttcggacaggcagaccccacacataaaccagcagctgtagtccagtcctcttagctagcatgaactagcagttgtagttcaatcctgaagaagccacccggcttgccagaagacgttgagccactgaagcagcaaaaagctacaggaacctcacgagcagttcttgggtgagtttctctcaatggcagcattactgcaagtggagctcaacaacgctatgcaaggtgaaccaggtcacaggtgtcattagcaaagaatagcaaggcagagcaaaccaaaccagttctcatgctccccgtttgtagggtaatatttacactcgtttatcatgcgctcttttatgtgtctgctatagcaagacattctttcacctgtgtgccccaggaaaacatcatttgacataatcgactttccaaagaaactagaagtttctacttcagccactttagaagcaaactcccaattagaggttcttcagtgtccaccatcttcttaaacttttttttttttaagattattatatgtgagtacactagctgtcttcttgctggaatttgaactcaggaccttcagaagagcagtcagtgctcttaaccgctgagccatctctctagccccaaccaccatcttcttagaggtagtattggggtgctggcaggagctctggcatgaaaagccttatgttattaaaacatcttaagagcggggcgtggtggcacacgcctttaatcccagcactcaggaggcagaggcaggcagatttctgagttcgaggccagcctggtctacaaagtgagttccaggacagccagggctatacagaaaaaccctgtctcgaaacgccccccccccccaaaatcttaaataccataatctagatctcaagactatatatagcatatctgaataggcaaacattgcttgtttctagctatttactatctacttaactttgaaaacatctacagaaggctaaataactcttatttctgtaattagctaaactaatatctgccatgaccacaatttgattaattactaacttgcatttcttaattatcctaaacagtttgtaatatagtatcatttaaggattttagaactttagatttttaaatgagttgctgtggtgcaatactctaaactagttgaaacatatacaatatgataaaacaaaaatataatcttgaatgcatatcagtatacaaaaatatactcatgggaaaatatttggcttattgatgttcattaatataaaagtagatttgggttggagagatggctcagtggttaagagcactgagtactcttccagaggttctgagttcaaatcccagcaaccacatggtggctcacaaccatctgtaatgagatctaatgacctcttctggtgtgtttgaagacagctacagtgtacttacatattataataaatcttaaaaaaaaaaaaaagagaagtagattcaataatctacccttttatcctatcatctctatattcccctttttcttttcaccacctctcctctcccttcccactaatactagataggagagaaaggaggaaagaaagagatccctgaatctaattttactgggtttcctggttgactaagaccattaacagcgtgtgacccatcccccttaaacaatgacaaatatccatcacccagcaaatgagcaaaacccacccatcccacctcttaggaatggcggcattgttctcttaaaatacttcctgagggcaacagcattttttgaggaccttaagaaaattgagatattggtcaagtcctgggagagctagctagttgtatcatttgtggtccagtccttgtgatgggaaagagcaaggcttaattgaagtcctggctggaacagcctgtgggactggagcatcttagcaaggcatgttgaagttgtcctggatacagatttttgaggaaataattcctgaggctgtgaagctggatcaccagggctacttgtctggtctttttgatctgaaaacacacaaagtgtgtataactacagaatcagcctttttagacttaaagcaatcacctattgaaatcccgaatatgtgtctatggtctggaacatatactctaattttttaaactctggaaaaatgaaacatactcatttgccaaatttctttggggggggggttccctttggaatttggagtttggttttccaagaacaaatgggacattgtatcattttcttggcttgttgccaagtgatagaagcctctttctttaaacctttgctgttagcatggagattatttttgttttttgagacagtgtttctctgtatagccttggctgtcctggaactcacgttgtagaccaggctggccttgaactcagaaatccacctgcctctgcctcccaagtgctcggattaaaggcatgcgccaccacctctcggccacaatggacgatttctatttctgcttatttgttgtagttgaataaatagtgacgttttaattctcaatcatttggaataagttgagcagtttcaatatgtaaaacaattctttctgcatagagaaactatattaagagatgcaggaatatcgaatattatacaggaatggcatacatctctgatttttaaattgaatcataagggctctgagccactttatttttcctgatttataacttgcccttcctgacttatttacataagtgtagaatgtgggggctccagaaatcggtttccctttaactatatgagggagaatctgattagtacttttcaaaaactgaagttgctttggggatatttattgttaatttctctagaaaattaccagaaactctttgccaatgttcattttctgcccataatgaggcaatttcagcattagtaaaaggtaccacaatttctgctgggtctattcctgctaattgacaaaatatcaattttcctttcaaaatcaatccagaaaccttttccacataggccttttttttcagttttttatcctaagaatatccatcctaagatagtatctctctctgcctgagggtgcctgtgggacaatgagtaataggaaaataggaaccaacagtccatatgtgcatccggtaattgctttgctttcctggaactcactctgtagacgaggctggcctcaaactcagaaatccgcctgcctctgcctggcctgtaacttcttttctatcagagccaataccttttcagctttagctgatttttaaattttatttattatatgtaagtacacaatagctgtcttcagacactccagaagagggagtcagatctcattatggatggctgtgagctactatgtggttgctgggatttgaactcaggacctttgggagagtagtcagtgctcttaaccactgagccatctctccagcccacctgataactttcatgaactgtttaagtcttaatcaccttgtaagctttgaaataaattacttagctcttgagtagttaattcaactgtggaatttagccaattaatatctcccaacaatttttgaaattccttaagtttataattgtctcctgatttgtacttactgtggttgaattttctataagctatcttatatcttaggtgattaatacaatctcctttggattttttccagaagcaaattgttatcaccaacaaggcaaaattctctttactgcatcaattttttaaaaaggatttattggactggtgagatggctcagtgggtaagagcacccgactgctcttccgaaggtcaggagttcaaatcccagcaaccacatggtggctcacaaccatccataacgagatctgactccctcttctggagtgtctgaggacagctacagtgtacttacatataataaataaataaatctttttttttttaaaaagggatttatttattggtatgttgagtacccagttgcttccttcagacacaccagaagaggcattggatcccattacagaaggttgtaagccaccatgtggctgctgggaattgaactcaggacctctagaacagtcagtgctcttaacctctgagccatctctccagcccctattttgtcaagtttttaaaggtatctccatccaaattagccagccacatatcatccagatgataaattttagattgaactgttatgaattatttcccacagctcttgtacaaaatattggcatgaggtatgactgtttaacattctttgtggtaagactttgcaattatatctctttattggacaactattataagtagcaaacctttttctatcctgctcatgcaaagggaagtctccacccagttggtgaagtctggatgccagcagctgtcacggctccagcagatccaaggggggtgttaagtcctgttcctacttcataaagggatgaatatctcatctctgactgtagcagagtttgtagcaccagttttgtcttctcttcctcctagagttaaactgggggaatagtcacaaccaggctacaaaaccctggcctggggggggggggcagggggggagcgggcagtgtctatgcagggagttttctttcctttacatcaaaggaagaaggggggcaatgtacaagtcaaggcagtaacagtctgacattggcagtctgttgggcccaggagaaacgcagaggtaggatagatatttgaccaaaaaggggaatgaatgtccagaaggtccggacaggtgtcatttcttttggctggtgttatcacagaagtatttgtgggctctgaagcaaggagggcggtaagtgctggaggtggaaaagaaacagtgggcaacatttaggtatgtggtgaatgggaaggagctggatgtgttgggtccaggatgctgttgatcacatttgttccactgtttcattttcctgtagaacaatgttatgatgagttaacacaggcttaactgtggtgggtacaaggagatatgcaagacagcagcaggatacctagcaaggaactgatagaacttgtccaacacccctgtacaccagcaatcatgctaggcgtcttcataatggattatggaatactccgcatcactgtcccgaaggtagatggatttcccctttttgcgattagtgggacctttagtaaagccctcttctagcctgcaggtgtacaatcagcacactctaagtatcagcatccttgagatgtgatggctctgtttgtcggaaaggaaaacacagtcattcaggacattgctgctataccacaatctgtggactcctggcattcctgctagtgagtagtctactgtcctgacagattcctcctctgacataaaaccaccagaccataccaggaagagaacagggctagccatagcactcgtgttaagcagggtaaaaagagccatcagaatgaaagagaaggatcctctgtgtggacatgtgtcattggccctgagggctgcaagggaaaccagaatggtggacagttttccaagtcttgtaaaaattttggaatagatgtctcctgaaagccggtctattatcagttttgatatggagagggacccccatagtagaaaaagtgaagaacacgagactggttagttttttaaaatgggaataggtatcaatggtaacaaatacaaatttttgcataccaaatgatgagatgtgggtgacatcagtctgctagaaggcactggccttaggacatcgggggttggtgcccaattgttgtatagcaggggtgtgaagtttatggaggcacaagaggagaaggcacaagaggaggaagttttgagaatgtgtttaaggtgttctaagggggtattgggaaatttaaactgtagcccatcaggttatttgctgatgtttcttttgtggtgagggatgaaacagtcaagggctcagttgtggcactcttactggctaaggagtgaccatgagagagcgggctggtctgggaaggatgaatgtaaccgtacttagcagaagaagatgggaagatgtttttgtttgaagtatttttggaatttattattaacatcggagaaagagggttaccaccaagtctcacataggagcaggctagctaggggaaaggggagagtagggtgaccacaattactatcaggtagtaaagaaggcaattaagattccaagctataaagagcccattagcgggggtcgggggggcatgcctttaatcccagcacttgggaggcagaggcaggcggatttctgagttcgaggccagcctggtctacagagtgagttccaggacagacagggctacacagacaaaccctgtcttgaagaagagaaaaacaacaacaacaacaacaaaaaagagcccattggctgaatcatctgatttatctgttaatatcctccattttcccgattcctttctttaaaataataaacatagaggaactccatggcctggtggattcttcattgtgttgagcatttagctgcttctgtaccatctgttttagtacctgtagtttattctaataccacaggcctttgtcctcccatataggtttgccagctattttaaaggtagggcagacggtacctttgaaaggccaataccgggctggagagatggctcagtggttaagagcagcgcctgctcttcagaaggtcctgaattcaaatcccagcaaccacatggtggctcacaaccatccgtaacgagatcagactccctcttctggagtgtctgaagacagctacagtgtacttacatataataataaataaatctttgaaaaaaaaaaaaggccaatacttgttgtgtccagcctatggacaacctggacagtctgggactgttcttgataatacctttttttttttccagaagccctcttcccttttttaaagatttacttactttatgtatgagcgtacattattgctcttttcaggcacaccagaagagggcatcagatcccattacagatggttgtgagccaccatgtggttgctgggatttgaactcaggactctggaagaacagtcggtgctcttaaccgctgagccatctctccagccctcagaagcatcttttattctatggactatttctgagattgagagaatgctaatctgtcttctattgctgcaacatatcatacccccataaatcatggctatcttggccacatagctttaatttttctatgtgaccttctgatccttaagtaagcatctcacactttgtttttttcctaggataatgtcccaacccctagaaacctcccaaataggccacaattctggtaaaatcattgttacatctgcttttgtgtctatcaagcctttaatttcagtatcatttacaataaaccctttaattttggcctctgatcatcagcttgtcaaaattcctgggtttttttttttttttttttttttttttttttgtctcttccaactactgaagccagtctgacgttgatatctgggctgttgatggctgtctccagaacagtgctgtttatacactctacagatgagtttctctggcctgaaatggttgacttgaattcaatacaggggccagcaaaaggttccctagggtctgtcccttgctggtgggcattcattagtccaatgccagcctttgccacatcttctgtgtactctagaaggctcaggccttcctttggaattagcttggctttgcctacaattcctttgaaatgaccttgcttgtcacagtgaagacatttgagtttgggttttgtttgtttgtttgcttgtttttcgagacagggtttctctgtatagcccaggctgctgtcccggaactcactttgtagaccaggctggcctcgaactcagaaatcagcctgcctctgcctcctgagtgctgggattaaaggcatgcaccaccatgcccagtgagtttgggtttacttaaaacctttggaggttacatctattacagttacataattagtacgagatccaatatcagctatatttttaatccatctatttattagtgctgatcttgcctttgaggactagtcacccttctgcacatagaactagcttttttttttttttaagatttatttattgattatatataagtacagtgtagctgtcttcagacacaccagaagagggtgtcagatctcattgcggatggttgctgggaattgaactcaggaccttttgaagagcagtcatttcccttaccagctgagccatctcaccagccccctggaactagcttttttttaaagtaaaagatttgattaatacttgcctagcttctgaatcagatactttaatttacatttgaagtcaatctttgtaaaatatcagtgaatgcttcctttggaccttgtataattttagtaaacacatcagaccattttttctgtttcttcacccttgttccaagcacttaaaagctgtggtgttgccagattcaacctgcatgtgtacctcagcatgctgaccttcagctagcagttcattttgggaactgctgatacctgagccccatttctgtcctatcctcttagcctctcctatcacaatcaccactgagactgagggcctgcctctaatactgctgttgtcttagttccagtcttggaggattactctattttgaactgcccaagaatttagtatctgcttcacatatggcagatacatgccatatgaaatgatggcttctttaaattctatatctagcatttctacagcatgccatccaagttcttcataaccttgaaggtacctatcatctgctgctatttgtcatatagtagccagataagctaagcttgattgtctaacaacttttggctgaccttcagtttcattatatggtggcaagtaggctctagtctcaattcctctgtctgtaaatatctttctcactctttctagttttttttttaagatttatttatttattatatgtaagtacactgtagttgtcttcagatacaccagaagagggcatcagatctcattacgggtggttgtaagccaccatgtggttgttggatttgaactttggaccttcggaagagcagtcgggtgctcttacccactgagccatctcactagcccctctttctagttttttgtgtaaggcttatgtcttatcaatctgcttttcacaattttacttttctctgtatttctaaagtctttcttttcgagggcacagaaagctactctggctattaagactcaaatatgagccatattgagccaatatcaataaaatcatctattctttttttctttctctaaagccatcatgatatttcccattcttatgcagcttattcttttttggttttttggttggttggtttggtttttcgagacagggtttctctgtgtagccctggctgtcctggaactcagagatctgcttgcctctgcctcccaagtgctgggattaaaggcatgccccaccacgcccggcttattctttttttaaagatgtatttatttgggctagaaagatggctcattggttaagagcactgactgctcttctgaaggtcctgagttcgaatcccagcaaccacatggtggctcaaaaccatcctaatgagatttgataccctcctctggtgtatctttagacagctacagtgcactcacatacataaaaaatatatataaaaaaaagatttatgcactctttccaccatgaatagtctagacaagcaaaggctgtctccttcctgaaagatggtcatgggggaagtccttggtcacacagaattgtacaagtctcccgcagaaggcctttccacgcttctagagctaaaccagcctcccaccaccctccagggctcatctcaggccctccactccagttttcaactcttcgcagttcccagcagcatctgtgcacccaggggtctgagagccacaggctagggcctgtgggaagccgttgcagagtggcagttacagagtgacagttacagagtggcagttgcagagtggcagttggctatagctggccaccacacatacatagccagtaaaggttcttttgccaaggtgaggttttgagaattgacaaaagttaaccaatcagatgagagacaagttaaccaatcagatgagagagaacgcctgtcctaggcctataaaagcagcaccagttctgggctcgaggtcttttcgcctctacaatcaagctctcccaataaacatgtgcagaaggatcctgttgcagcgtcgttcttcctggccagtcgagcgcgcgcaagaagggcccatatcttaggctgtgtgggtgatatgtcagaggcatcacatgaacccggcatcccagtggggagagagagatgcagcctggtgcccctgcgtttcacctccccaagcagcagtccaaccccccagcagagaaaccaaatgcagaaccacaggggtttctaccttggaccatttacttccctgataaaagaaacagacaacctttttatttataacaagccttaatcagcacaagagctgggcagatatctatcctctacactagtatgcctactccccagccagtaagccatcatctaattcctcatgtttccatctgggctgctcttaactccaattagccaacccacatagccatcattccacaactcacccaatccacgcccgcttcctcctcccttcaccttctcgccctcctggtgcttctcttacaaccccaagctcaggaaccctaaaccccaccctggtctctcctgcccagctatcgactgcaggcatctttattcaccaaccagaagtaacttagggtcaaggtcacataacatcacttgggtctctggggcaaccagggcttgcactaagcattccaaagcataacagaccaaagctcaatatatatcatctatataaaatggcaaccacaaagagacggcaggctctcttttccttttaaagccagttagatttccaactgtagttaggtaatttacaccaggattgtttgtgttcatagtgaggggtgtctgtacattttggactggtctgtaccttgaaggaaaaagcgccctttcataggggctttcttcaaattggctttagctaccccacccccaccccccgttgtcagggtggctgtggattggcagcccattcttttttttttccgagacagggtttctctgtgtagccttggctgtcctggaactcactgtagaccaggctggcctcgaactcagaaatccgtctgcctctgcctcccaagtgctgggattaaaagtgtgtgccaccacgcctggcttggctgcccattctttatggcttttctgagactaaacttaaggcctgacctagcagcctgtcttaaaatggagtaagttaggatcactctctctcaccttaagagtccatctcaaacccttgagcagaatccaagggttagcattttaatacgtgtcggatggtggagacctggtccttgatgaatccatcagggggttaacaacatagagcttgcatatcacaatgagtatgaccaggcccaagaatccagacagagttgcaataactgtaatccaaacaggctgaccctgttccatttcccttttcctgtcttctagacgtttcctcaccttagccaataactccctcgcatgctttaagttatccacaaaggtgcagcaccttttctccagaggtttacacagtccatcctgttgcagagattggaagtcttgttcccgctgatttcgaagggtggcctcccttagagaagttaatgaggttttggacttggataaggatttccctagttccattaaatctgcatctatggcagagcttaacactttataattctcatcttggaagtcgaaggctgatgcacccaggatctcggcaacagtagcccgcttcgctcgagcagctgggaatggagatagtctggtccccatctcatcagagtaacgaagaattctaggggcaagctggaccagtacacaaaatccatttgtgaggttaagagcctgggtgtggatgcaagatgtcagcccagtagtgcatgcccaccatgtattgatggggggtataatgtaagcatccctgtctggggcttcctggatgctattacataaatgtctatgggtgtggggtattgtcccaaagcagagtccttttccctgaattagctctaaggttagtctccctttacctggatactgccaacgacagtccttgttgtcagtagaggtggtataatttccaggaacggctattccttcataatagggtggctgggcatccaagcagagccaacatttacttgtaagctctggtttagaattattcaataccaggtaagtggccttaagcaagtcccatggcccagacctgggtctaagttcctgagaaatggggactgtgatttgtgattgagtcaccgtgagtccaacagggggcagggctggattggggcctaaaggcatgccctgtcgggcagagagttggatcgtaaacattactccatagtcatatccagacacatacagtcttagtccccatgtctttcccttttcccatcctacgctttcttttcccttgggggtgaactgaactgtaatggggttacagttggaggaacatggcccaccaagatcatttgcatccagcttgggctgcaatccatacccccatccttgggattccgatccagggctcctttggaccttgatgaggtcatcttttatcggtggcgtccaggagatggacccagtcgactcacaaccccaatatgcacagaagtatgactctggtcctccacaggtagccgtctgctgtggagacctcccctctccagggcagacatagaagggggttctccgaagttcactgtgttgagctggatgttgacacccccatacaaggcccaggggatcccaggtcccatccgctagtgcacacaaatcaaaaacaagatcaggaaaccaagttccaatgggggcgaggtgggaggtcgagttaattacagtccccgtagcagtgttagttataacccaagtaaggttatggggttcatggggcataggactagtttggaccacacctatagaaatcagcagagtcaagatctgcagtgtcttagctttagggaatcttatggttgtcattggagtcttgatcccgtctggggcacttggcagcatctcccgtctagtgatcgagacttcatcacctggttggaaacaatggtggttaggtgttgcatagatctgccatgtttggcccaatccatttctgtaccttgtaaaggacctgtaagcactcttgtctacagctggcatgaagtttttctgcctgatttctctctccgtgtacttttgaataatacaaagacaactttacacagtacacaagagattatcccaacaggcctttctcccccaacccccctttcttttcttcttctttttttcttggtttttcgagacagggtttctctgtgtagctctggctgtcctcgaactcaagaaatccgcctgcctctgcctcacactcataagtgctgggattaaaggcatgcgccaccactgccaggctagcagtggcctctcaaaagcactgcgaacttatgttttccctctaggtttgagctttagtagaccagctggagtgcttctgcctttgggtcccctgcccttccttcacgtgactccatgtcaggcagtgagcgctcagcacctagagcccctttcctgttttacctgggttagatccgaggatcttccgcgaactggcggtgcactccgttaccaaaagcctgacatggccttgagcacaccccatcagtacgctttctaaaagcataagataaatacaagcaagagacgcggaacgcacggtgcatttaccaacgccagggaattaccaacctttcattccctacagcaacagctcccagcagccaggcactgcttgtgaaagacccccaagctgggtagtcaatcactctgaggagaccctcccaaggatcagcgaggccacgattcggatgcaacagcaaaaggctttattgggaacacgggtacccgggcgactcagtctatcggatgactggcgcgccgagtgtggagtacttacccttcttatagggctagggctggggggcaaaaagcgcgggtacagaagcgagaagcgagctgattggttagtttaaataaggcttggggtatttcccggtcatttggggaacctgaaactgaggtgggactttccagaaactgttgctggtttcgctttatctgagtaccatctgttcttggccctgagccggggcccaggtgcttgaccacagatatcctgtttggcccctgtcccggtattattcagccttattctttaactaaacttccttgtaacttttgagaactcagctctggtactttatcatgccttgcaaaatggcgttactgcagctaacttgctaagccttatggtggggtctttcacttggagcagcccgagattctgcagctcgccctgcaaaaggcggaaacctgggaccaagcacgcactccagacactagggctttcgttttcccgcgctctagacacgaagaccccgggctgcccgcggcggcgcctgacccgagggccacgggctgcccgcggcggcgcctgacccagacccaggctcgtcccggcacgcgggttcccgtctccgtgctgcacgagccactctggcccctgcttgacccgtccggcagcggtgggccgcgcacggaccctcgggatcgcgcacgcacaggcggggcccggcagccctgcgaggacccgagtccggaaacgtgcgctgtcacaaagggggggacacgtgggtgccgctgtcccggccgcgttctcccgcagcgggagagctgcctccgctgcccgccacctccaccccggccgccctgtggggccgcggccgtagccggccgagtgtgatcgccgccttcagtacgtacacgcggcgcggcctcccctctccaacccgagggggcgggcgatcggggtcccgcctcctcctctaggcgctgctggcggctgctcggccaggcctagggtccggccgggcgcctgcgtaccttgcagcacgtgctcaggtcccctcctcaatctgccgggtctctcctcaggtcactgcaccgcgttcggaccctgcacagccatgtccatcctctacgtctctcctcaccccgacgccttccccagcctccgagccctcatcgctgcccgatacggggaagctggtgacggtcccgggtggggaggccctcacccccgcatctgcttgcagcctcccccgagcagccggactccattccctccgcctcgcctgcccgccctggagcagggacctggcggcctgtgggtgtggggggccccggctgtagctcagctgttgtggcctgcaggcctggggggacccgggggcagccgggcagctgtcctggtccagcagtgggtcagttatgctgacacggagctcatacccgctgcctgcggggcgaccctgcctgccctgggacttcgaggtcctggtcaggatccccaggtgagagggaagaaaaagggagaaattctgactacacaaacaggattgggaagaatcccacgaaaaaagtcggttgtcaggcggtcgcatctctgcctgccacctggtgtagtaactttgggaacactttttctcgagccttggtcttctatgtctgagaatgcaagatgatgaaatctcttccttcctccgggggtgtgatacatgagcgcccgggagtggagcgtggttttgctgttttcactacgtttggagtgtgtgtgtgtgtgtgtgtgtgtgtgtgtgtgtgtctttaaattaggtctcgctctaactgaatggctcggaacgaacgcactggtgtgccaagcattcccaagtctgtctttgggtttctgttgccatgtttaaaacaacatgaccaaaagcgccttgggaaagaaaggaattgtttcaggttacagctctccatcactgaaggaagtcagaccagtgactggaagcagaaacaaaaagaggccgtgcaggaccgatactttctggctcactttccacagcttgttcagcctgctcttttataccgtccagggctgctttgccaggggtggtatcccctacaatggactggccctccacataatcattagttaagaaaacgtccccacacgcttacccacaggccagtctggtggggacattttcccaattgagtttcccacttcgcacgggccactggcttaggtgaggttgacaaggccctggttggcaagtgtgacagggatgcaggatgtgagcggttttatgttgaactgtagggaagactggggagagatgacagttgctgggaggagtcagaggaggcttgaagttgaaggagaggcagggagaggacagatgacacaccagattgtttctgtgagtccttccagggtagacctgtgtctgccttaactgaaagctctgcccctgcagggtagggagtgcatggtggctgcctagtgtcacttcagtgggggaatgaaggaacagtgcagatggtgactggcttcttcatatctggatggactaacagagagcctcattcctgcggggtagtacttcagacctgcgctcatttgtggttttcctgggtgaaatctattgatgcccagacctggcagtcttgtttcatataatcctttaactttagagcctgatatgtttccttggactcataaacttaagtgtttagtgataacttgttggataaatcacacacacaaaatacttgtgtgtattcgatttttattttatgtgtataagtgttttgcttgtgtgtgtgtgtgtgtgtgtgtgtgtacacttgtgtgcctagtggccaaggaatctggggacatgtaacgaggtgttgtcataacttggtttacggactgttgtgcaccaccatgtgggctcctggaaccaaactctctaagagcagccagtgttacaaacgctgatctgtgtcattcacctctccagtcctataatgtatataatccaggggcctcctgcatgctatgctaagtactcttccttgaactttcagccagactagagttttgttgttgctttaagacagggtcttaactatgtagccttacctgaagtgggactggttgtgaagactagcaagctttaaaggcagatccctctgccttgccttcggagacctagggttaagggcatggtaccacacccaggagaactatttatagtgaccttgtgaatgaggaaggaaggagcctgagacctggtggtcagtggcttgagtctgggaaggtcgtggacaggtggagtagtggctcttaaccttgcccaggtcaggacccttctgaggggatggaaatgaccctttcacgggggtcaccaagaaaaacattgaaagacagatattaagattcattaccatagcaatgccacagttatgaagtagcaacaaacgtaatggggggtcatgaccctttcacaggggtcgcctaagaaaaacattaaaagacagataatgggctggagagatggctcagcaggtaagagcattgactgctcttccagaggtcctgagttcaattcccagcaaccacatggtggctcacaaccatctatctgtaatgagatctgacaccctcttctggtgtgtctgaaggcagctacagtgtacttaaatacaataaataaatcttttaaaaaaacagacagatattatattacaactcattaccacagcaatgctacagttatgaagtagcaacaaaagtaattttatggtgtgggagggtcaccacaacatggggagctgtagtaaagggtctccgcattgggaaggaccactgagataagggtgtaagcctgtcataacactatcctttcctgttaggctgcccttggggccctgggcaaggccttgaaccccttggaggactggctgcggctgcacacctacctggctggagatgcccccactctggctgacttagctgctgtgacagccttactgctgcctttccgatacgtgagtcactgagcttggggagggacaaatgcgtgcccgttagacctccttacacggtgactacgataattttttggttcggtttggtttctcgcaggttctggacccttctgcccgccggatctggggcaatgtgactcgctggtttaacacttgtgtccggcaaccggaattccgggctgtgctgggagaagtggccctgtactcgggggccaggtctgtcactcaacagccaggtgaggaggatgggagacagcagagggcacaagggccccttttatcttgaaatcctggggccaccatctggtgtggaggctttgtagggtgagggtcaaagatgatcatcaagcctgaggcaagagctttagactcagcactcttcacagagagtccccagcaaccttgagtctgaatttctgtgcttctctccaggctctgaggtcatcgcaccccaaaaaacacccgcgcagctcaaaaaagaggcaaagaaacgggagaaactagagaaattccagcagaagcagaagacccagcagcagcccccgcacggagaggtgggggggccaaaggctgagcgggagtgggtggagccttgttggggaggctcatgtttgggtctgtgtcttcttctgaacagaagaaaccaaaaccagagaagaaggagaaacgggaccctggggtcattacctatgacctccctaccccaccgggggagaagaaaggtactggctggggaagggcccagctccggccgctgctgtctcggttcctctgtgctgtgtgtgtcctggcttccaccctgcttagtgagggatggctagcctcaggctggcctgtggggagaatggcaaacggcctgttgcttctggcctaactcagagtctttactgagccctctccttcctcccagatgtaagtggcgccatgcccgactcctacagccctcagtatgtggaggctgcctggtatccatggtgggagcggcagggcttcttcaagccagagtacggggtgagtggttactgctgcccggacccagagggggctggaggtggagaagggtgggacttcaggagccgtgcctgggaagggatgccataggctttagcatctgcccctccttctccaccaccagcgtcctagtgtgtcagcaccaaatccccggggtgtcttcatgatgtgcatcccaccccccaacgtgacaggctccctgcacctgggccacgcactcaccaacgccatccaggactccctgactcgatggtgagctcctgtttgcgcctccagccggtgccttctgccctgcttggctccattctctctgctgactaggctccctctgccacggtggccttgtctggccctcaggggtgactttcctgcttgctcccctctgagcgttcagtagtctttcctgttcaagtaatctgtcagcgaggctgacttccacaggttccttctccctcccgtctgtctcctgagaaggatgttatagccccactgtcgaagcactggggaccgggcctgtggcctgtagacaccaagtgctcagtctaccttagacccgcaccattggtctgcagcaacgttcctttggattttaacggcatgagcagactcatgttgaaacgtacatctccttgaccttcctactcagacctcctcccttccagagtggagtccttgcttattagcagtcaaagtacatcttcccaggtctgtcctagctgtctctgtgtatctacatagaagtatcattcagtgttctttccaggatacactggtccttagatgctgagggggaagtagtagtttttctttcttttttttttttttttttttttttttaaagatttatttatttattatatgtaagtacactgtagctgtcttcagacactccagaagagggcgtcagatcttgttatagatggttatgagccaccatgtggttgctgggatttgaactccggaccttcggaagagcagtcaggtgctcttacctgctgagccatctcaccagcccctttctttctttttttaaaaagatttatttattttgtgtatgtgagtacactatagctgtacagatggttgtgagtcttcatgtggttgttggaaattgaatttttaggacctctgctcgctccagttcaactctgctcactcagtccctgcttgctccagcccatagatttatttattattatatctaagtacactgaagctgtcttcagacacactagaagagggcatcatatctcattacagatggttgtggccaacctgtggttgctgggatttgaacccaggaccttcggaagagcagtcagtgctcttacccactgagccatctcgccagcccttttgtttttttcaagacatggtcttactatgtaggtttatctgtgctgtcctggaaatgactctgtagaccaggctgactttgaactcacagagatctgtcttccttggcttctcaggtcctggagggaaggtagttgttattctagaccaatcttcccttctattttactttcaaagtttgacttaagttatatgtgtttatgtgggtccgtgcacatgagagcagatgctggcagcagccaagggtgttggatgctcctggagttaggggtggatgtgagccacgatgtggcgctggaaattgcactctggtcctttggaagagcaacacatgttcttgataatcactgagccatctccacaacccctcttccttcccttcccttcccttcccttcccttcccttcccttcccttcccttccctttcctttcctttcttttagtagaaaagatttaaatctaaccagcaaaagttgtaactccgagttggagaagtggttcggcagtaggtttggttcctaaaacaccaatatggtgacttgtcaagcgtccttaaccccatttccggatccatgtttgtgtaggcaaaacaatcatttataaagttaaaaaaagttttatagccaggtgtaatggcaatgctggcaggagtgtggccagttcaaggctagcctgggctatgtagtgagactatagctcaaagcaaatatacagagagttgttccttagctttttcccaatgttcaacggtttctcttgggcgtgtttacttgttcttgtatacaggctgactttgtttttattctttttttaatttaaaaatcatcttaaaagaattaattgtatgtatgagtatactgttgctgtcttcagatacaccagaagagggcatctgatctcattacagatggttgtgagccaccatgtaaatgctgggagttgaactcaggacctctggaagagcagtcagtgctcttacccgctgagccatctctccagcccctaatttaaatttttattaaatgtgtgaatagtattttactgtataatgtaccacatgagtgctttgtgcctgcagaggccagaagaggaagttggatccctcggaactacagttagagacatttgtgagtcactatgtgggtgctgggctttgaacctaggtcctttggacaggcagccagtgttctctttactgttccttgagttgaggtctcatgttggctaggctagcctgcagctcctggtgaccctcgctgttgatctcagtagctgcgactacgggtacatgctctggagtccagctcctgatttatttatctgtgcattctgttttctgttctttgtgtactagtgttttgcctgcgtttatgtctgggtgccgcttgtgtacagtcacagcaatcccttgggactggagttagatagtcgtgagcagccctgtgaagctgggagagagcctgagtccttgagatcctcagcccatgctcctgaccgctgcaaatgtaaatgagcaaaccttccccaggccaccctacccccattttggtttttgttttgttgtgtggcttttaaatttcctgtcttagaggttagtagcccagaatggcatcaggctaagtgagttaggtgaccaaggcagaccttgaactcccgacaatcctgccttctaacaaatatagggagtgcacacatgagctgttctaccctgattctttggtttggtttggtttttggtttttcgagacagggttgctctgtgtagccctggctgtcctggatctcactctgtaaaccaggccttgaactcagaaatctgcctgcctctgcctcccgagtgcggggattaaaggcatgtgtccggtacccagaatcatttttaacagtaatgataccaaaaggtgctgtttttctttttttgagttggatctattgtagaacttgctttgtagaccaggctggcctcagactcgcagtttgcctctgcctcctgggcagtgagattaagtatgtgcaccaccaccgcctcgtttcttaaagcgtattaaatgcttttaatacgttcctagcgtatgttaatgctctccaaggcgtttgtaaagacgacttcttgccatctataagaggatggactttctacaatctctttccacagaaaatctgagtgtagtgtgataagcaggcttgaatatattactcactttattatgtattggtcatggtccacaaacatgcgggagggagctgtgaagatggctcagtggataaagcacttactgcaaaaatatgcaaacctgagtttgaatccacaggacctatgcaaagctgagtgaacatctgtaatctcagaagccccagaggtcactggaagccagctaagcttgttacagtggggaatctgaccccagctcacggtggaagacggggaccgaggcctcggttcaccctctggctttcacgtgcactcttcagtcccttcctgtccttcccttaatcatctctcactttgcttagaagtactgatttggcttcttggtactttgtgtctgccgtttttggcaaatggactcgttgagacaggctttcctgagtatgtagtcctcagtggcctacagcttgctgtttagatcaggctggcctggaactcacagaaatccacctgcctctgcctcgggggctgggattacgaggccaccaccctggcacctagcacggtttcttttttgttgttgttgttgttgtttgtttttgtttttcaagacagggtttctctgtgtagccctggctatcctggaactcactctatagaccaggctggcctcgaactcagaaatctgcctgcctctgcctcccaagtgctgggattaaagacatgcgccactactgcccagactcacctaacactttcttgccagcgtatgacataatctatcatttgccctcccttaccatctgtgtgcctcatcctcctgctgatctcccgtttgttgttgcgaaccagtgaaccgagtgaaggcttgctcatttacagggccacaggcatcatgccgactccactgaggaacacttgtctctcctccctctgcagtcactgcctggggatccacagagccatgtacctctccctgtcctgtcttctctgtcggcacttggatgtgtgacagctcttaggacatgggggttagagcaagccctcttgcgtaatctgcctgggtccctgtggataaagtttgcatctttctgtggctttgttcctaggaccaccttgtgcttggctctctggctgggtgagacctcctgttccttttacagattgttctgctgggcttccaactcctttctcactacctgactttagaatgagaaggtttcaacccatctctttcttttttttctttttttttaagatttatttatttattttatgtatgtgagtacactatagctgtacagatagatgggttgtgagccttcatgtggttgttgggaattgaatttttgggacctctattcgctctggtaaacccctcccactccagcccaaagatttattattataatcttcagacacaccagaagagggctcattatgggtggttataagccaccatgtggttgctgggatttgaacccgggaccttcggaagagcagtgagtgctcttacccactgagccatctcaccagccccaacccatccctttcttaagcatccatgaacccctctggcagaaacctgagaccgctggtgtcagaatctgccagcctggcttaccctctggttttcatgcctcccacaggcaccgcatgcgtggggagaccaccctatggaacccaggctgtgaccatgcaggcattgccacccaggtggtggtggaaaagaagctctggaaagagcggggtctgaaccggcaccagctgggccgcgaggcctttcttgaggaggtctggaagtggaaagccgagtgagtacagtaaccccatggatgctgcaaccctggccgcaggctcaggcgtcccacggggctggccaaagggagaaacttgtcaccttcagaggggcaagtgggtccctcactacccaccagccacgggtggcagaaggcttatcctggactctgtccacttccaggaagggtgacaggatttaccaccaattaaagaaacttggcagctccttggactgggatcgagcctgcttcaccatggatcctgtatgcaggcagaaccttggggcagggcagggatgctccatgggtgggctcccctctaccaggaggtcagaacatagccaaggactcagccaccagcacccagaccctagctcaagttccctgctgacccagcatcaccaggctcatggcctgccctgggcgttctgtgctgggtgcaggactctaggataggcctggtggactcccttgcctcctgctgccttgggtaaattccttggcctccagggatcaaggtgtctctaacttccattatctttaccctagaaattgtcagcaactgtgacagaggcctttgttaggctccacgaagaaggggtcatctaccgtagcacccgcctggtcaactggtcctgcaccctcaactcagccatttctgacattgaggtgtgtctctgcccacagcccgtctccctctagtcttccccagcccaggcctggcccacttctctaaacacccatctcacaggtggataagaaggagctgacaggccgcaccctgcttcctgtgcctggctacaaggagaaggtggagtttggggtccttgtgtcctttgcctacaaggtccaaggctcaggtgggagctgaggacaccaggatgtgggttgggagaggtggggaggaggagagagctcagacacagagccttctgtcacctcagacagcgacgaggaggtggtggtggcaacaacccggattgagaccatgctgggagacgtggctgtagctgtgcaccccaaggatcccagataccaggtgggacggggacattcgtgcctgtgtcctgggatggctgctggctcaggataggctctccccgggtgagggcccagccctgtgtcaaccccactgtttccctccctgttaagcacttaaagggaaagtgtgtcgtccacccattcttgtcccggagccttcccatcgtcttcgatgactttgtagacatggagtttggcacaggtgagcaacggccgtggcctgttgggagaaaagactgaaaaattgtcttcagtcccgtaggcaggactggggctggatatgagaactgttcggacagcagagtggtctgagagctgtgcccttcggcacaagaggtgtggaagggaaccccagcatgcagtagaaccctggcacgccccgtgtcctccaggtgccgtgaagatcaccccggcacatgaccaaaatgactatgaggttgggcagcgacacaggctcgaggccattagcatcatggactcaaagggggccctcatcaatgtgcctccacctttcctggtgaggtgtcgggactgtggggacctgcgtgggaggtggagagctaacgtgggcattgccatgatgaagccttttccatccagggcctgcccaggtttgaggccaggaaggctgtgctggcggcactgaaggagcggggcctgttccgtggagtcaaggacaaccccatggtggtgccgctttgcaagtgagactggggcaggcgcacatggggaagggtggggttcctctgggtgctcagcacatgacctctcgatggccgtcttcctactacagccgctccaaggatgtggtggagcctctgttaaggccacaatggtacgtgcgctgtggggagatggctcaggctgccagtgctgctgtgacccggggtgacctccgtatactgcctgaggcccatcaacggacgtggcattcttggatggacaacatcaggtgtgtatggcccctgggtggggaggctggccagctgaggggcccagcctgagccccctgactgacccttgctgtttgtagagactggtgcatctctcggcagctgtggtggggccaccgaatccctgcctactttatcaccgtccatgacccggcagtaccccctggggaggtgagaaaggctatagctagctgtttcctggctgtggtttgttgggcccttggttggcagaggggcttggcaaagtaaaggaagcctttttgataaccctctctgttaggaccctgatgggcggtactgggtaagcggacgcactgaagcagaggcccgagagaaggcagcacgggagtttggagtgtcccctgacaagatcagccttcagcaaggcagggatgggcacggaggatggggacagtcgggattcttcttttctgttcctgtccctctaatgtctgaccctggctttccccagatgaggatgtgttggacacctggttctcctccggtctctttcccttctccatctttggctggcccaatcaggtatgctagagtgaggggaggggcactgaaggggaatgcttggctcatctccttgccctcgtcctcccgcagtcagaagacctcagtgtgttctaccctgggaccctgttggagacaggccatgatatcctcttcttctgggtggcccggatggtcatgctcggcctgaagctcactgggaagctgcccttcagagaggtgtgactatggctaagccccgcccctgccccgtgtctccttacctgccttcttgggtggctgttggctaagattagcttgatgggtctgcctcagggcttcctcttcccagaggagggggcacttgtcagatacactatgaccagccagtagggccactaggaacctatgattccaagtgggcagactggggagggccttcaagactgggaggagcatggcctcacagttcatcacgcctcgtgccctcaggtctatctccatgcaatcgtgcgtgatgctcatggcaggaagatgagcaagtctcttggcaatgtcatcgaccccctggatgtcatccatggagtttccttgcaggtggggtactcagtgggtcagctagaaacaactttgcagctgtggctagaacgatctgacacctcccatctcctccctagggcctctatgaccaactactgaacagcaacctggatcccagtgaggtggagaaagccaaagaaggacaggtaaggggtgtgctccgggggtgggctgcagcaggtgggcaggagtgggtgtaccccaggggtgggctgcagcaggtgggcaacctgaccaccctgctcaaccgccctgttcctctgcatcctctgtagaaggccgactttccagcagggattcccgagtgtggcactgatgccctacgctttggactctgtgcctacacatcccaaggtatggtctcccgacctccctcagttgcctgttttcttccctgcaacccgtgatctcctctaccttgggccttcagaggacctctctgagagggcaactggggtggaatcacagacatccctcctctaggtcgagacatcaacctggatgtgaacaggatcctggggtaccgtcacttctgcaacaaactctggaatgctaccaagtttgccctgcgcggccttgggaagggctttgtgccctcagcaacctccaaggtgaggatcttgtcggtggccatcggtgacagtgcctgtgcccacacagtctgtggctccctccactcaagtgtcaagactaaagctgctgtcccctgagatcctccttttagtcagggacagacaggtggaagttgatgattgatggggaagtctctgcagcttggggcccatgaggaatgcagattctcctgtgtcccactctactccctgccctggtagtgtgatggcctactgcctgacgtttctcttcccccaagcctgaagggcacgagagcctggtggaccgctggatccgcagccgcctaactgaggccgtgaggctcagcaatgaaggcttccaggcttacgatttcccggccatcaccaccgcccagtacagcttttggctctatgagctctgtgatgtctatttggtgagcagctggggcggggcctgggcacgtgtagctctgacctcagcttggccctgaaacttgaggtcaccttttggtttttccagtcctaatgggggcagcaggggagggggctccaggtgggacctggccctgagccttctgcctccccatctctaggagtgtctaaaacctgtgctgaatggagtggaccaggtggcggcagagtgtgctcggcagaccctctacacctgcctggatgttggcctgcggctgctctcgcccttcatgccctttgtcacagaggagctattccagagactgccccggcggacaccaaaagcgcctgctagcctctgtgtcaccccgtacccagagccctcagaggtatgaggtgtggcctggagggcatgctgctgccctcctgagcagtccttttctcccactctgccccacagtgctccccctttccccactctgccccacagtgctccccttttccccccgtctgactcacagtgctccccctccccccactctgccctagtgctccccttttcccccagtctgactcacagtgctcccccttcccccactctgccccacagtgctcctggaaggaccctgaagctgaagctgctcttgaactagctctgagcatcactcgagctgtgcgctccctgcgtgctgactacaacctgacccgcaccaggcccgactgtaagcctcagtccctcgcatccacctatcccctgacctccacggccccttggggagcccagggtctggctgtacctcggcttccctttcctctttttccccctcttcgctctacaggtttcttggaagtagccgatgaggccacaggtgccttggcctcggcagtgtcgggctacgtgcaggctctggccagtgcgggcgtggtggctgtcctcgccctgggtgctcctgcaccgcagggctgcgctgtagctgtggcttctgaccgctgctccatccacctgcagctgcaggggctagtggacccagcccgggagttgggcaagctgcaggccaagcgaagtgaggcacagcggcaggctcagcggctgcaggagcgccgtgctgcctccagctactcagcaaaggtgccccttgaggtccaggaggcagatgaagcgaaggtgtgtggaggggatggggtatgccagtgggtcgtgtgtggccaggcagcctgtggactcctgcacaggtggggttgtgtgtgctctggggccctttgtcactgtactctcaagaaaagaagcgctctagaggaagagggacttggaagcctgctgccctgacttcctggtcatcctcacccatctcatcaccctgggctcctctgctctctgggtgtttaggctccttgggtatggggtcaccacatacctcttttgcacccccagttgcaacagacagaggcggagctcaggaaggtggatgaggccatcgccctgttccagaagatgctgtgaccccccattccaaccctcagcccccagtgttccatcaacagggatggcagcaataaaatattttcccaacattcgctcttgtctgtgtggtgtgggcagggggcgtacagaggcctgacctagggcttcccaggagcagcaggagaactgccggctcctgcctggccctggtcctggtgggcagcaggatgcgtctgcctgggtctgtctctggcacagtgccaggcctgagtcacggtgctctgctgccagctccccgcctccccagttcacctcactgcactgcactgcgctgtgcctgcggaggcctgccctgggcccgccccggctttcgctacatcttgtggctctgggttggcacgaatggaagatggccaggggctgcctgtcctcaatccccttgctaagagcctccctctcactctactgaggcccagttaattataactctgacctaagtgccctgtgacgccacacccaggccagcgctgcccaggagacccagcttccaggtaggagaggcctgaggagacttctatccgtggggcagggccagggctcagtctggtgtaggaagagaatcttgagagggtcctccggggcctccgatggcagctgtgggctccactggcctgacaggcccttagtttgtatgggttgcaggcacagggaggggagccaccctggagtatatggggcacctgaagcaaagcaggaagtgggcaataggtgccatggtgacagcaggcctgccctgcaattggtggcttttttttcccttgaggtgcttctgttccctaggtccatgcttcagccatgctccctgtggaggtacccctgtcccacctgggccccccaatactgcttctgcttcagctgctgttgcccccaacatctgccttctttcccaacatctggagccttctggctgcccctggctctgtcactcaccaagacctgactgaggaggctgcactcaatgtcacccttgtgctctttctggagcagccacacccaggccgaccacgcctccatgtagaggactacagggtgagcaccttagattcacctggggtttctcagaggatccttgtcacaaggaggctccacctttgacttgctcacttccacacttgtccctcctcaattctggtcctgaaatcctgagtcgccagtcttgggaaagtctgtaccaaaagagatgtgatggcacacacctgtaatcccagcacttaaggagaatggaagttaaaggccagcatgggctgcatagtgagcgccaggctagccgggtcagccagtccaaatctgctgctcccctgtgggtctcttggcccaccattctattcagccttgagtgccagttgctagggtacttcaggcagtggtagcatccaaacgacttctggagtttctttccctaaccactcttgctagggccggaccctcctcgccgatgacatctttgctgcctactttggacctgggttttcttcccggcggttcagggcagctttaggagaggtgtctcgtgccaatgcagcccaagacttcctgccagctttcaagagcaaccccgacctgcacttcgatgctgaacggctggtccaggggcgcactcgcctggtgggggcccttcgggaaaccctggtggctgccagagccctcgaatatacattggcccgccagcgcctaggggctgcacttcatgccctgcaggtgactctcagggcttttaacctgcctcctcttgctttgggaggagggtacacacccctcctgagctggcagcacttcctctgtctggccaggagtagctacaaagaagggaaggagagaggaagacctggaggtgggggtgttaatttatcctagaaagcctgtggcttagactccaagaagggctggggtgtgtccatttgggcaaagctctttcctgggcaatgagaacagaagaaacaggggtacagaaacaggagcagcagggtggaattctttgtttggtgggaacaccaagtcagggcatatgggctgcacgactgggtgttgggctctgggtggcatatcaaggtgttgaccctgactagatcaaatcagtgcctgttgccccttccttctctcctggggaccaggatttctatagccacagcaactgggtggagctgggggagcggcagccacaccctcacctcctctggccaaggcaggagctctggagcctggcacaaggtatggccatgatgcagtttggaggtaggaagggactcagggagcttcctgtcccccgtgtcaccttctgcagtcccaggatgggacgatgctttatccctgcagggactctgaggtccttgagtagctaatggctacaagagttcagtggctttctcagccttacacaggaaagcatccaggatcagctcagacctcacgccagtcttgcccgtggcttgttctatccacctgtgacagccctgtggccttctcttgaagcccccctcatgctcccaactcactgggagctacgtcgtcccatagcaactcactatgggatggtagcctgcctgtggttcacagagagcagaatccccaaagaattggtacccacacttgtgggttgtgcacatcagacatccaaatggtgaccctgaggtttagtgcaaaaagcctggccaaggtgtctgtggcctttcctatctttccagctgagaggaggatgttaggtaaccaggggtaaacccagcaggttgaggcttggattccagcccctgccctctgttcccagtgggcgatcctacatgctctgactgtgaggggctgagctgccctgggaatatgctggactcgacactgctcacctctggctacttcggaatgcatccccccaaacctccaggtaccagatgcgaaggctcccagaagggacaggagaaagtcactttctttgacttttttttttaagatttattttatttatatgagtacactgtagctgtcttcagacacaccagaagagggcatcagatcccattacagatggttgtgaaccaccatatggttgctaggaattgaactcaggacctctggaagagcatctctccagccccactttctttgactttaacacaagatcctggttgcatctccttaggagagggtggtgggatagaaccagaatgggtggatgaaggctgaggagacattgtcattctctgttgcctgggcagtgttggggaatggatacagagaaagactctgaccactctagatgcacacagaatgtcactaagtgctgatgctatcgtgctgaggggtggaaaaatcaaaccaggtcagaaagggtccccacagtctttagtaggcaccgagcctttgactcttacgtgccagatctcttcccttaatctagggaaatgtagccatgggggccattttgaccagagcagctcccagccaccccggggaggcatcaacaaggacagcacatccccaagtttctccccgcaccataagctgcacctccaggctgcagaagtggcactcctggcctccatcgaggccttcagcctcctgcgaagccgcctgggcgacaaggctttctccaggtgggtggcctcgtggagtcaggttccctgatgggtcggtgctcctgcctgcgggaactgaggcattggggacgaatgcggaaacttcactgctcctctgacttgaaccacaggctgctggacatcaccccagcttccagcctgagctttgtcctggacaccacaggcagtatgggcgaggaaatcaacgcagccaagatccaggctcgccgcattgtggagcagcgtcaaggcagccccatggagcctgttttctatatcctagtgcccttccacgacccaggtagcggtggtctacagggatgatggtgggaaggagggcccaggacagaggctggagacctccctctggtgaggaggtgggcctcagggccattctacctgttgtcctgagtgagcctctactgggtacctgtgtccatgtagacaataagctgcttctgctatctctaaaatggtagggggcggtttcacaagtccagtgcctgcttaggcacctgcctctggcttccttagacttctgtgctaatagaatatgccttttgggttcattttgttttaaaccatgggtggctgtttcgctttcatgttggtctgcgtatgcacacgtatgaaggacaggaaagggtgtcagatccccaggaaccggagttggtggtcccgagtcacccaatatggagtggaactcaggtccttgccccagggcagcgacactcttacctgctgagccggctccccaggctgctgctgaggtcagagataagatgaaggtgtatggggaggttagagagtaccactcctacatcttcccactgagcccaaagtcagcttacctggcctggctctactggtcttactgcacccttgggctggccatctaatccttctgtgctatagcctctataaaacggccagtgcgtgcatgcctggtttaatcctcctggatggagaactcctgcagggggtagtgtctgatattaagtagggacttagtgtgtgcttgtggacacatgcgtcaataacctcatagtaccgtgtccgggcgtttacaaatgcagggctcattcagttctcccaaggagcctctcaggtgagttctatccttttctctcattacattttaaaggtgaggactggagaaatggttcagcagctaagagtacttgctactcatgcagaggacctgggttttggtgttccgtattggtatccacctcggggtagaggctcacagctccctcatggcctctattctcctcatccacctgaccttcctttcctcttgcccagggtttggccccgtctttacaaccagcgacccggacagcttttggcagaaactcaacgagatccatgccttggggggtggagatgagccagagatgtgcctgtctgccctggaggtcttgactctccttccctctcccgcctctccctttccctcttatcctgcccttccaggccccgccaccagggggagccaagagctagcaaggctcccttctcttcccccaaccccctcaccatcctcccatcctctatcctcggttccatcagcttccttccagggtcatgctcccttgcgctctgccctccctggctctaccctgctgcccaccattttaaacatgagcctttttttctcctggccagctagccctgttgcacacccctcccctctctgacatctttgtcttcactgatgcctcacccaaggatgctcttcttaccaaccgggtggaatccctgactcgggagaggcgctgcagggtgagcgcagtgctagcaacctgagtatgggaaagagagcggcctcatgggaggtcacccccctcccccgccccgcatctttaacagagtgggaggccgtgcccttattaaacggatccaataagccattcagagaacccgttgtgagggtctctgcagtcaccagggtagacagaaacaagtgaagcaagaaggttcaacatctagaatctgaccctgggtggtttattttaggcattttaaatatccagtgcaactctggggaaaggtttcctggtaacacttgtcacagtaaagctttgcaaaggacatgtgacttcctacttaagactgggaaacaatgctcaagataaggaaggcctagggaggaagagtctgagataaaccccgaagtctggaagactcggtgaggcctggccctacagatagcacagagcacaagctcttaactcccacttccccagaggatagacatctcttccgttgaggagacaaccattcattcccggtatctgcagagcttatctgtgagcacaaggaccttttccctctgcatcaggcaaacaagggcgggcctagctctgtggtgtctaaatctgatgcactgggtctaatccttagtacaacacacaagaagtaaatgccagtatgtataaagacagaattaaaacaaagccaggaaatgaatctctgggaaactccttccgaggtgtggttagagctctcccaagggctgtttcactgggaggtcccacccaccacatcagcatcagccagggggcagacagctctagaaaaagtggggcagaaggaccttcattaacaatgcaggctgattgggaggcagaggcagtcagatttctgagttcgaggccagcctggtctacagagtgagttccaggacaaccagggctatacagagaaaccctgtctcgaaaaatcaaaaaaacaaaaaacaaaaaaacaatgcaggctgagcttcctggatacaccaagcattgggttgtcatttggccacaggtgacatttctagtaactgaagacccatcgaggactgggggtcgacgacggcgagaggccttgtctcctctgcgttttgaaccatatgaagcaatagcccgggcatcagggggagaggtgatcttcaccaaagaccagtacatccaggatgtggctgccattgttggggagagcatggctggtctggtgagtgaggggaagctagagggggccatgcccttagccagagctgaggtctgaggtgctgtgtgcttcttctcaaaggaggagttctgacagaagtcaggaagggactcttctcttcccagacatccctggtttcttaccttccaccctcttttcatgtcaataactgcaccctggcctctagggctgccataggaaatcagttgtttgtttgtttggttggttttttagttttgtttttgaggcagggtctcactatgtagtcctggctagcttggaattcactgtgtagaccaggctggccttgaactcacagagatctatctattcatcatctttggagggggaacaagaaatgatgacgtcattctaaacccctcacctccaagcacagtccctggtccatgcagggcccgctgacatccatcctccctgtgggaggcagtcttcccatgtccctcctctgcctttgcttccacctccctccatgctttccctctttctctcctttggacctgatcgtatctccaggtgactcttcccctggatccccctgtcttcacccctggggagccgtgtgtgttttctgtggacagtctgctctggcaggtcacagtccggatgcacggggacatcagtagcttctggatcaagagccctgcaggtattctaatgtggggctcaaggcagacagaagattcaagagtggagaacaggagaaggactgtgcagagagaaaagaattacttcatgtgacagtgtcttgttcccatccaaggggtgtcccagggcccagaggagggcataggtcctctgggtcacactcgtcgctttgggcaattctggacggtgaccatgactgaccctcctcggacagggacttgggagatccaggtagcagctgcgggaaccccccgggtgagagtacaaggtaaggagaagggttcctaggcacagcagtggtgagccgtggtggccgtggtcctcacaaagtctaagactgcttcccctcccagcccagacctctctggacttcctctttcatttcggtatctctgtggaggatggaccccaccctggcctctaccccctgactcagccagttgcaggtagtactattccctgacctctgcccccttggtctgctgcagtgtaactgtgtgtaaatgaacacatgtgtgccttgtttgatgagtaaagcatgctagggagagtccagcttcatgggcctcacgtggtgcactcctatgaccctgcccagctgagaccagacattccttttaccctgcacctccaacagtttcatgcagaccaactcctttccctctcacctcaggtctccagacccagctgctggtagaagtgacagggctcacctctagacagaagcttgtgggtggccagccacagttttcccatgttgtccttcgaagggtcccagagggcactcagctgggccgggtgtccttggagcccgtgggaccccccgttcgaggcctccttgctgcctctctaccacccacactgctgtcagtctcttctcccttctccctggagctggtcggccagggtggagggggagagagcctgcggaggactgcgccccagccttgcagtgtggcccccgtgcttctggaggtgaggagaagtgtggccagggctggggaggttagaaccagctgttctgacccgcccttccttccctgcagctgagtggccctccagatttcttgactccgggcagcaaggcccctctcagcctccacatcgtcagcttctctggcccccaggatcttgatcttaggacatcggtgaaccctagcttctccctcacctccaacctctccaggtgagtcttttgtgaacctctgcttgggttctggctctacccagtcagttctacaggggctggggctcaaggacttgacccttagccctcaatcctgtcctatgattggcttcaccccttggctgggacctaccagggaccttctgtctgatctgaggttgtccactcttcagggctcgcctgggactgaatgagtctgcctggggacgcctgtggctagaggtcccagattcagcagcccctgacagtgtggtgatggtgactgtgactgcagcaggtcagggagccagccaggtgcccccaacccatgccttcctccggctgctggtgctggcccagtcctcgaaggtaaggtgcttcctcaccccctcggcagcgggggggggggggggggcggggatgtggggggagtggaggctcagcactcttcaccttctccctcctctccacaggaccagctggacggccctgcccactctgctgcccctgtgctgcccccggtcagccctgccttgcttccttccactttggtgactcagggcagagctgggggagggatggcaggcaaagcctggtggggaacagttggaggggtgctgttcctgctaggctgcacttcctggtaacacaggaacaggacatcaagccccattttcagtctgccctatgggtgggatggtcacgtggattcagggccctgccctgcctacctggtttggggatttttcttccatcacatttgtccctcctctttctttctttctttctttctttctttctttctttctttctttctttctttctttctagatttatttatttattatatgtaagtgcactgtggctgtcttcagacacaccagaagaggggggcatcagatctcattacagatggttatgagcaaccatgtggttgctgggaattgaactcatgacctttggaagagcagtcaggtccctttatctttctttagaccaggctggcctcacagagatctacttgcctctgtgtgtcttcctactgctggaatcaaaggcatgcaccaccgctgcccggctcccttatctttctaaaatgagtagattggcataaccaggtggtacctacctgtcgcccctgtaacagggaggactgctgctgtgagttcaaggccagcctgagctatgagaccctagctcaaaatccacatcaaccaaaaccagaagattgggatggagggtgatgggtttcccttggggtgcttttcctaaccccaacaggagccaaaccaaaggcttttttttttttttttttaataaggaagctgctgtttattccagaagcagagcaggacacagggcacagagcagcacacagggcaccagatggtgcagttttcactctaagttccagggcagctgctttagcttagttttgcttcagctgaaggcgagtctcagtgatcacgccttctcaccggatacactggggaaagacggttgttgtgcacgatgaaggggagagcgagggcaaggggcatggagaaggagctgtggacactttgtaccaagctgtctgaatttctcagcctggagccccctggctgctgagttaagatgaaatcggggtggggaggggcaggagtattctgactccaaccccaccccttactcaccagtgcagggaggggagggtctcctgtctacacaagggtaggcccctttggccctgggggagcctggctgaccacctctcccgtggactgggtgactcctttccgatgctttgacaactcctaaaagagagagaatccgccctgccctgcaccgtcccccaccctgtgggtttgtcagccagggctcaacccctgtcagttcacccattgccaccagaagttctcttgccctgacattccctcctggtcctggactgtgtgggagtcagccagtgcatatggacgaggctaaatcagtacgactccaaaagccccgtccgtccccgaagtctagaaattaaaactaataaactcaccttgtgaatcagactgtgcagacaagcattcaccttttgaatccgggttagcagaggggcatcagtctctgaatgcaagtccatcagaaagtcctaggcagggttggagggtgaccgagtgctggagacaggtcctcctgccacttcctcaccacgggccactcttctcatcagattcccttctcccagaacccttccagacagatggaaaggccagccctctctctccacctccatctttttttttttttggtttttggagacagggtttctctgtagccctggctgtcctggaactcactctgtagaccaggctggcctcgaactcagaaatctgcctgcctctgcctcccaagtgccgggattaaaggcgtgggccaccactccacctccatctcttaaaattgcagtcagttcctttcaaactacctggaaccccacccccaccctggaaccccacccccactcctgttccttatgggtcttctcccccccccccccccccgcccagtgagagacacccaggccactctctgattctctgtggggtgctcagatggagtccctctgaatggaatgcagaccacttcttccagcacaatgaacactggtggctgtcctgtcatttccaatgctattccagttcatgtgccctgcccgttaaccctccccttcccagagcagcctttgaggtccacgtgctccctgcttctccctccccagcccgtgcacagatggctgccccctacctcgccaagggcccccagctgcagctgccgttgctgggtctccctcagccggtctgcgaaccaggcctggccatgctccagcagctccaggccctgccacagagcatcttgttccctctccagagcctgcatcatctgcagctggcttagaggagacacaggccaaaacgactcactcccagcaggaccctaggtatggcaatctcctctaaagggctcactatctgatcactaaggatttgaataacaaaggaatacagagggaaagtctgtgggtcctcagttgagaggaaatatatgcagggtacatcatgggaggggatgtcccaagactgagttggtctcgcactcacccagaggaagaagttctgggctcctgggtcttggcggcttgtccccagtggtagcagcagaacagtgtagggcgcttgcaccaggggtggcccaccaggacctgggctctccatggctctaaagtaggaagggcggagccggggctggtccctccccaacccatcttctacccatcctgccttgtctttcagcctgtgaggccaggataattcttaaggcaacagatagcttcccttaaactgtgtgtcaaccttcatttttgtttttgtggtttttgttttgtatagtctgttggaagtagcttgtcctcattgagtcccaaggacagaatctaaatttatttaaagttttatttagagtcagggtctccctgtgtagcctagctagcctggaactcactatatagaccaggctggcctcaaactcagagctccacctgcctttgtcttcccagtggtagaattaaaggtctgcaccatcgtacctgcatttatttatttataccaccattcctgactaaatgtaattatctacttgttagtttatgtgtatgtgtgtgtccatgagcttatgtgtattccatgtgtcccagagcccctgagactagaagagggtgtaagatcccttggaactggagtgacacacagatgtgacccaccatgtggttcttggaaccaaagccactttctctgcaagagcagtcagtgcctctaatcactgagcatctgcccagcccaactgtcactatatttgagtgacttccccctgtcccagctgatgtcctgttgggtgacctaaggaaacaccttgatctaaaataagctttattcctagcacaagacacaatgtgcaaactgagaactctgcagtaagggaaaaaaaaaaacccgaaaaggcatggtaggccccggggctcaggtagctgggaggcgagggaggactctcagaggatggtgggagcagcgggcagcacttcctgactaatgaaaatgtccaggtccagggtgggatcctccaagtccagttttagaaacgtatccaccagtgcctggcagctgcaggagagacagaacagtgtggctgctgggtcgccaactccctatgcagacagaggggagggtctgtgcgggaaactgcagccttggagagccaagcgtttcaaaagcagccatggatactgagttggttttgtttgtcgttttctgacaaggtcttgctatgtagctcaggctggccttgaacaccaggcttacaggcacgtgcccgtgccacacctgacttggtgaggagaaggaggaggaggaggaggaggaagagagcacggggctgcagaaagatggctggggccaggtgagggagaacacaggagctgtggggagcatggcaggccccactcacttttccatttggttcctcctcagaagctcattcgtggccttgatgggtttcccactgcggatcaagtctgagacctagcacagaggcacagggaggatggggtgaggacgttagggaagaggggaaatgagggtcagaagtcctgtgggcctacaagtccagagtttcaagaggaacaggaaagagggtgcagagatgcctcagcagctaagagcactgactgctctctcagagaacctgggttcaattcccagcacccactggcagctcacaaaccatctgtaactccagtgtcctgagattgatgtcattttctgacctccacagacaccaggcacatgtggtgtatttatataaattcaaggaaaacactcatatgcataagataagtcaatcttaaacttagcaagaggagcagtgtacttgagggggcccgaggtggggtggtgctcattacctctttgccacgagcaatgagtgggtcaggaagcccagcctgggccgctgtgtgggaggcgtggctggcactggcgacgccttggcaaagctggtagaagaagacaaggtcttccccatcctcacaagtctccatggtctgtaaagagaacaaacaaggaccctgtgccttcagtgccctggggctgagaagagggaaaggccatgggcagagtgacttcctcaccaaatactgcaccaggggtccttgcggcagcagctgcagctgaacaaggctcaggaagttggtggctacaaagacgtgggggcagctgggtcccagtgctagccagtgacggagcacagcagccagaagtgccaggccatccacctgtcaaagggcagaggtgagagggcagagccaggacagctgtgtccttctgtagctgtcatggcttctcctcccctctgtgcacgcatccccctttcttccctgaggtcagtctcctgctcccgtttctcattttcctccatcccccctccttagcatctcttcattttcctccgcaccgagttggtccccttcccgaattcatcgatcaggaccagcgagtgctctgtggcattgttcactgctttcgccacctgctgggcatgaaggggacaagggagcttccttagcggtcagggctgtgatgtgtcatcatgaataaactccttacatgtgtctacccctaaaccgcaacctttttaatagcgatgctcttatatgcccggccttcccctgccagctgagaggtcacacaaaggagcctcaagaactgtggatggtgttgcagcctccagactctgacctcagtgctccctggcccccagtgcagatgcatgcattggaggaaatgtgttcctgcccaagacttccaggggcacagtttctacccggtcacccttcccccagcccctgctgctccctttaacctggttgagatcaatcatgaaggtggagaggccgagggagatggattcgcagctgtgaattcgagtgaagatggcgtcgattaccccaatctcggcctcctctgcaggcacgaaactgcccaccagggccatgaaagtgatcaagcctacctgggccagggcaggggagacagaacatgggcctgttgccgtactatgggcaaagtaaaaatgggtctcaactaaagacatgaacgacagattaaagagaggacacagtgtaacagaagaacagagccctggaacacagtttcctgggtcctgacttcacaattaactccagagtcaaggttgtgggtaaaggcatactagggaacggcatggatctatttggaggttggtccagggacttgtggccacggaaaggtctatcctaaagaacagactgtccctcagaggacaaggacatgtgctgtatgtgtgtgtgggggggggcagaggatctggcctggcggccagaggagatggagatgtgggcccccagcttggcggcccctcacctgcttgagatatatgcttttccctgaggagttgggcccagtgatgactttgaccctgccctggtccccaccacagtccgtggagttgggcacgaaggttcgtgcacacagttccatcagaggatgcctgtggtgggtagagagacactgcatggtttgtggacccagggaggatccccacagcaggacatggggccccttctcacctgccattcctgattcgtactccatggatacagggagagtaatgcggtctcgaatagccgtagtcccgggcagcactggcaagagccaacaggacgtccaggcgggaggcaaggtccaatacccgagtcaagaccgaagcccgtgccagcacctggcactgcagctggtacatcaacagggtctcctggtctggagaaggagagagagaggtgcctggaaagatctggaagcaggcatgcagatgcagtgtgaccatgcagaggggccagcatgagagcagggtctgttagagcagcctgccccctcctaccacgggctcctcaccccggatctcgcagtgcaggtctcccagcagcgtgtccagctccttggtccgggcgctacgatagtgcagcttgtcctctgagagaaactgggtgcaaggagtccaaactctgggcttgttgtctcttggtttttgttttgttttgttttgggacaggatttcattctatagtcctggctggcttgcaactcacagagacccacctgcctctgtctccagagtgctgggattaaagctgtggtcacaagccaggcagcagtagcgcacaccttcaatccccagcacttgggagtcacaggcaggcggactctgtgagtctgaggccagcctggtctacagagtgagttcccgccaccatcatgcccacaccaccacgcccacaccaccatgccccacaccaccacgcccacaccaccatgccccacaccaccacgcccacaccatcacacccagtggcttttcatcccttgggtcctctctcctctccacctgcctggacctcctccagcccctcacccccaactcctcttctgactcaggactggaaaagcttcttaagctacaaaaatgatcccacatcgtgttcagtgctgatgggttgagacccttcttccagtttcttatcacactcctcccaccaccgagaggtcaagacacttaccatgaagtccagcccctcaatctcaaagtcactagcttccaccatgaaaggcaagcggggaatggaaagaaggaagccaatctggggagagaacggagtcagagtgggcaggaacctactgaacaccctctggtccaagagagagcccagacaccactgggatcctccattcctctgtcagagactgaagtcaccaaagatattcatgtcactgggtttaaggacagagggtgcctgtcgcccactgactgccagggttaatttagctcatctggctgggtgtcacttcctcccttaccactccacatgagacctatggatgcattcatgagcagctgtcctgccctccagcgttgaaaggctttcatgcatcaccatgaaactcctggggacttcactgctcccctgcactcgctcaccagagggatgtagatgacactgcatgaggggatgcgagagtccaggttctccagctccttctgagcaacttcagtgaggaagctcggaagccctatcagccttcgcttctctaggtgtgtagagaattacatgcatataaaaacgaggtatgggaggattaagggttcaaggccaactttggccacatgaaactatttttttttaagaattcctgattatccaggcgtggtggttggtgcatgcctgcaatccaagccctccagagtgggagcaggaggatcaggagtttgaggctgtccttagtgatatactgagtttgaggctagcccatgctactggagactctaaaggaagagctgtcttctggctgcaggctgattcccatgtatgtgtctagctgcgcatggatcgaatgactgccccaggacccccaggcctaggacccacacgcccagcactcactggcatctatgtcagggtctatgttagggaggactgtgaagcgattttcagcaagactttcctcaaagtccacctgaggagacagattagacgtccagtttcttagcacccagcaccctctctctgttctgtcccccgttctccttgggccccagatccccctttaatcctcatcttcagacacttcctctcagcatgttgttttgtcaccaggtgaccccacatctcccatgcgcccccacctcccacaccacatactccaaaccccagaccctttctactcctccatcctgtcccactcctcctcctcctcctcctcctcctcctcctcctcttcctcctcctcgggtgtctggcttccgctcaccaccttcccgatgaggctggcaatgtgatgcaggtcgtcagagaactcctgggcaatgtcctgaaaaagctggatggactgtggcagagaacggcaggcatccctcaggccgagagcactgtacacagtctgtgggagagatgactgagaaggtgaccccacccaggtgacatggacagaacgagaaacacaaggaccaatggtgaacactgagaggttaccaaaggcaagaacacacacacacacacagagacacagataaaaaaacacatagagagtggggcatgatggtgcatgcctacaatttcagcacttgagtggcagaggcaggtggatctctgtgagttccaggacagcctggtctacagagtgagttccaggacagccagggctacacaggagactctgtctcaaaaaacaagcaaagagaaagaaagaaagagagagagagagagagagagagagagggagagagagagagagggagagagagagagagagagagagagagagagagagagaaaagaaagagggctggagagatggctcagtggttaagccatccaaaggtcctgagttcaaatcccagcaaccacatggtgactcacaaccatctgcaatgagagctgatgccctcttctggtgtgtctgagacagctacactgtactcgtataaataaaaatcttaaaaaaaaaaaaaaaagggggctggtgagatggctcagtgggttagagcacccgactgctcttccgaaggtccagagttcaaatcccagcaaccacatggtggctcacaaccatccgtaacgagatctgactccctcttctggtgtgtctgaagacagctacagtgtacttacatataataaataaataaatctaaaaaaaaaaaaaaaaggaagagaaaggaagaaaagaaagaaaggaaaaactgatggagagaacagattattattaaattaaacttctgcaagaggaggacaagaaaacaatcaggctgggcatggtggtgcacgcctttgatcccaacacttgggaggcagagacaggtggatctctgtgagttcgaggccagcctggtctacagagtgagttccaggacagccagggatacccagagaaaccctgtctcgaaaaaaacaaaaccaaaacagaaaaaagggttttagaagtcagggaggaagtatgttagaagaagcagagggaggccagtgtccctgagaactaattctccagggcaaggtcagtgactgggagctgatgtgaccacaggttaagctatggacagagcatagcaccagagggagtgtccttctgtagaagagctgcataaactgcacaagtcaagggctgggacgagcagcagactcagtagccccgataaagcagacagagttgaacaactgacaaagtccaagaatttcaaacaagtctggcggggcctgaggctgtttgggaagccttttcacagaaaaggaaaggaggtgggccttaaataaaaactccaaagtcagctgagaacagagagcaaatgaacagaggagggagacgcagtgacccgcactgatgtgccagaccacgttctagatttccttgtgagtcagagccctgtggggtgggcactacctccaccttcagctcacagatgagcagtctgaagcacaagaggcaatggcagcctcaactctcacagcaaattaatgaactggggcttgaatcaagggtgtagctcccttgttctcattgtcaccatgcaccactgctaagctgcaacccaacgtttactctgagataatatcacactaagttacccacacagctttcttttttttttttttttttttttttttttttttaaagatttatttatttattatatgtaagtacaccatagctgtcttcagacacaccagaagagggcgtcagatcttgttacagatggttgtgagccaccatgtggttgctgggatttgaactctggaccttgggaagagcagtcgggtggggtgctcttacccactgagccatctcaccagccccccccacacagctttctaactcactctgtagcccaggatagccttgtgcttcttttgctttgggggcacaaggtctcaattgggtgtgatgtcgaaagtctgtaatcccagcactctggaaactgaggcagatggatcacaaattcaagatcaactgagatgtcatagtgcatttaaggctagcctgtgctatattttgagatcctgtgtctaagcaaccaaacaagcaaatgtcagtgcttataaggcagagacaaaagggctgctacaagtttgaggctagcctactctacacagcaagttacaggaagtacattgctacacagtgagaccctgtctcaagctagacaataaaaatttttgttgttgttgtggtttttgtttgttcgagactgggtttctctgtagccctggctgtcctggaactcactctgtagaccaggctggcctcgaactcagaaatctgcctgcctctgcctcccaagtgctgggataaaggcatgtgccaccaccgcccagcgtctctatgaatgttaagccattagggtttacataaagagttctattccagatagggctacataatgaaaccctatctcaaaacaaacaaacaaacaaacaaacaaacacaaagttagtaaataaatgtggtggtttgaatgaaaatggtctccataggcccacaggttgtcgtcttgttggtagaagtgtgtcactgggggtgggctttgaggtttcagatactcaagccaggcccagtgtcactctctctccctgttgtgttaagagcttttactgccacttctaggaatttggcatttgtcactgggctcagacaatataagaaacagtgactgtactggctggttttgtgtgtcaacttgacacaggctggagttattagagaagggagcttcagttagggaaatgcctccatgagatccagctgtggggcattttcttagttggtgatcaaggagggagggcccacagtgggtggtgccatccctgggctagtagccccgggttctataagaaagcaagctgatcaagccaggggaagcaagccagtaagtaacatccctccatggcctctgcatcagctcctgcttcctggcctgcttgagttccagtcctgacttccttaggtgatgaacagcaatgtggaagtgtgagctgcataaaccctttcctccccaacttgcttctaagtcacgttgtttgtgcaggaatagaaaccctgactaagacagtgaccatggggctttgttaactcctttgcttaatgatcaccttgtgtgtttactacttctctccattactttgtttgtgctctaagaactgcccatgtgttttggatgtacctagaataatatcaaagcaggcctgagagaaataaacccgcttcagcctcagtgcaggctggagtcttgtaattttgtccgtttttcatttttcttttttcaatcctcactccctcccttttgagaccttgttgactgactgagctggcttggtcactgctgtctgaggactgggatgtggagttctaagttacatctctagtaccatgtctgactggatgccgccatgctcccaccacaataatggacagaacctttgaactgtaagcgaggcccatttaaatgtttttctttttttcttttaaaagttgctgtggtcatggtgtctcctcacagcaatagaacattgtctaaaacataaaatgaaaaatttaactttgctttataattatgtaaaaacatttgcagtcatatcaacccagacaagttaaacatggacacattgcacagacagaggcaaacactcctatatatcaaataaagctttacaaattaaaaagaagccaggttgagctgggtgtggttgcacatgcctttggtcccagcatgtaggaggcagacagatctctgtgaggtctgtgaagtcaaagacacagcctagtctacaaagagagagagccaggagagccagggctacacagagaaacattttctcaaaaaaccagatagataggtggatgggtgggtggtaggtggatagataggtgggtggatggatggacagatagacagactgactgatggatagatagatagatagatagataaagtaaataagtaaataaaaataaataaaaagctagccacggtggctcactcctttaatcccagtactcgggaggcagaggcaggtgggtctctgagtttgaggccaacctggtctacagagcgggttccaagatagctaggactacacagagaaaccttgtctctaaaacccctaaaataaataaataaaaagcaagcaggcaaggaagacaggtgagtggaccaacacggggtaggggcagacctcatggttctgatattgagtagcaacatcagtaccaattctgtgatggtcacaatgaagtgtttatggctagaataatctaatggttggagatgaagagattgttatgagtcaaagggttgaagctgaatgtgtagctaaaagaggagtaattgcactattcacctgcaaacatttttgctatttctgcaacacagagtttaaaggaaccatcagtgagtaaagtgtggcatggtgtcccaagagaagaaggtctagacacaggccacgaaggcttgacttgaacttctgatctaaaacgggaagcctgtaatcccagaactcaggcacaggggcagggagatactaagagtgaggctctcctaggctataccttgatgccaacaaaacagcggccttaccttgtagaggacctgccagtcactgaccttggtgtgggacaacttcatgcgtttcagaatcagctataagcatacaagagacagctatggtttgccatttgacatctgatgcccctcaaggcattatcctccaggtgggagagatggtctttcagaggaccaaggtttggttcccagcacccacaacaggaggttcacaaccacccataactctagctttagatgattcatcatcctgttttagactctagtcacccacactcacatgggtgttctccacacagagtcacctaatttctgttttgttttgttttgttttgtttgagaaggttccattaggtagccctgactgaactggaactcactatatagaccaggctggcttcaaactcacaggcctggctatagatgcatataattgaaaataaaataaatattgggctgggtgtggtggtgcatgcctttcatctcagcactccagaggcaggtggatctctgagttcaaggccagcctggtctatatagcaagttctagaatagccagggctacatagtgagaccctgtctcaaaaccaaacatccaggtgtggttgtacacagctttactcccagcactcaggaggcagaggcaggtagagctctgtgagttctaggccatacatgggtacatagtgagaccctaacccaaatcaaaaacaaaacccaaccaaatgaaattttaataaaataagctgtctaagtttttctataagccatcactcttattccccaccctgatctctgccctgggctcacaggcacattcttgatgtggctcaggagtcggtgcagcatctgggccatgtccaggttctgaggcatcaggaagaactgaatgacatccagtcgggaattgagttcccttagctcccgggttggacgtgtaaaccacagcctggagggttgggatgggaaggttacaagtcacttcttctctcatcttgactttgcttggggagcgtggtactgtatacctgcagcacacaatgggaaagaactaactggcaagtgcaggccccgtgtatataggctcgtttatatgtgcattttgaaaactgccggagtagtcaccatgactgtgtctatactgagaccatgtgccgctccgtgtggatatgtggtggagcatgtcagtgttttagagaggaacagtgtgttgtggtatttgtgtgccaacactgcagctctctgcagctgagtgggagatggtgagtcaatatgagtttgctatctgtgtgtagccgccacagtatctcagggaaacagcatagacaaaatatcatctttccctgctctttctgcataaatgcctgtaactaattttcatcctcacccccaccctccagactccttccatccttccttctttggcaaagtttagggctgtgtcaaggcttactcactacctaaataacaaactactgagacacacccccagcctgcaatttttttttttttttgagacagggtttcacatcattcagactgttctcaaagttgctatcaaggctgacctagaactcctgctcctctagcttctatttcctagaatttgctggaattacaggtgtttgccacaggcctctttccagcctgctgttctttgtgtgttgtatgtattgtacatatgtgaacacagatgtcatggcactggtggagataactctgtggagttggttctgtcctgcctttatgtgtgctcaggttgctaggcctgtgcaggaagcacctttacctgtggagttggtccatcagcccctagcctacaattccttctttttcttttcttatttccttaattctctcttgctttttgtaaaggtgtgtgtgtttgtgtgtgtgcgtgtatgcatgtgccatatgcataagatggaggacaacttgtatgacttggttttctgcttcttccatcatgagttctgggactcaaactcaggtcatcaggtttagtggcatgtgcctctattcactgagctatctcactacacgccaatctactcagcttacaattctattttgttttggttttggttgttttgagacaaggtctcattatgtagccttggctgacctggaactcatagagatctgcctgcctctgactcggatgggattaaacacatggtggccatgcccaattcatcttacaacactttttaaaaaaataattatttattttatgtatataagtacatcttagttgtcttcagacacaccagaagagggcatcagattccattacagatggttgtgagccaccatgtggttgctgggaattgaactcaggacctctggaagagcagtcagtgttcctaacagctgaggcatctctcttacaactctttttgttgttgttgttttgttttgtttttcaagacagggtttctttgtgtagccctggctgtcctagaactcactctgtagaccaggctggcctcaaactcagaaatccgcctgcctctgcctcccaagtgctgggattaaaggcatgccccaccaatccaggcttctcttacaactcttaataatggtcatcccttagtagagaaagctgatttgaccctcaactatgagagtaaatgttgacagggtgatgagaacacagggtctatctagtagaaaggacttacaaggagtttgaggacgagatcctgaaaattaaaatccaggtaaacagaggtgccatgatcagtttaattgtcaactagacacagtctagaactactctgagggattatcttaatcacattctttgatgtgagagaacccacccactataagcaacatcattccctgagcttgagaacctagactgaataaaaaggagaaagctcgttatccacagggatgtttgcagctgtgctgacaacataaaatgaagactatcctagctactccactaggttctagatgggcagttggtatttctagtactgctaattgaaagagtgttttgtagactgctataataggcctcagtactcagtgtgtatttgtttataaacccatgtctgaggccactcacctgagcagcttctgtccccacttacagcggcatctgttgaggattcctgtggggtatgcatagggagtgaactggttacaaggtaggctggtctggaggcccagatacagaacacaaaagcagccaaattagtgcctagctacaaattggattatcatgtgagggtaggaaaagggatggacacccagtcaggaaaatatataaacgggcatagccacaactcacaaaagcccatcacaatatctaaaactaaagcataacccagtgagaagctagacacttggagtcgcatccctaatagactccaaagccagaagaaccagataaaaactatatgaagcagttctaaaactcctcagtggacacacccatacaaagccagagactgaacacagtattgttaaggtcacagctgacaagcaaattacactatatggctagagtctatgaaggagcttgagtcaggtgggcacagagactgagatacctgggctggtgtggaaaggccatctggcaatgtcagtaagccagaagagagagagagagagcgcgcctgcgcatggctatagagtcaattgcttttctagcatgttttcagagtgtggggtgagcaacaggccacacctaccaaaaaggctgagcccctccttcagcccactggctactttgtacaccgaggggtgagactcactcttgaaaatctgtagaacgctgtcacagagaaatggaggagtctgaatttctatgcatcccacatgtggtatatgcatgcatgtacatatttggttttgagacagggtctctctatgtagccctggctatcctggaactcactatgtagacgaggctggtctcaaacgtatagaaatcccactgcctctgcctctcagtgctgggattaaaggtgagccaccacacttgcctgtgtgcacatatttgtatgcaggtgaacatgctcctgcacatatatgcagaggctttgttctctccatctcgttctcttttaagacaaggtctctcattgaacccggaattccccattttctttggctaagctggcagccagtaaaacccagcagtcctgtctccacccacgccagcaccaagactgcaggcacacaaacctcagtgagtggctttttatgcaaatgcttagaatccaaactcagatcctcaaacttgtgtagcaaaagcttttactcactaaactatgttcctatacatactgtaatggatttttttttttataataaagggagttgagaattaagaatagaaatgtgatttaagttaataataagcttcttatttcatgtctagcaacagtaatctcgatgtgtggtttattactcacttctttttttttttttttttaaagatttatttattattatatgtaagtacactgtagctgacttcagacacaccagaagagggcatcagatctcattacgggtggttgtgagccaccatgtggttactgggatttgaactcaggacctctggaagagcagtcagtgctcttaacccctgagccatctctccagtcccctattactcacttctgtttgacttagcttctccccttaggaaaggaaagaaaagccaggtatggtgtctcataccagtaacccaagcactcagaacgcagaggcaggcagggagtttgaggccagttggtctagatagtgggttccagaacagccagggttacatggagagaccctgtctcaaaaaagaaaggaggaaaaaagaaaaagtaaacaaaaggaaggaaggaaggaaggaaggaaggaaggaaagaaaagggtgggttgaagttgtcactcaagagcagggtacttgggctggagcgatggctcagtggttaagaacactgattgatcttccagaggtcctgaagttcaattcccagcaaccacatggtggctcacaaccatctgtaatgagatctgacgtcctcttctggtgtgtctgaagacagtgatggtatactcacatacatgaaataaataaatctttaaaaaaaatagggggggtttcttgtctagcaagcaccagtccctcagttaattcaaagttgcaaagaggaaggacggtgattcctctgtgccccttcctttcctttaccctcatccacacaatcacccttttctttccttcaagccccaggattaaagaagccagagcctcagtaggattatcagagccttaaactcgtcctttgtttctgcaggcagcagcttgtctccacctcacctgtaagtgtcttgatctatgctcaccagatgggtcctaagaacagaaaaagccgcaagaacggtgtctgaggcatgaggagtcactactaaactactcctctctcccaatacctctccaagcaatccaatctttctggttctcctgtgccagagcacacttcagccactgcccttcatctgtacacatacagacacacacacacactcacacagagaaatacacatgcacacaccctttcttcttagacctcccagtcctctttactttggttgaggtttggatgagtcacctacaatacaaacttcttgaatcccaggatagggacgccaacatcatagtcttccagttcaaccccaattcttcttcgactcaggaacttgagcagtcctccaagtgcccggacctgggaggggatcagcagtgaagttggatgaagcctagtgttccagaagctagacgacactactatcaggaaaaccgtgatttttacagggctgggcagtccagtcacaattctgccctgtggctgggagctcatgcctttccccagctgcatcctagccctcaccccatgacccagctcaccgtgaggacacagtcaaagggaataatggaggagaggaaaaggattttctcagtagcagtcatggagtctgagatgaaggagtagtttccggaaaggagacgctgtttgcttatctctggacctatttggggaggatggggagtgaaaggtctcataggtccattctcttgccccagtttaacaataagaacaaatctgaccatatggcttgtccaaggccatagaggtggttaaggaaagctaaagaaagaatcaaaacatacagatgtagtctcattctttcggttctttatgtaatgtagatgtctggctataagcacaacctgagagagacaagtgactcaggaggcaaaaggtcaagatggcagggattgtggatatatatggaactaatactaatactgcccatttcctgtatttcctgtcctttttgcaaacctggaagtttgcaaagtagggagtgccttatggaccatggttcccagaaacagtgccgtttcctttttaatgcaacaaaggactttggctccaacgttcaaaactgtttttaagagtatcatctctccattttcttccactcccccattcctcacatacctaaccccatagtttctcagtaagttgtactaaagatagtaaaggtgcaggagagatctcggggagactcatgatggggtgtgggaagcagcttccagataagagtacatgaggcaaggtttctgataataggagctcctttcagagaaaggacagaggccctgggatgggggctggtaacataaaaggaaggagctaccaaaatccacgcttggcagaagtatgatttcaggtccctttggctctctgtgctcctcggaggctgtggaggcaagaggccacagtcttgtacagttcaggcctgtcattgtcccatgctccctcccagccaaagatttagtagtctatttccatttccccttctctatgcaatccttaccaagcttccctagaaatcgagtcatagcctcatcctgtttggcacttgtgacaacagactgggggttgatttcatccagaacttgaagagagaaggaaggtgcctgatgagtcgaaggatatctccactgtgactaagaggaaagtggtggctctcttcctgccactgtctgttctcctcaagaatttatcatcttagccaggcatggtgacacatgcctttgaccccagcccttgggaggcaggggaatctccatgaaatcaaaatctgcctgatctacacaataggtttcaggataaaccagagccacatagtacatagtgagaccctgtctcaaaaaaaaaccaaaaccaaactaaagttattttatgtctgtaagggatggtctgcacactgggagcgcagtggaggagacttagtaaaggcacggtgggaaggaacaagagtgtcctggaaactatcactaaaatcatggtttgccagggacaaaatctgtagcatttcacatacagtaggtacctaataaagttgtgttgaataagtaattcctaaatcaggtgactataaactacatgaggatgtatgagtgaacacacgtcccaccccagaggagaggaatgcagggctctgtcgcacctctctggagaagctttaggctctcgtggtctggggcatctggcatgaagtggatagtggagtcactagtgtcatagtaagcaatgcccaggtatcccgagctccacagcacgcacagatggatctgtcccgcgaggaaggaaaggggatcgatatctgaatccctcggaagggcatggattggtcatcccttattattaatcatttaaccaaatctcggagataggcactcatggggagtttagtcaacgtgaaacggggtcggctcagcctctcccagccctcccaactccgcggacacctcggcgggctcctcctcgtcctcttcctcgatacctccaggccccgccattgggggctgagggctggggaagctggctgaggggattccggatctgggtccgggtcccggcggcgtccggcctggggtcgctctgaaagccatgggctcggcgactctgaggacatgggaggggaaggaggtccaaaagcagcattcagcaggcggctggtgcgcgggctttcaggggacaggcgcgggggctgcccctttaggttgggagcagctccgagctactgtcccaccctcattcctgtcactcgaagccgtgctttggaagctctgacggagcgcgctgtcacgtcaagtctctcccgccacccacgcccttacagccgcagcgcgctctcctgacccacccccaccccctcccccacccccaccccactggagccgccaattcgcagcactttcccgccttttccgtcacctcagccgcgacgatccacctccctgaccaaccgccgcaagcctgcagcctgtaggcgtgggccgcgcgcgctcgccccgcccccttagtggctgaaagggtctgcttagccatccaacgggaaggcggtgaggacgctagatgtcgattggtccgtttgcattgggggcggtgctagagtgttcaaaattcccgccctctgccgttgctaagcagccgtgtcgcagagctttgattgatgttgggcggagcccgccctaaactaaggtccccgcctggaaaaagggtctgggccacggtggagaaggccagagcccggaggccgaccttaaagggccagaggcagtcttcctccttcctgaagagtggagagaagcaacattaattcaacctgtgccaacagcaacaactccgttaatatttgtacccactaagttaatatttataaaaaactcagaacagtgcccagcaaacaaagtaagcgctacatgagtgttttaaataaataagtggaagtaagaactaactcctgtttcagacctactttgcctgtttcctgattgcaggagatgatcaccaaccacaggagcaacacggggcttgattattttggaacccctgaagaggaagtataagtatctgaaagacactaaattctgaaagtagacctaattagaacacatacaaccatactacagtcctctcacccctaaggacacagggtatctctgactgatgatccatctggatgggaaagaaggagaaggaaggagtttctgagggaaagaaaaaaaaccatactttacaaaacaataaacaaacaaacaaaaacttcagtgctggcttagcaggtaaaagtaccttcaattttgtacccagattgtcctctgtcctccatacctgtgctgcttgtacacacacactcacactcacactcacacaccacataattttttaaaaactgtcctagctacactaaactaagatcctgcctgaggttcagaaatggcatgaggtgacctggctctgctgatcagcgactggaaaaatcatctccaaaggggagcaggtctaagggctgggttaatggttactcttggcaaattattgagcaatggagttatggaaacctggaagaggttattggtgacagctctggggagtggcagagagggacacaccttccattctggggtatgaagagattagggactggtttaccaagaagaggaagggatcagacccaggaagagaaggcagcagaagaaaagaagagctggagaaagaagaagtgggagctcagagataagatggcagtctacattttttttttttttaatgcagtcctggcttcaaatccaggagcactgctgggtgatcccaggacagagcagcctggaaaataaaggcaagataacgcatttgtgtgtgcgtagcaattcaggcagccacagtcaatagtgatgatcagagcagctgcggccataaagacagccaagagaggcagccaaacctgtttgaatctaaagtttctcccagcgatctaactgtgagcagattgcccgctggccacttgctgaccagagggaaaaaaggaggtgtctgggaaataaggcttcatggaagccatctctcttatcagtgggaagaggctctggtgcagatctcaagggtcagcatagagggaacattcttaggactcagctggggctcacgcatgagtcagtctacaactgtctgcaacgctgtagttcaaaagcccaaacaccaagtatattctgccttcctggggaggagagagataagccagcccccccccccgccccccgcgccccagggaggagaaagctctcagaagaggaagcacaagaggaaaccactaactaactgctgttccagcaaacatgcatcaattcacctcagccctccctgcctctttcagtttccagcccagtctttcctctctgtctttccaagtccagaatattttcttcaaatatgtctgtatatgtgtgtgtgtgtgtgtgttagactgagaagaaaagaaagctagtcgatagtagagaaatttagagtgtggaggttagatattggcagacatattttaggtttttttgttttttgttttgtttttgttttttgttttttcgagacagggtttctctgtatagccctggctgtcctggaacttactttgtagaccaggctggcctcgaactcagaaatctgcctgcctctgcctcctgagtgctgggattaaaggtgtgcaccactatgcctggttttgtttttgtttttaaaaaaggtagaagtagtaggcagggcagtggtggcgcatgcctgtaatcccagcacctgggaggcagaggtagtcggatttctgaatttgaggccagcctggtctacagagtgagttccaggacagcctcaaccacacagagaaaccctgtcactaaaaaccaaaatacacaaacacacacacacacacacacacacacacacacacacacgagtagtagagctctcttcagtagcactcacactaaaattggaaagatacagagattagcatggcccctgcggaaggatgtcacgcaaatttgtaaggcattccatatttttatttgcctgtgctttatctaaaatcatttgtataggtgggtaattaaaagatactgaagtgaaaaaaaataggtagtaaagttctatctttatactaaagttagaccctccagaatcaggcctcccagggaaagtgacacccccccccccgtcttatttacttagatgtcccccaagactctgctcacactgacctgtctgacctttcctttttttcttcccgtgatagaaccagtcgctaccccacccaccccaggatgagcccagtgctatcctagaaccaggataagtctccaaggcaacattcaccattgagaggagggtaaggggcccgggttggggaaagtctaagcttggccaggcttccagaggaccgccccctgccgtgaagcctgggctcagcttcctgtttcccctgcatcaggcctcctggcctctggcggcctgtgtcttggagtgccctttgccaatctgtagtcctcagctcactatctccttcccgatccccctccacacacgcagagcacagtcccagcaaaccagaagactgactggataactgaaaacaaaacaaaacaaaacaaaacgcagaacccttgcatcctcaaactccctttctgcacctccgtggtcacttcgctagtgtcaagcttccctgtttgatagtggccacccttctcattaacgaagcgaggaaactgaggcacgaaaatgctgctgtgtttaggagaggtcagggaatcgaggaagcccccggacttcccttctaacaccttatccctgggatcccgagacctctgacttcagtcccggacccactcccacgatccccgtgtcccctcccctcatgtcccctccccaggtccagggcggggccggccggtgagtcagctgctccctgagctaaacaggagggaccgaggtggagcagctgggtgtggggtgcactgggcctgtggggaagccgtgttggagcgaactaaagggtctgatcggacttgggttgcgccgaagggacagccctgagtctggaggagagaatcccggaccgtgagacccgccttcccttgccccggcccctcccagttcccccagcgacggctgcttcctggtccccgacgcaaccatggctgaagaacaacctcaggtcgaactgttcgtgaaggtaagaacactttctctttctttggccactctcaggatttctgcaagtcagtcctcccctctcctgggctccagctcaaactccccaaccccttcctagttgaccccactcccagccccggtgctgttttgctgggggcagggtgggcgggcagacacgacttgtgagacggtaatgggagtgctagactcgccctgggaagtttatagaagaggtttccagcagcctaaaggggagaaaggtaaagtttcaggaatgagagagtgaatggggttcggtgcgggatccagggggatccagacaagagaaggaccgagaaagggacagtggtggggttcgagaaggtcaggagagttggctacgacgtgtggccctatgaaagaaagagaaaaatgaaagatggggcagagagaacgtggggagagagcgagaaagagggagggagagagggagaaaaagaggagaggagaggagaggagaggagaggagaggagaggagaggagaggagaggagaggagaggagaggagaggagaggagaggagaggagaggagagagagagagagagagagagagagagagagagagagagagaatgaataagaatctggtagggttgaaggatgagtgtgtcgcatggtggaaggaaagagccttcacgaagaagcagcacaatcagtggctgagatgactatcccccagctgaggcaggaaggagattaaagtttagcagtagttacttatgaagagaggagaggagagctccaaaggggacaggagactccttgggttagatgtaaatgccgggctgataccgggctggaagaaaaagccccctccccctgagccctccccctttacactatcctctctgtgggtgtggtcaaggcaagctggtgctaggcttttggcttttgggttcggaaaccgctggctgccgcgctcctcctctgcccaggcctgtcccctccggcaccacccttccccatacccccgtgcacgtcccttcctctccgggcaggtcctctcccagtgagcacattcgcactcgttttttgcttctcggcctctcttggcattcctttcgttcttcccgtctgtgtgtcggtgttctgtttttctctatcttttttttctgggttttctctggctgcctttcccagtttcttggcccagggaagtgaactggagcctgggtccgccccttaggtgtggtccccacctcacactcgcctccaggggctatttttaccctgggcgaggagatgctgtagagatcctggccctttgtcctgtgacgaggatcttagaggaccagaggacatgagtgagggtttggggaactgtaggagtggcggcagctgcctaggcctctgtgtgtataggtccctgcccccacttccttgccaggaaggaagctgctgaggcctgaagacacagaaatgaatcaccctcagtctagggggaggtgtcccttgggggatgaggtcacctgcatccaattagagagttgccccctttgttaggtcctccatatctctaagttgagattaggtcctctgcctgccaggcccagcttccagaaacagcttagatagaggcttagtgaatggggaccctagtttccaaaccctgccctacctctctgctccgctgaagatctagtattcaattccaaccccaccaagtcccctattctttgccttttgaatcctgattctcaaagctagccaagacttaactgttcctctgcctcctcccaactgtcaatcactgggatgccaccagcctacctttgctgatccagaaccaaaggagaaaataatcctgctctcggggtagcttagagattgtggcagtgtatgtcagaattagcaagaggttcagagagcagcctggtaaggatggggtgcgccaagcctaatgctgagtgtgttatttttttcaggctggcagtgatggtgccaagattgggaactgccccttctcccagagactgttcatggtgctctggctcaagggagtcaccttcaacgttaccactgtggacaccaagaggtgggcccacttctgaccctctaaacatcctttgtgtggatgcacgtgcactgcatgcgcatgcgcactctcgtgaacccgtcttccttaaaccggttctctctctcgtctgggacaaatctctccattctatgtccctgtttctgacacacacacacacacacacacacacacacacacactcacacacacacacacatcccttttcctttcttgacgtgtgacctcttctccagacggacagagactgtacagaagctctgccccggtgggcagctgccgttcttgctctatggcaccgaagtgcacacagacaccaacaagatcgaggaattcctggaggccatgctgtgccctcccaggtacagggacacttggaaagaaaaggggagctgggaggctttgcagaacaaaggtcgaagaggggagacagggaggcagggggaagtaccccagaaaggacttcactttatccctctgccttccaggtacccaaagctggctgccctgaaccctgagtccaacacctcgggactggacatatttgcaaagttttctgcctacatcaagaactcaaacccagccctcaatgacagtgagtcttatgggtcaggggcttaggtcctacagggttggggcatctgtgagctcggctgtcagggagatggagaggatgttttcagacccccgctgagcctccattaacctcctggctggctggctgtctcattgctaagacttttggagtttagggtttgttttggttttttgtttgtttgttttttggtttttttgagattttatttatttcttatatacatgagaagtatttgtctgtatatctctgcatcaagagcatgaagtgcctgaaggccaagagagagtattagagtccctgggattggagttacagatattggctgggaatcaaacttcaggtcctctgcaagaacagccaatgttctcaaccactgaatggtctctgcagtcccaagaccagctttttaattgggtactggcatttgaagctgagttcttatgtttatacatcacatgttgtacccacaaatcatctctcaaccgtctagagttcactcttttgttttgttttggttggtttttttgtttggttggttgtttttttttttttttttttttttcttttggctttttgagacagggtttctctgtgtagtccaggctgtcctggaactcactctgtagaccaggctgaccttgaactcagaaacccgcctgcctctgcctcccaagtgctgggattaaaggcgtgcgccaccacatccggctcctacaattcattcttaaagtgcatcctcatgtgtgcagcccacaccaccacgtgtactgcagcattgccaggctcacagacatgagagctcttttactattttgcatacatactttgctctttggtagaactggggaatgaccgattctgggaaggtctcggcatctctctaaatctgcttccagatctatagagtggggataacttccattttctcgatttgcgtgagaagtgaatggctgagacttgtttctagactgactgggaggctgagggagaggatgtgactcagtgtacagagcttgacttgtgtgcgaaatcctaggctccatttctagcgcttgtggggataaattgcaagccggcggctggggagatggatgtcacctgtgtgcttgctactaaagtctgagtaccgagtacttgcgtttggatgctcagtggtcaaaatctttgaatgatgggcacctctgtaatcccagtgctggggaagcagaggtaggagggccctgggctcaggcgatcagctaatagagtctgattgataagctctaggttcagggagagagaccttgcctcaaaaatacagtggggactggaaagatggttcactggttaaaagctcctgccgaggactgggttttggtttccagcatccacctgacagctcacactacctgtaactccagtttctgggggtctgaacctgtttctgaccgccaccaccaccaccaccacgcctgctaggatcctacatgtacaagatgtgtgtatacttctatggcctcgcacacatacacataaataaaaatatataaataaaccttagagagagagagagagagagagaggctggagagatggctcagcagttaagagcactgactgttcttccagaggtcctgagttcaattcccagcaactacatggtggctcacaaccatctgtaatgggatccgatgccctcttctggtgtgtctgaagacggcgacagtgtaatcatatacataaaataaataaataaatcttaaaaaaaggaaaaaaaaaagaaaggaaggaaggaaggaaggaaggaaggaaggaaggaaggaaggaaggaagatggagaaccattgaggaagacaatggatattgacctctgacccacacacacacacacacacacacacacatgtaaacacctgcacacatatgtcacacaaacatttaataatagcattcacaatgaagttgttttgtttgtttttcttaatcaaatccagggtctcacacatgctaagcaggctctttctctctgagctatgccctcacccctccacagctcttcttgggcaggttatttaaggttctgatcagcttcctctcttgcacagacctagagaagggactcctgaaagccctgaaggttctagacaattacctgacatcccccctcccagaagaagtggatgaaaccagcgccgaagatgagggcatctctcagaggaagtttctggacggcaatgagctcaccctggctgactgcaacctgctgccaaagcttcacatagtacaggttggtggtcaccgtggggggaaacgggatcaatctctaaagtgattttttttttttttttgagacagggcttctctgtgtagtcctagctgtcctggaactcactctgtagaccaggctggcctcgaactcagaaatccgcctgcctctgcctcccgagtgctgggattaaaggtgtgagccaccacgcccggcggtgattttttttttaatcaccttttatttattacaaagggcaaaacacaaacggggtttggaaatcaggagaaaactggggggagtcagttttctcccctccaccacgtggatcccaaagattgagccttggtcacctgacttggctgcagatgtctttactctgctgagcagtcttaccagtccagtgttgctgtccatcatgcctaggcttttacgtggatgctgagcatccgaactcaggcacgcatacttgcacagcaagcatccactttctccgaatggtttccatagattgctaccagtgatgggcccctgcccagggaccacacccttagcaattgttgcccaagtaagagaattacatcccatgacacaagccatgcttatacagcatccattaaagagtggcattcagttttactgtatgtctgtgttttatctgtgtacatgtgtgtctgtatgtgcctgtgtatctgtgtgcatatgtttgcctgtgtgtatgggtgtctgtatctgtgtgtgcctgtgtgtatgtgtgtgcctgggatgtgggggagggtgcgcctgtgtgtatgtgtgtggctatgtgtctgtgtgtatgtgtgtgcctgcctgtttgtatccagggttgcttccaagaaggcttttcctgcaactgtatcactgccaaatgtacaaatcaaacaaaggattctcccaggtctatgatgtaactcttcacatagtccaggcttcccaggttctctcaaggactcttcctgagttgctgtttcctttctgtcctggccttaaagttacagtgtaatttagtaaaacacagagccactatcactccaccatcaactgagaggcaccagacagtaagaaagctgggcagagcgtctcctctcctgcctgtaatcctcagcgtgctgcaggctgaagtagtaagttcagtgcgagttggaggccagccttagctacagaataagagcctggttcaaacaccaccactaccaccactaccccacacacacaaagaaaaagaaaaaagagaaagcagccaggcagtggtggcgcacgcctttaatcccagcactcaggaggcagaggcaggtggatttctgagtttgaggccagcctgatctacaaagtgagttacaggacagccagggctacacagagaaaccctgtcttggaaaaccaaaaaaaaaaaaaaaaaaaaaaaaaaaaaaaaaaaaaaaagaaaagaaaagaaaagaaagcaacgagaaaagaagcaaatgatgttcatttagttcttgcccacaaacagttgaaattgaattgaagagaaagcattcacatcaagggatgtgatggcttgcagagatggctcagtggttaaaagcaccggctgctcttccagaggatctgggttcaattcccagcacccacatggcagctcccaactgtctataactccagttctagagaatctgacatcctcactcaggcatacatgcagtcaaacaccaatgcacaaaatataaaacaaacaaacaattaaaaaaacccatccacatcaaggtactaaaagatgacctgaaagaagtcaagtgaaaaggagacaaagttctctacaaaaatgtttactctttgccaactctctctctctctctctctctctctctctctctgtgtgtgtgtgtgtgtgtgtgtgtgtgtgtgtgtgtgtgtttactgataatggagcccaggactaaccacaaccctagcctgtgtttgtctctgcctctgtatatattcagttttaatcacacgtgagtgcaggtgcctttggagtgcagaaggcggtggtagatcccctggaactggagctgcccactgagggcgctggaaaccaaacatctacagcaggtgttcaccactgagccatttcttagcctgtctctctctttatctgtttcttgagacagggtctttctatttagttttagtgaccatggaacttgctagtgagttctaggactgccaggtttacacagagaaaccccatcttgtgatgtgtgtgtggggggggagggggtagtgaggatggggatggaagggaaagaatcggtgagaagtctctagaggggctggctcaggggttaagagcactgactgctcttcttgaggaccatgttcagttgccagcacccacatggcacctaacagcatctgtaactcagaccagaggatctgatgcacagtcatacaaataaacaaacgcccatcctcataaaataaaaataagtctagccagcatggtggtgcacgcctttaatcccagcccttaggaggtagagatgggtgcattccagagtttgagaccagcctggtctacagagcaagttccaggacagccagggctacacagagaaaccctgtcttgaaaagcctaaataaataacatctaaaagtacaatttttaaagactctaggcacagggtacatgtctgtaataccattccagagatcatccagcctgcgtgacacagagagaccctatctcaaaagaacggcagcaaaaaagcttgtaagaatcgaagttcattggtctggcatcctggacagggctcggttttttgtttgttttgttttgttttttgtttttgttttttacctggcagaccctaaaggaagagaaagttcaacagcttcaaagcaaagcaagcatgccttccctggggctaacacactgacctctctctaacctttaggtggtgtgcaaaaagtacagaggcttcaccatcccagaggcattccgtggagtgcatcggtacttgagcaacgcttatgcccgggaagaatttgcctccacctgtccagatgatgaagagatagagctagcctatgagcaagtggccagggctctcaaatgagcactttttaaagggtccttcacccactctgtgctctccacagaggcttcggggggcttccacgttgctgcataatggacacactccgaaattgccagtgggcaggaaatccggggcacttgtggggtggggctgtgggataaggcaaaggatagtggacaagggtaagatttttattttgggatggggtgggtaggacaacgcatttcagtaataaaatacagaatggaaaaaaatctagtgttgtttttacacaaagacggcttaaagtgtagtgggctccaaatgaagagaagggtcagtgatcttttaagctcagtgtaactgctgggaatttgggaggggagtgtttgtacaggggctgccatgttttgtatctgtccataagagggctgtttcgtggtccacatcgcgattgtaaccccacagccagggctagcaggatcagggtgggcctggacactgctctgggggcggtgcctgagttcagggaaggggggcactcagccctggaggaatgggcgcggcctagcagggcggaatgggcgtggtcttagaaggtcccgccccgtcccgcttagacaatgccccggagccgccagaccatcgcgcccccgccccatcgtcgtacgtgagctcgcctaccctgggaacccctcaaaagcccgagctatccgtctctccagcttgaggactcagactcccatcttcattaactctctcctctgggggtggggccaaggctgaaatcacaaggctgcaggagtggaggtggccgctggtgagtctgaagagggggctgtctccgaaacggtatggggagactggagtttgggggtgctgcatacgtccctgcaccggacggagtgtgcgaataacctgcacttgggcgaagagctgggcggggtagactttagaagatggacaggggtacggagttctgaagcctggagtggctccacgccgcgcccctcccccgtttctccgccctccagatttcgtttaagacctctcccagtcctccgggactaagtctggtttgtactgggtcgctcgaagggcagcgtgacgacccggggtggagagagaggatgcttaactccttagattctagttcctcgggtttgcgcccgtttccctttctccttcgagcatacgggacttcacaccccaggtcctgattcaccacccagttttcttttccagtcctataacctctgcgggtccctggcaggactttgggtccctcctgctgcgagcagctgagggttaagggggcggggccgccgcatttttggggagtgagcgcatcctagcggctgccaggaggggcgcggggcagggacctagcggagccccgagcaggggcaacaggtgtttgggagattaaggatccaagtcttgttcccttgggcgcctcttaaagcagatccccggtgaaaggaggctgagcggggtccacagagccagaaggtttggagggggtggggcagagtgggaggggcgcctggagatggcaggaggaagacctaggagcttttaacactcaccccttctcttctttgcatgtctttctcttctacccccacccctttctgtttcttctcttagaccagtgaagtcaagagaaaaaacaaaacagcttggctggaaggggcttcggtgtggatgtgatggggacgccgggggaggggctgggtcgttgttcccatgccctgatccggggtgtccccgagagcttggcatccggggaaggtgctggcgctggtcttccggctctggacctggctaaagctcaaagggagcatggagtactaggaggtaaactgaggcaacgactagggctgcagctgcttgaactgcctcctgaggagtcactgccgctgggaccactgcttggtgacacggctgtgatccaaggagacacggccctaatcacaaggccctggagcccagcacgtaggcctgaggtgaggacccgggtgcgggtgtgttggagaacaggattgcgcggggtggggtggaagaaggtcgaggggaggaggctggatgtctgggattggcagaaaatagggagtgaggatgtctctgcctgctctctcttctccaaggttgatggagtgcgtaaagccctccaggacttgggactccgaattgtggagatgggggatgagaatgcgacgctggacggcaccgacgtcctcttcaccggtgaggcaacggatggtcagcacctatgtgggcttggcaagggaagcagagtttgggagactaggagatctcaggtcttgactccaaactcactctggccttcaaaccttcgcatcctcccgggattcaggccgggagtttttcgtaggcctctccaagtggaccaatcatcgaggagctgagatcgtggcagacacgttccgggtgaggaaccagagaagtttggagaatgagtgcggccaatatgccagagggcttgacctgtgctccttacgcacccctaaatcctcctgctcttctaggacttcgctgtctcaacggtaccggtctcaggctcctcgcacctgcgcggcctctgtggcatggggggacctcgcaccgtggtggctggaagcagcgaggctgcccaaaaagcagtcagggtgaggcggggcagggctatggtgaaggcgtggtcaacaaagtgggcgtggtttagggcacagaagttgcctagctctgggaattgcccacaacccttgtcttcactaagatgttcagctaatacaaagggacaccctagtaatgatggggtacagaagaaacaagagtaggggaggggttgcaaaaggcagcggtagctaaagtgcctgagacgactgtgtctgtcacagcaggcattatatacttgctgtcattatctcacaaaagtaacccttaccccaagatcttactttgtggtaggtcaggcatactcagctgtggagacctcaatgccctcttcctctctccaggcaatggcagcgctgactgatcacccctacgcctctctgaccctcccagatgatgcagctagtgactgtctctttctgcgtcctgggttgcctggtgccacacctttcctcctgcaccgcggaggtggggacctgcccaacagccaggaggtgagggagggaaaaaccgccccaccaaccagagaaagcagtctgtctttcccagtgggagaaaggaggggcccatcctcagaagcctgggtgggacccactgggaactggctagaggggggttgtggctctgttatcaaaacatccttggggtcctccctgcaggctctgcagaagctctctgatgtcaccctggtacctgtgtcctgctcagaactggagaaggctggagctggcctcagctccctctgcctggtgctcagcacacgcccccactgctgagggcctggatttggggatcccactggttaggaatagagctgtatagtgggtagaatcagctaatagaggctgggtagttgtggggagatgccccaggatagggaaggacttagtgtggaaaagaatagaagccattgggtgagtcctctctgtcaaaaccaataaaataaaattgaccttttagataagggctgtgactggtttctcgatgggacaaagggataggatggggctgaaatccaaaactggggtcggtgctagagagacagattctttagcttttttttttttttttttttttttttttggttttttgagacagggtttctctgtgtagccctggctgtcctggaactcagtttgtagaccaggctggcctcaaactcagaaatccgcctacctttgcctcccaagtgctgggattaaaggcgtgcgccaccacacccggctgattctttaacttttgttaaagacaatgtttgtgtagctgtcctggagctcactctgtataccaggctctcctcaaactcaaattcacctgtctctgtcttctgagtgctaggattaaaagcttgagccaccaccgcccagctggactctttcattttaaaaaccctctttatgggcttcctgactcacccctaccgtgccacaggactcaggtacaaagagagacaggagaccgtgtctgttgtggggaatgcaggcagggtggaaagggaattatgctgtgtgcatgggtggatcactttctgaatttacctaaatgtcaggtgactcaggctcaggattcaaagtctgcctttatttgataaagaagaaagggatgttttccaggacgctcattcctggtccaaggctgaactcatacttcctgatattttcttataaactgacagacgtgttgtaatggggtgatttatttgctggagtctggttctataaggaaaaaccagggggtttctagagcatcaaattttgtgcagaagtaggcaagcatttgggatgaggctatctcagaaaaagctacatttgaatgaaatcctggggggcaattatccaggtgggaagggaggaagaggaaaaaggaatccagaaaacagaatttatttcaaagagccaacgagtattgtagggagaacattgtggagaaactctctgagggcgtgtgcacacagaaaatgatggataggcctaaaaaggggcccagtgctggagcctgaacacagagcccacgctcacagccccagagctgaaaccttgggaaagaaccagtggttgatcctgggaatcttgctggctttgagtaggaagagcagggcacgaggatcagaggggtctgtgggtcatcatgaagcaaatgatgacagtacattctggtggcagcattggacatggagaaaggtacagaggacagtgacctgctacagacaaggccttgtgtggttggtggtgcatgcttgcaatcacttttatccaaacactgaaggtagacataaggagagggagatttcaaagtcagcctcagccataagagagcctgtctttaaaaggagtggaagcggcgggttagggtcctgataagtccttgtgtgggagaggagatggtgtactctgaccccagtttctggcttggacaacttgggttcttcaagagctttcagaggaggacccaggggatggcttaagaaatgactgagtcaggtttgacaagccttgggcaaaatgcctgaggccatctggacagagtgaactaggtctgagatctggcttggaaggactactgtgtatcatttgggggctgaagattgattggccagcttcctagaagagtacggaggagaaacaaagagatgagcagaacagtgctactacaaagacaatagcaacaaagtaaaccctggtttaaacaggggtgaatgttatctggtggtaggacatcactggcaacctttcagtggatgagcagcagcagcagccaatgagcagggggagtgcagtcagaccggtaacgaggcaggtctatgtctaaagtttgaaatgcttcattgggccaggcagtgcctttaaactcagaagttgggaggcagaagcagatgaattctctgagttcaaggccagcttggtctatggtctacggagttccaagacagccaaggctatacagagaaatcccagtcttgggggggggcgcgaagaaaaagcttaacaggcagcttgatcatctttagagagggcagcagtggagaaggaaagatgttacagaagatggaagtgacagaaaaaaagttgaccattggcagaacgcaaaccaggtagtcagtcgcctctatcagtgcctctgtccatggtgcctgccccttctagctgaggttatacaggggagccccagatagaaagggttggcagaacaagggcttccctttatactacaaccgcatagacagtggaggcatcaccagaaaccactgtggacatccggcggtgcctcccgaggacagagtggtccagatcagcgtagtgcaggctctggaggaggtagagagaggacggtagtatggtctcatcaatctgaccccttagcttggaatcaaagctttttttcttttccccactgtgggtaaaggcgactatgtagcaaagcctggctttcatcttcctgcttaaacctccagagtgctgggattacactgctgctccacagcaacctagctaggctgctttcttggccctctttcctccgggattctggcctttttcccatccccaggcccttaccggctcctggtccaggtggcctgagatcttggactcctgttctaaaggcctctgtggctcagcatttatgactggagctatgtgggagggagggacagaactgaagggtgaagagaaaatgaccaactttgtctgctcctcgcaaggactcagactcaagaactcagggttagggattgtgcgggtaggatagggaaattgagtgcagttaatctcaccaaatgtgggtagtggtccaggcgggggcgggggtgggggcggaggcgagagtctgtaaggaagtccttgaagagtcaccaatctcgtcttactggttcctcggccgcaggcggcgccaataagaactgtgcaaactgcagtctccgaccagcaaaacaacttaggctcctctggaaccaggtcccgcccactctctgggctttctgacctttcagtttggtttctgcagcctcggttccctactccagcctgtagcctagcacccaagggctgacctctcacattcttttaacaaacgccaggcctccagacacttctttttctttgtaaaggttagggctctgccccacctctttccccacgccctgactggtcacaccctgtttcaagccaggctccgcctccagtgctcacctgcgccgccgccagaccacgcccgcgactcccagtcccagcacaagcccaacgcccaggagcggaatcaggaccttgggatactcgtaccctagggagaccgcgaggcagggagagatgccgtggtcaggggtacagacaatagggttctactaactttaccgctctgtaagattccgccccgctttttgtcccgcgtgtgtgtccaaattaagtgcccctgtgcatctgggcctcgacctaccgtgggtagatcctgcaggccggcaacctgccttgtcccctaacacttgaagcactgtgcgactctcattctcgtggcgtcctttgcagaaaaaggttccagaatccccggcgctcagcagcagctctagccgcttgataccattgtccagggaacgcaggcggccagagaaatgctggagcacagttggggtccctctcgtcgaggcccacaagatcggacggcgccctttagacaagcccttgcatgctgggaaactaggcgcccaagcccagcgggtggggtcggagacccctatgcaggagagattcacccggtccccagggctgccctccaaagaaactaggaaaggtgggggagggagatagttagggctagtttatcaggcgctctctctgtctctctgtctctgtctctctcccacccccccgccccgccgcgtgcgcgcgcgcgcgcgcacacacacacacacacacacacacacacaaggtgatcctagttgtctcagtctttctttgcccatcacaacttcccagggatggcctaccctcgggattcccctggacctttgagagcaacaaaggcagcagcggcaggaccaaggccatggttgggatgtggtggtcagaggcgggagtgaatcagcagggagaccggagggaagcagaggggcagggatggggatcctgccggactgggggctcagataagagcgagtgtgaggccaccaatttctggatccctctaggaatcctcaagccaacatgcagtcttcgaactttgaaatgattttaaattttgtgcttgaaaccttttgcaaaggcgctatgcttttttgcgaacaactaccatggccccaaagtaccacaaatactgaagctcagcttcctgagttcgaatcctgagttcatccagccacacagtagctgggtgatgtgagcaagccacttaatccctgcctgttccctcatcaggaaaatggagctaaaagcatgtgagaaccaaatcaatacattgagagagtctaaagcaatagatgacatatgggagtactcagtgtatgacatggtcattattatctctatcttagttcctatctcacctctgtgtgcccatgatgttcctccctacccaatgggctcctccctacccaatgggctcctccctacccaagggactcctcccaaacccaagtccaagtggggtgagggcttccttggcatgttatacagcctcagccccgcctctctgacatcacagaaaccctccatgatgccataagcccttctttatcttgtggctgagcatcagttggaagactggctggggaatggatctacattaattcacctgtttcgtcaatttctgctggaatgtctgatcaatgtcctcccatccctaggcacccaaagttgctgaaacaactcagttactgtctgtttcctgggaactagcagaaactagtgttgtggctgtctaagtcacagaggcttttctgatgctctctccagggcagggggccctcagctgggggtcctgaatccaagatccaaaatggagttgcccactagtgacactttccccctcctatctccctctcctgtctccccttcctacccaggccctcttggaccctttccacctctagtctttctctttagtcagctgggaataaattgggagggaggtgcccacagagggcacaggcccggaagggaggagccattgggattataaattctgaaaagaccgctcaactctcagagctggagacctgggaatctgctgtcaactgctggggctgtggacattctcaggaccctcaccatgaaacacctcctgttgctcaccctgtctgccctactctactgctgggtctcaggtgagtgctgaggggccccaggccccatcccaatccaatacttcatatttccctctactcaggatcccaagaagtcccaaccctacatggcttagtctcgccaatgcctccaggatcagatatctctgagcatgatgggctctagttttcttactcctctccttcttttccttcctccttccatttcttgtattcttttttgggggggggtgggtcagagtctcctgctttgaactcctgatcctcctgcctctaccttctaatgctgggattacacatgtgtatcacacctaggctctttggtccattatcaaggacccaggtctccattgtcattttctcctagccctgtattggcctagcctactcttcttacctggcattatggcactaggtctctatacacctttcctgcactctataacacccagatgtctgtctagccagttccaaatgcacttctccaagattagcatacctgccttcactcacctactgccctgagctgagggtcctcatagaaaggattccatgccactcctctcagagcctcagcgatggcccttcctggctcgttccctgctccttcctgagttacacacctatatccctaccggggatgcaaagccttctgggtggacccagcgactgctacagccttcccaagaatcagaaactttccctgagccttctacagagaagatgctgcagcacttaccctaaagtgagatgcgtttccgggaggctctctaggctcctctacccctgggctgaccattcctgcgccgcactttgtgtgcttacatctggagttagccaacccgggaatcctggcgtgtcatccgctgcacccaccctctccatttctgttagatgccagtgatacgtctaccaggtgttagttttccttttttccatagagcaaacgccagaagagtagagatgattcattcagtaatctccccaatctgttggcagaggtccagtccacttctaaagctagtcaccggtgactttcttgcagccgtggtttcatgccatctctgaaggctctgtggactcctttaacttctttctccctatttcaagtctcaccctggaccccaacctctgctccatccctctttccacacatctcagccatcctcagctccgggtttttgttagtacaattcttgactcatctcaggcaggcttgggtggaggaaggtatttaggtttccacatagaacaaacacctccgcccagtgcccacaccactttgcactttcgccggattgctcacctgttctgtctctgaacagagcaggtgtgaggcactgactgcgcaatgcggcagttctccatcctaacccctaactcaactcatcctcgggtgactcagatgtcccattacctcaacccaacttgttgcctccccagctgatactcgatgtcactcctgctacaaagtccctgtgctgggctgtgtggatcgccagtcctgccgcctggagccgggccacaaatgcctgacaacaaacgtgtaccttggtaacaccccctcttctctgggaagggaaggccgggtgggagatgatctctttgtctgaagcccatccagggatagttcagagagttacaacagtgatgtcggatggccaaagatgattatgtgccaggcgctgacctgtgtctctactgtgttagtggatgcttgtgttgaccccaggaattggtcttaggagaaggggcatggctcatgctcgggaaggatgatgagactttgaggagagaagggagaaaccaagcagtttggggaagggagagtagccaatggacacaggcaagccagtgcccactgctgatcctttctccattggctcctgcctctagggaagatgtgggttttctctaacctgcgctgcggcacaccagaagagccttgtcgggaggtcttcaacgaaaccaaccataagctgggcctgaactacaacaccacctgctgtgacaaggataactgtaacagcccggctccacggcccacacccgcactggccctcatctccctcacctccttggctggcctcggcctctggttattgcattgagactagctccatggctacaatcttaccacctgctatagcctgagcctttctccctgtgtcctcagagctccagctttccagaatcttctctcctcccacccccttcttctgaagatcatgtccctagtcctataccatttatttcatgggactgtacctggagtggcctttctagccaccgctcctctccctcacttgtcaccttccactccattccacccacacacagacacacagacacacagacacaaagacacacacacacacacacacacacacacacacccagtcctttcccatttccttctagaacactctacctcctccactggccactgaaaggctcccctccttggacgcacactgctgtgcctctgggatctaagtctggaagaactcctgtcttgtctccagggagtgattccaaaaggcgctggcctcattgcatgggcctggcttaccagaccctctgcttgtccccttctatcttgagaaataaacatcagtgtctaataagcgttgtgtttattgttagaagatataagtgctaatctgggtacggtggctttcctctgtaatctcaacaactggggggcagaggcagtaatagttcaagaccatccttgactccatagcaaattcaaggtcagcatgaaccacaataagatcctgtctcagaaagaaagaggctggtatggccaggcagcgcaagtctttaatcccagcacctggggaggcaaaggcagacagatctctgagttctaggccatcctggtctacagagagagtttcaggacagccaagctatatagagaaactttgtttcaacaaaacaaaacaaaacaaaacaaaacaaaacaaaacaaaacaaaacaaaagaaacaagaaaagaaaaggaaagaaagaaagagaagggaggaagaatggaaggaaagaaggaagaggtgggcatggctctatgcccttttaatcctagcacttgggagacagataggtggtaggggaggaggatggggaggggaggagcaggaggtgggggagggactctctccatggaaatctcactccctgcacctcacccttatgcttgcctaagaggcctcctggggggggggagtttcactgaccaaatggaagagcaggttttgcaagcctttctcccttcccacacctgagggtggcacactggcaccagtgtttggagaaacagcccttatcctaaatttagacgacacccgccaagaagcaggtgcagcctagctagagggtgccccagaaaggaaatgtgtgtacagacagagctggctcttgtctccacctctgaggtgcctttctaactgggttattcaagcaactttcctttctcagttcgaaatactccttcctattgagagccaagttatgaaatacagaagggaaaccatggactctggataaccctccacccagccttccctccagggaccctcactgtgaaggagtctgcctagcagcaggtcccgggagccactgaggacagagtcaccattgttttgaggagtgtcgttggatcaggtccactgatggactgttgttaactgaactagaagccagataagtgcgttatcccttcagtcttctcaacagaaggcactcagaagctaggagcaatgacacatgccttttggagctggagacagaggcaggtggatctttgagttcaaggccagcctggtctacagagtgacttccaggatggccagggctacctagagaaaacttgtctcaaaaaaaaaaaaaaaaaaaaaaaaaaaaaaccaaacgaacatttcggtagtggtggctcacacctttaatcccagcaccttgcctcaaagaatgaaaaacaaatatttttacagtcctctctagaatagttcatcaattttaacggcacacaagggagtgtttgtcttcttaccttagatgtccagctccctcactcccttggtccttcagtccttaccctgccctcggtcccaccctccagctctacagtcctggcaagagccaggccagggtggtaacagctgcagccaagatgcacaagggggtcacggagcttgccacggcgctgttgcacagatctccaaaacagcagtttttctgggttgtgaaagtcacgtctcccactgactctatggccacttgattgcaatgatgcgtggccacgcaggctgggtagtgatggctcaaggtcgcggaggctgggagaaagaaggcactggggtgttaaactcctgactgcaggacttgccttccctgagagagagcagttacaaagtctaggggctcagaaaggatactctgtcctctcaaaacaaacaaacaaaacccacctctctgaaaccaagggaccagggccaacaggcggacctaaacagggatgctaggatgttccaggagccctggaccattcatgtctcagcctcaacacggggaaatcacagctaaatacaaaagggacagtttgtaagatagcccagcactgttacctaaatgaaaccaaagatgggcatactggtgcatgccagtaacagctattcaggaggctgtaacagcaatcatcaggattgccaggagttcaaggtcagtctgaccaacccattgagttaaaggcgaccctgggctataaaacgacaccctgtctcaatgcaacagtggatagagctgggagtgaaactcacttagtaaaatgcttgcctagtgtgcacaagtcctggattcaacccacaccaccaaaacacaccaaaataaacaggacatggggagctagagagatggctcagtggttaagagcattgactgctcttccagaggtcatgggttcaattcccagtcaccatatggtggctcacaaccatctgcaatggggacccgaagccctcttctggtgtgtttgaagacagccacagtgttcacatacatgaaataaataaataaaaccgggctggagagatggctcagtggttaagagctccaactactcttcagaaggtcgtgagttcaaatcccagcaaccacatgatggctcacattattaatgagatctgacgctctattctggggtgtctgaagacagctacagtgtacttacattaaataaacaaacaaacaggacgtggtaacacacgccatgggagaaaaggagaatcccaaggtttaggtcatccttggatttctagcaagttggaggtcagcctgggagatgtgagatgtgagaccatgtcttaaaaataagtaaacaatatagagctgaagagaagctcagtggttaagagtgcttgctgctcttccagggacctgagttctcacagcaggttcacccgtctaactcaagctccagaggatctgatacactcatgcatgtagcatacactcagacagacatacactcagacatacactcagacatacatacgtacatactcatatgtaaaaataaatctttcaaaaataatagtaaattggggctggggagacagctcagtggtacagagcacccaatgctctcccagaggacgtggttcagctcccagcatccttaatcgcccataattccagttctgagggctttgatcctctctcctgacctccacaggcagcaggtaggcctgtggtacacaaatctacacacaggagaatgttcatgcacataaaataaattttaaaatgtaataagattattttaagcatggtggctcatatctgcctttaatcttagcattcagaaagtagaagtgagcagatctttgtgagttcaagtccagcctggtctatgtagtgagttccaggccagccaagaatataataagccaggcagtagtggcgcacgcctttaatcccagcacttgggaggcagaggcaggcagatttctgagtttgaggccagcctggactacagagtgagttccaggacagccagggctacacagagaaaccctatatcagaaaaaaaaaaaagtataacgatgtcctgtttccaaccaaccaaccaaccaaccaaatcaaccaaaaatacaagccaaataaacaaaataattactacaaataaatacatagtcagtggggtgtctgctttgagcctaggccagaaaaaaaagatgtgtgttccacagaatctaagtcgaggttaattagtatggaactttcattttctgatcttggtaatgacatgggaatgttacaaccttgaagctgggcacaaatgggaactctgtacattccttttgtaattttcctgtcaaaccaaatcatttcaaataaaaatattacacacacacacacacacacacacacacacacacacacacacacagagcataccctttccagaacttagtcagtccctgacagttgaagtttggagtttggcttaacattttattttgcctctgatcttaggagacagcttcttccttagtgagggtctgtgtggagagcaaagacagggatgcgagagcctgaggagccttgggcttctcaaggtacagcaccaggagccagtgccctgcttcaacagtggtgccatctgcctagagccagattgaactgggaagaggggaaacccgcaggctggttggaagcagtgggaaggggcaggtgggcgaccaaggactcacgttgcttggcttgctccgagctgggttgaggtttgcggtccaggaagccgcagcgctcgccctcaccacaagtgatcactgtctgcttgcaggagttgctggggcctccaccgcagtcatagcaccgcgtgcggtgtcctggcaggaaagaagaggtactggatccggcccttggaggagtcctggcagagggaaggacaggcctgtgccagaggcaatctagcttcctccttacccagggcaacccctagcagggcgctgagcaggatcccgaccaactgggagttcatcacgttcgtttatgcctgtagtgcctctccctcccgcccacccccctctcaggctctactccaccagccttatatctcccttgccttgagctttatggggcaataaaaagcgaggaaagagtctcccccaacaagcaccagggccccaatggggatagggagggggcccccatgggtggctctgagcatcctctgaagccctaagccccaaactcccccttcgcagcagtagttccagaatagtggcccaagaccccccccccagggggccctaaagccctccagaaatccaaagggtcttcctttttcagttctccatctgagggttgttcttgttcatccacattccagatgcaatgcagaagtaggttatgaaaacccagccacctttgattataaccaacattaaacggattctcagaaatgtaaaacaatgccattctcccgtctgggtcattgagggtaattattatttttcactaagctatgtcttcctaacataaatattatttttatttgttaaatgaatgtaaccatacttaattatttatttagctagctgagtgttgctggttttgagatgaggctggctttgaactcctgatctgcctgcctcagagtctcaagtgatgggattacatgcatgaatcaccaccacgcctggcttcgataccaagtttaaatgtcctgtttattttattttattgtgtgtgtgtgtgtgtatacacatatatatatacacacacatatagtgtgtgtgtgtgtatgtgtgtgtgtttgcttgttgaggcatagcctaggctagcctgctgcttagcatgtagttcaggctagcctcaaatttctgacaatcctcctgtctcagtctcctatgtgctgggattatagggcacatgccatcacacctggcttcttattttaatttttttcttaaaatgatttattgtatgtgaatgaacattttgcctatatgtaatgtgcctcacgtgtgttgctggtgccagtggaagccagaagcagcagcacccaatgcccacccaatgccctgggactggagttaacagttggttgtaaaccaacatgtgggtgctgggaatcaaactaaggtcctatgcaagaacaacaagtgctctaaactgttgagccatctttccatcccccttactctcttttacagtagaatttatttagggctaaaaagatagatatctcagtagataagaatacttgctgcttttttggaagaacagcttcccttcctaatatccatgttgaacagtagttaccatggctccagaggatccaatctctctctctctttctttctctctctctctctctccctctctctctcactctctctctctctctctcacacacacacacacacacacacattagagagagagaactaaaaataaaaagtcttttaaaatagccctgtccagaggcaggtggatctttgtgaattcaaggccaacctggtttacatagtgagttcccggccagtcaagaaggcccaaaaaataagttgataaaaatgtattatttgtttccttgcgagtgcaggtaaatgcatgcatgctgtcgcgcacttgtggggaccagaggatggctctgtgcagctggctctctcttccacctttatgtgggtcccagggctcaaactcgggccctcagcttataccacaggcacctttacctactgtactatctcaggagctcgctttaattgctgatatggtaaatattgatagaattcatacaaaggaaagctctgtggggcctccagtcattttcagggtacaggtgctcagatgcccactaccctcttccttgctgtgaccctctcggtgaaatgtttcttttgttggtactgggactggaacccagggctacccaggagttcgataaaccctctaagactccctgtccccacccagtctcttaatccctaacctcctttacagccacagttccatcctggaaaccaattccttcttagaggaaggacatacatccttccagcccagcccctgattaattctttcctgcaaggtagtaaagccaggagggataattatggggtagcactgttgaagcttggggttaggggagtgaatcaaaggcatgtgagccaggcggggtagggcccccagcagctgggcttggtgggtggggtggggtgtgggaatgggtgggtgggctttgttgataccacactcaaacaggactctgacctctagggcaggggagggttcagagtgtgaccttgagaggctctgacgggaaacagacaaaacaaaaagcaaaacaaaacaaaacaagaaaagaagaagaagaaaaaaaacaaaaaacaaaaaccccaactagaaccgggaacagcttgagcgggagggagggaggaggcaggagggaaggcggcagacacaactctagagagacagacagctgggctgagttaacaaacagggagaaaacagaagaccctcaaggcaagcatgggcaggagtgagagcgcctcaggtgtgggaacatagggctgttgcatgaggcaggccaatctctcgcttctccacaccccagattctctccctctgtctgtttgctggttcctccctgtcttgggcaggtgtgtctgactcggggtactggtttgtgctctcccagactgctgagctgtacctgccatgggcccgtccagcgccttccttggcgtcctgttcctctcggggacactgggtaagggtagactggggaatccacagggagtgggggtggggaggcgggaagggctgggacgggtggggtggggggtgggggcttgtgcaagggctgagaatggaaaggaagctgacagaggtgaagagaaatggggagatttgaggacattgggagtctgaggcaggaggatctggactcccaggctagtctgggctatgtagagctatgtctcagaaggccagagggagggggagcatttgagaagagctctctgttcccaccacttgtctctcacatccagctctgtgctcagtgcctcagtcaacgccaacccctgtttcctcctgtctttggcaccatctacttcacttttgggtctccctgtcctgctcatgtggctttttgctgacttgtttctcttggcactttctgctcctgtctgtctgtcctggtcactgtgtccctcttgtttctgcctgattctctgcatcgtctttcctttcgtggctattttatattcctgtctgctccttctgttttatggctctctccctcctttgtgtgtcagccctcagtgagtcccagaggtctgggctttgcctctttaccttcttttccttcctgccttgatctctgctgacccaacaggtctcaccacttccccagccagaggacggctccgctgttacacctgcagctttgccaagccctgcgaccctgttcccagggagtgtcgagaggacgaggtgtgtggtgtcagtgttggcacctcaggtaggactctggtgcgatggcccttctcgaggtggctcctctctttcctgcccatggctgccgccgcaacctcagcattcctcttttgtcccacagagcaaaaggaggaggaagtcattgagcggaaaggctgcctccccagagcccagtgccctctgctgggccatgcgacctactggtcacggtcctactctctccggcaccagtgctgtgagcaggacctatgcaatgctgccgcctcacagccaccccccaaccttcccctcatgactctgctaccccttgcagccatgatcggctggggagtacatgacttcctctagcccatgcgtccacggctgtcaacccaggattccagtgtcaccgtggacatgaaaacacctgcacagatgctgtggagacacgctcaggagcgtgactttgtataaaaacctcagtcaccgatcccagtacctctacagacccggttcctacagccagagttctgttccctgcatgcaagtggccgcaacctcacctcataagagacccccatatttaaacctagatgtcccaggtcagctaccctgcatgagctgtggggtcccaggcaaagggacagaagtgtcactggggctggtgagaaatagagttctgctagggggcatgtagcttggtacccgagggtctcaagaacagagggctaccttggggaaccataaagagtgtatttaataaaagggctattgggtagagatatagctcaatgatagagtgcttacctaccatacataaggccctgggttcaatcccctacaccataaataaataaataaataaataaataaataagctgtaaaagtagttaagagagctgtctgtgagcctggtaacagtgcacgtggtggtggtggtgaaacacacaagggtgcactttgagaatgaactacggtgggcgggcagaacaggtgtctccccgagccccagtatgtgctgtctacgcacaagagccttgtttcggatgtaggcacccattttaagagctcggtgattaagagcactggttgttcctgcagagaccctgaggttcagttctcagcacccacatgattcacacagatctgtaattccagtcccagaaaatcccaggccctttctggcctcctcaggcactgaacacagtgtagtccacagatgtacatgggggcaaaacaccagtacacataaaaattttaaaaaataaaattaaaaagccaggtatagccacacaccagtgcttgtaactctagggccaaatgaggggcagagaccatggatctgtggggattgctggccaccagggtagttccatatttagtgaccctgtctcaaaggaataaggacatcagatatcttctctatgtgcactggtatgtacatgtgtgcacttgtatgtatacaacacacacacacatatgcacacatacacacatgtacacacgcacacacacacacacacacacacacacacacacacacacacacacacacacacgaatggactatagctgcttggctggttgcgctgctgccacacttgaccctcatggtgctccccaaccccggtttccacctgtgtggctgtcatcctcttccctgccaacacctgggcttcaactgctgcaggttgaaggctgcaggcaagatccaagaagcctgctctctcctttgagagccaagcttgaaggaagagtcaaaaggggcacctggccaagcggcttgtgacgggtgtgcgtgcgtgtgtgtgtgtgtgtgtgtgtgtgtgtgtgtgtgtgtgtgtgtgtgtgtggtgagggggtgtggtctggatcccagctgggtttctttctggaggctagagattggttttgttgggagcatcgacaccctctttcacttcctctgttcggacagcctggaggtactcactccacttgggatgtatctgccaacccttgggtatgcttccgctacccagggagatagcaagagactggttggttcgagggtcagagctcacagtgagtcagaaatgataagccgctcgctcgtgctgatagagaaccacagaggatcatgtcagaggagcgtgtgtgctggataaggacagcgccccagtggggctgaggacaggcattcccatttgggcttggtttctgcctttccataagaggaagaagactggggcctgactaaagaggtcttgttccctctcagcttctctttgtgcgaagaacacggggaggggacgtcagatttactcggaagcctgttaagtcttctgttttctctgtctcccacatctgccatttacggctgcttttttccagactctgcatctgggtctgtcgacatctctctccctcattccaccccacacacagatgacagatgccccaccacttctcatgtctctgggctcgcctgtccctcctgccaacagatccagaccccataatcctccctcctcactcggagtcacactgatgtgctggctgctttgagacaggagaccacacctttttttgtaggtgacatcacagaacgatgttactgcttccttatgttgtccctggcccctccagcttggtggctcttggaagcccagtacgagtcacaccctcagccaccccttggccaaagttccagggaatccttcaggtgccagtggctagagacacaggactgtgggtcaggcttttccacgcagatatctccaggatcacctggtcttgtggttaggtgggctatagagtttgcaaaagcaaaaggctgtgttaaagctcctggactttctcaggaaccaggatccccatctgcaatccaaccccaggttccaggcaacaatctcccatgctcagcccctctgggatttaaggctacttacctaaggcgggccaagtggatgttctcatagacctggacttcaggcttgaaatgaggaactgagggttctggggaaagggagccatgtgatgtgaagtcaccagataattcaggggtctattccctgcctcccctcccctagagcagacagggtctagatgtgagcctaaccctttacttcacacccaagttacctctctgggaaaagactcacctctgtcccgagacccctgggccctccgccgcctccagagcactatgctgagggctatgatggtgaccccttggcctgctgtgaacaacagcaccagtatccagggcacgtcccagctcgggagagtggcacaaacagtaggggagggttccgcggaagctgctgtaacagcagaagaatgacaggcagtgcccccacacatgcaaatatgcatgcacacatgcaaatcacacacatggaaaatgagtttgcatttctccagttccttatgcattcctcatcccctaggccttgcggagaacaatggccgtttgctcctctctctgactgataaatgggttattctccatctggtagcgttcattctcaatataatggccttttattttgtctaacaattataagctttctgcttcaaccttccctatcaataggccattataccgcagtatatgtgaccccttctttggaagtcagcaccccagaaatccctatgtatttcctctctgccatagaaaaatccacatagaatagatctgtctgtgtctttcctccatttgttcttgagacagggtctcctggagtccagactggccttgaacttctgtgtcagccttccaagttctgggactgaaggtgtgtaccactatgcttggctcgtattctggtctatttcttgtctttttttggtggaggtagggagagtgaggggactagacctctctctatatccctggttggcctaaaattggctatgtagaccaggctggccttgaactcacactattgactgcctctgtctcctgagagctaggattataggcacaggcctatttcttttcttttggtgattttacttatttttagagaagcagggtcacatgtaatgcaggctggctttgaacgcactatatagccaaggctgtcctcaaactttttatcctccttctaattcccatgtgctaacagggatgtgccaccgtgcccagcctatttctttcttgtgtgtgtgcgagcatgtgcctgtgtgtgtgtgtgtgtgtgtatgtgtgtgtgtgtgttcacgtgagcgtggtggctagcagacaactgccagtgtttttcctcaggagatatccaccttatttttggaggtagatactggcctggagttcataaagcaggccaggctggctggctcgagagccctggggcccgtccgcctcgactctctagtgttgggactataagtctgcatcatcatgattggcgctttgtgtgtgtgtgtgtgtgtgtgtgtgtgtgtgtgtgtgtgtgtcgtgtctcctgagttcaaactcggattctcatgtttgtattacaagcatttttacttggctgagctctctcttcacccctagattaacctctttcttttttctttccttcctttttttttttttttttttttttttttggttttttgaaacagggtttctctgtatagtcctggctgtccaggaactcactctgtagaccaggctggcctcgaactcagaaatccacctacctctgcctcccaagtgctgggattaaaggcatgtgccaccacacctttctggtttggaaaactaggaagttggtttaagcgactattctttgtgatagatgtggtcaatgagggagatggggaagccttcccctctaggaaagttaccccctagttactggtgtgattatttgtcctctttcccaactttgagagaggggttggagagaattcctttccccattccctccctgattttacctgccaggctaaagctgaatcttttgttctgaggcaaaaggcagcggatattccttggcctacgggccctggtctcaggaagcccctctgtgggacacaccaagagcagggcagccccttctccccagaagtactgagcatgtcctctcacggtattccttccctctagccaggtcacagagtccagacgcctggcagggaccacagagcacaggagggcagcgcaagaggggccatctggagacttcacagagaactgggatcctggcaggagagagaggctcctcagtctcttgtgtgtttttcctgatctccagtatccagagaggtcttcagacccacctcacctttaagcacagagacgtcatacaccctccagttctggtacttgtggttctgatccatcacagtacaccagtaccgtccagcgtcttcatctctggacccctccagccataaggaataatttccgaagagtttgtacctggaatcgggttcgggtttccgaaggtctgagactggcttgtctacttggacctgagccaccaagatggtggaggagcctgctacagggctgcggaaccaggttaaaagctggcccccaagtagggagggaggtgagggacacggcatctccactgactccccggaggctacgtagatggtctggatgctgtctgtggggaaagagcagtgtcagagcagagctgtccaggccttcagcaagcccctacattagctccaactcctgtcttcctttccttctggggtctttgtttgtttgttttttgtttttgagatagggtttctctgtgtagccctggctgtcctggaactcactctgtagaccaggctggcctcgaactcccttctggggtcttcaagcactggtgagcacacatttctcaagtgactgttatgaagaaagggaggtgggctgagaatgtagttcagtcggtaaagtgcttgcccgaggctttgggtaggtggcagagcgcttgccttttgggcgtgacttgggtccccagcgtggcttaaacccataaactgaatgtgggggtgcagacctgtgatcctagcacccagagactggaagatcagaagtttggcgtcatccctcggctgctcaaggagaccctgggatacacaactaccagaaagagttggggaggggaggccaagagtggaaaggctttctgtctttccttgccagtgagaagctctgtcttggtcaggctgcaatcgttggtgacatcccttatctcatcccacatttcctcatctgtttttatttcctaaggggaacaaagccaggtagcacacaagtgatatgagcacttgggaagctgaggaggaggggttgcatcatataataaacatatttcatatctctataataaaatatataacagccaaggctgctataaagcaataccctgtctcaaaacaaacaaataaaaaaaaaaaaaaaaaaaaaaaagggaagctggaagctgggtgtggttgcacacctgcagtcacagagttcaaagacagatggaggaaggaatacttgacctgagagttcaagaccagctgggctactgtgacttttctcattgatttgacaaaaatgccaggctaaagcaacttaagagagttagatgaatatcatctctatcaccaccaccaccatcatcaccaccatcaccatcaccatcaacatcaccaccatcaccaccatcaccaccaccaccatcatcaccaccatcaccatcatcaccaccatcaccatcatcaccaccatcaccatcaccatcatcgccatcaccatcatcgccatcaccatcaccatcatctctctgtgcagcactggccatcctggaactcactccttatgtagaccaggctgtccttgaactaaaagatccacctgcctctgcctcctaaatgctgggatcaaaggtctgcatcaccactgccctactggaagtaagatctcttttggctcatggtttaaggccatcatagcaggaaatttacggtggttgaaacaggcttgcatttggatggacttccgagcagagaaggggaaatgcctatacctgactgactttgccttttccccgtttgtatttaacttatcaccccagccccctgggtggtgccacccacattcaaggttgcttttccctcctcagtgaaaactctggagatgccctcacagacatgcctaaagctatgtgcactagaagctatgcggatgacacccccatagcctcatagcaggaccctcatcttgtgtcctcaaattgaccacgtagttgaggatcacctggaccgcctgcctgatcctggggctatctcctccgaggtgctggggttacaggcatagcacgctgtggtcacaaggatcaaactcttgcctggagatgagctgagaactctacacgtgctgggcaagcactctgagctagatcccagactcgatgtttatctcttttaaaagagataaacaggattatatacaggaaactgtatataatctaggcccttggtacctctgggtctccctaggactaaacgcagagagggttcaagcaaggtcagtctgtcttcttgagtcacgtgcattttgagaagcactcagagaagcagcattggttcctcccagcctaagtagctgcagaatcccccacctcctgccccttacctgcaacagcctgggagtgcccacataagaacaggaggaatactacaactgccatggaagagcctgccaagttcccctgctccaaagatctggtgtttccagagaggaggaagaacacagataaaggcccacagttcctgggctgaaccacttggcctctggaagcatgagactgcagagagataaaactcagttgcatgtcatctggaggaaggggagcttccatctttgtgttcttggagtcaggtcccagcgcaggctgctctcgtgtgcaaggaatcccagatgccttcctcttcttcacggagaagcctcttgtagcttctcatctatccctgagacatcagccaagagccaaatgactctgcctgatctttggcacctggcacaaaactgtacatccaggccataagtgatctcaaacaactgttgctgtggttacctgacatatcagaggctgttccagagcccccaggagcatccttagctgtcactcaaggagaagcacagaggggacctcgattattcatgggtgcctgggctcagaagcaaatttttatacactccttctgattgtttataatgtgtgttatttaggtagttagtagctttttggcttttttttttttttgagacaggttttctttcttttttttttttaatagttaaatgcctcttttttttttaaagatttatttatttttattatatataagtacactgtagctgtcttcagacactccagaagagggagtcagatctcattccgaatggttgtgagccaccatgtggttgctgggatttgaacttcggaccttcggaagagcagtcgggtgctcttacccactgagccatctcaccagccagagacaggttttcaagttgaggtttcaaattgtccaggtcggcttcaaacccaccgtgtagttagagatgaccttgaattcttgatcatctgcctctcaaggtatgagatgatgggtgtgcatcattatgaacagatagctacttattttaaacataaagcaagttattggccgcggagggagttatattcccagccattaaactattttcttttgggctggtgagatggctccaagggtttttgcctttgccaacaagcttagcaaccagagctcaatcctcagaatccacgtggcgggaagaaagaactggctccccaaactgtcctctgacctccacatatgatgcgctggctagttttcaatgttaactttacacagaaagacaccgagggaagagcaaatcttaatccagaaaatgcctccgtaggatcagcctgtggcaagtctgcagggaattttcttggttggtgatgcaggagggttcggctcactgtggatggtggcacccctgggcacatcgtcctgaatggtataagaaagcacagagtgcttactcaagcccctgagcagcactcctccatggtctctgcatcagctcctgcctccaggatcctgccttacctgcgctcctgccttgactcccttcagtgacagactgtgatgtggcggtggaagatggagttaagcttttcctcgagttgcttcacgtcatggtgtttcatcacggcaatagaaaccctcactaaggcaggtgaggtgcatgcacctttaatcccagccctcgagaggcacaggcaggcggacctctatgagtttgaggccagcctggtggcacccctgggcacatctatgtatagtgagttccaggacagccggggctacaaagtgagattccatcttggaaaacaaacaaacaaaaaccaagagtcctaataaagtgtctaagttcgggttaatattgctttgatgaaacaccatgaccaaagcaacttggggaggaaatagtttattgggcttacactttcatatcatggatcaccagtaaacacaagatgggagctcacacagatcaggaacctggaggcagaagctgctacagaggccatggaagggtgctgcttaatggcttgccccttttcattgtaggtctgcattaagagtgaccactgacgggctggagagatggctcagcggttaagagcactgactgctcttccgaaggtcctgagttcaaatcccagcaaccacatggtggctcacaaccatccgtaatgagatctggcgccctcctctggtgtgtctgaagacagctacagtgtacttacatgtaataaataaataaataattaattaaaaaaaaaagagtgaccactgacaagcacatgacagaatcaatacgaggctgccttaaaatctcctctgccaatggcattaatccaaaactctcagacatgcaggctgtgtacatatatggatacacacacaaatgtaactttacaaaaggggaagggattcggtgtggtagtgatgcatgcctttactcctagcactttaagatttatttctacatatataagtactccatctgcttgtatatctgcatgccagaagaggggatcagatcgtaagccaccatgtggttgctgagaattgtatgtaggacatctcgaaaagcagccagagccctttaccaccaatcctagcactttgaacaaggtttctctatatagccctgagtggcctggaactcactacatacgtagactagggtggcctggaactcaagaggccaatctgtcaacacacccatctctctctgtctctcaagtgctgggattaaaagcatgtggcactacatcgggcagattacttctttcaaaaacttaaaaaaaaaaaaaaaagattcttgggttagagagatggctcagtggttaagagcactgactgctcttccagagatcctaagttcaagtcccagcagcaaccacatggtggctcacaaccatctgtaatgagatcagacgccctcttctggtgtgtctgaaaacagctacagtgtattcatatacataaaataaaaaaaaatttaaatagaattcttcagaagaatcctgctatttcttctaatggatacatccctagatctgtgtgtgtgtgtgtgtgtgtgtgtgtgtgtgtaacccagggctttcagcatgttaagcaaagttttctatacttgagctacaaatccagacactagcctcttttcttgaagttcttgtcaaatgctcctactctcacccagtgatcgttccagaacatgctgacattaagggatagggtttaaaaaaaaaaaaaaagtgaagccaggcgtggtagtgcacgcctttaatcccagcactggggaggcagaggcaggtagatttctgagttcgaggccagcctggtctacagagtgagttccaggacagccagggctacacagagaaaccctgtctcgaaaaaccaaaaaaaaaatgtgggggctggagaaatgctcagcggttaagagcattgactgcgcttccgaagatcatgagttcaaatcccagcaaccacatggtggttcacaaccatctgtaatgagatctgatgccctcttctggtgtgtctctagacagtaacagtgtactcacataataaataaagaaatctttaaaaaaaaaaaagtaaccacctggggcattcattttcttaagtttctctattcagaacagctctaactaactgtagaccgtcgcccactgcgctgaacactcctctcgtggcgagaacgtcaggctggaccctgccacggggcgagcggatacgtgctgacgtcacgcacgcacacaggcacgcacgcagcgcccaggagactacgggcggggccggcggggacctgaggccgagggacagcggcccgagccggggctatggcgaagctcctgagctgcgtcctcggcccccggctctacaagatctaccgggagagggacacggaccgggccgcgtccagcgtccccgagactcccactgccgtccccgccgcctcgtccagctcctgggtgagtcgggttccccccggcggagagcatggtatggtccagaccgcgctgcgctgcgctcgcccggcgcgcccactgtgggaaacgggggagcccgcttgtcaagtggcggtcgcgcacgcagtccgcctgcgggcgcttcccggccccctcccagtcacgcagggcccagccgccgcctctctttacggctttggaggaactcttcctcacacccaccaccacgcttgcttttttttttccccctccgcgtcccccgaatggctggtcccagttgagtttggagtcccctgcgcgcactggcttttgcccacctactcccaatttctcccaggatctgttgactttctctttaacctgacttttcagtttcaaaaatgacttcatgtcatgtatttacttgtgcgccttgtgcccccaaacctagcctgaaaaccagggtcttgcattcagggtggtagtttgctctccaaaatgtttttgtttttattggttgcaaattatgtttaggaggctttccgaagtgcccacacttagcgacatttttcccggaatcgacttacatcaaatgctattccttccctcttagaattgtcatggagtctctcattttcaattttactcctctccccatagtgacacaatcatatctgtatggagtttcaggggaaaaaaaggaccggttttccaaatgctgctttcacttttgcttccaagcctagcaccccgtgattgctttcagggtgaggggtgaaatgtcagggggggggagttgaagcttcagcagaaacaggagtatcccaggagaaatggccacagaggctagaaccaggtgacacaggttagggcttctggaagggaggaagggcctgtcagctggcaaggtccttaacaggccatctgttttggcaggatacgtattatcagcctcgggccctggagaaacatgcagacagcatcctggcactggtaagtcagtcccagcatctgccagtcaaacccagccctggagaaggcaacaccccaggaggtgaggggtgctctgggccctgtacagcagagcttgcacagaggaccagcgtatcccaagtgcctaccgttggcattctcccttaggaagtcccctttgtgtaccataagctttttccagagcagtttgagccctaaggataacaaatttacctttgggatcggctgcaggtctctgagtcttgcagtccctagtgacctatcctccctggcaactaacttctgtggctgcagagacagctgttctccctggcagagttcctatggtgatgttctcacatctcttggggacaaaacctgggactctcttgagtggggccaactgggaaggcttgattaacttgagtagacagagaggctggtgttttcaacgccatgaatctgtgtcagtctccgttactgtactctgacacactatgaggcaacaaatatttgggtccctactgtgtgcacggtgtcagagccaggcccggagagggcagaacagcgtgtgtgtgcacaggcgtattgatcgcctgtctgtgtctctgctgcaggcttcagtcttctggtccatctcttattactcctctcccttcgccttcttctacttatacaggaaaggtcagtgtggttccaaaggtggtggtggggcgtgtgtgggtggggacaaaccgggcaggtcattggccagtgaatctactccccgaagagaaagggggtgatcctgtgagaacggcatatttgacttccctcttctaccaggagtcttgccctataagctggtggttctcaaccttcccagtgctgcgaccctttaacacagttccacatgtcgtggtgacaccccccccccccccgcaaaaccataacatcatgtcgttgctactggataactgtgattttgctactgttatggttcatgatataaatattttttggagatacaggtttgccaaagggttcccaatgcacaggttgagaattgctggtctgatagaatggctaacccaaagacagagtgtgggtagaggttgtgggcaagctgggagaaccactcagaggtcctgtgagagcgtctcataactgtccctcttccctcccaggttacttgagtttgtccaaagtggtgccattttctcactatgccgggacattgctgctgctacttgcaggggtggcctgcctccgaggtaggtagagaggagcctggtttgtatctgtgactggataagcgttaagcactttttgcagacgtagtttggggctgggccctgtagggagtaactgaggacagtccaaggtgtagctcttcctcaagtgtcttacatcctacattgcatgtgttcatcagcagtgtacaggaacaagaagaatacagcacacaccgcctgagtctcagctggagacagcagcagactttaacatgcacagagacctcacacagtgtaaccacactgtgggccctgcaccggagtacagatcagaggtgcactgttagtggaagacattcgagcaatgtgtcctctatcccagcgcttgggtaagagggaggtagaaagtctcacgttccaggccaacctgggccacatagtgggactctgtctcaatcagtgaataagtgtgtaagtgaaggtcaaaacagaggggtgtgtgcgtgtgcgtgtgcgtgtgcgtgtgtgtgtgtgtgtggtgtgcgcgtgtgtgtgtggtgtgtgtgtgtggtgtgtgtgtggtgtgtgtgtgtggtgtgtgtgtgtgtgtgtgtgtgtgtgtgtgtgtgtgtgtggtgtgtggtgtgtgtgtgtgtgcgtggtgagacccaggacacagaggggtgtggttcgtgaccgcactttccctccctcttctcctcccagtattttcctctcccctccccatccccatccactcctcctccgtttcccatggatatcaaccagccttggcatatcaagttgcagtaagactaggcgcatcctctccttctgaggctagacagggcagcccagttaggggagagagatcccaagggggacagtgtagtcagagacagcccctgctcacactttaggagtctcacatgaagaccaagctgcacaactgtaacatatgtgcagaggggccaggcccaccccgggggcggtccctgaatttgcttatgtgtatcacatgtgcagattgcctcagaggccggtggacatcatagcctggaagctgagttgtcaggcaacaggtgtagacactaggaactgaacctgtgtcctccgcaagagccatctgcccagctccttgtttgtgtttctaactgtatgtcatctgtggacaccaagactgtaggtgtacccaccacaccctgctgcattacattgtgccatagatgtgagcacccatgtgaaacactctcgtcccgctcgcttccttctctcagtatgctctggacgttccgattgtttccctggcagcccttaccttcatctggtgtttgctggtttgtgaggggagtgacgagaagagaggcgtcagtcctgatgccagagttggcaccagagccagaggttaggagcctgcagaaagccagcttgggaacgcctgaggcagacaggaaggaaggccactgttagcaggcgggtggtaaggttgcagactgttttaacatgggtgaagatgatctgcaagataagagacttgctagttcacaaagaaagaaactgccaggccacccagcccttgggaggctgaagccgggagaattgtcgagacttctgggccatcctagcctgtataataaactgcaggccagcctggggcacagagtaaaaccccgtttcaaagactaaacaaacaccaggcagtggtggtgcatgcctataatcccagcactcgggaggcagaggcaggcggatttctgagtttgaagccagcctggtctacagagtgagttccaggacagccagggttacacagagaaaccctgtcttggaaaacaaaacaaaacaaaaaaaaaacaaagactaaacaaataactgaagcggtgttgggtgtggtgtggtagtgcatgcctggaatctatgatgtgggagattaaagcaggtgactctgggaccagacttacgacttaatgagctccaggccagcctgagttccatagcgagaccaagtttcagacaaaagcagcagcagcacagcaaacccaagcaaacggaaagggctggtgacatggctgagcaggtgaacgcgcttgttgcacaaacccggtgactgagttccgtccctccaagatcccacaggcagaatagagagctgactcttgaagttgtccccgatctccgctgtggcacttgtctgcccccactcacacacacacatacacaatgtaataacaacaacaataatggtagttaaaacaataaagccgggtagtggtagcgcacgcctttaatcccacccagcacttgggaggcagagacaggcagatttctgagttcgaggccagcctggtctacagagtgagttccagaacagccagggctacatagagaaaccctgtctcaaaaaaacaaaaataaataaaaataaaaacaataaacctgagactcagaagaagactcagcagccagttgctctgtgcaggaaagaaaagttctgtttcccacctaagaggccctgctgctgccgccgccgcctcctcctcctcctcctcttcctcctcccagggcctcctactctcctcttacttcctaggcatctggctccccagcttacctgatagttttctctccccaggcattggccgctggaccaacccccagtaccgacagttcatcaccatcttggaagccacacataggaaccagtctgcagaaaacaaggtgagaggttctagagagggcgggcacctgtgcgagccctgctgcccagagctccaggtaccggcttctgtgtgtgtgtgtgtgtgtgtgtgtgtgtgtgtgtgtgtgtgtgctgtgcactgccatggcctctcgaagtgaactgtccttcctacttctagtggatacttggcaggacattgctctccttccccactgccatgctcaagagttacacaggcacagaagcatctgcagccttgagaagtgtctgtcctcgcttctttgcacgcctccaacaccacactggctttgtttggggatggaacaaaacgggcaggtgtagccccttccgtgcccggcagcagagggtgccagcagggcacagtgtcctttaagctgagccagggctgctgttactggagagtggttatctctgaaatggggccgtgaggggatcaaacagacctggtcaccatggccgcactgtgctcatgctgcacagccgaccatttacagctttgttatcacatctgtggagcaccttgtaggctgcacaccctctccagcccctcaccaccacatgaggtggcacagtgaccaccaattttacatctgtggaaagaggaagctaagtggcctgcctcagagtgaggaagtgaccaaggacagacctggacctaggctcgaggcgagcctggctttcgttttatcatttacatttctagggtttttgttttttttttctgttgctctttcttaggcaagttatttttgaaaatgtcaaaagcatttctctggcaaaaaataaaaaataatcctcaaaatagagatcttggagctctgggtgtgggggaggacagtggattatgaagacgcagccctactgcctgcagcatgtgcccagtgcggtgtggagtcggatggtttacagggtagccctgggcctgggtgtcatgatgaggggagggaggtggctatgcacacagggctgtaagagaggaaaagaacctactgtgggcatgaacgcctgcctcttggttatgggaccgtcatttggtttgctgggggggggagggatctctctcattaatttttttccccgaagacatggtatagccctgtctgtcctgaaactctctctgtagaccaggtgtggaggataatataaccagccagcagcccaccctctgcccccatctttacacagaagcaccgcccgtggggtgggtcagccttagatgtttctgtgttttgggattttggggtttttccttttgttttctttgttgttttccaagacagggtcatgctgtgcagcccaagcttgcctggtatttctggtgatctgcttgcctcctgggtgaggtcacagtatttgctaccacgacctgtcctggaaataggatctcactccacgtcccaggccagccttaaatttcactgtgtatcccaggctggccctcacttgtagtcctgcctctgctgctgagtggtgggattggtaggcgtgtgctgctaggcccaacttttttttttttgtttttttgagacagggtttctctgtgtagccctggctgtcctggaactcactttgtagaccaggctggcctcgaactcagaaatctgcctgcctctacctcccgagtgctgggattaaaggcaggtgccaccactcccagctaggcccaactttttaacactttcttggtggcattgggaattgaagccggggcactgtgcgtacaggacaagcaggacggcagggttaagagttctggacgtgggtcaatgagctagtggtggaggcggctgccttgtcccaagtcgtggtcatagtagacctttccattcagaggagagggcgctgacctccctttggtctccctgtgacgaggggccatcctgtacagtgaaggtgttgagggtggatggctaggacttccctcagctctctgtcctcccttcctttgtttccctgtccacagaggcagctggccaactataacttcgacttcaggagctggccagtcgacttccactgggaggagcccagcagccggtgaggccccagctgcttagctcttccccttccttgccatcgctttacccctccgagtggagtcccaggcctactgctcctgactctgtttcccttcccctcaggaaggggtcccgaggtggcccttcccgcaggggtgtggccctgcttcgtccagagcctctgcaccggggaacagcagacacgtttctcaacagggtcaagaagctgccctgtcagatcaccaggtaggaaacggggggtgtgggaggccagctggggtgaggggacacagaccagggcctctggactgtcaacttggatgcagagtagctgtcgcttgtagggcctagtgctgtttgtgccctgtgttgcctgtggggacggctttgcgaggcccaggcctcaggagtgcttggagaggcagaccgccagtgggcctgctgctgccaacctctcagcatcttctgctcggcctctaacgggagcagaggtgccgcagcagggagaaggccgtggctgtcactgtgcaggttctagtcctgtgctcacggctgtgtggaattgtcctggtccctctgctccctgatatccccctcggcccccaccgtgcctcagttcctccctctgcttagcctgcctcactgggattgttgagatgagctggcagcaccggggacggctgtgttgtagggtttcatttctgggaagagacgccgtgactgcagcagcagctcggacccaggacaacaggctcagaggctctcaggttcagagcttcagtccaatggcgggaagcatggcagcagcatgcaggcaggcatggggctggagaaggagccgagagctctgcatctcaatccacaggcagcaggaggtgaccgagagccacactgggcacagcttgagcataggagacctaaagccacccccacagtgatacacttcctcctacaaggccacactttctaatagtgccattccctgagtggctcattcctactcaagcaaccacagctgccgtttaccagcctgccagcaaactcagtcgtgggtgctgatggtgtcaggccggccttagactcacagacaccacccaccctgctgagcgctcacccatgcgtcacagcccatgctgtggccgagcaccagggaaggggtctgacttagctttggctgctgatgtcctgaatcggtccaggggaaagcagaccaggtttttgtggccgtgctggcaggcggtgcagggcggtgcaggaacacagcctagtggccaagctgttgtccatagcagtgcctacctcacctaacctatctgtgtgagttacacttagtatctgttgagctccttggatagctcttatgtggtaggcattatcaccgtgaacataacagccaggtctctgccctgacaagagtgtgacccagtttggtatctttcagactacagatctttagagaagtatttttgtgggtgggtttttgtttttgccccactcttcctgtattggtcctgaatagaagacacagaaatctagcatttttatatcgactgggcaggttctgagctattataacctggctaggctattaatgaccacataaatgccccattccttgccaagtgagtctcgcccgcttgttctgttccgtatgtgtcctgaggaccagcttctcttggccatgtgctcatagcccaacctgcctggaagcaaatgcccttacccaacaagtcatcttgttgacccttgaaaactttttataagttgtatttttatgcattaactgaatgatatttaacagctgtatttatttattgtacctagaaattatctcataaaactctcagaattatatccaagcagtcccaaatttgctacagctcattttggattgtttgcctagcctagcgagggccagcgtgctccatggtgccaggcaggggttgtgaaccccggcccccaccagcaggagggctcgtcccttaggttatggggtcaagtgcacttgcagcctgtcatttccaccttctgatgttattttgggacatgaccctttgtaagtccaggaggatctgtgtaattgtattgcacaggagaaaatggtttgagttggtttttccagtgctgggctcaaaacgaggggctcacacatgccaggtaaacacgctgccactcagctgtgaccccatcgctaagtggacattttccagtcatagttatggaaaagcttaggagatgccaggctcgccttgctcactgagctctgaatgcaagggccaagatgaatccctgggccctgctgaggttggggttcaccaacccatgttccccacagctatctggtggcacacaccttggggcgccggatgctgtacccaggctccgtgtacctgctccagaaggccctcatgcctgtgctgctgcagggccaggcccgactggtggaagaggtgaggccccctacctgctgctggccctgtcttctgaggggtgtggccccctgccccttgtgaccaggtctttgcatctctcttgcttagtgtaatgggcgccgtgcaaaactgctcgcctgtgatggtaatgagatagacaccatgtttgtggaccgacgggggacagctgagccccaggggcagaagctggtgagaggggccagggagccgaagggggtggtaggtggcagtagagactctggggtccccagtgctacagaacctttcctgtggaaccacagttaaaaatgttattttccagcttcacccaaggacctcttcatcagaaagctctgagagggaagtaggtagggggatgtagctcaggtcgtgcacgaagccctgggttcatcccctgtcccatatacactgagaattggattgtgtacctataatcccagcacccaggagtggagagaagaggatcagaaggtcaaggccagggccagggagatggttcagcgggtgcagtattggccatgcaagcgagaggacctgagttctgatcttgagaacctgtatagagcctggcatggtgaccatgagagctgtagtcctagctctcctgtgggagggaggcgggagacgccttgggcaccccctgccagctggcgtatacagcactgccacaggcaacaaagaccccgccttgaagtcggaggcaaggggacacaggggaggagtccttctcaccgccacacacgtgctgtggatcacgtttaaggtcttggctacatggtgagtttgcagtttaaagatgagaatatcatagccgactggggatgtcactgtagctagcgtatgcagcctgatggcagttcatcaccaaagtgaagtcaggtatagagagttgcctagtgttcgggaagctcacctaaagccaggcatggtgcactctgtcatccctgggctgagttcaaggcctgaccaggataaacaggaccctgtcctacagtaacaaagagtagatgggatggtgatgaaggaggcccaggcaagattctgagttccagtccaccctgaactacctagtgagaccctgcctcaaggaaacagaaatagcattgacacatgcaggaccagcctgtgggttatctcaggtgctctcacagctgtgaggagcagcgctgcagcctgagctgaggagcccagctcttaaggcagcactggcctgggaaagcactgcacaggcggccagggggctgggttctgccgcccgcctgcctgcccgcccgcccacttgccaccatccccatgctttggcttgaacaccatggagggcggtgaggagaaaaatgtttgcacacctaggagagtcccagacacatcctatcagagctctcctggtggcatctcaggggaagtggcctccgtgctggtgacttagactcagcctccatagtctcctgaggccctgtgcatagcacctgcagtccctttgtcccgcccagcactctcccacccctcctctctctgctccccaggtcatctgctgtgaggggaatgcaggattctatgaggtgggctgtgtctccacacccctagaaggtaggctcgtggttcacacctcttatctctatcctttggaccggaatgtgtctcctcccatgctgacctccctcccgttcatcagggtgaggtccaggtctctcccttgtgagggaaggtccactcccgccctgttaaccttgcattcattcctgggctgcttgtcctcctgccccgctgcacggtcgcatgattcccccccactcctccctgactccctccctcctccatgtgttacagctggctattctgtcctgggctggaatcatccaggctttgcaggaagcacggtgagaccctctctgaagacctgtccttttccagaatcctgtgcatcttcctctgctcagccatgggaagaaggaacctggtgcccatttctgcagggccttccacatgcctgtgacctgggtgctggggagctgcagcccctcctcccatggcagtcagggacacaatgggtcacatgatctctgtgtgttacagggtggctgcaagaagggtccacactaggtgagctgtcttctggctgacccctcctgcttctgcccgtctccctgcagggggtaccattcccacagaacgaggccaatgccatggatgtggtggttcagtttgccatccaccgcctgggcttccagccccaggacattgtcatctacgcctggtccattggaggcttcactggtatctgcctcccctactcgtccctgcgcagtagagatgggcacaggcacgggtggctcccaaggagggggtggatggaggcttttagagagagaggttggaagttgataagacctacatgaagggacccaatcctatttcttgttctgcttctagccacgtgggcagccatgtcctacccagacatcagtgctgttatcctggacgcctccttcgatgacttggtgcccctggccttgaaggtcatgccggacagctggcgtgagtgcagctcccttgccggtccttcccgtcagggtggcagcacaggcctcttctgactgggtgctgtcactgtgtgggtggagataggccaccccgttgccgagaaacagggcttccctgcccagtggacagtgggacctttaggttaggcaagctcttcatctctgcgtccttcacggtgtcccactctaggagccctggtcaccaggacggtgaggcagcatctcaacctcaacaactcggagcagctgtgcaggtgagggtggccaccgtgcccgtcagcaagcggccactacccgagctggcccaacccttcccggggctaggagaccctgaattggctcactcatgatgtggcagtgaagggttaagcctggctcccagggccagactgagcccgtgcccttaaatctgttactcctgcctcagctgtggcctggctctctgagctctccctccctgctgctctgttctgaaggttccagggccccgtgctgctggtccggagaaccaaggacgagatcatcaccaccacgtgagtacccgggctctgccttctctcccacctttgtcaccatggacacagcttcccaaccagggttaactcttgtcgcccccagggtccctgaggacatcatgtccaacagaggcaatgacctcctgctgaagctcctgcagttccggtgagctctgtgggaatacacacctcagtgctcacaccagtgaggcggaaggatgaggtgggggctgaggcgtgaactggttccattctgtcccaccctgtgctcctcccaaggtaccctcgagtgatggtggaggaggggctcagagctgtgaggcagtggctggaggcctcttcccagctagaggaaggtgaggtatctagaggctggtgggcaggggcctggcaaggagagcttgctgaggacactcctctgttcccatcagcctccatttacagtcgctgggaggtggaggaggactggtgtgtgtctgtgcttcgctcctaccaggcagagcatgggcctgatttcccctggagtgtgggtaaggatggtgtggagggctgggaggagaggaaggacgtcccaggcagggatttgagggcctcttctccctgtgcaggggaggacatgagtgcagatggacggaggcagctggcgctgttcctggtgagctcaggagctgggtgaggggcagggcaggggcagggcctgcacagcggggctcatacagagctcatgcagggatcctagagtgggtggatgcgtggcttctgaggggaagctgccctgttctgacctgacctgcctttctcaggcccggaagcatctacacaactttgaggccacacactgcactccactcccggcccagcacttccagatgccctggcacctctagaaccacgtgtagactcttctggaaggacaagataggaggagacacgaggggatacttttgtgactctcttcatgttgctgtttatagtctggcccgtggggacatcccctctggccaacagtcctcctgcacgtttccctcatctatgtggaagcacttagctcccctcatacacacctgcattaaatgaatgatttattcctaataattaataaaaaggtattttttctacaatctggctgcttatgagatgttccagatgtccagacagctagtggcttgggcggggagagcagaagccctggagggtgagcttgggagaccctgctcaggccagcttaacttccctgcctcagctatcgtgcactttgggcccacagtccagacccttcgtgtgctgtgcccagtgttcacacagccaccatggctctcactgcccacccgggccttcctgcgggcggctgcctgggtggtggtcagaatgcccctcctttataactgtactttgtggtgtacggttctgcctcctcagcttctctcctgaaggtgcttccccatccccacccccattttccttgcttagctctgcttatctccacagcaaaccgaccccatgcctgtccttggcatgacaccaagtctgtccatataccatgtttgcaagtcagcacacagccagtggggaccaagggccaggccccaagcactcctgtcctcccagcgcagagcctgaggaaatgggatcctcacaactcttactctgatctttggacccgtgggatggaatgacaggctggcctccttcccttggaaacttgtgtcaggtgaaatcaccccagctccgcctcctgggccactgccccgaccatggtgctgaacaaagacatcagtgctttctgctggctacagcccctggtttctgtgtgccctctgacccccatatgtgaccctctaaccgtggggctgtgtgtctgtcagctcagcagcttggtgacagtgtgctgaatcttaaagaggaggagggccgagacagacaggtgatatgactaaggtctggggttagaagaggggagacagccatagtttctgaatgttctgcctttggcaaacagcaggcagagagctagccacccaggaggcagacaggctgggatgtgtataggaagaacctactctgggtagagcatggtagcagcatacagtcaggcagaggcagttctgagttcaaagccagtctgttctacatagcaagttccaggtcagacagggctacatagtgagtgagaccctgcccccaaagccaacgtgagcatgctcacggaccttacccttccatcccaggacagattcctggaactatgtctgttcagcccagggaccctctaatcttatctcccataccttggaacacaagatcgcccccttcttggacagccatcagggaatgtcacctagccaccttctaagagagatcagatggagaccttaggtatcttaagcggaagtgagaggtagttgctcaccatgtgccttgtggactccctcaccggggctgtagtctactacagatcgctctccgtatcccgtaacctcagggtggggactggctgtctgcccctttctccctaaggctcagcaagctgaggcaaagtcagctgacgtgcccctacctcagctactagggctctgggccctgctgctctagacaccaggttgggcctccctgatacccaggcacctgggaagctaggccaatggaggggaggtctctagaaagatagctgtttaaaaaaaaaaatcttaataaaaggggtggtctctaaaccctggacaagaacacgccttatcttactgttgctgaccagcatctaggcctgcactgttcccggggtaggttgaagatttactccaagccccatgaagagaaaatgtttcctctaaagctgtcaaaactgagcaaggaccctgagggagggggcccagctatttcagaaagccgggtctccacttgtctgactatccatccatgcatgcatgcatccatccatccatccaccctacctccttattaccatcgtcattaacattcagctaaaggctctgtttctctgagtgacttctctcacagaagctggctctccaaggccgtcaccacagcctgcccctcgtgtgtgtatctcttgttatctgtgatggcgcacattctgccctcagatcctgcctacttcatcgcttggctgttcaccttctgatatgccttgtcacaaggctgggcactcagcatggatgtggtcatctgctactgtcctgtgatgactgttgcagcaaagctgttgtcctagaaacaatgggtctgggccctgggtcctcactagccgtctccaacagctgacagaaaagaaacaaggggctggggtggtcaggagatgactcggtgcctctagcccacctggagttggagtctcagcactctcatggtggctcacaactgtctgtaaccccacaccatcttctgtctttttttttcttcttcttcttccctgagacagggtttttctgtatagccctggctgtcctggaactcactttgtagaccaggctggcctcgaactcagaaatctgcctgcccctgcctcccgagtgctgggattaaaggcgtgctccaccacgcccggctccatcttctgtcttttacaggctctgtgtacactgtgtacagacacatccatgtgtgtaaactttttaaaaacattaatagaaaaagaaacgagggctctaggcacactacaaagaaaggcccactgtcccctgaaacacttcagggctgtggccatccctggctcgtctcatggccctgagctccagagtgactccttttctccctttgcaacagggactgaacctttgcagcttcagagtgtgtcctgtgctgatctctggcactaagtgctcattcactcaacagacatttgcatcttgtatcagagggtgccagatatctccccagtcctccaacattttaggtttcattccggccgggcagtggtggctgcacacctttaaaccctgtctcaaaaaaccaaaaaagtttcattctggttctcagaaacataaaccccagctcctccccttcccactgctcccccactccccacccatctttcagggcttcggtttgtcctccattggtttcccccttagtcctgagatgtgatgattcctggaaagttcttacagtatgggggtggggtgggggcactagagcaggaagtgtctcctgtaatgcaatgtggcttaaagaaaaaacaaacaagtctcaagcctacaacctacctggggagaaggaaagaagcaagcaagccatctagccggggcctagagaaggacatgtttgcagcgccaccttctgtggacgcagcattaatcgctgagcacccactggtgcactggctccaaaaaaaaaacgggtgtccatgcctgccaatcacctacggggctgcgtttgtaggggggccagccagagcagatgctctgtgagccgtggagtggggactctgagcactgagtctgtgggcgtggagactggatgtctgacctgcagcccagcttcgctgcacacaaagaaaggtcctggtcacttctttgtgcaagtctgctccgcctctttcatcccaacttctggttctgcctgacaccaaacctgtccatcagttttaaccctgaatctaccgtaaagctcagggttatggttcctgtcatcccagtttagggagactatggcaggagggtcagtccaaggggagcctgggttactcaggccagatctgttacagagtcagatcttgtctacaagttttaaaaaacaaaacaaataaacaaaaaaccacaatatttacttctatccattcataacaactccgacatcacctcgcttccaaaaacctatgtcaccatcttatgactaaacttgggaattgcactctggtctggtttcacaccctatactatggatttaacctaggtctgtgatgtatgctaggctaagcaagcaacacaactaaccaggctggcctcagattcacagtgatgacagaattaaaggtgtgtccccacacacccagcttattttacttttattttgagacaagtttacacgaaattatccaggctccttttggacttgtagctcagtcaagtcctttttttttttttttttttcttttttcgagacagggtttctctgtgtagccctggttgtcctggaactcactctggagaccaggctggcctcgagctcaaaagtctgcctgcctctgcctcccaagtgctgggattaaaggcgtgcaccaccatcagtcaagtcttaaaacctgcagtcctcctgagcatccgagtagctggaaagacaggccagtgccagtaggcagactggcttctgctcttaacccctttattatccaggatgcaacaaaggcagaactttacaaaacatctggccacattacctctctgtcttaaccttatgggattcatagccgctatgtctcaagatctccatgccaggatccaggtcagaaacttgtctctcccagcctccagattaggatcacacgtgatccttccaattctggattgtagttcattccagatatagtcaagttgacaaccaggaatagctactacaacatgtatgtgcatacatacagacagacacacaaacacaccaactgaagggggtcagagagagtgagacccagcatgaccactcactgtccttgactctggttgccatatggccggttctcaaaagtgtctgtggctgtgacttccctaacaggatggactgtgacctggaataacaagctaaaataaaatagccccctttgccgatcatggtggcacacgcctttaatcccagtactcaggaaacagaggcaggtggatctctgagtttgaggccagcctggcccatggagtaagttctaggacagccagggctacacagagaaaccttgtctcaaaaagtaataataataataataaactgggcatggtggtgaacacctttaatcccagcacttgggaggcagaagcaggcagatttctgagttcgaggccagcctggtctccagagtgagttccaagacagccaaggctacacagagaaaccctgtctcgaaaaaccaataaataaataaataaatagatagatagatagatagataaatagataaataaataaataataaatgatgatgacaatgatattaacatcaaaacatataaataaaaataaacactccccccccccccggttgcttttgtcagagcattttacggagcaccaggaagggaaacagacaggacaagtccttctgagttcctgcctccagccaggatattaggcccaaggagctgtgggttgaggacactctgcacgccctgttctggtgccgttctccacctcagactggtgccccactccctcctcttggccagcctctctccaaccaatataagcccaggctggaggctggagagaagccatgctttttatggcaggccctgcagccagctggtccctgaggcccctgggactccatggcgtcccccaagccttgtgtgctgtcctcttaacagtgctggtcatgaagaccttggttctcggtaagtggctccggagcagtggttccaccttctttttttcttttcttttgttttttcgagacagtgttttttctgtgtagccttggctgtcctggaactcactctgtagaccaatcctagaggcttcgaactcagaaatccacctgcctctgcctcccgagtgctgggattaaaggcgtgcgccaccacgcctggcggggttctaccttcttgatgatgcaaccctttaatacagtttcctcatgttgcagtgaccccccaaccataacataacactttttttttttgctacttcataactgtaattttgctactgttatgaattgtaaggtaaatatctgatatggggcccttgtgaaaacgtcatctgacctccaaaggggtcgtgtcccacaggttgagagacactgctccaaagagtcagaagggtcaccaggccagctaggtggaaaaactctggtttctccaagggtctcgacacttctgtccatgatgtacaggtgaaaccagcggtaggcatgataccatgggcagatgtgtggggatgtccaggaactgtgatggcctgatagtcagtcactgcaaaggagagttctcgggacggccttgttgctgtagcaacaacagtgttctggagatactaaagggatcgtttccagctctaagggcaacacagaatagggaggactggggttgtgtcagattctcctcaaccagaattcacaaggcagaagcctttgcactacactaagctatccgttagtcctgggctcttaacatgtggactggaccaagggccagggaagagggggctgcatatgcaggcagttcatcgggagacagaacagggcctttggtagctaacctccaacatgagcctgtccagggaccagacagcagaataaggcaggtagcatggggctattgaggtctcctgtctacaggacacagactctctctgtcttctcacggaccttgttctcacctctgaagaccacattgtaaaaaaagcatgttggaactagacaggggtggcaagtgcccaggaggtagaggcaggaggatctcttagtttagcgctatcctggtgtatagagtgaattccaggacagccagcgctacacagataaactcttgaaaaactaaattaaaaaggaaaaagaagaaaagaaagagtgcccactggtggcaattctagtactcaggcaagaaggttgaaagccaggtgtgtgtgtgtgtgtgtgtgtgtgtgtgtgtgtgtgtgtgtgtgtgtgtgtgtgtgatgtttaaaagagaatacagagggctgaggacctgagttcaattcccaccaaccacaaccacatggtggctcccaaccatctataaagggatctgttctctcttctggcatgtaggtatacatgcagatagagcactcagacacacacacacacacacacacacacacacacacacacatacacagagagagagagagagagagagagagagagagagagagagagagagagagagagaatgaactactacatttcttcaattctaacccatgacttcaatcctagcatagcacttagatcagatgagataccaaatgactaggtcgtagaggaaaggctgttctaacagggctggatgacagcttcagcttcttcccctgtaaatgtaaattaggcttatggaatgctgggtataggttagcccttcaaatatatgccaaatggatacatttctttgggggtataacatagaatggaggtttggttttttgttgttgttgttgttgttgttttgatagctttttttgagacagatcgacagatctcacactatggagccctggctgtggcctcctactttgtaaaccagggtggcctccaactcatagatctgcccaaccctgcccttgagtatggggtttaaagaggaggaagcacccgccaaacaggcaagggtggtgactttttttttttttttttttttttttttgagacagggtttctctgtgtagctctggctgccctggaactcactctgtagaccaggctggcctcaagctcagaaatcagcctgcctctgcctcccaagtgcttggattaaagcccaggcagcgtggtgacatctaacaagaggtctgttaaggcagacggctttcaagaagcagagatcttatagttgcagagccacagttccgttggaatggggcagggacatctgagccatggaattcctcttcgttctaggtgataccaagctcgaggaccttcaccctcagtccctcccactaaacaagtacctgaattgctaccgatgtctgctggagaccgaagagctggggtgcctcctggggtctgacacctgcctgacacctctgggcagcagctgtgtcaccctgcacataaagaacagtaaggggacccctcctcccaccttgggccagctccccaccaggcctcctcccagtgtcctttgcgatacaccaagtcttgtttccaccgcccttagatgttccagcactttcagtcagatccacaccagtctttactcctgtttcctcctcagctcagagccacggcctcccactttcctacggtgtccagccttgaagggccctcgctctccagaggcactgggctgtcccgagtggtcagcatcccctgctgtcagagactcttctcctggccttgcagacgctttggggctcatgctcaggcggcttggtcctttgttctgacatacacatcacagaggcccctggccttaactagcagcctcagacttcttcctagcttagaaggcttcttctcgtcccctgatccccattcgggtgtccccatcatttcctgcttggaggactgatcgcatcctccccccacccccgttctcttcttctacccaccccctacctttactaaattaatagctgctcagtgtggacaacgtggaggcgcacctgagctctgtaagagcattaagcactcttgaccactgagccatttccccagtctctttcgcctgtgatgctagaactcagactcaggacttgtgcctgcgttctaccgctgagctgcacggccccgttctatcctcttctgctgtagaaaaccacaccctgtctgcagcccacccttcagtatacctccatgtcttcctcctcatggtccatgacgcttggtctccttgcatggtcacaacttcaaatcccccaagcttcccacgcaaagcttccgttgcccaggctcaccggccgtcccgtccggttctctatggctttctctcggttcagggaagggtgtaagggtggtagtggtatccgtgtggactgagggtgagtttgggggctctgtgcctgtgctccctccctaggcagcggttttaatgtcatggtgagcgactgctacagcaaggagcagatggtccattgttcatatacccgtgcttccccggtgtttggcttttggatattctatcagtgttgcttcctggatttctgcaacaatccggacaacagaaagaatagcatgcactagaacagcaggaacccgccacgtcctgccagctaacctccagactggatttccatctgagtccatcaggacaactccaaagccctcactcactacagcctacatcgttgaccccacgttggcctcccctctccatccatacccacaatgctctcctacctctattttgttcagccccctctttggtccaaagttccccccaaataaaatagtatgcaaagtgtttgtcgtctcctctgatggaggtgaagctagcctgtcatagggctcctctactgcccagccctagacatataaaattgaagagggactggatgcccttctatggtgaaggttgggttggttagattaataggtatcctgagaggtactgtcagaccagagctgcgtgggggagaactgagagggctcgggggctatattggcaagatacagaatatgagcaataaggatatataagcaatataagcaatcccagggaggagggatgctcgaggaggacactggggaggggacagtaggctcaagaaaaccaaggaactctctgacttccaagtttagtgtcccttcctaaacctggctcccagtttgaccaattaaatctgaccagggtccatctggggagggagtgtggattttactgtagaccccttactggcctccaactccagattgtccctcctcaatttcccgagtgttaacattacagcctgtaccacacaagctgctgtgagcagcgtctacctgagttaggagtgctcaggaagggcatgagggcacactgggtgctggccaaggcgtctgctgactgaatagcttcctctggtgtgtttgtgacaagcttgtgctctgctgggaacgtttacaaaacaggctgctgttcctggaggctcgtgcacctgatctattcagaaggctgaggcaggatggcctggagatcaaggccagcttgcatcagcgtgagaccaaatcgcaaaaggccaaaacaagccccaaagctctatgtatgtatgtgtccctaagcacacatacctgcgcatgcatgatctggacatccgtgacctccagcgcaggtgaccacttcaaactacagggctgacttactcaggcattggagcaaaggggagtggacctcttgctgcagagtgactctagcactgggctgagagggggtgagcttgatcacactcacctggaagtgctggtgacccctggatgacattagtgtcctctgttgaataatagagtaatgcaaagcatccatgagtgctttctccgtgcttgaaaattttcaatttattatttgagatgaatttgtaattgactgtcctggaattcactatatagaccaggctagcctcaaactcaagagatccacttgttttgtttcccaagcacagggatcaaaggcgtacaccaccatgcctgatc

SEQ ID NO: 17 - DNA, 2017 bp, Mus musculus
gagcagctacacgaagcctgtgtttctatatcgtgttgttctctttccctcccccctcttccgagtgagaaaaaaaaggggggcctaaattcccaccaacataaactaatgacatacaatgatgaaattctgttttcacctctgcctgtgacagggaatgcaaaaatagcaagtggcctatttccacgaatccccgcctccctgccctccccgctcccgctgggtttggatctctaagaatggaggctaacgcagaggcaggagccagccgggatcagccccccgaacccgagggtgatgtcagtcggcgggagcggacgcagcctggccagggaggcagaggttcctagggagcagggagggcgggtggcctggatgggcggaccgtgcgcccggccgctccgtcgcctggagacggccctggcggcgctgtgttgctgtaaacagccgcttcgctgttactatctatagaaagatcgcctagctcccgggtgcggcggcccgaggcaggtttgtggttctgcaccccttgcgcccctaaagatccatccgctgtcctaatcctctctagtccgtcttggaatgcccaccgtttggcttgacccttgggaccctcagaatggagacgagggggacacatgagttctgcgaggatctgcggtttcctatcccagaggtgaccggcccagtcagtctaacccggcttgtcctctgcggaaggacaggaggccgagggcaagtaggggtgtgtttgttctacactgaagcacctgacctcttcaaagttccatcttccaagactcaaagctgttctcaggtcccagacgccaaaatctcggcacagctgggaacctttcttcccgtcccctctgcgcccccaccccccttcccaagtccgatctggaaaatcacccgctgcaggcgggttccttgtaagcgcagtttccaggctgcacgtattcagacccccatctccccagcaccgacttgctttctcctccccccccccccccgagctcacctcactttgtaattctgagctccccccctgccccgactcgccctctggtctcagctcaaaactaaacatacgaccccttcaggcatacttgtagggtggttttgcacaatgtttatccgtcagtgtcaacggggactgtcgccttgatagctctaagtggctaagggtcggggagtaggtgctgccgtcctttaaaacacgaatttatgaatgaacccagtactgtagttaaatcaggttattgtacacttatttacaatccttcacttgctgcttccaacctcagtcctaaagtttctccaggcaaggagctggagagaggggctgagaagctgaccccccctttttcttctctgcactgatttgggatggggggctgatgtgggcaagctttcctttaggaacagaggcttcgagcctttaaggctgcgtacttgcttctcctaataccagagactcaaaaaaaaaaaaaaagttccagattgctggacaatgacccgggtctcatcccttgaccctgggaaccgggtccacattgaatcaggtgcgaatgttcgctcgccttctctgcctttcccgcctcccctcccccggccgcggccccggttccccccctgcgctgcaccctcagagttggctgcagccggcgagctgttcccgtcaatccctccctcctttacacaggatgtccatattaggacatctgcgtcagcaggtttccacggccggtccctgttgttctgggggggggaccatctccgaaatcctacacgcggaaggtctaggagaccccctaagatcccaaatgtgaacactcataggtgaaagatgtatgccaagacgggggttgaaagcctggggcgtagagttgacgacagagcgcccgcagagggccttggggcgcgcttccccccccttccagttccgcccagtgacgtaggaagtccatccattcacagcgcttctataaaggcgccagctgaggcgcctactact

SEQ ID NO: 18 - DNA, 6640 bp, Artificial Sequence
  Synthetic - Inducible gene expression vector pENTR-EUI ecdysone receptor and kanamycin resistance protein genes, complete cds (Accession No. KU664676.1)
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgataccgctcgccgcagccgaacgaccgagcgcagcgagtcagtgagcgaggaagcggaagagcgcctgatgcggtattttctccttacgcatctgtgcggtatttcacaccgcatagttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatcgatatgaagctacagtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtagagttcctggatatggccgacttcgagtttgagcagatgtttaccgatgcccttggaattgacgagtacggtgggctgcagatgaggcctgaatgtgtcatacaggagcccagtaaaaataaagacaggcaaagacaaaagaaagacaaaggaatattattacctgttagtacgaccacagtcgaagaccacatgcccccgatcatgcaatgtgatccacctccgcccgaggccgccaggattcacgaagtcgtcccgaggtatctttcggagaagctgatggagcagaacaggcagaagaacataccaccattgtcggcgaatcagaagtctctgatcgcgaggctcgtgtggtaccaggagggatatgagcagccctccgacgaggatctcaaaagagtaacgcagacttggcagtcggatgaagaggacgaggaatccgatctacccttccgccagatcacggagatgacgatcttaacggtccagttgatcgtcgagttcgccaagggtctaccgggcttttcgaagatatcacagtctgatcaaatcaccttattaaaagcctcgtccagcgaggtgatgatgctgcgggtggcgaggcgatacgacgccgcgtccgacagcattctgttcgccaacaacaaggcgtacacgcgcgacaactaccgcaaggcgggcatggccgaggtcatcgaagacctcctacacttctgccggtgcatgttcgcgatgggcatggacaatgtgcactttgcactgctcacggccatcgttatattctcagatcggcccgggctcgagcagccgtcgctggtagaagagatccagagatactacctgaacacgttgcgaatttacatcatcaaccagaacagcgcgtcgtcgcgctgcgccgtgatctacggcaggatcctgagcgtgctgaccgagctacgcacgctcggcacgcaaaactccaacatgtgcatctcgctgaagctgaagaacaggaagctgccgccgttcctcgaggagatctgggacgtggcggaggtggccacgacgcatcccacggtgctgccgcccaccaacccggtggtgctatagtctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgccggggatgcggtgggctctatggcttctgaggcggaaagttgagaggacattccaatcataggctgcccatccaccctctgtgtcctcctgttaattaggtcacttaacaaaaaggaaattgggtaggggtttttcacagaccgctttctaagggtaattttaaaatatctgggaagtcccttccactgctgtgttccagaagtgttggtaaacagcccacaaatgtcaacagcagaaacatacaagctgtcagctttgcacaagggcccaacaccctgctcatcaagaagcactgtggttgctgtgttagtaatgtgcaaaacaggaggcacattttccccacctgtgtaggttccaaaatatctagtgttttcatttttacttggatcaggaacccagcactccactggataagcattatccttatccaaaacagccttgtggtcagtgttcatctgctgactgtcaactgtagcattttttggggttacagtttgagcaggatatttggtcctgtagtttgctaacacaccctgcagctccaaaggttccccaccaacagcaaaaaaatgaaaatttgacccttgaatgggttttccagcaccattttcatgagttttttgtgtccctgaatgcaagtttaacatagcagttaccccaataacctcagttttaacagtaacagcttcccacatcaaaatatttccacaggttaagtcctcatttaaattaggcaaaggaattgctcaactttgtatagaaaagttggtaatacgactcactatagggcgaattgggtaccgggccccccctcgaggtcgacggtatcgataagcttaggcctccaaggcggagtactgtcctccgggctggcggagtactgtcctccggcaaggtcggagtactgtcctccgacactagaggtcggagtactgtcctccgacgcaaggcggagtactgtcctccgggctgcggagtactgtcctccggcaaggtcggagtactgtcctccgacactagaggtcggagtactgtcctccgacgcaaggcggagtactgtcctccgggctggcggagtactgtcctccggcaagggtcgactctagagggtatataatggatcccatcgcgtctcagcctcactttgagctcctccacacggagcttgatttaggtgacactatagaatacaagctacttgttctttttgcaggatcccatcgattcgaattcacgtgctagcgcttaagaaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcagggccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 19 - protein, 504 aa, Homo sapiens
MDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQAMRNINPSTANSPKCQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 20 - protein, 504 aa, Homo sapiens; identical to SEQ ID NO: 19

SEQ ID NO: 21 - DNA, 4152 bp, Mus musculus
gtttgggcgtctggagccaaagtttctgtctcagttgggacgccgccgcggccgggggcaaagaaagggaggaaggaaggagctcgcggaggggaggggaggagaggggaggcggcctcgggcaaggagtgcagggcgatgcgggcgcgcgtcgcagccccccgaacctgagcgcagtgccccgagcgtcgaacgaggccgcagccatggagcccgcgcaacagccgccgccccagccggccccgcaaggccccgcgccgccgtccgtgtctccggccgggacccccgcggccccgcccgcacccccggccggccaccaggtcgtgcacgtccgcggggactcggagaccgacttggaggcgctcttcaatgccgtcatgaaccccaagacggccaacgtgcctcagaccgtgcccatgcggcttcgcaagctgcccgactccttcttcaagccgcctgagcccaagtcccactcgcgacaggccagtactgatgcaggtactgcgggagctctgactccacagcatgttcgagctcactcctctccagcctccctgcagctgggtgccgtttctcctgggacactcacagccagtggcgttgtctctggccctgccgctgcccctgcagctcagcatctccggcagtcctcctttgagatccctgatgatgtaccactgccagcaggctgggagatggccaagacatcttctggtcaaagatacttcttaaatcacaacgatcagacaacaacatggcaggacccccggaaggccatgctttcgcaactgaacgttcctgcgcctgccagcccagcggtgccccagacgctgatgaattctgcctcaggacctcttcctgatggatgggagcaagccatgactcaggatggagaagtttactacataaaccataagaacaagaccacatcctggctggacccaaggctggaccctcgttttgccatgaaccagaggatcactcagagtgctccagtgaagcagcccccacccttggctccccagagcccacagggaggcgtcctgggtggaggcagttccaaccagcagcagcaaatacagctgcagcagttacagatggagaaggagagactgcggttgaaacaacaggaattatttcggcaggaattagctctgcgcagccagttgcctacactggagcaggatggagggactccgaatgcagtgtcttctcctgggatgtctcaggaattgagaacaatgacaaccaatagttccgatccctttcttaacagtggcacctatcactctcgagatgagagcacagacagcggcctcagcatgagcagctacagcatccctcggaccccagacgacttcctcaacagtgtggatgaaatggatacaggagacaccatcagccaaagcaccctgccgtcacagcagagccgcttccccgactacctggaagccctccctgggacaaatgtggaccttggcacactggaaggagatgcaatgaacatagaaggggaggagctgatgcccagtctgcaggaagcgctgagttccgaaatcttggacgtggagtctgtgttggctgccaccaagctagataaagaaagctttctcacgtggttatagagctgcagggagccactctgagtctgtgagggatccacagagcctaagatgtgcacgcctgaaattcagataagtcagtgggggttctctggctaacacagaaaacagatgaaccagtgtccatcgttgttccgcttttctctgcccgtcgctgctcttacgttggttgctgacctcttcacggccggctctaaagaacccgaaccgcagacagattcctttgttaactctgctatgataactacgttctctgggattgctgggggatggcctgctggataatggatgttctgccttttgtccggtggtcctttcaccatcactttaactgaacacacagactgggaactgaatgctctagaacattgttcaagaggtggtttcttcagctgccttgggtccaacaagccagaggcattgcgtctgatctcgtggaggacggaggggacccacgctgaagactggtgaactttccattcttctgttagcgatgccgttaggccatagtgacctgggtcttatcttagacgcttatgaggcatgagacagcttccatagaaatatattaattattaccacatactctagattaggtttgaatgaatattttctgtgggtgttttggttggtttttctctgcccccccccccttttttgtggttggtcttggtggaacgtaggcaaattaatgaattcgtttatagctgtagcttggggtgggcaataccattcttttggtgggaaatctgtatttcttggttttttaacatcctatttaaatcttaaatcttggttatctcctctctacatatatacacactcttattatgtctatggtagtgtgatagcagaatatatctttataaacatttctttcctttccagtttattttttaaatggtagccttgaacatatacatttagacacagtagtttatgtttcacaaatactttaaattctggttggagcaattgcaggtgtttgagggagggtgggcagtgttatagaaagggctctagtgggtaaagccaagaggcgcctcttgccctctgtgcactgtggtgacaacagaagccaacagtgagtggccttacttcccttagcctgatttcatgccatgtagtgtctgagctgtaagctttagagaggcaggcctgccttctgcctccacctcagctttgggtgtacactcagatactgagactgataatccacgaggtcaggttgagtgtcacctacaagatcatgcttaggaaacctcctcccgtgtctgctgagtggaccctggggtagtgtgcagaatgctgcgaggtcataggtaaagtgatcagactagcaagtgtggggtagttggactgagcagagctacaatgaagcatgattatggctttatttttgcaagattactagagtatgtgtctccaggagtaataatggtttccaaagagtattttttaaaggaacaaaataggcatgaattaactctgtgtataagctatgaagtaatagttggttatgaattatagtggcaccagctagcacctctgtcatttaagggtctttgagtgcttctagaagaatcccttattttcaagggttcataatgaacaacaattttcccccctggctaaagctgctactataaaatcctcagcttaggaagatagaaatcttttggccctttgatttagtatagcacaagaatgaagtagaaaagttttattcagtactttgagtgctcaaaaaatacaatcagtgtattatatgtgattcataggtcagtcatttgaggccattgactctaaggcttttaattagaaactagttgaagactcacccttgaattaagtctggggaaatggcctcccggaggatgcagtgactgtagaacagagatgaaattctactctagttgacaattgagcagatggatgatccacaacagtgcgtggggaatgggcagatgataggcgtgccgagcgcctatgattggccagcagctgcagaataaaggatggcgtcttctgtgggcagactagctgtagtgtggggagggggaggggaggtgggcatgagctgttgaaaggttaccgggctatgatatagaaaagcttttatgaacttgtttctcttcagtaaagacttatctcgctctcagtaaagaagttcctaagtttgcagctgcttaacctcccccacaaggtggatgtgccgtcttcctcctccctcagcatggtgcgccttgttatacctcaatgtccagcagcccctggtgcctgacagtgctctctgaccttatcaaaccctccatgctgactgtgggagatttaagcaccctgactctcactaaatttgaatcacggcctgaacgtagtctttcttgttgtgtccagcagtgtttaatgcttcctggctgtagtaggtagcatacaggtaagaacttaactggttattcttgaatttcttttacaataaaccaatttttataatcttaaaaaaaaaaaaaaaaaaaaaaaaaaaaaaaaa

SEQ ID NO: 22 - DNA, 4200 bp, Mus musculus
gtttgggcgtctggagccaaagtttctgtctcagttgggacgccgccgcggccgggggcaaagaaagggaggaaggaaggagctcgcggaggggaggggaggagaggggaggcggcctcgggcaaggagtgcagggcgatgcgggcgcgcgtcgcagccccccgaacctgagcgcagtgccccgagcgtcgaacgaggccgcagccatggagcccgcgcaacagccgccgccccagccggccccgcaaggccccgcgccgccgtccgtgtctccggccgggacccccgcggccccgcccgcacccccggccggccaccaggtcgtgcacgtccgcggggactcggagaccgacttggaggcgctcttcaatgccgtcatgaaccccaagacggccaacgtgcctcagaccgtgcccatgcggcttcgcaagctgcccgactccttcttcaagccgcctgagcccaagtcccactcgcgacaggccagtactgatgcaggtactgcgggagctctgactccacagcatgttcgagctcactcctctccagcctccctgcagctgggtgccgtttctcctgggacactcacagccagtggcgttgtctctggccctgccgctgcccctgcagctcagcatctccggcagtcctcctttgagatccctgatgatgtaccactgccagcaggctgggagatggccaagacatcttctggtcaaagatacttcttaaatcacaacgatcagacaacaacatggcaggacccccggaaggccatgctttcgcaactgaacgttcctgcgcctgccagcccagcggtgccccagacgctgatgaattctgcctcaggacctcttcctgatggatgggagcaagccatgactcaggatggagaagtttactacataaaccataagaacaagaccacatcctggctggacccaaggctggaccctcgttttgccatgaaccagaggatcactcagagtgctccagtgaagcagcccccacccttggctccccagagcccacagggaggcgtcctgggtggaggcagttccaaccagcagcagcaaatacagctgcagcagttacagatggagaaggagagactgcggttgaaacaacaggaattatttcggcaggcaatacggaatatcaatcccagcacagcaaatgctccaaaatgtcaggaattagctctgcgcagccagttgcctacactggagcaggatggagggactccgaatgcagtgtcttctcctgggatgtctcaggaattgagaacaatgacaaccaatagttccgatccctttcttaacagtggcacctatcactctcgagatgagagcacagacagcggcctcagcatgagcagctacagcatccctcggaccccagacgacttcctcaacagtgtggatgaaatggatacaggagacaccatcagccaaagcaccctgccgtcacagcagagccgcttccccgactacctggaagccctccctgggacaaatgtggaccttggcacactggaaggagatgcaatgaacatagaaggggaggagctgatgcccagtctgcaggaagcgctgagttccgaaatcttggacgtggagtctgtgttggctgccaccaagctagataaagaaagctttctcacgtggttatagagctgcagggagccactctgagtctgtgagggatccacagagcctaagatgtgcacgcctgaaattcagataagtcagtgggggttctctggctaacacagaaaacagatgaaccagtgtccatcgttgttccgcttttctctgcccgtcgctgctcttacgttggttgctgacctcttcacggccggctctaaagaacccgaaccgcagacagattcctttgttaactctgctatgataactacgttctctgggattgctgggggatggcctgctggataatggatgttctgccttttgtccggtggtcctttcaccatcactttaactgaacacacagactgggaactgaatgctctagaacattgttcaagaggtggtttcttcagctgccttgggtccaacaagccagaggcattgcgtctgatctcgtggaggacggaggggacccacgctgaagactggtgaactttccattcttctgttagcgatgccgttaggccatagtgacctgggtcttatcttagacgcttatgaggcatgagacagcttccatagaaatatattaattattaccacatactctagattaggtttgaatgaatattttctgtgggtgttttggttggtttttctctgcccccccccccttttttgtggttggtcttggtggaacgtaggcaaattaatgaattcgtttatagctgtagcttggggtgggcaataccattcttttggtgggaaatctgtatttcttggttttttaacatcctatttaaatcttaaatcttggttatctcctctctacatatatacacactcttattatgtctatggtagtgtgatagcagaatatatctttataaacatttctttcctttccagtttattttttaaatggtagccttgaacatatacatttagacacagtagtttatgtttcacaaatactttaaattctggttggagcaattgcaggtgtttgagggagggtgggcagtgttatagaaagggctctagtgggtaaagccaagaggcgcctcttgccctctgtgcactgtggtgacaacagaagccaacagtgagtggccttacttcccttagcctgatttcatgccatgtagtgtctgagctgtaagctttagagaggcaggcctgccttctgcctccacctcagctttgggtgtacactcagatactgagactgataatccacgaggtcaggttgagtgtcacctacaagatcatgcttaggaaacctcctcccgtgtctgctgagtggaccctggggtagtgtgcagaatgctgcgaggtcataggtaaagtgatcagactagcaagtgtggggtagttggactgagcagagctacaatgaagcatgattatggctttatttttgcaagattactagagtatgtgtctccaggagtaataatggtttccaaagagtattttttaaaggaacaaaataggcatgaattaactctgtgtataagctatgaagtaatagttggttatgaattatagtggcaccagctagcacctctgtcatttaagggtctttgagtgcttctagaagaatcccttattttcaagggttcataatgaacaacaattttcccccctggctaaagctgctactataaaatcctcagcttaggaagatagaaatcttttggccctttgatttagtatagcacaagaatgaagtagaaaagttttattcagtactttgagtgctcaaaaaatacaatcagtgtattatatgtgattcataggtcagtcatttgaggccattgactctaaggcttttaattagaaactagttgaagactcacccttgaattaagtctggggaaatggcctcccggaggatgcagtgactgtagaacagagatgaaattctactctagttgacaattgagcagatggatgatccacaacagtgcgtggggaatgggcagatgataggcgtgccgagcgcctatgattggccagcagctgcagaataaaggatggcgtcttctgtgggcagactagctgtagtgtggggagggggaggggaggtgggcatgagctgttgaaaggttaccgggctatgatatagaaaagcttttatgaacttgtttctcttcagtaaagacttatctcgctctcagtaaagaagttcctaagtttgcagctgcttaacctcccccacaaggtggatgtgccgtcttcctcctccctcagcatggtgcgccttgttatacctcaatgtccagcagcccctggtgcctgacagtgctctctgaccttatcaaaccctccatgctgactgtgggagatttaagcaccctgactctcactaaatttgaatcacggcctgaacgtagtctttcttgttgtgtccagcagtgtttaatgcttcctggctgtagtaggtagcatacaggtaagaacttaactggttattcttgaatttcttttacaataaaccaatttttataatcttaaaaaaaaaaaaaaaaaaaaaaaaaaaaaaaaa

SEQ ID NO: 23 - protein, 348 aa, Homo sapiens
MRWRRAPRRSGRPGPRAQRPGSAARSSPPLPLLPLLLLLGTAALAPGAAAGNEAAPAGASVCYSSPPSVGSVQELAQRAAVVIEGKVHPQRRQQGALDRKAAAAAGEAGAWGGDREPPAAGPRALGPPAEEPLLAANGTVPSWPTAPVPSAGEPGEEAPYLVKVHQVWAVKAGGLKKDSLLTVRLGTWGHPAFPSCGRLKEDSRYIFFMEPDANSTSRAPAAFRASFPPLETGRNLKKEVSRVLCKRCALPPRLKEMKSQESAAGSKLVLRCETSSEYSSLRFKWFKNGNELNRKNKPQNIKIQKKPGKSELRINKASLADSGEYMCKVISKLGNDSASANITIVESN

SEQ ID NO: 24 - protein, 422 aa, Homo sapiens
MRWRRAPRRSGRPGPRAQRPGSAARSSPPLPLLPLLLLLGTAALAPGAAAGNEAAPAGASVCYSSPPSVGSVQELAQRAAVVIEGKVHPQRRQQGALDRKAAAAAGEAGAWGGDREPPAAGPRALGPPAEEPLLAANGTVPSWPTAPVPSAGEPGEEAPYLVKVHQVWAVKAGGLKKDSLLTVRLGTWGHPAFPSCGRLKEDSRYIFFMEPDANSTSRAPAAFRASFPPLETGRNLKKEVSRVLCKRCALPPRLKEMKSQESAAGSKLVLRCETSSEYSSLRFKWFKNGNELNRKNKPQNIKIQKKPGKSELRINKASLADSGEYMCKVISKLGNDSASANITIVESNATSTSTTGTSHLVKCAEKEKTFCVNGGECFMVKDLSNPSRYLCKCPNEFTGDRCQNYVMASFYSTSTPFLSLPE

SEQ ID NO: 25 - DNA, 2166 bp, Homo sapiens
gctcgcagccccagcagccgccgcagcatcacttcacacaaaggactgtcacccgcggagcagctcctccacctccacgtcctcctccggtgacagcagccccgacagcagggctcgggcgcggcggcggcgcggggggtggggggacctgtcactccctgtaactgaggcggcggcagcggtttttttgccttttctattttgccttttttttttttgcccttatacctcttcgcctttctgtggttccatccacttcttccccctcctcctcccataaacaactctcctacccctgcacccccaataaataaataaaaggaggagggcaaggggggaggaggaggagtggtgctgcgaggggaaggaaaagggaggcagcgcgagagagccgggcagagtccgaaccgacagccagaagcccgcacgcacctcgcaccatgagatggcgacgcgccccgcgccgctccgggcgtcccggcccccgggcccagcgccccggctccgccgcccgctcgtcgccgccgctgccgctgctgccactactgctgctgctggggaccgcggccctggcgccgggggcggcggccggcaacgaggcggctcccgcgggggcctcggtgtgctactcgtccccgcccagcgtgggatcggtgcaggagctagctcagcgcgccgcggtggtgatcgagggaaaggtgcacccgcagcggcggcagcagggggcactcgacaggaaggcggcggcggcggcgggcgaggcaggggcgtggggcggcgatcgcgagccgccagccgcgggcccacgggcgctggggccgcccgccgaggagccgctgctcgccgccaacgggaccgtgccctcttggcccaccgccccggtgcccagcgccggcgagcccggggaggaggcgccctatctggtgaaggtgcaccaggtgtgggcggtgaaagccgggggcttgaagaaggactcgctgctcaccgtgcgcctggggacctggggccaccccgccttcccctcctgcgggaggctcaaggaggacagcaggtacatcttcttcatggagcccgacgccaacagcaccagccgcgcgccggccgccttccgagcctctttcccccctctggagacgggccggaacctcaagaaggaggtcagccgggtgctgtgcaagcggtgcgccttgcctccccgattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacagtacgtccactccctttctgtctctgcctgaataggagcatgctcagttggtgctgctttcttgttgctgcatctcccctcagattccacctagagctagatgtgtcttaccagatctaatattgactgcctctgcctgtcgcatgagaacattaacaaaagcaattgtattacttcctctgttcgcgactagttggctctgagatactaataggtgtgtgaggctccggatgtttctggaattgatattgaatgatgtgatacaaattgatagtcaatatcaagcagtgaaatatgataataaaggcatttcaaagtctcacttttattgataaaataaaaatcattctactgaacagtccatcttctttatacaatgaccacatcctgaaaagggtgttgctaagctgtaaccgatatgcacttgaaatgatggtaagttaattttgattcagaatgtgttatttgtcacaaataaacataataaaagga

SEQ ID NO: 26 - DNA, 1443 bp, Mus musculus
ttgcagaggaaaaagtgactgagcgcaagaaggacaaagaggagtggttgggggtgggggtggaggcagggcggggaagggagttaccgcccctcctggctcctcctgccctcgcatccttgtgcaatggtactgtttgccgtgatgctcagagggcaggcacctgctgctctgtaatgattcagcctctttcagccgctgcgttaacacgacaggatgctgttgctactgtcgctgctgcccctcctgccgccgccgctgctgccgccgccgcctcctctggtcttgcttttgcttttacttctcctgcatgacagttgttttcttcctctaagcagacaccagcttcagacgcttgaggtgagaaacatgcctttcagtttgggatactggtttacttaatcagctaggcatcagcttgcttcctcttttggtcccctgccttcttgaaccaaccgggatggtttggagaagcctttgaaagaactgaaaaagtgtcccagaaacaacagctcaagatatttcggtacacttctatttcatagttgctagaagccctttctttttcgtttctttttcttttttttttttctttttttttttttttttttttttatctttttcttgcttcctcttaagctctcttactttggataatggccttggacttgggtgccttatcgatttcccccttcaagatgctgtatcatttggttggggggagctctgcatggtaatgcactgtgagagaggccgggccttctggaggtgatccggatggagatttatcccccagacatgtctgagggagctggcgggaggtcctccagcccctccactcagctgagtgcagacccatctctcgatgggcttccggcagcggaacatatgccagacacccacacagaagatgggagaagccctggactcctgggcctggccgtgccctgctgtgtctgcctggaagcggagcgtctcagagggtgcctcaactccgagaagatctgcattgttcccattctggcttgtctagtaagcctctgcctctgcattgctggcctaaagtgggtatttgtggacaagatattcgaatacgactctcctacccaccttgaccctggggggttaggccaggaccctgttatttctctggatccaacggctgcctccgctgttctggtctcatccgaggcatacacttcacctgtctctaaggctcagtctgaagctgaggctcatgttacagggcaaggtgaccatgtcgctgtggcctctgaaccttccgcagtacccacccggaagaaccgactgtctgcttttcctcccttacactccactccaccgcccttcccttctccagctcggacccctgaggtgagaacacccaagtcaggaactcagccacaaacaacagaaactaatctgcaaactgctcctaaactt

SEQ ID NO: 27 - protein, 222 aa, Mus musculus
MEIYPPDMSEGAGGRSSSPSTQLSADPSLDGLPAAEHMPDTHTEDGRSPGLLGLAVPCCVCLEAERLRGCLNSEKICIVPILACLVSLCLCIAGLKWVFVDKIFEYDSPTHLDPGGLGQDPVISLDPTAASAVLVSSEAYTSPVSKAQSEAEAHVTGQGDHVAVASEPSAVPTRKNRLSAFPPLHSTPPPFPSPARTPEVRTPKSGTQPQTTETNLQTAPKL

SEQ ID NO: 28 - protein, 169 aa, Homo sapiens
WSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 29 - DNA, 15442 bp, Homo sapiens
tctgcggagaagatccatctaacccctttctggccccagagtccagggaaaggatgatcactgtcagaagtcgtggcgcgggagcccactgggcgctttgtcacattccaccgaaagtcccgacttggtgacagtgtgcttcccttccctcgccaacagttccgagtgagctgtgctttagctctcgtgggggtgggtcaagggaggatttgaagagtcattgccccactttacccttttggagaaatggcttgaaatttgctgtgacacgggcagcatgggaatagtccttcctgaaccctggaaaggagctcctgccagccttgcacacactttgtcctggtgaaaggcagccctggagcaggtgtttttttggaactccaaacctgcccacccaacttgcttctgaaagggactctaaagggtccctttccgctcctctctgacgccttccctcagccagaattcccttggagaggaggcaagaggaaagccatggacaggggtcgctgctaacaccgcaagttcctcagaccctggcacaaaggccttggctacaggcctccaagtagggaggagggggaggagtggctgcctggccacagtgtgaccttcagaggcccccagagaaggacacctggcccctgcctgcctagaaccgcccctcctgtgccccctggccttggaaggggtatgaaatttccgtcccctttcctccttggggcccaggaggagtggagggtcccgggagaatattgtcagggggaaggcagggggtgtcatgggaatgggtgagggggctgaggtgcagaatccagggggtccctgcaggagccgcagtggtaagctgtccagctggaagcctggtaactgttgttttctcttgagaggggcttcctgtgaccttggctgtctctgggagcagggctggggtacctgagtggggtgcatttggggtgtgtgggaaggagagggaaagaaagatggacagtgggactctcccctagcagggtctggtgttccgtaggctagagtgcccctctgctctgcgagtgctgggcgggaggggagttggtgagagctggagacccccaggaagggctggcagaagcctttccttttgggtgctgtcaggtccgcatgtcttggcgtgttgaccttcacagcttctggcgaggggaggaatgatctgatgcgggtggggagggttagaggaggcctcaggcctaaggtggtgcagggggccccctaggggctgggcagtgccaaggcataaaagccttccctggtccctggtggcatttgaaggtgcccaggtgagaggggcttggcacctcctcaccctgggagggagaagaaaccagggaacaggtaggagtgggagacaggtgaggctttggaaatctattgaggctctggagagatttgtgtagagaggaaaatgtggttctcccccagggtctcctcctgggtttttaccctctaagcaacctgtgggcatgctgggttattcctaaggactagaagagcttggatgggggagggtggttggtgcccttcggtcctcggcacccccctccgtctccaacaccagctcaccctggtatttgtcatgtcagcaggagaaggtcaccatgttgtttttctcgcccctagtccttccttcctgccccagtccaaatttgtcctcctatttgaccttaatacttaccatggctttggaccagggaactagggggatagtgagagcagggagagggaagtgtggggaaggtacaggggacctcgacagtgaagcattctggggttttcctcctgcatttcgagctccccagcccccaacatctggttagtctttaacttcctcgggttcataaccatagcagtccaggagtggtgggcatattctgtgcccgtggggacccccggttgtgtcctgttcgactcagaagacttggagaagccagaggctgttggtgggagggaagtgaggagggaggaggggctgggtggctgggcctgtgcaccccagcccctgcccatgcccatgccttgctctctttctgtcctcagtggtcccaggctgcacccatggcagaaggaggagggcagaatcatcacgaaggtgagtccccctggctgttggatggggttccctgtcctctcaggggatgggtggatggcctaattcctttttcttcagaactgtggggaggaaggggaaggggcacaggaatataaggatcaagaaagaaagagctgggcaccacgaggttcaccctcagtttcgtgaggactctccgctgttcaggtctctgctagaagtaggacttgttgcctttttcttctgctctttccagtaaaattttatttggagaaggagtcgtgcgcacagagcaggaagacagtgttcacggatcctaggtgttgggggaagtgtcccttgtttcccctagctcccaggggagagtggacatttagtgtcatttcctatatagacatgtcccatttgtgggaactgtgacccttcctgtgtgagctggaggcacagagggctcagcctaatgggatctcccctcccttccctggtttgcattcctttgggggtggagaaaaccccacttgactatgttcgggtgctgtgaacttccctcccaggccagcagagggctggctgtagctcccaggcgccccgcccccctgcccaaccccgagtccgcctgccttttgttccgttgtggtttggatcctcccatttctctggggacaccctggctctccccaccactgactgtggcctgtgctctccacctctggggagggaaggccctggggtcttccttcccgcgagtttccctgacctaaatctggcgtggctgggtagtggccagcagtggtgatgcccagcctgttctgcctcctccttccccaccccaggagccctttccttggcctaggacctggcttctcagccactgaccggccccctgcttccagtgcgccacttaccccttccagcttcccagtggtctctggtctgggagaggcaggacaaatggtctttgtttgctggagaaaaggttgtctgcgataaataaggaaaaccacgaaagcctggttgttggagtgtacgtgtgtgctcccccaggcagtggaggccagccctccttggaggggcggctgcctgatgaaggatgcgggtgaggttccccgcctccacctcccatgggacttggggattcattccaaggggaagctttttgggggaattcctaccccaggtctttttaccctcagttaccaaccccttgcccaggccagaccttcctgctatcccctcctgggccacaagcctggccctcctctgtcccaattgtgatgaaggggcagttcaaaacttcttgattagtcatcttctcccctatcgacttggctttaaaaaatgaccttttcagacttctagtctcgttcactctttttgatgatgctttgccgtaacccttcgtgggtagagaaggattctgtgcccattggtggtctggataaaagaaatagagacctcacaggaagcagtggactggcctgtttccccactgttctttctgttttcacacctgtggccttctccccaccttcttcccaatcaacctattgtgtacatagcccccctcattgtcctttattcttctggaaagcagaccttggagggaggagtgagggggaggctcagctgtggtctctggggggtgggggttgggagctggggtggaagtccacgaagcatacacttaagatgctttggtgaagttctaaacttcatattacccaggctgaaaaaagagcacttgttcctagggctggaaatggaagccaaaacaccacctttttcagcctgtttcagcatctttagagatcagcccaacccacttacacagttgagcagagttggaggcctagagaggggagggactggcccaaggtcataccaactcatggccagagcctgggcctcctcactggccaggtgttatttcttccctctgggtagggaacctatttcagggacaggattgctatgtggtagtggtggtggggtgcgataggcgtggcaggctgggccacaatttggagtagtcatgccagagtcctgcatttatttattctcaagggccccgcctctgtggcccagaattaccccttcatgctccagtgcaccccaggcttcgtggccagcctgggaaactgtctctaccctggtctcccttcagatcagcttctagaaatgtttcgtggctacagtggcagcactgttttttccatgatgcaagcagtttgccctcttgggcggggttatcagtggctggcagggctggcacagcgtgtccgcccactgccacctgtgggttccaggagggcccagcccctgtgctgatgcccaccaccttctcagctcatgtctggggaagaggactggcagggggaaaggtgcctcctcctgaaaggtgcctcctctgtttttgcctaatataggcttgggaacactttgatgtcagctaattctgactcctttacttactagctgtgcggccttggggcaacttacttagcctctttgagcctcctgttccccatctgtaaaatggaatctcaatagtgtctaatagtaccatgtggagaaacttgtgtgaaatgatagctgtggactactgtacacagtactcaggatgtagtaagtgctcaataaacagctgttggtatggttgacgttatggtagtggttgtggggaggacgtaggaaactggagactagcttggcaaagctggctcttcctccttttagggaaagcttagagcatccccatggggtatacccatactcagactgtcctctggcatcgaggttggcccaggattcagttcagctgtcacagtgaggtggcgggatcagatgtggcaggccatgtcccttggaacttgagtacatcgtgtgatctctggaatgaaaacaggccttcaccagtgttgatggtggaaagcttagggaagtgcttcaaacacagtaggagggacttacgttagattttggaaggacttgcctgattcggaagctccaaagagtggcattacagagctgggtggagagaggggctagccatcttttgtgtcgcccaccgggctcatgtgtcatcgcctctcatgcagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcaggtgggcatctttgggaagtggggcaagggggggatagggaggggggtaacactttgggaacaggtggtcccaggtcgtttcctggctagatttgccttgtctggctcctgcccctgagttgcacaggggaggtatggtggggtcttgccttctgtggagaagatgcttcattcccagcccaggttcccagcaagccccaaccatctccttctccctgatggttgcccatgggctcaggaggggacagatggatgcctgtgtcaggagcccctctctccctctcttggagagagtcctgagtgcccccccttcttgggggctttgtttgggaagctggatgagcctggtccatggagagtttaaaaagtcttttggtgttacctggtaatggggcacatctcagcccagatagggtgggagggagctgtgaaacacagggagggggttgctttcgggtatctactaggagtcagggtgaagcctagagaggatgaaagaaggggaggggatggggagtggtaagaacctaggatttgaattcccagcctggccaacccttgcagccatgtcttggcctcaagtggaacaagggctccttgaggccagcagggttgggggagttggggtgggcctgagcctctttcctgctagagctcttggtcctccctgcctccaccacccatccctgctctgcagaacccctgggtgctgagtggcaggagccccagggttgtcccatctgggtatggctggctgggtcactaacctctgtgatctgcttccttcctttccagattatgcggatcaaacctcaccaaggccagcacataggagagatgagcttcctacagcacaacaaatgtgaatgcaggtgaggatgtagtcacggattcattatcagcaagtggctgcagggtgcctgatctgtgccagggttaagcatgctgtactttttggcccccgtccagcttcccgctatgtgacctttggcattttacttcaatgtgcctcagtttctacatctgtaaaatgggcacaatagtagtatacttcatagcattgttataatgattaaacaagttatatatgaaaagattaaaacagtgttgctccataataaatgctgtttttactgtgattattattgttgttatccctatcattatcatcaccatcttaacccttccctgttttgctcttttctctctccctacccattgcagaccaaagaaagatagagcaagacaagaaaagtaagtggccctgactttagcacttctccctctccatggccggttgtcttggtttggggctcttggctacctctgttgggggctcccatagcctccctgggtcagggacttggtcttgtgggggacttgtggtggcagcaacaatgggatggagccaactccaggatgatggctctagggctagtgagaaaacatagccaggagcctggcacttcctttggaagggacaatgccttctgggtctccagatcattcctgaccaggacttgctgtttcggtgtgtcagggggcactgtggacactggctcactggcttgctctaggacacccacagtggggagagggagtgggtggcagagaggccagcttttgtgtgtcagaggaaatggcctcttttggtggctgctgtgacggtgcagttggatgcgaggccggctggagggtggtttctcagtgcatgccctcctgtaggcggcaggcggcagacacacagccctcttggccagggagaaaaagttgaatgttggtcattttcagaggcttgtgagtgctccgtgttaaggggcaggtaggatggggtgggggacaaggtttggcggcagtaacccttcaagacagggtgggcggctggcatcagcaagagcttgcagggaaagagagactgagagagagcacctgtgccctgccctttcccccacaccatcttgtctgcctccagtgctgtgcggacattgaagcccccaccaggcctcaaccccttgcctcttccctcagctcccagcttccagagcgaggggatgcggaaaccttccttccaccctttggtgctttctcctaagggggacagacttgccctctctggtcccttctccccctcctttcttccctgtgacagacatcctgaggtgtgttctcttgggcttggcaggcatggagagctctggttctcttgaaggggacaggctacagcctgccccccttcctgtttccccaaatgactgctctgccatggggagagtagggggctcgcctgggctcggaagagtgtctggtgagatggtgtagcaggctttgacaggctggggagagaactccctgccaagtaccgcccaagcctctcctccccagacctccttaactcccaccccatcctgctgcctgcccagggctccaggacacccagccctgcctcccagtccaggtcgtgctgagcaggctggtgttgctcttggttccgtgccagctcccaaggtagccgcttcccccacaccgggattcccagaggttctgtcgcagttgcaaatgaaggcacaaggtgcctgatacacagccctccctcccactcctgctccccatccaggcaggtctctgaccttctccccaaagtctggcctaccttttatcacccccggaccttcagggtcagacttggacagggctgctgggcaaagagccttccctcaggctttgccccctgccggggactgggagccactgtgagtgtggagacctttgggtcctgtgccctccacccagtctcggcttcccaccaaagccttgtcaggggctgggtttgccatcccatggtgggcagcgtgaggagaagaaagagccatcgagtgcttgctgcccagacacgcctgtgtgcgcccgcgcatgcctccccagagaccacctgcctcctgacacttcctccgggaagcggccctgtgtggctttgctttggtcgttcccccatccctgcccaccttaccacttcttttactccccccaccgcccccgctctctctctgtctctgtttttttattttccagaaaatcagttcgaggaaagggaaaggggcaaaaacgaaagcgcaagaaatcccggtataagtcctggagcgtgtacgttggtgcccgctgctgtctaatgccctggagcctccctggcccccagtacaacctccgcctgccattccctgtaaccctgcctccctcccctggtccctccctggctctcatcctcctggcccgtgtctctctctcactctctcactccactaattggcaccaacgggtagatttggtggtggcattgctggtccagggttggggtgaatgggggtgccgacttggcctggaggattaagggaggggaccctggcttggctgggcaccgattttctctcacccactgggcactggtggcgggcccatgttggcacaggtgcctgctcacccaactggtttccattgctctaggcttctgcactcgtctggaagctgagggtggtggggagggcagacatggcccaagaagggctgtgaatgactggaggcagcttgctgaatgactccttggctgaaggaggagcttgggtgggatcagacaccatgtggcggcctcccttcatctggtggaagtgccctggctcctcacggaggtggggcctctggaggggagccccctattccggcccaacccatggcacccacagaggcctccttgcagggcagcctcttcctccgggtcggaggctgtggtgggccctgccctgggccctctggccaccagcggcctggcctggggacactgcctccgggcttagcctcccatcacaccctactttagcccaccttggtggaagggcctggacatgagccttgcacggggagaaggtggcccctgattgccatccccagcaggtgaagagtcaaggcgtgctccgatgggggcaacagcagttgggtccctgtggcctgagactcacccttgtctcccagagacacagcattgccccttatggcagcctctccctgcactctctgcccgtctgtgcccgcctcttcctgcggcaggtgtcctagccagtgctgcctctttccgccgctctctctgtcttttgctgtagcgctcggatccttccagggcctgggggctgaccggctgggtgggggtgcagctgcggacatgttagggggtgttgcatggtgatttttttctctctctctgctgatgctctagcttagatgtctttccttttgcctttttgcagtccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcaggttggttcccagagggcaagcaagtcagagaggggcatcacacagagatggggagagagagagagaaagagagtgagcgagcgagcgagcgggagagcgcctgagaggggccagctgcttgctcagtttctagctgcctgcctggtgactgctgccttctctgcttttaaggcccctgtggtgggctgcaggcactggtccagcctggcggggcctgttccgaggttgccctggttgcctgagtggtaggctggtgtggcttagtgtagtggtgtggacgcaagctgtgtgttgtgtcctgtggtccttctgctcatagtggctgttggtcctgatgttattactacctctggtagtaatgctgagaagctgaaagccgattccaggtgtggacaatgtcaacaaagcacagatgctctcgctggggccttgcctcggccctttgaagtctgcatggctgggcttctcactcactcagtgtttcttgctgggggaaggaattgagtctcccacttcagactgggcctccctgaggaaagggttgtgtctccccactcagactgaggttccctgagggtagggctgtgtctctcccctccgacctgggctccctgatagggctgtctccccgctcagactgaggctccctcaggccagggctatgtctccctcctcagactggggctctgagggcaaggggtctggctgttcgtttaggatggggcagttttgcctacacactgaaggagctgtagcatccaagaatactagatacctttaatcctccaccagtcatggtgacaaccccaagcagcccacacattttcaagtgcccccaggatgcgtggagggaggggtctgtgcccattctcctgacattagcctgtgagctccgtaagcccgggcctcgtttacgtacctttgtgagccccgggcatctgtacctctttcctttgcccatactggggaccaaggaagtgtcaagtgcatgagtgaatgtgtgactcagttcagagggtgaggtcaggagcacagggtcgggacaggtggctggcatcttttaatgccttagcttatgttctttataccaacttggcctgtgctcagagtgagggaggccctgggggtcagggtaagcgtcagtcagggaggcaagactttgtggggatttcctagacagggccaaggcacccccagctcaccccgaggctgcgttagggaagtccttggagtgtctcccctcccccagcaatgttcttgtggcttgtgtgtgctcaggggatgctgggaaccaggcctgggtagttggtgtggggtgctgtctgtcttggccctatgtgaaaccaagagggcgtatattagtgctggggtgggggctctgcctaacttcagggctggatgaggggagtctcagttccccaggggtccttgggaaagataagggacttgacattttagggtttttaggtgattattctgctgatgggggtttgtgtgaagtgacctgggagctaactgaagttactctaacctcccaatacctttacccaacccccaagctggctgtatctgggaatatcagtttccaaaattggaggcttaggactccgtttcggggctccccagaagggtagggcctgttctgcctccttctcacaatcacccaggggcaggggcatgctgagaaagttcttggaggccccctttgcttcagctggagtagtgaagccgccgaattgtctctccccatcctaagtgaagcagcatatttgaaaggaaagacaacctgttacctgggcctgcaacctccaggcagctcaagagagatgaggcctacagccacactgggaggggacatggggaatggagatggtccctcaccttcctggggcctcctgctctacgctaccccctcgggagcctcctgtccccagggcaggcccttgccattgttggtcacccggccaagcctctctgcctcaggcgttctcccagaagatctgcccactctcttccccacaccagcccctagagactgaactgaaaaccctcctcagcagggagcctcttctgattaacttcatccagctctggtcacccatcagctcttaaaatgtcaagtggggactgttctttggtatccgttcatttgttgctttgtaaagtgttcccatgtccttgtcttgtctcaagtagattgcaagctcaggagggtagactgggagcccctgagtggagctgctgctcaggccggggctccctgagggcagggctggggctgttctcatactggggctttctgccccaggaccacaccttcctgtcctctctgctcttatggtgccggaggctgcagtgacccaggggcccccaggaatggggaggccgcctgcctcatcgccaggcctcctcacttggccctaaccccagcctttgttttccatttccctcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggaggccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaagctcctcttggtttctccttctccattgaatccttgctctagacttcctcccgccccctttccctcttcctctggggaacatggcatttgtcttgggtctgggaaaggtacgtctaatgtgtaggatatggggtgacccaccttgttgtgctgggggcaaagtccttccattttggctgagctggtcctgggggaacccatccatcctgtcttgatatagaaggtgggaagctctgggaatgggtggagggggagaaacagcttaggagccaagggcccttgcaattggtagtgctgccttcaggaattagatgatccaggcccctgtcccttagccagggaaagaactggccatgtctccaagcttgctgcccaggagacaggagagagctgtttttgtctgtgggggtcttgttggctgcaacaggctgggagtgggagggggatgctgctggagggctgtgactccagggtgtaacttaattttacatagttttacaccctggagttccttgagcctctggaagatggacccataggtttggtcaccactgatgggactccctgccccagcttgccataagctcttctctcacgtcctgctcctgggtaaggtggcacctatccaggtctttgacactgaagggcagtgcttccaaattcacttcctctagcctctcatttattcttgcaaaccatagatatggcttgaataaatattgagccagtcattgtgctgctatgaataagacacaattccacccctcaaggatctggtgaggatgggtgggtggggagacccaaaactataaatccatgagcagaaaaatacataaaatgtgctgggggcatctgatctagcttgggagtgggagttgtattgggggtggtggctggggggtggtgtcttatgacattatctctaggctgccacttaaagtatggtttgaagacagggagaacggggcggcggagtgaaagggttgaggacatcccaggcagaagggatagtgtgagcaaggcatgaaggtggcacttgggcaggggcagggagtgggtgggcagggtggaggctggagacacgggcaagggttagctccaagggagcctgcagcaccatgccaagagcctagggcctgtcagggatgagcagcagccgaggggttttaaacactgagcggctcagtcagatagacttttccataggtgcctggcaacag

SEQ ID NO: 30 - DNA, 2250 bp, Mus musculus
tgagccaggctggcaggaaggagcctccctcagggtttcgggaaccagacctctcaccggaaagaccgattaaccatgtcaccaccacgccatcatcgtcaccgttgacagaacagtccttaatccagaaagcctgacatgaaggaagaggagactcttcgaggagcactttgggtccggagggcgagactccggcagacgcattcccgggcaggtgaccaagcacggtccctcgtgggactggattcgccattttcttatatctgctgctaaatcgccaagcccggaagattagggttgtttctgggattcctgtagacacacccacccacatacacacatatatatatattatatatataaataaatatatatgttttatatataaaatatatatatattcttttttttaaattaactctgctaatgttattggtgtcttcactggatatgtttgactgctgtggacttgtgttgggaggaggatgtcctcactcggatgccgacatgggagacaatgggatgaaaggcttcagtgtggtctgagagaggccgaagtccttttgcctgccggggagcaagcaaggccagggcacgggggcacattggctcacttccagaaacacgacaaacccattcctggccctgagtcaagaggacagagagacagatgatgacacagaaagagataaagatgccggttccaaccagaagtttggggagcctcaggacatggcatgctttgtggatccccatgatagtctacaaaagcaccccgcccctctgggcactgcctggaagaatcgggagcctggccagccttcagctcgctcctccacttctgaggggcctaggaggcctcccacaggtgtcccggcaagagaagacacggtggtggaagaagaggcctggtaatggcccctcctcctgggaccccttcgtcctctccttaccccacctcctgggtacagcccaggaggaccttgtgtgatcagaccattgaaaccactaattctgtccccaggagacttggctctgtgtgtgagtggcttacccttcctcatcttcccttcccaaggcacagagcaatggggcaggacccgcaagcccctcacggaggcagagaaaagagaaagtgttttatatacggtacttatttaatagccctttttaattagaaattaaaacagttaatttaattaaagagtagggtttttttcagtattcttggttaatatttaatttcaactatttatgagatgtatctctcgctctctcttatttgtacttatgtgtgtgtgtgtgtgtgtgtgtgtgtgtgtgtgtgtgtgtatgaaatctgtgtttccaatctctctctcccagatcggtgacagtcactagcttgtcctgagaagatatttaattttgctaacactcagctctgccctcccttgtccccaccacacattcctttgaaataaggtttcaatatacatttacatactatatatatatttggcaacttgtgtttgtatataaatatatatatatatatatgtttatgtatatatgtgattctgataaaatagacattgctattctgttttttatatgtaaaaacaaaacaagaaaaatagagaattctacatactaaatctctctccttttttaattttaatatttgttatcatttatttattggtgctactgtttatccgtaataattgtgggggaaaaagatattaacatcacgtctttgtctctagagcagttttccgagatattccgtagtacatatttatttttaaacagcaacaaagaaatacagatatatcttaaaaaaaaagcattttgtattaaagaattgaattctgatctcaaagctctccctggtctctccttctctcctgggccctcctgtctcgctttccctcctcctttggggtacatagtttttgtcttaggtttgagaagcagtccctggagtagaatatggggtgacccatccattcctgggcggaggggagatggctcctttgccaagggtcctcacactacgtggtactctgttccttgtcagacaaggatgggggcatgtctccaggtgctaactggagatcggagagagctgttggctgcagctggccaggatttgggcatgccggggacatgggaggctgtgagcccagcatgcagtttacttctgggtgctaaatggaagagtccagtaaaaagagtcttgcccatgggattccattccgctttgtg

SEQ ID NO: 31 - protein, 123 aa, Mus musculus
MNFLLSWVHWTLALLLYLHHAKWSQAAPTTEGEQKSHEVIKFMDVYQRSYCRPIETLVDIFQEYPDEIEYIFKPSCVPLMRCAGCCNDEALECVPTSESNITMQIMRIKPHQSQHIGEMSFLQ

SEQ ID NO: 32 - DNA, 1515 bp, Homo sapiens
atggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggacgcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccgctactgatgcaggcactgcaggagccctgactccacagcatgttcgagctcatgcctctccagcttctctgcagttgggagctgtttctcctgggacactgacccccactggagtagtctctggcccagcagctacacccacagctcagcatcttcgacagtctgcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcaggtcctcttcctgatggatgggaacaagccatgactcaggatggagaaatttactatataaaccataagaacaagaccacctcttggctagacccaaggcttgaccctcgttttgccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggcaatgcggaatatcaatcccagcacagcaaattctccaaaatgtcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgaggctacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttataa

SEQ ID NO: 33 - DNA, 1461 bp, Homo sapiens
cccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggacgcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccgctactgatgcaggcactgcaggagccctgactccacagcatgttcgagctcatgccgctccagctgctctgcagttgggagctgtttctcctgggacactgacccccactggagtagtctctggcccagcagctacacccacagctcagcatcttcgacaggctgcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcaggtcctcttcctgatggatgggaacaagccatgactcaggatggagaaatttactatataaaccataagaacaagaccacctcttggctagacccaaggcttgaccctcgttttgccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgaggctacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttatag

SEQ ID NO: 34 - DNA, 2459 bp, Mus musculus
tctgaacgctgtatcatttggttggggggagctctgcatggtaatgcactgtgagagaggccgggccttctggaggtgatccggatggagatttatcccccagacatgtctgagggagctggcgggaggtcctccagcccctccactcagctgagtgcagacccatctctcgatgggcttccggcagcggaacatatgccagacacccacacagaagatgggagaagccctggactcctgggcctggcggtgccctgctgtgtctgcctggaagcggagcgtctcagagggtgcctcaactccgagaagatctgcattgttcccattctggcttgtctagtaagcctctgcctctgcattgctggcctaaagtgggtatttgtggacaagatatttgaatacgactctcctacccaccttgaccctggggggttaggccaggaccctgttatttctctggatccaacggctgcctccgctgttttggtatcatccgaggcatacacttcacctgtctctaaggctcagtctgaagctgaggctcatgttacagggcaaggtgaccatgtcgctgtggcctctgaaccttccgcagtacccacccggaagaaccggctgtctgcttttcctcccttacactccactccaccgcccttcccttctccagctcggacccctgaggtgagaacacccaagtcaggaactcagccacaaacaacagaaactaatctgcaaactgctcctaaactttccacatctacatccacgactgggaccagccatctcataaagtgtgcggagaaggagaaaactttctgtgtgaatggaggcgagtgcttcatggtgaaggacctgtcaaacccctcaagatacttgtgcaagtgcccaaatgagtttactggtgatcgttgccaaaactacgtaatggccagcttctacaagcatcttgggattgaatttatggaagcggaggagctctaccagaagagggtactgacaattactggcatctgtatcgccctgttggtggtcggcatcatgtgtgtggtggcctactgcaaaaccaagaaacagcggcagaagcttcatgatcggctccggcagagccttcggtcagaacgaaacaacatggtgaacatagcgaatggccctcaccatccaaacccaccaccagagaatgtgcaactggtgaatcaatatgtatctaaaaacgtcatctccagtgagcatattgtggagagagaagtggagacctccttttccaccagtcactacacttccacagctcatcactccacgactgtcacccagactcctagtcacagctggagtaatgggcacacagaaagcatcatttcagaaagccactctgtaatcatgatgtcatcggtagagaacagcaggcacagcagcccagctgggggcccacgaggacgtcttcatggcctgggaggccctcgcgaatgtaacagcttcctcaggcatgccagagaaacccctgactcctacagagactctcctcatagtgaaaggtatgtatcagccatgaccaccccggctcgtatgtcacctgtagatttccacacgccaagctcccctaaatcgcccccttcggaaatgtctccacccgtgtccagcatgacggtgtccatgccctctgtggcagtcagcccctttgtggaagaagagaggcctctgcttcttgtgacaccaccgaggctacgggagaagaagtatgatcatcacccccagcaactcaactcctttcatcacaaccctgcacatcagagtaccagcctcccccctagcccattgaggatagtggaggatgaggaatacgaaacgacccaggagtatgagccaattcaagagcctattaagaaagtcaccaatagccggcgggccaaaagaaccaagcccaatggccacattgccaataggttggaaatggacagcaacccaagttctgtgagcagtaactcagaaagtgagacagaagatgaaagagtaggtgaagatacaccattcctgggcatacagaaccccctggcagccagccttgaggtggcccctgccttccgtctggctgagagcaggactaacccagcaggccgcttctccacacaggaagaattacaggccaggctgtctagtgtaatcgctaaccaagaccctattgctgtataaaacctaaataaacacatagattcacctgtaaaactttattttatataataaagtatttcaccttaaattaaacaatttattttattttagcagttctgcaaatagaaaacaggaagaaaaaaacttttataaattaaatatatgtatgtaaaaatgtgttatgtgccatatgtagcaattttttacagtatttcaaaaaaacgagaaagatatcaatggtgcctttatgttatgttatgtcgagagcaagttttataaagttacggtggttg

SEQ ID NO: 35 - protein, 700 aa, Mus musculus
MEIYPPDMSEGAGGRSSSPSTQLSADPSLDGLPAAEHMPDTHTEDGRSPGLLGLAVPCCVCLEAERLRGCLNSEKICIVPILACLVSLCLCIAGLKWVFVDKIFEYDSPTHLDPGGLGQDPVISLDPTAASAVLVSSEAYTSPVSKAQSEAEAHVTGQGDHVAVASEPSAVPTRKNRLSAFPPLHSTPPPFPSPARTPEVRTPKSGTQPQTTETNLQTAPKLSTSTSTTGTSHLIKCAEKEKTFCVNGGECFMVKDLSNPSRYLCKCPNEFTGDRCQNYVMASFYKHLGIEFMEAEELYQKRVLTITGICIALLVVGIMCVVAYCKTKKQRQKLHDRLRQSLRSERNNMVNIANGPHHPNPPPENVQLVNQYVSKNVISSEHIVEREVETSFSTSHYTSTAHHSTTVTQTPSHSWSNGHTESIISESHSVIMMSSVENSRHSSPAGGPRGRLHGLGGPRECNSFLRHARETPDSYRDSPHSERYVSAMTTPARMSPVDFHTPSSPKSPPSEMSPPVSSMTVSMPSVAVSPFVEEERPLLLVTPPRLREKKYDHHPQQLNSFHHNPAHQSTSLPPSPLRIVEDEEYETTQEYEPIQEPIKKVTNSRRAKRTKPNGHIANRLEMDSNPSSVSSNSESETEDERVGEDTPFLGIQNPLAASLEVAPAFRLAESRTNPAGRFSTQEELQARLSSVIANQDPIAV

SEQ ID NO: 36 - protein, 190 aa, Mus musculus
MNFLLSWVHWTLALLLYLHHAKWSQAAPTTEGEQKSHEVIKFMDVYQRSYCRPIETLVDIFQEYPDEIEYIFKPSCVPLMRCAGCCNDEALECVPTSESNITMQIMRIKPHQSQHIGEMSFLQHSRCECRPKKDRTKPENHCEPCSERRKHLFVQDPQTCKCSCKNTDSRCKARQLELNERTCRCDKPRR

SEQ ID NO: 37 - DNA, 2848 bp, Mus musculus
cgggattgcacggaaacttttcgtccaacttctgggctcttctcgctccgtagtagccgtggtctgcgccgcaggagacaaaccgatcggagctgggagaagtgctagctcgggcctggagaagccggggcccgagaagagaggggaggaagagaaggaagaggagagggggccgcagtgggcgctcggctctcaggagccgagctcatggacgggtgaggcggccgtgtgcgcagacagtgctccagccgcgcgcgcgccccaggccccggcccgggcctcggttccagaagggagaggagcccgccaaggcgcgcaagagagcgggctgcctcgcagtccgagccggagagggagcgcgagccgcgccggccccggacgggcctccgaaaccatgaactttctgctctcttgggtgcactggaccctggctttactgctgtacctccaccatgccaagtggtcccaggctgcacccacgacagaaggagagcagaagtcccatgaagtgatcaagttcatggatgtctaccagcgaagctactgccgtccgattgagaccctggtggacatcttccaggagtaccccgacgagatagagtacatcttcaagccgtcctgtgtgccgctgatgcgctgtgcaggctgctgtaacgatgaagccctggagtgcgtgcccacgtcagagagcaacatcaccatgcagatcatgcggatcaaacctcaccaaagccagcacataggagagatgagcttcctacagcacagcagatgtgaatgcagaccaaagaaagacagaacaaagccagaaaatcactgtgagccttgttcagagcggagaaagcatttgtttgtccaagatccgcagacgtgtaaatgttcctgcaaaaacacagactcgcgttgcaaggcgaggcagcttgagttaaacgaacgtacttgcagatgtgacaagccaaggcggtgagccaggctgcaggaaggagcctccctcagggtttcgggaaccagacctctcaccggaaagaccgattaaccatgtcaccaccacgccatcatcgtcaccgttgacagaacagtccttaatccagaaagcctgacatgaaggaagaggagactcttcgaggagcactttgggtccggagggcgagactccggcagacgcattcccgggcaggtgaccaagcacggtccctcgtgggactggattcgccattttcttatatctgctgctaaatcgccaagcccggaagattagggttgtttctgggattcctgtagacacacccacccacatacacacatatatatatattatatatataaataaatatatatgttttatatataaaatatatatatattcttttttttaaattaactctgctaatgttattggtgtcttcactggatatgtttgactgctgtggacttgtgttgggaggaggatgtcctcactcggatgccgacacgggagacaatgggatgaaaggcttcagtgtggtctgagagaggccgaagtccttttgcctgccggggagcaagcaaggccagggcacgggggcacatttgctcacttccagaaacacgacaaacccattcctggccctgagtcaagaggacagagagacagatgatgacagagaaagagataaagatgccggttccaaccagaagtttggggagcctcaggacatggcatgctttgtggatccccatgatagtctacaaaagcaccccgcccctctgggcactgcctggaagaatcgggagcctggccagccttcagctcgctcctccacttctgaggggcctaggaggcctcccacaggtgtcccggcaagagaagacacggtggtggaagaagaggcctggtaatggcccctcctcctgggaccccttcgtcctctccttaccccacctcctgggtacagcccaggaggaccttgtgtgatcagaccattgaaaccactaattctgtccccaggagacttggctgtgtgtgtgagtggcttacccttcctcatcttcccttcccaaggcacagagcaatggggcaggacccgcaagcccctcacggaggcagagaaaagagaaagtgttttatatacggtacttatttaatagccctttttaattagaaattaaaacagttaatttaattaaagagtagggtttttttcagtattcttggttaatatttaatttcaactatttatgagatgtatctctcgctctctcttatttgtacttgtgtgtgtgtgtgtgtgtgtgtgtgtgtgtgtgtgtgtgtgtgtatgaaatctgtgtttccaatctctctctcccagatcggtgacagtcactagcttgtcctgagaagatatttaattttgctaacactcagctctgccctcccttgtccccaccacacattcctttgaaataaggtttcaatatacatttacatactatatatatatttggcaacttgtgtttgtatataaatatatatatatatatatatgtttatgtatatatgtgattctgataaaatagacattgctattctgttttttatatgtaaaaacaaaacaagaaaaatagagaattctacatactaaatctctctccttttttaattttaatatttgttatcatttatttattggtgctactgtttatccgtaataattgtgggggaaaaagatattaacatcacgtctttgtctctagagcagttttccgagatattccgtagtacatatttatttttaaacagcaacaaagaaatacagatatatcttaaaaaaaaaagcattttgtattaaagaattgaattctgatctcaaaaaaaaaaaaaaaaaaa

(19) United States
(12) Patent Application Publication
(10) Pub. No.: US 2024/0026355 A1
(43) Pub. Date: Jan. 25, 2024

(54) COMPOSITIONS FOR AND METHODS OF ENHANCING TISSUE REGENERATION

(71) Applicant: Duke University, Durham, NC (US)

(72) Inventors: Kenneth Poss, Durham, NC (US); Ruorong Yan, Durham, NC (US); Junsu Kang, Durham, NC (US); Joseph Goldman, Durham, NC (US); Valentia Cigliola, Durham, NC (US)

(73) Assignee: Duke University, Durham, NC (US)

(21) Appl. No.: 18/037,054

(22) PCT Filed: Nov. 16, 2021
(86) PCT No.: PCT/US21/59449

§ 371 Date: May 15, 2023

Related U.S. Application Data
(60) Provisional application No. 63/114,432, filed on Nov. 16, 2020.
(60) Provisional application No. 63/182,974, filed on May 2, 2021.

(51) Int. Cl. C12N 15/113; C12N 15/86

(52) U.S. Cl. CPC C12N 15/113; C12N 15/86; C12N 2320/30; C12N 2750/14143; C12N 2750/14171

(57) ABSTRACT

Disclosed herein are methods are compositions and methods for enhancing regeneration in stressed damaged, and/or injured tissues.

### CROSS-REFERENCE TO RELATED APPLICATIONS

This application claims the benefit of U.S. Provisional Application No. 63/114,432 filed 16 Nov. 2020 and the benefit of U.S. Provisional Application No. 63/182,974 filed 2 May 2021, both of which are incorporated by reference herein in their entirety.

### STATEMENT REGARDING FEDERALLY SPONSORED RESEARCH

Parts of this invention were made with government support under Grant No. R35 HL150713 and Grant No. R01 HL136182 awarded by the National Heart, Lung, and Blood Institute (NHLBI).

### REFERENCE TO THE SEQUENCE LISTING

The Sequence Listing submitted 16 Nov. 2021 as a text file named “20_2002_WO_ Sequence_Listing”, created on 16 Nov. 2021 and having a size of 299 kilobytes is hereby incorporated by reference pursuant to 37 C.F.R. § 1.52(e)(5).

### BACKGROUND OF THE INVENTION

The capacity for tissue regeneration is distributed unequally among species and tissues. Non-mammalian vertebrates like zebrafish display potential as adults to regenerate tissues like heart, spinal cord, retina, and major appendages (Poss K D, et al. (2002) Science. 298: 2188-2190; Becker T, et al. (1997) J Comp Neurol. 377:577-595; Vihtelic T S, et al. (2000) J Neurobiol. 44: 289-307; Sehring I, et al. (2021) Cold Spring Harb Perspect Biol. a040758). By contrast, the capacity for this level of regeneration is diminished during the progression of developmental stages in mammals and largely absent in adults

Regeneration involves the rewiring of expression of hundreds to thousands of genes, shifting programmatic focus of an organ or appendage from pure function to morphogenesis (Goldman J A, et al. (2020) Nat Rev Genet. 21:511-525). Understanding how these changes in gene expression are orchestrated and interpreted is one of the greatest challenges in the field of regenerative biology. Distal-acting regulatory sequences, or enhancers, can direct expression of their target genes and have been predominantly studied as a means for stage- and tissue-specific regulation during embryonic development. Recently, an enhancer linked to the leptin b (lepb) gene (“LEN”) was identified. LEN directs gene expression during fin and heart regeneration in zebrafish, revealing a new class of regulatory sequences—Tissue Regeneration Enhancer Elements (TREEs)—which are identifiable by chromatin profiling. Whereas some enhancers appear to respond to stress or trauma itself, other elements track regeneration weeks after the initial injury or can direct gene expression in response to mitogenic stimuli in the absence of stress or injury (Kang J, et al. (2016) Nature. 532:201-206; Goldman J A, et al. (2017) Developmental Cell. 40:392-404).

Investigations of TREEs can help elucidate upstream binding factors and downstream target genes key to regenerative events. Moreover, regulatory sequences represent potential control modules for manipulating expression of pro-regenerative factors in injury sites. Such factors are typically developmentally potent, or even tumorigenic, and would ideally be delivered in a spatially targeted fashion that is initiated by injury and ceases after resolution of damage. Whereas this type of transgenic rescue is capable of overcoming a genetic defect in regeneration in zebrafish, the cross-species recognition of TREEs has only been superficially explored and it is unknown whether they can be employed to address barriers in mammalian regeneration. Critically, because of the nature of the genes that are manipulated, methods for safety are required that limit effects of these highly potent factors to when and where they are needed—that is, transient targeting to the damaged, stressed, and/or injured tissues.

Accordingly, there is an urgent and previously unmet need to spatiotemporally control gene expression and potentially improve tissue regeneration in damaged, stressed, and/or injured tissues, and especially in adult mammals.

### BRIEF SUMMARY OF THE INVENTION

Disclosed herein is an isolated nucleic acid molecule comprising three or more sequence elements including a noncoding sequence that can control the ability of a minimal promoter sequence to direct gene expression in living tissues during stress or injury (called a TREE); a minimal promoter with little or no basal activity in zebrafish tissues; a sequence encoding an enhanced green fluorescent reporter gene; and a 3′ UTR noncoding region that stabilizes the RNA message and enables translation.

Disclosed herein is an isolated nucleic acid molecule comprising a nucleic acid sequence encoding a tissue regeneration enhancer element (TREE); a minimal promoter with little or no basal activity; a reporter transgene; and a 3′ UTR noncoding region.

Disclosed herein is an isolated nucleic acid molecule, comprising: a nucleic acid sequence encoding a tissue regeneration enhancer element (TREE); a minimal promoter with little or no basal activity; further comprising a reporter transgene; and a 3′ UTR noncoding region.

Disclosed herein is an isolated nucleic acid molecule, comprising a nucleic acid sequence encoding a tissue regeneration enhancer element (TREE) and a minimal promoter with little or no basal activity.

Disclosed herein is an isolated nucleic acid molecule, comprising a nucleic acid sequence encoding a tissue regeneration enhancer element (TREE), a minimal promoter with little or no basal activity, and a 3′ UTR noncoding region.

Disclosed herein is an isolated nucleic acid molecule, comprising a nucleic acid sequence encoding a tissue regeneration enhancer element (TREE), a minimal promoter having little or no basal activity, and at least one polypeptide having biological activity.

Disclosed herein is an isolated nucleic acid molecule, comprising a nucleic acid sequence encoding a tissue regeneration enhancer element (TREE), a minimal promoter having little or no basal activity, at least one polypeptide having biological activity, and a 3′ UTR noncoding region.

Disclosed herein are plasmids that comprise a disclosed isolated nucleic acid molecule.

Disclosed herein is a vector comprising a disclosed isolated nucleic molecule.

Disclosed herein is a vector comprising an isolated nucleic acid molecule, comprising a nucleic acid sequence encoding a tissue regeneration enhancer element (TREE) and a minimal promoter with little or no basal activity.

Disclosed herein is a vector comprising an isolated nucleic acid molecule, comprising a nucleic acid sequence encoding a tissue regeneration enhancer element (TREE), a minimal promoter with little or no basal activity, and a 3′ UTR noncoding region.

Disclosed herein is a vector comprising an isolated nucleic acid molecule, comprising a nucleic acid sequence encoding a tissue regeneration enhancer element (TREE), a minimal promoter having little or no basal activity, and at least one polypeptide having biological activity.

Disclosed herein is a vector comprising an isolated nucleic acid molecule, comprising a nucleic acid sequence encoding a tissue regeneration enhancer element (TREE), a minimal promoter having little or no basal activity, at least one polypeptide having biological activity, and a 3′ UTR noncoding region.

Disclosed herein is a lentiviral vector or a recombinant lentiviral vector comprising a disclosed isolated nucleic molecule.

Disclosed herein is a lentiviral vector or a recombinant lentiviral vector comprising an isolated nucleic acid molecule, comprising a nucleic acid sequence encoding a tissue regeneration enhancer element (TREE) and a minimal promoter with little or no basal activity.

Disclosed herein is a lentiviral vector or a recombinant lentiviral vector comprising an isolated nucleic acid molecule, comprising a nucleic acid sequence encoding a tissue regeneration enhancer element (TREE), a minimal promoter with little or no basal activity, and a 3′ UTR noncoding region.

Disclosed herein is a lentiviral vector or a recombinant lentiviral vector comprising an isolated nucleic acid molecule, comprising a nucleic acid sequence encoding a tissue regeneration enhancer element (TREE), a minimal promoter having little or no basal activity, and at least one polypeptide having biological activity.

Disclosed herein is a lentiviral vector or a recombinant lentiviral vector comprising an isolated nucleic acid molecule, comprising a nucleic acid sequence encoding a tissue regeneration enhancer element (TREE), a minimal promoter having little or no basal activity, at least one polypeptide having biological activity, and a 3′ UTR noncoding region.

Disclosed herein is an AAV or an rAAV vector comprising a disclosed isolated nucleic molecule.

Disclosed herein is an AAV or an rAAV vector comprising an isolated nucleic acid molecule, comprising a nucleic acid sequence encoding a tissue regeneration enhancer element (TREE) and a minimal promoter with little or no basal activity.

Disclosed herein is an AAV or an rAAV vector comprising an isolated nucleic acid molecule, comprising a nucleic acid sequence encoding a tissue regeneration enhancer element (TREE), a minimal promoter with little or no basal activity, and a 3′ UTR noncoding region.

Disclosed herein is an AAV or an rAAV vector comprising an isolated nucleic acid molecule, comprising a nucleic acid sequence encoding a tissue regeneration enhancer element (TREE), a minimal promoter having little or no basal activity, and at least one polypeptide having biological activity.

Disclosed herein is an AAV or an rAAV vector comprising an isolated nucleic acid molecule, comprising a nucleic acid sequence encoding a tissue regeneration enhancer element (TREE), a minimal promoter having little or no basal activity, at least one polypeptide having biological activity, and a 3′ UTR noncoding region.

Disclosed herein is a pharmaceutical formulation comprising a disclosed isolated nucleic acid molecule and a pharmaceutically acceptable carrier.

Disclosed herein is a pharmaceutical formulation comprising an isolated nucleic acid molecule, comprising a nucleic acid sequence encoding a tissue regeneration enhancer element (TREE) and a minimal promoter with little or no basal activity, and a pharmaceutically acceptable carrier.

Disclosed herein is a pharmaceutical formulation comprising an isolated nucleic acid molecule, comprising a nucleic acid sequence encoding a tissue regeneration enhancer element (TREE), a minimal promoter with little or no basal activity, and a 3′ UTR noncoding region, and a pharmaceutically acceptable carrier.

Disclosed herein is a pharmaceutical formulation comprising an isolated nucleic acid molecule, comprising a nucleic acid sequence encoding a tissue regeneration enhancer element (TREE), a minimal promoter having little or no basal activity, and at least one polypeptide having biological activity, and a pharmaceutically acceptable carrier.

Disclosed herein is a pharmaceutical formulation comprising an isolated nucleic acid molecule, comprising a nucleic acid sequence encoding a tissue regeneration enhancer element (TREE), a minimal promoter having little or no basal activity, at least one polypeptide having biological activity, and a 3′ UTR noncoding region, and a pharmaceutically acceptable carrier.

Disclosed herein is a pharmaceutical formulation comprising a disclosed vector and a pharmaceutically acceptable carrier.

Disclosed herein is a pharmaceutical formulation comprising a lentiviral vector comprising an isolated nucleic acid molecule, comprising a nucleic acid sequence encoding a tissue regeneration enhancer element (TREE) and a minimal promoter with little or no basal activity, and a pharmaceutically acceptable carrier.

Disclosed herein is a pharmaceutical formulation comprising a lentiviral vector comprising an isolated nucleic acid molecule, comprising a nucleic acid sequence encoding a tissue regeneration enhancer element (TREE), a minimal promoter with little or no basal activity, and a 3′ UTR noncoding region, and a pharmaceutically acceptable carrier.

Disclosed herein is a pharmaceutical formulation comprising a lentiviral vector comprising an isolated nucleic acid molecule, comprising a nucleic acid sequence encoding a tissue regeneration enhancer element (TREE), a minimal promoter having little or no basal activity, and at least one polypeptide having biological activity, and a pharmaceutically acceptable carrier.

Disclosed herein is a pharmaceutical formulation comprising a lentiviral vector comprising an isolated nucleic acid molecule, comprising a nucleic acid sequence encoding a tissue regeneration enhancer element (TREE), a minimal promoter having little or no basal activity, at least one polypeptide having biological activity, and a 3′ UTR noncoding region, and a pharmaceutically acceptable carrier.

Disclosed herein is a pharmaceutical formulation comprising an AAV or an rAAV vector comprising a disclosed isolated nucleic molecule and a pharmaceutically acceptable carrier.

Disclosed herein is a pharmaceutical formulation comprising an AAV or an rAAV vector comprising an isolated nucleic acid molecule, comprising a nucleic acid sequence encoding a tissue regeneration enhancer element (TREE) and a minimal promoter with little or no basal activity, and a pharmaceutically acceptable carrier.

Disclosed herein is a pharmaceutical formulation comprising an AAV or an rAAV vector comprising an isolated nucleic acid molecule, comprising a nucleic acid sequence encoding a tissue regeneration enhancer element (TREE), a minimal promoter with little or no basal activity, and a 3′ UTR noncoding region, and a pharmaceutically acceptable carrier.

Disclosed herein is a pharmaceutical formulation comprising an AAV or an rAAV vector comprising an isolated nucleic acid molecule, comprising a nucleic acid sequence encoding a tissue regeneration enhancer element (TREE), a minimal promoter having little or no basal activity, and at least one polypeptide having biological activity, and a pharmaceutically acceptable carrier.

Disclosed herein is a pharmaceutical formulation comprising an AAV or an rAAV vector comprising an isolated nucleic acid molecule, comprising a nucleic acid sequence encoding a tissue regeneration enhancer element (TREE), a minimal promoter having little or no basal activity, at least one polypeptide having biological activity, and a 3′ UTR noncoding region, and a pharmaceutically acceptable carrier.

Disclosed herein is a method of treating stressed, damaged, and/or injured tissues, the method comprising administering to a subject in need thereof a disclosed isolated nucleic acid molecule, a disclosed vector, a disclosed pharmaceutical formulation, or any combination thereof

Disclosed herein is a method of treating stressed, damaged, and/or injured tissues, the method comprising reducing inflammation in stressed, damaged, and/or injured tissues in a subject in need thereof by administering to the subject a disclosed isolated nucleic acid molecule, a disclosed vector, a disclosed pharmaceutical formulation, or any combination thereof.

Disclosed herein is a method of treating stressed, damaged, and/or injured tissues, the method comprising improving cell survival in stressed, damaged, and/or injured tissues in a subject in need thereof by administering to the subject in need thereof a disclosed isolated nucleic acid molecule, a disclosed vector, a disclosed pharmaceutical formulation, or any combination thereof.

Disclosed herein is a method of treating stressed, damaged, and/or injured tissues, the method comprising stimulating cell proliferation in stressed, damaged, and/or injured tissues in a subject in need thereof by administering to the subject in need thereof a disclosed isolated nucleic acid molecule, a disclosed vector, a disclosed pharmaceutical formulation, or any combination thereof.

Disclosed herein is a method of treating stressed, damaged, and/or injured tissues, the method comprising stimulating cell division in stressed, damaged, and/or injured tissues in a subject in need thereof by administering to the subject in need thereof a disclosed isolated nucleic acid molecule, a disclosed vector, a disclosed pharmaceutical formulation, or any combination thereof.

Disclosed herein is a method of treating stressed, damaged, and/or injured tissues, the method comprising inhibiting cell death mechanism in stressed, damaged, and/or injured tissues in a subject in need thereof by administering to the subject in need thereof a disclosed isolated nucleic acid molecule, a disclosed vector, a disclosed pharmaceutical formulation, or any combination thereof.

Disclosed herein is a method of stimulating cell proliferation in stressed, damaged, and/or injured tissues in a subject in need thereof, the method comprising administering to the subject in need thereof a disclosed isolated nucleic acid molecule, a disclosed vector, a disclosed pharmaceutical formulation, or any combination thereof.

Disclosed herein is a method of improving cell survival in stressed, damaged, and/or injured tissues in a subject in need thereof, the method comprising administering to the subject in need thereof a disclosed isolated nucleic acid molecule, a disclosed vector, a disclosed pharmaceutical formulation, or any combination thereof.

Disclosed herein is a method of enhancing cellular regeneration in stressed, damaged, and/or injured tissues in a subject in need thereof, the method comprising administering to the subject in need thereof a disclosed isolated nucleic acid molecule, a disclosed vector, a disclosed pharmaceutical formulation, or any combination thereof.

Disclosed herein are methods of generating a disclosed viral vector.

Disclosed herein is method of generating a disclosed lentiviral vector comprising seeding cells onto a gelatin coated dish, co-transfecting a DNA expression plasmid with psPAX2 and VSVg, collecting and concentrating the supernatant, and centrifuging and harvesting the concentrated lentiviral particles.

Disclosed herein is a method of generating a disclosed lentiviral vector comprising co-transfecting into cells a recombinant construct containing a pAd-DELTA F6 helper plasmid, a serotype-specific plasmid AAV2/9, and an AAV plasmid containing the cassette of choice, harvesting the transfected cells, and purifying the concentrated AAV particles.

Disclosed herein is a method of identifying one or more putative TREEs comprising isolating the nuclei from a first population of cells and a second population of cells, analyzing chromatin structure and function of the isolated nuclei to obtain a chromatin profile for the first population of cells and for the second population of cells, and comparing the chromatin profiles of the two populations of cells to identify one or more putative TREEs.

Disclosed herein is a method of identifying one or more TREEs comprising obtaining a first population of cells, isolating the nuclei from the first population of cells, analyzing chromatin structure and function of the isolated nuclei to obtain a chromatin profile for the first population of cells, obtaining a second population of cells, isolating the nuclei from the second population of cells, analyzing chromatin structure and function of the isolated nuclei to obtain a chromatin profile for the second population of cells and comparing the chromatin profiles between the two populations of cells to identify one or more putative TREEs.

Disclosed herein is a platform comprising compositions and methods for identifying and/or validating one or more putative TREEs that can spatiotemporally target stressed, damaged, and/or injured tissues for gene expression required for tissue regeneration.

### BRIEF DESCRIPTION OF THE FIGURES

FIG. 1A-FIG. 1F show Zebrafish TREE regulatory sequences direct injury-induced gene expression in adult mouse hearts.

FIG. 1A shows transgene constructs to evaluate the ability of individual zebrafish TREEs to direct expression of a minimal promoter (hsp68) in adult mouse tissues upon injury.

FIG. 1B shows whole-mount images of X-gal-stained adult mouse hearts, with staining (arrowheads) appearing in the area of LAD ligation (asterisks) but not in uninjured hearts. The abbreviations are as follows: a=atrium; v=ventricles. In this experiment, LEN-hsp68::lacZ: n=9 (uninjured), n=11 (3 dpi), and n=6 (7 dpi), and runx1EN-hsp68::lacZ: n=7 (uninjured), n=6 (3 dpi), and n=5 (7 dpi).

FIG. 1C shows section images of X-gal-stained adult mouse hearts, with staining appearing in the large infarcts, and restricted to the injured site (asterisks). High-magnification view (right) of box in left.

FIG. 1D (left panel) shows a schematic for virus delivery and ligation of LAD while FIG. 1D (right panel) shows the experimental design used in FIG. 1E-FIG. 1F.

FIG. 1E shows section images of hearts after sham or MI injury, from adult mice infected pre-injury with AAV9 harboring a zebrafish TREE, an hsp68 minimal promoter, and an EGFP cassette. Fluorescence was induced by injury and restricted to CMs near the MI (asterisks). Staining with the Tnnt antibody showed non-specific background in injury site when stained with fixed heart samples, which was not observed in unfixed heart samples (FIG. 5B). In these experiments, the following applied: (i) LEN-hsp68::EGFP: n=6 (sham), n=4 (3 dpi), n=5 (7 dpi), n=5 (15 dpi), and n=3 (50 dpi), and (ii) runx1EN-hsp68::EGFP: n=6 (sham), n=4 (3 dpi), n=4 (7 dpi), n=3 (14 dpi), and n=3 (50 dpi). In these experiments, the following applied: 2ankrd1aEN-hsp68::EGFP: n=4 (sham), n=3 (3 dpi), n=5 (7 dpi), n=3 (15 dpi), and n=3 (70 dpi). Following MI injury, LEN-hsp68::EGFP, runx1EN-hsp68::EGFP, and 2ankrd1aEN-hsp68::EGFP were detectable.

FIG. 1F shows that TREE-driven EGFP fluorescence co-localized with Tnnt+ cardiomyocytes. Scale bars represent 2 mm (in FIG. 1B); 500 μm (in FIG. 1E); 200 μm (in FIG. 1C); 50 μm (in FIG. 1F).

FIG. 2A-FIG. 2D show Zebrafish TREEs direct injury-induced expression in rodent, and human cardiac muscle cells.

FIG. 2A shows the experimental design used in FIG. 2B.

FIG. 2B shows images of neonatal rat ventricular myocytes (NRVMs) transduced with lentiviruses carrying TREE-hsp68::EGFP or control hsp68::EGFP after in vitro ischemia/reperfusion (I/R) injury. LEN-hsp68::EGFP and runx1EN-hsp68::EGFP are detectable after I/R injury. Right, Quantified EGFP expression efficiency. ANOVA Dunnett's multiple comparison test. In these experiments, n=3 (no lentivirus), n=6 (hsp68::EGFP), n=6 (LEN-hsp68::EGFP), and n=6 (runx1EN-hsp68::EGFP).

FIG. 2C shows the experimental design used in FIG. 2D.

FIG. 2D shows images of human cardiomyocytes transduced with lentiviruses carrying TREE-hsp68::EGFP or control hsp68::EGFP after I/R injury. runx1EN-hsp68::EGFP is detectable after I/R injury. Colors represent same constructs as in FIG. 2B. Right, Quantified EGFP expression efficiency. ANOVA Dunnett's multiple comparison test. In these experiments, n=3 for no lentivirus, n=5 for hsp68::EGFP, n=5 for LEN-hsp68::EGFP, and n=5 for runx1EN-hsp68::EGFP, respectively. Scale bars represent 100 μm (FIG. 2B, FIG. 2D).

FIG. 3A-FIG. 3K show that zebrafish TREEs directed injury-induced expression in porcine cardiac muscle cells.

FIG. 3A shows the experimental design used in FIG. 3B-FIG. 3G, in which pigs were infected by intracoronary (IC) perfusion or several intramuscular (IM) injections throughout the ventricles, prior to ischemia/reperfusion injury (FR). Regions of the ventricle sampled for histology are indicated by a-e.

FIG. 3B-FIG. 3F shows the section images of hearts after I/R injury, from pig infected pre-injury with AAV9 harboring a zebrafish TREE LEN, an hsp68 minimal promoter, and an EGFP (FIG. 3B-FIG. 3C, IC perfusion) or mCherry (FIG. 3E-FIG. 3F, IM injections) cassette. Fluorescence is induced by injury and restricted to CMs near the MI. White dashed lines indicate injured area.

FIG. 3D and FIG. 3G shows section images of liver and skeletal muscle after I/R injury, from pig infected pre-injury with AAV9 harboring a zebrafish TREE LEN, an hsp68 minimal promoter, and an EGFP (FIG. 3D, IC perfusion) or mCherry (FIG. 3G, IM injections) cassette. Fluorescence is negligible in liver and skeletal muscle.

FIG. 3H shows the experimental design used in FIG. 3I-FIG. 3K, in which pigs were infected by intracoronary (IC) perfusion 1 week post I/R injury. Regions of the ventricle sampled for histology are indicated by a-e.

FIG. 3I-FIG. 3J shows section images of hearts after I/R injury, from pig infected post-injury with AAV9 harboring a zebrafish TREE 2ankrd1aEN, an hsp68 minimal promoter, and an EGFP (IC perfusion) cassette. Fluorescence was induced by injury and restricted to CMs near the MI.

FIG. 3K shows section images of liver and skeletal muscle after I/R injury, from pig infected post-injury with AAV9 harboring a zebrafish TREE 2ankrd1aEN, an hsp68 minimal promoter, and an EGFP cassette. Fluorescence was negligible in liver and skeletal muscle. White dashed lines indicate injured area. Scale bars represent 100 μm (FIG. 3C, FIG. 3D, FIG. 3F, FIG. 3G, FIG. 3J, FIG. 3K); 200 μm (FIG. 3B, FIG. 3E, FIG. 3I).

FIG. 4A-FIG. 4I show zebrafish TREEs paired with epigenome editing tools controlled the expression of endogenous genes in injured murine hearts.

FIG. 4A-FIG. 4B shows the experimental design for in vivo modulation of endogenous gene expression, involving transgenic mice enabling Cre-based expression of modified Cas9 enzymes, and AAVs containing TREE- and U6-directed instructions.

FIG. 4C-FIG. 4D show section images from ventricle of Rosa26:LSL-dCas9p300 (n=3) or Rosa26:LSL-dCas9KRAB (n=3) mice injected with AAV9 carrying a zebrafish TREE, an hsp68 minimal promoter, and a Cre recombinase. Cas9 protein (green) was detected in CMs (red, Tnnt) near the injury site (asterisks).

FIG. 4E shows representative Western blot images of AGRN and GAPDH protein levels in hearts of Rosa26:LSL-dCas9p300 mice injected with AAV9-2ankrd1aEN-hsp68:Cre containing a scramble or Agrn gRNA and sacrificed at 14 dpi.

FIG. 4F shows the quantification of AGRN protein levels from 3 independent experiments; all samples were included in the graph and color-coded for each experiment. Each point represents one mouse. Mann-Whitney rank sum test. Here, n=10 for scramble gRNAs and n=17 for Agrn gRNAs.

FIG. 4G shows representative images of section ISH for Agrn mRNA in hearts of Rosa26:LSL-dCas9p300 mice treated with AAV9-2ankrd1aEN-hsp68:Cre containing a scramble (n=3) or Agrn gRNA (n=5) and sacrificed at 14 dpi.

FIG. 4H shows representative Western blot images of SAV1 and GAPDH protein levels from hearts of Rosa26:LSL-dCas9KRAB mice injected with AAV9-LEN-hsp68:Cre containing a scramble or Sav1 gRNA and sacrificed at 14 dpi.

FIG. 4I shows the quantification of SAV1 protein levels from 3 independent experiments; all samples were included in the graph and color coded for each experiment. Each point represents one mouse. Unpaired t-test with Welch's correction; n=9 for scramble gRNAs and n=15 for Sav1 gRNAs. Scale bars represent 500 μm (FIG. 4C, FIG. 4D), 100 μm (FIG. 4C, FIG. 4D, magnified area); 1 mm (FIG. 4G).

FIG. 5A-FIG. 5I show TREE-mediated Yap5SA delivery selectively boosted cardiomyocyte proliferation in injury sites.

FIG. 5A shows the experimental design used in FIG. 5B-FIG. 5I. An AAV with LEN directing EGFP or a Yap5SA cassette was delivered one week before myocardial infarction.

FIG. 5B-FIG. 5C show section images of 14 dpi hearts from adult mice infected pre-injury with AAV9 harboring LEN-hsp68::EGFP or LEN-hsp68::HA-hYap5SA. EGFP or HA is induced at the site of injury (asterisks) in LEN-hsp68::EGFP or LEN-hsp68::HA-Yap5SA hearts, respectively. High-magnification view of box in left.

FIG. 5D (left) shows quantified CM Ki67 indices in the border zone in adult mice infected pre-injury with AAV9 harboring LEN-hsp68::EGFP (n=6) or LEN-hsp68::HA-hYap5S (n=7). Unpaired t test with Welch's correction. FIG. 5D (right) shows section images of border zone stained for the CM marker Tnnt (red), WGA (white), and proliferation marker Ki67 (green).

FIG. 5E shows quantified CM Ki67 indices in distal myocardium. Mann Whitney test.

FIG. 5F (left) shows quantified CM EdU incorporation indices in the border zone. Unpaired t test with Welch's correction. FIG. 5F (right) shows section images of border zone stained for cardiomyocyte marker Tnnt (red), WGA (white) and EdU (green).

FIG. 5G shows quantified CM EdU incorporation indices in distal myocardium. Mann Whitney test.

FIG. 5H (left) shows quantified CM dedifferentiation indices in the border zone. Unpaired t test with Welch's correction. FIG. 5H (right) shows section images of border zone stained for CM marker Tnnt (red), and dedifferentiation marker αSMA (green). αSMA marks vascular smooth muscle and immature CMs, and CM staining is greater in the border zones of experimental animals.

FIG. 5I shows quantified CM dedifferentiation indices in distal myocardium. Mann Whitney test (n=6). The values in FIG. 5D-FIG. 5I were from 2 independent experiments. point represents one mouse and each color represent samples of an independent experiment. Scale bars represent 500 μm (FIG. 5B); 20 μm (FIG. 5D, FIG. 5F); 50 μm (FIG. 5C, FIG. 5H).

FIG. 6A-FIG. 6D show that zebrafish TREEs directed injury-induced gene expression in several adult mouse tissues.

FIG. 6A shows the whole-mount images of X-gal-stained adult il11aEN-hsp68::lacZ mouse hearts with staining (arrowhead) appearing in the area of LAD ligation (asterisk). In FIG. 6A, then=3 for both uninjured and 7 dpi.

FIG. 6B shows whole-mount images of X-gal-stained tibia of adult LEN-hsp68::lacZ mice 2 and 21 days after tibia fracture. Staining (arrowhead) was evident in fractured site (asterisks) at 21 dpi. In FIG. 6B, n=5, and 7 for 2, and 21 dpi, respectively.

FIG. 6C shows whole-mount images of X-gal-stained digits of adult LEN-hsp68::lacZ or runx1EN-hsp68::lacZ mice. Staining (arrows) was evident in amputated digit tips only (asterisks). In FIG. 6C, n=6 (3) for LEN-hsp68::lacZ and, and n=8 (4) for runx1-hsp68::lacZ digits (animals).

FIG. 6D shows whole-mount images of X-gal-stained tibialis anterior (TA) muscles of adult runx1EN-hsp68::lacZ mice 1 and 3 days after BaC12-induced injury. Staining (arrowheads) was evident in injured TA muscles. In FIG. 6D, n=5 for 1 dpi and n=3 for 3 dpi. In FIG. 6A-6D, the scale bars represented 2 mm.

FIG. 7A-FIG. 7C show that 2ankrd1aEN directed regeneration-associated expression in zebrafish hearts.

FIG. 7A is a schematic representation of the transgenes used for CM genetic ablation and assessment of 2ankrd1aEN injury-induced expression. Diphtheria toxin A (DTA) was expressed in cardiomyocytes after administration of 4-hydroxy-tamoxifen (4-HT).

FIG. 7B shows the experimental design to examine 2ankrd1aEN-directed reporter gene expression after induced CM ablation in zebrafish.

FIG. 7C shows that 2ankrd1aEN-cfos:EGFP hearts displayed little or no EGFP fluorescence when uninjured. By contrast, CM ablation induced strong EGFP signals in CMs at 14 dpi, still detectable by 30 dpi. In FIG. 7C, n=4 and the scale bar in FIG. 7B represents 200 μm.

FIG. 8A-FIG. 8E show that Zebrafish TREE constructs delivered by AAV directed gene expression in injured cardiac tissue.

FIG. 8A shows section images of uninjured/sham or injured hearts from adult mice systemically injected pre-injury with AAV9 harboring zebrafish TREE-hsp68::EGFP. Asterisks indicate the infarct area. For il11aEN-hsp68::EGFP: n=5 (uninjured/sham) and n=4 (7 dpi). For 22sema3aaEN-hsp68::EGFP, n=3 (uninjured/sham) and n=3 (7 dpi). For IN13zgc:136858EN-hsp68::EGFP, n=3 (uninjured/sham) and n=3 (7 dpi). None of these 3 TREEs directed detectable expression in mice.

FIG. 8B shows that TREE-directed EGFP co-localized with Tnnt+ CMs.

FIG. 8C shows section ISH for EGFP mRNA in hearts from adult mice injected pre-injury with an AAV9 harboring LEN- or runx1EN-hsp68::EGFP at 7 dpi. Staining (violet) is evident near the MI, mimicking EGFP fluorescence.

FIG. 8D shows section images of livers after MI injury from adult mice injected pre-injury with AAV9s. Moderate EGFP fluorescence was detectable in runx1EN-hsp68::EGFP and 2ankard1aEN-hsp68::EGFP livers whereas negligible EGFP was present in LEN-hsp68::EGFP livers. In FIG. 8D, n=3.

FIG. 8E shows section images of hearts 7 days after MI injury (asterisks) from adult mice injected pre-injury with AAV9 harboring TREE-cfos::EGFP or control cfos::EGFP. In FIG. 8E, n=3 for LEN-cfos::EGFP, 4 for runx1EN-cfos::EGFP, 3 for 2ankrd1aEN-cfos::EGFP, and 4 for control cfos::EGFP. Scale bars represented 500 μm (FIG. 8A, FIG. 8E); 200 μm (FIG. 8C); 50 μm (FIG. 8B, FIG. 8D). Brown dashed lines in c indicate injured area.

FIG. 9A-FIG. 9B show that tests of zebrafish TREE fragments to direct expression in injured mouse hearts.

FIG. 9A shows viral constructs evaluated for the ability to direct gene expression in injured adult mouse hearts (left panel). FIG. 9A shows section images of hearts after MI injury from adult mice (n=3) infected pre-injury with AAV9 harboring a full length LEN or LEN fragments, an hsp68 minimal promoter, and an EGFP cassette (right panel).

FIG. 9B shows section images of hearts after MI injury from adult mice infected pre-injury with AAV9 harboring either full-length 2ankrd1aEN or 2ankrd1aEN fragments, an hsp68 minimal promoter, and an EGFP cassette (n=3). The scale bars represent 100 μm.

FIG. 10A-FIG. 10D show tests of zebrafish TREEs to direct expression in adult mice when delivered by AAV post MI.

FIG. 10A (top panel) shows the experimental design.

FIG. 10A (bottom panel) shows section imaged of hearts after MI from adult mice infected 1 day post-MI with AAV9 harboring TREE-hsp68::EGFP or control hsp68::EGFP. EGFP fluorescence was detectable in the injury sites (asterisks) in each TREE-hsp68::EGFP group—(i.e., LEN-hsp68::EGFP (n=5), runx1EN-hsp68::EGFP (n=3), 2ankrd1aEN::EGFP (n=3), and hsp68::EGFP (n=4)). The box from the left image was magnified in the right image.

FIG. 10B (top panel) shows the experimental design.

FIG. 10B (bottom panel) shows section images of hearts after MI from adult mice infected 7 days post-MI with AAV9 harboring TREE-hsp68::EGFP or control hsp68::EGFP (n=3). Only 2ankrd1aEN directed EGFP fluorescence in these experiments.

FIG. 10C (top panel) shows the experimental design.

FIG. 10C (bottom panel) shows Section images of hearts after MI, from adult mice infected 30 days post-MI with AAV9 harboring TREE-hsp68::EGFP or control hsp68::EGFP (i.e., LEN-hsp68::EGFP (n=5), runx1EN-hsp68::EGFP (n=4), 2ankrd1aEN::EGFP (n=4), and hsp68::EGFP (n=3)). Only 2ankrd1aEN directed EGFP fluorescence in these experiments.

FIG. 10D (top panel) shows the experimental design.

FIG. 10D (bottom panel) shows section images of hearts after MI from adult mice infected 50 days post-MI with AAV9 harboring TREE-hsp68::EGFP or control hsp68::EGFP (n=3). No EGFP fluorescence was detected in these experiments. Scale bars represent 500 μm.

FIG. 11A-FIG. 11D show the controls for tests of zebrafish TREEs to direct expression in in vitro injury models.

FIG. 11A shows the images of neonatal rat ventricular myocytes (NRVMs) transduced with lentiviruses containing TREE-hsp68::EGFP or control hsp68::EGFP without treatment (i.e., hsp68::EGFP (n=3), LEN-hsp68::EGFP (n=3), and runx1EN-hsp68::EGFP (n=3)).

FIG. 11B shows images of NRVMs transduced with lentiviruses containing TREE-hsp68::EGFP or control hsp68::EGFP one day after a heat shock. EGFP fluorescence is detectable after heat shock in each case (i.e., hsp68::EGFP (n=5), LEN-hsp68::EGFP (n=5), and runx1EN-hsp68::EGFP (n=5), respectively.

FIG. 11C shows images of human CMs transduced with lentiviruses containing TREE-hsp68::EGFP or control hsp68::EGFP, without treatment (i.e., hsp68::EGFP (n=3), LEN-hsp68::EGFP (n=3), and runx1EN-hsp68::EGFP (n=3).

FIG. 11D shows images of human CMs transduced with lentiviruses containing TREE-hsp68::EGFP or control hsp68::EGFP one day after a heat shock. hsp:68-directed EGFP fluorescence is detectable after heat shock in each case, an indicator that CMs were transfected effectively in each experiment (i.e., no lentivirus (n=3), hsp68::EGFP (n=5), LEN-hsp68::EGFP (n=5), and runx1EN-hsp68::EGFP (n=5)). In FIG. 11D, the scale bars represent 100 μm.

FIG. 12A-FIG. 12M show the AAV-based gRNA delivery to Rosa26:LSL-dCas9p300 and Rosa26:LSL-dCas9KRAB mice modulated endogenous cardiac gene expression.

FIG. 12A shows the experimental design for in vivo modulation of endogenous gene expression involving transgenic mice enabling Cre-based expression of modified Cas9 enzymes and AAVs containing CMV- and U6-directed instructions.

FIG. 12B shows Western blot showing Cas9 expression 2 weeks post AAV9 injection in cardiac protein lysates from Rosa26:LSL-dCas9p300 mice injected with phosphate-buffered saline (PBS) or AAV9 containing a CMV promoter-directed Cre recombinase.

FIG. 12C shows Western blot showing Cas9 expression 2 weeks post AAV9 injection in cardiac protein lysates from Rosa26:LSL-dCas9KRAB mice injected with PBS or AAV9 containing a CMV promoter-directed Cre recombinase.

FIG. 12D shows section images of hearts of Rosa26:LSL-dCas9p300 mice 2 weeks after systemic injection of AAV9 containing a CMV promoter directing Cre expression. Cas9 expression (green) is detected throughout the ventricle.

FIG. 12E shows section images indicating co-localization of Cas9 and Tnnt in Rosa26:LSL-dCas9p300 mice 2 weeks after AAV9 injection.

FIG. 12F shows the experimental design for in vivo modulation of Agrn gene expression in Rosa26:LSL-dCas9p300 mice.

FIG. 12G shows the Agrn mRNA expression in heart tissue lysates isolated from mice injected with PBS, or AAV9 containing a CMV promoter-directed Cre recombinase and either a control non-targeting or an Agrn-targeting gRNA.

FIG. 12H-FIG. 12I shows the Western blot and protein quantification in heart tissue lysates isolated from mice injected with PBS or AAV9 containing a CMV promoter-directed Cre recombinase and either a control non-targeting or an Agrn-targeting gRNA.

FIG. 12J shows the experimental design for in vivo modulation of Sav1 gene expression in Rosa26:LSL-dCas9KRAB mice.

FIG. 12K shows Sav1 mRNA in heart tissue lysates isolated from mice injected with PBS or AAV9 containing a CMV promoter-directed Cre recombinase and either a control non-targeting or a Sav1-targeting gRNA

FIG. 12L-FIG. 12M shows the Western blot and protein quantification in heart tissue lysates isolated from mice injected with PBS or AAV9 containing a CMV promoter-directed Cre recombinase and either a control non-targeting or a Sav1-targeting gRNA.

The analyses in FIG. 12F-FIG. 12M were performed 14 days after systemic AAV delivery. Quantifications in FIG. 12G and FIG. 12K were the results of 3 independent experiments. Quantifications in FIG. 12I and FIG. 12M were results of 2 independent experiments. All samples are included in the graphs and color coded for each experiment. Each point represents one mouse.

FIG. 12H and FIG. 12I show representative western blot images. Tests were Mann-Whitney (FIG. 12G and FIG. 12M), unpaired t-test with Welch's correction (FIG. 12I and FIG. 12K). The horizontal dashed lines in FIG. 12G and FIG. 12K indicate the average value of scramble gRNA samples. Scale bars represent 500 μm (FIG. 12D—top panel) and 100 μm (FIG. 12D—bottom panel) and FIG. E7E).

FIG. 13A-FIG. 13B show TREE-mediated Yap5SA delivery at 3 dpi.

FIG. 13A shows section images from 3 dpi hearts of adult mice infected pre-injury with AAV9s harboring LEN-hsp68::HA-hYap5SA (right) stained for Tnnt (Red) and HA (green) (n=3). High-magnification views boxes in left are shown on right.

FIG. 13B shows section images of 3 dpi hearts from adult mice infected pre-injury with AAV9s harboring LEN-hsp68::EGFP (n=3) (top) or LEN-hsp68::HA-hYap5SA (n=3) (bottom) stained for Tnnt (Red) and the proliferation marker Ki67 (green). TREE-mediated Yap delivery did not noticeably increase the number of cycling cells at 3 dpi. Scale bars represent 500 μm.

FIG. 14A-FIG. 14B show TREE-mediated Yap delivery boosted proliferation selectively at injury sites.

FIG. 14A-FIG. 14B shows section images of 14 dpi hearts from adult mice infected pre-injury with AAV9s harboring LEN-hsp68::EGFP (top) (n=3) or LEN-hsp68::HA-hYap5SA (bottom) (n=3) stained for Tnnt (Red) and the proliferation marker Ki67 (green). High-magnification views boxes in left are shown on right. The border zones of experimental animals showed much greater evidence of cycling at 7 dpi. Scale bars represent 500 μm.

FIG. 15A-FIG. 15C show the limited effects of TREE-mediated YapSA delivery at 35 dpi.

FIG. 15A shows section images from 35 dpi hearts of adult mice infected pre-injury with AAV9s harboring LEN-hsp68::EGFP (top) or LEN-hsp68::HA-hYap5SA (bottom) stained for Tnnt (Red) and EGFP or HA (green).

FIG. 15B shows section images from 35 dpi hearts of adult mice infected pre-injury with AAV9s harboring LEN-hsp68::EGFP (top) or LEN-hsp68::HA-hYap5SA (bottom) stained for Tnnt (Red) and the proliferation marker Ki67 (green).

FIG. 15C shows section images from 35 dpi hearts of adult mice infected pre-injury with AAV9s harboring LEN-hsp68::EGFP (top) (n=3) or LEN-hsp68::HA-hYap5SA (bottom) (n=3) stained for Tnnt (Red) and the dedifferentiation marker αSMA (green. No evidence of induced gene reporter expression or obvious differences between groups were observed at 35 dpi. Scale bars represent 500 μm.

FIG. 16A-FIG. 16B show TREE-mediated Yap5SA delivery boosts indications of dedifferentiation selectively at injury sites.

FIG. 16A shows section images of 14 dpi hearts from adult mice infected pre-injury with AAV9s harboring LEN-hsp68::EGFP (n=3) (FIG. 16A) or LEN-hsp68::HA-hYap5SA (n=3) (FIG. 16B) stained for Tnnt (Red) and the dedifferentiation marker αSMA (green). High-magnification views boxes in left are shown on right. αSMA marks vascular smooth muscle and immature CMs, and CM staining was greater in the border zones of experimental animals. Scale bars represent 500 μm.

### DETAILED DESCRIPTION OF THE INVENTION

The present disclosure describes compositions, isolated nucleic acids, fusion products, pharmaceutical formulations, and methods of using the disclosed compositions, isolated nucleic acids, fusion products, pharmaceutical formulations thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, vectors, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “approximately” or “about,” as applied to one or more values of interest, refers to a value that is similar to a stated reference value. In an aspect, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction of the stated reference value unless otherwise stated or otherwise evident from the context.

As used herein, the term “in vitro” refers to events or experiments that occur in an artificial environment, e.g., in a petri dish, test tube, cell culture, etc., rather than within a multicellular organism. As used herein, the term “in vivo” refers to events or experiments that occur within a multicellular organism.

As used herein, a “biomarker” refers to a defined characteristic that is measured as an indicator of normal biological processes, pathogenic processes, or response to an exposure of intervention. In an aspect, a biomarker can be diagnostic (i.e., detects or classifies a pathological condition), prognostic (i.e., predicts the probability of disease occurrence or progression), pharmacodynamic/responsive (i.e., identifies a change in response to a therapeutic intervention), predictive (i.e., predicts how an individual or subject might respond to a particular intervention or event). In an aspect, a biomarker can be diagnostic, prognostic, pharmacodynamic/responsive, and/or predictive at the same time. In an aspect, a biomarker can be diagnostic, prognostic, pharmacodynamic/responsive, and/or predictive at different times (e.g., first a biomarker can be diagnostic and then later, the same biomarker can be prognostic, pharmacodynamic/responsive, and/or predictive). A biomarker can be an objective measure that can be linked to a clinical outcome assessment. A biomarker can be used by the skilled person to make a clinical decision based on its context of use.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration. In an aspect, a subject can be a human being. The term “subject” includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have stressed, damaged, and/or injured tissues, or be suspected of having stressed, damaged, and/or injured tissues, or be at risk of developing stressed, damaged, and/or injured tissues.

As used herein, “effective amount” and “amount effective” can refer to an amount that is sufficient to achieve the desired result such as, for example, the treatment and/or regeneration of stressed, damaged, and/or injured tissues. As used herein, the terms “effective amount” and “amount effective” can refer to an amount that is sufficient to achieve the desired effect on stressed, damaged, and/or injured tissues. For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed composition that (i) treats the stressed, damaged, and/or injured tissues, (ii) attenuates, ameliorates, or eliminates one or more symptoms of the particular stressed, damaged, and/or injured tissues, or (iii) delays the onset of one or more symptoms of the particular stressed, damaged, and/or injured tissues described herein. The specific therapeutically effective dose level for any particular patient will depend upon a variety of factors including the disorder being treated and the severity of the disorder; the specific disclosed compositions and/or a pharmaceutical preparation comprising one or more disclosed compositions, or methods employed; the age, body weight, general health, sex and diet of the patient; the time of administration; the route of administration; the rate of excretion of the disclosed compositions and/or a pharmaceutical preparation comprising one or more disclosed compositions employed; the duration of the treatment; drugs used in combination or coincidental with a disclosed compositions and/or a pharmaceutical preparation comprising one or more disclosed compositions employed, and other like factors well known in the medical arts. For example, it is well within the skill of the art to start doses of a disclosed composition and/or a pharmaceutical preparation comprising one or more disclosed composition at levels lower than those required to achieve the desired therapeutic effect and to gradually increase the dosage until the desired effect is achieved. If desired, then the effective daily dose can be divided into multiple doses for purposes of administration. Consequently, a single dose of a disclosed compositions and/or a pharmaceutical preparation comprising one or more disclosed compositions, or methods can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In an aspect, a preparation can be administered in a “prophylactically effective amount”; that is, an amount effective for prevention of a disease, a disorder, an infection, a symptom, and/or a complication.

“Control” as used herein refers a standard or reference condition, against which results are compared. In an aspect, a control is used at the same time as a test variable or subject to provide a comparison. In an aspect, a control is a historical control that has been performed previously, a result or amount that has been previously known, or an otherwise existing record. A control may be a positive or negative control.

As used herein, the term “diagnosed” means having been subjected to a physical examination by a person of skill, for example, a physician, and found to have stressed, damaged, and/or injured tissues that can be diagnosed or treated by one or more of the disclosed nucleic acids, the disclosed vectors, the disclosed compositions, the disclosed pharmaceutical preparations, and/or the disclosed methods. For example, “suspected of having” can mean having been subjected to a physical examination by a person of skill, for example, a physician, and found to have stressed, damaged, and/or injured tissues that can likely be treated by one or more of the disclosed nucleic acids, the disclosed vectors, the disclosed compositions, the disclosed pharmaceutical preparations, and/or the disclosed methods.

The words “treat” or “treating” or “treatment” refer to therapeutic or medical treatment wherein the object is to slow down (lessen), ameliorate, and/or diminish an undesired physiological change, disease, disorder, injury, pathological condition, or disorder in a subject. As used herein, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Treatment may not necessarily result in the complete clearance of an infection but may reduce or minimize complications, the side effects, and/or the progression of a disease, a disorder, an injury, an infection, a symptom, and/or a complication. The success or otherwise of treatment may be monitored by physical examination of the subject as well as cytopathological, DNA, and/or mRNA detection techniques. The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the undesired physiological change, disease, injury, insult, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating stressed, damaged, and/or injured tissues can reduce the severity of stressed, damaged, and/or injured tissues in a subject by 1%-100% as compared to a control (such as, for example, a subject not having the disease, the disorder, the injury, the infection, the symptom, and/or the complication). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of stressed, damaged, and/or injured tissues. In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms. It is understood that treatment does not necessarily refer to a cure or complete ablation or eradication of the stressed, damaged, and/or injured tissues. However, in an aspect, treatment can refer to a cure or complete ablation or eradication of the stressed, damaged, and/or injured tissues.

A “patient” refers to a subject afflicted with stressed, damaged, and/or injured tissues. In an aspect, a patient can refer to a subject that has been diagnosed with or is suspected of having a disease, disorder, infection, symptom, and/or complication that results in stressed, damaged, and/or injured tissues. In an aspect, a patient can refer to a subject that has been diagnosed with or is suspected of having an established disease, disorder, infection, symptom, and/or complication that results in stressed, damaged, and/or injured tissues and is seeking treatment or receiving treatment.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease, disorder, infection, symptom, and/or complication is intended. In an aspect, preventing stressed, damaged, and/or injured tissues is intended. The words “prevent” and “preventing” and “prevention” also refer to prophylactic or preventative measures for protecting or precluding a subject (e.g., an individual) not having a given complication associated with stressed, damaged, and/or injured tissues from progressing to that complication.

As used herein, the terms “administering” and “administration” refer to any method of providing one or more of the disclosed isolated nucleic acid molecules, disclosed pharmaceutical formulations, disclosed vectors, or any combination thereof to a subject. Such methods are well known to those skilled in the art and include, but are not limited to, the following routes: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed nucleic acid molecule, a disclosed vector, a disclosed therapeutic agent, a disclosed pharmaceutical formulation, or a combination thereof can comprise administration directly into the CNS (e.g., intraparenchymal, intracerebroventriular, inthrathecal cisternal, intrathecal (lumbar), deep gray matter delivery, convection-enhanced delivery to deep gray matter) or the PNS. Administration can be continuous or intermittent.

As used herein, “concurrently” means (1) simultaneously in time, or (2) at different times during the course of a common treatment schedule.

The term “contacting” as used herein refers to bringing one or more of the disclosed nucleic acids, the disclosed vectors, the disclosed compositions, and/or the disclosed pharmaceutical formulations together with a target area or intended target area (i.e., stressed, damaged, and/or injured tissues) in such a manner that the one or more disclosed nucleic acids, vectors, compositions, and/or pharmaceutical formulation can exert an effect on the intended target or targeted area (i.e., stressed, damaged, and/or injured tissues) either directly or indirectly.

As used herein, “determining” can refer to measuring or ascertaining the presence and severity of a disease, disorder, injury, infection, symptom, and/or complication or the presence and severity of stressed, damaged, and/or injured tissues. Methods and techniques used to determining the presence and/or severity of a disease, disorder, injury, infection, symptom, and/or complication or the presence and/or severity of stressed, damaged, and/or injured tissues are typically known to the medical arts. For example, the art is familiar with the ways to identify and/or diagnose the presence, severity, or both of a disease, disorder, infection, symptom, and/or complication, or the presence, severity, or both of stressed, damaged, and/or injured tissues.

As used herein, “CRISPR or clustered regularly interspaced short palindromic repeat” is an ideal tool for correction of genetic abnormalities as the system can be designed to target genomic DNA directly. A CRISPR system involves two main components: a Cas9 enzyme and a guide (gRNA). The gRNA contains a targeting sequence for DNA binding and a scaffold sequence for Cas9 binding. Cas9 nuclease is often used to “knockout” target genes hence it can be applied for deletion or suppression of oncogenes that are essential for cancer initiation or progression. Similar to ASOs and siRNAs, CRISPR offers a great flexibility in targeting any gene of interest hence, potential CRISPR based therapies can be designed based on the genetic mutation in individual patients. An advantage of CRISPR is its ability to completely ablate the expression of disease genes which can only be suppressed partially by RNA interference methods with ASOs or siRNAs. Furthermore, multiple gRNAs can be employed to suppress or activate multiple genes simultaneously, hence increasing the treatment efficacy and reducing resistance potentially caused by new mutations in the target genes.

As used herein, “CRISPRa” refers to CRISPR Activation, which is using a dCas9 or dCas9-activator with a gRNA to increase transcription of a target gene.

As used herein, “CRISPRi” refers to CRISPR Interference, which is using a dCas9 or dCas9-repressor with a gRNA to repress/decrease transcription of a target gene.

As used herein, “dCas9” refers to enzymatically inactive form of Cas9, which can bind, but cannot cleave, DNA. In an aspect, a disclosed dCas can comprise dVQR, dEQR, or dVRER.

As used herein, “Protospacer Adjacent Motif” or “PAM” refers to a sequence adjacent to the target sequence that is necessary for Cas enzymes to bind target DNA.

As used herein, “CRISPR-based endonucleases” include RNA-guided endonucleases that comprise at least one nuclease domain and at least one domain that interacts with a guide RNA. As known to the art, a guide RNA directs the CRISPR-based endonucleases to a targeted site in a nucleic acid at which site the CRISPR-based endonucleases cleaves at least one strand of the targeted nucleic acid sequence. As the guide RNA provides the specificity for the targeted cleavage, the CRISPR-based endonuclease is universal and can be used with different guide RNAs to cleave different target nucleic acid sequences. CRISPR-based endonucleases are RNA-guided endonucleases derived from CRISPR/Cas systems. Bacteria and archaea have evolved an RNA-based adaptive immune system that uses CRISPR (clustered regularly interspersed short palindromic repeat) and Cas (CRISPR-associated) proteins to detect and destroy invading viruses or plasmids. CRISPR/Cas endonucleases can be programmed to introduce targeted site-specific double-strand breaks by providing target-specific synthetic guide RNAs (Jinek et al. (2012) Science. 337:816-821).

In an aspect, a disclosed CRISPR-based endonuclease can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In an aspect, a disclosed CRISPR-based endonuclease can be derived from a type II CRISPR/Cas system. For example, in an aspect, a CRISPR-based endonuclease can be derived from a Cas9 protein. The Cas9 protein can be from Streptococcus pyogenes, Streptococcus thermophilus, Streptococcus sp, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. In an aspect, the CRISPR-based nuclease can be derived from a Cas9 protein from Streptococcus pyogenes.

In general, CRISPR/Cas proteins can comprise at least one RNA recognition and/or RNA binding domain. RNA recognition and/or RNA binding domains can interact with the guide RNA such that the CRISPR/Cas protein is directed to a specific genomic or genomic sequence. CRISPR/Cas proteins can also comprise nuclease domains (i.e., DNase or RNase domains), DNA binding domains, helicase domains, protein-protein interaction domains, dimerization domains, as well as other domains.

The CRISPR-based endonuclease can be a wild type CRISPR/Cas protein, a modified CRISPR/Cas protein, or a fragment of a wild type or modified CRISPR/Cas protein. The CRISPR/Cas protein can be modified to increase nucleic acid binding affinity and/or specificity, alter an enzymatic activity, and/or change another property of the protein. For example, in an aspect, nuclease (i.e., DNase, RNase) domains of the CRISPR/Cas protein can be modified, deleted, or inactivated. A CRISPR/Cas protein can be truncated to remove domains that are not essential for the function of the protein. A CRISPR/Cas protein also can be truncated or modified to optimize the activity of the protein or an effector domain fused with a CRISPR/Cas protein.

In an aspect, a disclosed CRISPR-based endonuclease can be derived from a wild type Cas9 protein or fragment thereof. In an aspect, a disclosed CRISPR-based endonuclease can be derived from a modified Cas9 protein. For example, the amino acid sequence of a disclosed Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

As used herein, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced.

“Tissue-specific promoters” are known to the art and include, but are not limited to, neuron-specific promoters, muscle-specific promoters, liver-specific promoters, skeletal muscle-specific promoters, and heart-specific promoters.

“Neuron-specific promoters” are known to the art and include, but are not limited to, the synapsin I (SYN) promoter, the calcium/calmodulin-dependent protein kinase II promoter, the tubulin alpha I promoter, the neuron-specific enolase promoter, and the platelet-derived growth factor beta chain promoter.

“Liver-specific promoters” are known to the art and include, but are not limited to, the α1-microglobulin/bikunin enhancer/thyroid hormone-binding globulin promoter, the human albumin (hALB) promoter, the thyroid hormone-binding globulin promoter, thyroxin binding globulin promoter, the α-1-anti-trypsin promoter, the bovine albumin (bAlb) promoter, the murine albumin (mAlb) promoter, the human α1-antitrypsin (hAAT) promoter, the ApoEhAAT promoter composed of the ApoE enhancer and the hAAT promoter, the transthyretin (TTR) promoter, the liver fatty acid binding protein promoter, the hepatitis B virus (HBV) promoter, the DC172 promoter consisting of the hAAT promoter and the al-microglobulin enhancer, the DC190 promoter containing the human albumin promoter and the prothrombin enhancer, and other natural and synthetic liver-specific promoters.

“Muscle-specific promoters” are known to the art and include, but are not limited to, the MHCK7 promoter, the muscle creatine kinase (MCK) promoter/enhancer, the slow isoform of troponin I (TnIS) promoter, the MYODI promoter, the MYLK2 promoter, the SPc5-12 promoter, the desmin (Des) promoter, the unc45b promoter, and other natural and synthetic muscle-specific promoters.

“Skeletal muscle-specific promoters” are known to the art and include, but are not limited to, the HSA promoter, the human α-skeletal actin promoter.

“Heart-specific promoters” are known to the art and include, but art not limited to, the MYH6 promoter, the TNNI3 promoter, the cardiac troponin C (cTnC) promoter, the alpha-myosin heavy chain (α-MHC) promoter, myosin light chain 2 (MLC-2), and the MYBPC3 promoter.

As used herein, a “ubiquitous/constitutive promoter” refer to a promoter that allows for continual transcription of its associated gene. A ubiquitous/constitutive promoter is always active and can be used to express genes in a wide range of cells and tissues, including, but not limited to, the liver, kidney, skeletal muscle, cardiac muscle, smooth muscle, diaphragm muscle, brain, spinal cord, endothelial cells, intestinal cells, pulmonary cells (e.g., smooth muscle or epithelium), peritoneal epithelial cells, and fibroblasts. Ubiquitous/constitutive promoters include, but are not limited to, a CMV major immediate-early enhancer/chicken beta-actin promoter, a cytomegalovirus (CMV) major immediate-early promoter, an Elongation Factor 1-α (EF1-α) promoter, a simian vacuolating virus 40 (SV40) promoter, an AmpR promoter, a PγK promoter, a human ubiquitin C gene (Ubc) promoter, a MFG promoter, a human beta actin promoter, a CAG promoter, a EGR1 promoter, a FerH promoter, a FerL promoter, a GRP78 promoter, a GRP94 promoter, a HSP70 promoter, a β-kin promoter, a murine phosphoglycerate kinase (mPGK) or human PGK (hPGK) promoter, a ROSA promoter, human Ubiquitin B promoter, a Rous sarcoma virus promoter, or any other natural or synthetic ubiquitous/constitutive promoters.

As used herein, an “inducible promoter” refers to a promoter that can be regulated by positive or negative control. Factors that can regulate an inducible promoter include, but are not limited to, chemical agents (e.g., the metallothionein promoter or a hormone inducible promoter), temperature, and light.

As used herein, “operably linked” means that expression of a gene or a transgene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, an “enhancer” such as a transcription or transcriptional enhancer refers to regulatory DNA segment that is typically found in multicellular eukaryotes. An enhancer can strongly stimulate (“enhance”) the transcription of a linked transcription unit, i.e., it acts in cis. An enhancer can activate transcription over very long distances of many thousand base pairs, and from a position upstream or downstream of the site of transcription initiation. An enhancers can have a modular structure by being composed of multiple binding sites for transcriptional activator proteins. Many enhancers control gene expression in a cell type-specific fashion. Several remote enhancers can control the expression of a singular gene while a singular enhance can stimulate the transcription of one or more genes.

As used herein, “expression cassette” or “transgene cassette” can refer to a distinct component of vector DNA comprising a transgene and one or more regulatory sequences to be expressed by a transfected cell. Generally, an expression cassette or transgene cassette can comprise a promoter sequence, an open reading frame (i.e., the transgene), and a 3′ untranslated region (e.g., in eukaryotes a polyadenylation site).

As used herein, an “isolated” biological component (such as a nucleic acid molecule, protein, or virus) has been substantially separated or purified away from other biological components (e.g., other chromosomal and extra-chromosomal DNA and RNA, proteins and/or organelles). Nucleic acids, proteins, and/or viruses that have been “isolated” include nucleic acids, proteins, and viruses purified by standard purification methods. The term also embraces nucleic acids, proteins, and viruses prepared by recombinant expression in a host cell, as well as chemically synthesized nucleic acids or proteins. The term “isolated” (or purified) does not require absolute purity; rather, it is intended as a relative term. Thus, for example, an isolated or purified nucleic acid, protein, virus, or other active compound is one that is isolated in whole or in part from associated nucleic acids, proteins, and other contaminants. In an aspect, the term “substantially purified” refers to a nucleic acid, protein, virus or other active compound that has been isolated from a cell, cell culture medium, or other crude preparation and subjected to fractionation to remove various components of the initial preparation, such as proteins, cellular debris, and other components.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of glycogen branching enzymes and amylases. It should be understood that sequence with substantial sequence identity do not necessarily have the same length and may differ in length. For example, sequences that have the same nucleotide sequence but of which one has additional nucleotides on the 3′- and/or 5′-side are 100% identical.

Disclosed are the components to be used to prepare one or more of the disclosed nucleic acids, the disclosed vectors, the disclosed compositions, and/or the disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Compositions

### 1. Isolated Nucleic Acid Molecules

Disclosed herein is an isolated nucleic acid molecule comprising three or more sequence elements including a noncoding sequence that can control the ability of a minimal promoter sequence to direct gene expression in living tissues during stress or injury (called a TREE); a minimal promoter with little or no basal activity in zebrafish tissues; a sequence encoding an enhanced green fluorescent reporter gene; and a 3′ UTR noncoding region that stabilizes the RNA message and enables translation.

Disclosed herein is an isolated nucleic acid molecule comprising a nucleic acid sequence encoding a tissue regeneration enhancer element (TREE); a minimal promoter with little or no basal activity; a reporter transgene; and a 3′ UTR noncoding region.

Disclosed herein is an isolated nucleic acid molecule, comprising: a nucleic acid sequence encoding a tissue regeneration enhancer element (TREE); a minimal promoter with little or no basal activity; further comprising a reporter transgene; and a 3′ UTR noncoding region.

Disclosed herein is an isolated nucleic acid molecule, comprising a nucleic acid sequence encoding a tissue regeneration enhancer element (TREE) and a minimal promoter with little or no basal activity.

Disclosed herein is an isolated nucleic acid molecule, comprising a nucleic acid sequence encoding a tissue regeneration enhancer element (TREE), a minimal promoter with little or no basal activity, and a 3′ UTR noncoding region.

Disclosed herein is an isolated nucleic acid molecule, comprising a nucleic acid sequence encoding a tissue regeneration enhancer element (TREE), a minimal promoter having little or no basal activity, and at least one polypeptide having biological activity.

Disclosed herein is an isolated nucleic acid molecule, comprising a nucleic acid sequence encoding a tissue regeneration enhancer element (TREE), a minimal promoter having little or no basal activity, at least one polypeptide having biological activity, and a 3′ UTR noncoding region.

In an aspect, a disclosed isolated nucleic acid molecule can comprise a coding sequence that is less than about 4.5 kilobases. In an aspect, a disclosed isolated nucleic acid molecule can further comprise a reporter transgene. In an aspect, a disclosed isolated nucleic acid molecule can further comprise a 3′ UTR noncoding region. In an aspect, a disclosed 3′ UTR noncoding region can stabilize the transcribed RNA message. In an aspect, a disclosed 3′ UTR noncoding region can comprise a polyadenylation (polyA) sequence and/or a structural element that stabilizes the transcribed RNA message.

In an aspect, a disclosed isolated nucleic acid molecule can comprise inverted terminal repeats such as, for example, those ITRs derived from an adeno-associated viral (AAV) genomes.

In an aspect, a disclosed tissue regeneration enhancer element (TREE) can be a noncoding sequence that is sufficient to direct expression of a encoded polypeptide or endogenous gene when introduced into stressed, damaged, and/or injured tissues.

In an aspect, a disclosed TREE can be a noncoding sequence in zebrafish. In an aspect, a disclosed TREE can be a noncoding sequence in a mammal.

In aspect, a disclosed TREE can be LEN, which is a noncoding sequence about 6 kb upstream of the zebrafish leptin b gene. In an aspect, a disclosed LEN can be from a zebrafish. In an aspect, a disclosed LEN can be found at Zv10 coord/mm10 coord chr4:19,050,039-19,051,709 in NCBI sequence ID no. LR812066.1. In an aspect, a disclosed LEN can comprise a sequence having about 70%, about 75%, about 80%, about 85%, about 90%, about 95%, or more than 95% identity to the sequence set forth at Zv10 coord/mm10 coord chr4:19,050,039-19,051,709 in NCBI sequence ID no. LR812066.1 or a fragment thereof.

In aspect, a disclosed TREE can be runx1EN, which is a noncoding sequence about 103 kb upstream of the zebrafish runx1 gene. In an aspect, a disclosed runx1EN can be from a zebrafish. In an aspect, a disclosed runx1EN can be found at Zv10 coord/mm10 coord chr1:1,356,353-1,357,617 in NCBI sequence ID no. LR812063.1. In an aspect, a disclosed runx1EN can comprise a sequence having about 70%, about 75%, about 80%, about 85%, about 90%, about 95%, or more than 95% identity to the sequence set forth at Zv10 coord/mm10 coord chr1:1,356,353-1,357,617 in NCBI sequence ID no. LR812063.1 or a fragment thereof.

In aspect, a disclosed TREE can be 2ankdr1aEN, which is a noncoding sequence about 2 kb upstream of the zebrafish ankrd1a gene. In an aspect, a disclosed 2ankrd1aEN can be from a zebrafish. In an aspect, a disclosed 2ankdr1aEN can be found at Zv10 coord/mm10 coord chr17:23,439,928-23,441,143 in NCBI sequence ID no. LR812585.1. In an aspect, a disclosed 2ankdr1aEN can comprise a sequence having about 70%, about 75%, about 80%, about 85%, about 90%, about 95%, or more than 95% identity to the sequence set forth at Zv10 coord/mm10 coord chr17:23,439,928-23,441,143 in NCBI sequence ID no. LR812585.1 or a fragment thereof.

In aspect, a disclosed TREE can be il11aEN, which is an il11a-linked enhancer. In an aspect, a disclosed il11aEN can be from a zebrafish. In an aspect, a disclosed il11aEN can be found at Zv10 coord/mm10 coord chr16:12,705,344-12,706,517 in NCBI sequence ID no. LR812053.1. In an aspect, a disclosed il11aEN can comprise a sequence having about 70%, about 75%, about 80%, about 85%, about 90%, about 95%, or more than 95% identity to the sequence set forth at Zv10 coord/mm10 coord chr16:12,705,344-12,706,517 in NCBI sequence ID no. LR812053.1 or a fragment thereof.

In an aspect, a disclosed TREE can control the ability of a disclosed minimal promoter to direct expression of a disclosed encoded polypeptide. In an aspect, directing expression of a disclosed encoded polypeptide can comprise activate, maintain, and/or alleviate expression of the encoded polypeptide in stressed, damaged, and/or injured tissues. In an aspect, a disclosed encoded polypeptide can be a cell survival factor, a growth factor, a transcription factor, an angiogenic factor, an innervating factor, or an combination thereof.

In an aspect, a disclosed TREE can control the ability of a disclosed minimal promoter to direct expression of a disclosed endogenous gene. In an aspect, directing expression of a disclosed endogenous gene can comprise activate, maintain, and/or alleviate expression of the endogenous gene in stressed, damaged, and/or injured tissues. In an aspect, a disclosed endogenous gene can encode a polypeptide having a pro-regenerative activity. In an aspect, a disclosed endogenous gene can be a cell survival factor, a growth factor, a transcription factor, an angiogenic factor, an innervating factor, or an combination thereof.

In an aspect, a disclosed TREE can control the ability of a disclosed minimal promoter to direct expression of a disclosed reporter gene. In an aspect, directing expression of a disclosed reporter gene can comprise activate, maintain, and/or alleviate expression of the reporter gene in stressed, damaged, and/or injured tissues.

In an aspect, a disclosed minimal promoter can have little or no basal activity in stressed, damaged, and/or injured tissues. In an aspect, in the absence of an enhancer, a disclosed minimal promoter cannot direct detectable RNA polymerase II-based transcription. In an aspect, a disclosed minimal promoter can direct minimal or no expression unless guided by additional enhancer DNA sequences.

In an aspect, disclosed stressed, damaged, and/or injured tissues can comprise mammalian tissue. Mammalian tissues are known to the art and can comprise mouse tissue, porcine tissue, and/or human tissue.

In an aspect, a disclosed minimal promoter can comprise a mammalian minimal promoter. In an aspect, a disclosed minimal promoter can comprise a human minimal promoter or a murine minimal promoter.

In an aspect, a disclosed minimal promoter can comprise a Hsp68 minimal promoter or a fragment thereof. In an aspect, a disclosed minimal promoter can comprise a murine Hsp68 minimal promoter or a fragment thereof. In an aspect, a disclosed murine Hsp68 minimal promoter can be found at Zv10 coord/mm10 coord chr17:34,971,928-34,972,798 in NCBI sequence ID no. CU457784.5. In an aspect, a disclosed murine Hsp68 minimal promoter can comprise a sequence having about 70%, about 75%, about 80%, about 85%, about 90%, about 95%, or more than 95% identity to the sequence at Zv10 coord/mm10 coord chr17:34,971,928-34,972,798 in NCBI sequence ID no. CU457784.5. In an aspect, NCBI sequence ID no. CU457784.5 can comprise the sequence set forth in SEQ ID NO:16.

In an aspect, a disclosed minimal promoter can comprise a cfos minimal promoter or a fragment thereof. In an aspect, a disclosed minimal promoter can comprise a murine cfos minimal promoter or a fragment thereof. In an aspect, a disclosed murine cfos minimal promoter can be found at Zv10 coord/mm10 coord chr12:85,473,820-85,473,917 in NCBI sequence ID no. AF332140.1. In an aspect, a disclosed murine cfos minimal promoter can comprise a sequence having about 70%, about 75%, about 80%, about 85%, about 90%, about 95%, or more than 95% identity to the sequence at Zv10 coord/mm10 coord chr12:85,473,820-85,473,917 in NCBI sequence ID no. AF332140.1. In an aspect, NCBI sequence ID no. AF332140.1 can comprise the sequence set forth in SEQ ID NO:17.

In an aspect, a disclosed minimal promoter can comprise an AAV elb minimal promoter. In an aspect, a disclosed minimal promoter can comprise the sequence set forth in SEQ ID NO:18 or a fragment thereof, or a sequence having about 70%, about 75%, about 80%, about 85%, about 90%, about 95%, or more than 95% identity to the sequence set forth in SEQ ID NO:18 or a fragment thereof. In an aspect, a disclosed minimal promoter can comprise the sequence set forth in GenBank Accession No. KU664676.1 or a fragment thereof, or a sequence having about 50%, about 55%, about 60%, about 65%, about 70%, about 75%, about 80%, about 85%, about 90%, about 95%, or more than 95% identity to the sequence set forth in GenBank Accession No. KU664676.1 or a fragment thereof.

In an aspect, a disclosed endogenous gene can encode a polypeptide that promotes tissue survival, promotes cell division or tissue patterning, stimulates proliferation of heart muscle cells, reduces inflammation and/or scarring, or any combination thereof.

In an aspect, a disclosed endogenous gene can encode a transcription factor, a modified transcription factor, or a recombinant transcription factor. In an aspect, a disclosed transcription factor can stimulate division in heart muscle cells.

In an aspect, a disclosed endogenous gene can encode a secreted factor, a modified secreted factor, or a recombinant secreted factor. In an aspect, a disclosed secreted factor can stimulate proliferation of heart muscle cells.

In an aspect, a disclosed endogenous gene can encode a secreted angiogenic factor, a modified angiogenic factor, or a recombinant angiogenic factor. In an aspect, a disclosed secreted angiogenic factor can stimulate survival and/or proliferation of heart muscle cells.

In an aspect, a disclosed encoded polypeptide having biological activity can comprise a reporter gene such as, for example, LacZ, green fluorescent protein, or mCherry.

In an aspect, a disclosed encoded polypeptide having biological activity can comprise a pro-regenerative activity. In an aspect, a disclosed encoded polypeptide having biological activity can promote tissue survival, promotes cell division or tissue patterning, stimulates proliferation of heart muscle cells, reduces inflammation and/or scarring, or any combination thereof.

In an aspect, a disclosed encoded polypeptide having biological activity can comprise a transcription factor, a modified transcription factor, or a recombinant transcription factor. In an aspect, a disclosed transcription factor can stimulate division in heart muscle cells.

In an aspect, a disclosed encoded polypeptide having biological activity can comprise a secreted factor, a modified secreted factor, or a recombinant secreted factor. In an aspect, a disclosed secreted factor can stimulate proliferation of heart muscle cells.

In an aspect, a disclosed encoded polypeptide having biological activity can comprise a secreted angiogenic factor, a modified angiogenic factor, or a recombinant angiogenic factor. In an aspect, a disclosed secreted angiogenic factor can stimulate survival and/or proliferation of heart muscle cells.

In an aspect, a disclosed encoded mammalian YAP1 can comprise the sequence set forth in SEQ ID NO:19 or a fragment thereof or a sequence having about 50%, about 55%, about 60%, about 65%, about 70%, about 75%, about 80%, about 85%, about 90%, about 95%, or more than 95% identity to the sequence set forth in SEQ ID NO:19 or a fragment thereof.

In an aspect, a disclosed encoded mammalian YAP1 can comprise the sequence set forth in SEQ ID NO:20 or a fragment thereof or a sequence having about 50%, about 55%, about 60%, about 65%, about 70%, about 75%, about 80%, about 85%, about 90%, about 95%, or more than 95% identity to the sequence set forth in SEQ ID NO:20 or a fragment thereof.

In an aspect, a disclosed encoded mammalian YAP1 can comprise the sequence set forth in SEQ ID NO:21 or a fragment thereof or a sequence having about 50%, about 55%, about 60%, about 65%, about 70%, about 75%, about 80%, about 85%, about 90%, about 95%, or more than 95% identity to the sequence set forth in SEQ ID NO:21 or a fragment thereof.

In an aspect, a disclosed encoded mammalian YAP1 can comprise the sequence set forth in SEQ ID NO:22 or a fragment thereof or a sequence having about 50%, about 55%, about 60%, about 65%, about 70%, about 75%, about 80%, about 85%, about 90%, about 95%, or more than 95% identity to the sequence set forth in SEQ ID NO:22 or a fragment thereof.

In an aspect, a disclosed encoded polypeptide having biological activity can comprise a mammalian YAP5SA or a fragment thereof. In an aspect, a disclosed mammalian YAP5SA can comprise a human YAP5SA or a fragment thereof. In an aspect, a disclosed human YAP5SA can comprise the sequence set forth in SEQ ID NO:32 or a fragment thereof. In an aspect, a disclosed human YAP5SA can comprise a sequence having about 50%, about 55%, about 60%, about 65%, about 70%, about 75%, about 80%, about 85%, about 90%, about 95%, or more than 95% identity to the sequence set forth in SEQ ID NO:32 or a fragment thereof (see, e.g., Varelas X. (2014) Development. 141(8):1614-1626).

In an aspect, a disclosed encoded polypeptide having biological activity can comprise a mammalian YAP8SA or a fragment thereof. In an aspect, a disclosed mammalian YAP8SA can comprise a human YAP8SA or a fragment thereof. In an aspect, a disclosed human YAP8SA can comprise the sequence set forth in SEQ ID NO:33 or a fragment thereof. In an aspect, a disclosed human YAP8SA can comprise a sequence having about 50%, about 55%, about 60%, about 65%, about 70%, about 75%, about 80%, about 85%, about 90%, about 95%, or more than 95% identity to the sequence set forth in SEQ ID NO:33 or a fragment thereof. In an aspect, a disclosed polypeptide having biological activity can comprise a mammalian YAP8SA or a fragment thereof (e.g., Addgene plasmid #27371; Zhao B, et al. (2007) Genes Dev. 21(21):2747-2761)).

In an aspect, a disclosed encoded polypeptide having biological activity can comprise a mammalian Neuregulin 1 or a fragment thereof. In an aspect, a disclosed mammalian Neuregulin 1 can be a human Neuregulin 1 or a murine Neuregulin 1.

In an aspect, a disclosed encoded mammalian Neuregulin 1 can comprise the sequence set forth in SEQ ID NO:23 or a fragment thereof. In an aspect, a disclosed encoded mammalian Neuregulin 1 can comprise a sequence having about 50%, about 55%, about 60%, about 65%, about 70%, about 75%, about 80%, about 85%, about 90%, about 95%, or more than 95% identity to the sequence set forth in SEQ ID NO:23 or a fragment thereof.

In an aspect, a disclosed encoded mammalian Neuregulin 1 can comprise the sequence set forth in SEQ ID NO:24 or a fragment thereof. In an aspect, a disclosed encoded mammalian Neuregulin 1 can comprise a sequence having about 50%, about 55%, about 60%, about 65%, about 70%, about 75%, about 80%, about 85%, about 90%, about 95%, or more than 95% identity to the sequence set forth in SEQ ID NO:24 or a fragment thereof.

In an aspect, a disclosed encoded mammalian Neuregulin 1 can comprise the sequence set forth in SEQ ID NO:27 or a fragment thereof. In an aspect, a disclosed encoded mammalian Neuregulin 1 can comprise a sequence having about 50%, about 55%, about 60%, about 65%, about 70%, about 75%, about 80%, about 85%, about 90%, about 95%, or more than 95% identity to the sequence set forth in SEQ ID NO:27 or a fragment thereof.

In an aspect, a disclosed encoded mammalian Neuregulin 1 can comprise the sequence set forth in any one of Accession Nos. AF491780.1, NM 013962.3, or EF410152 or a fragment thereof or a sequence having about 50%, about 55%, about 60%, about 65%, about 70%, about 75%, about 80%, about 85%, about 90%, about 95%, or more than 95% identity to the sequence set forth in any one of Accession Nos. AF491780.1, NM_013962.3, or EF410152 or a fragment thereof.

In an aspect, a disclosed mammalian Neuregulin 1 can comprise the sequence set forth in SEQ ID NO:25 or a fragment thereof. In an aspect, a disclosed mammalian Neuregulin 1 can comprise a sequence having about 50%, about 55%, about 60%, about 65%, about 70%, about 75%, about 80%, about 85%, about 90%, about 95%, or more than 95% identity to the sequence set forth in SEQ ID NO:25 or a fragment thereof.

In an aspect, a disclosed mammalian Neuregulin 1 can comprise the sequence set forth in SEQ ID NO:26 or a fragment thereof. In an aspect, a disclosed mammalian Neuregulin 1 can comprise a sequence having about 50%, about 55%, about 60%, about 65%, about 70%, about 75%, about 80%, about 85%, about 90%, about 95%, or more than 95% identity to the sequence set forth in SEQ ID NO:26 or a fragment thereof.

In an aspect, a disclosed mammalian Neuregulin 1 can comprise the sequence set forth in any one of Accession No. NM_013962.3 or EF410152.1 or a fragment thereof. In an aspect, a disclosed mammalian Neuregulin 1 can comprise a sequence having about 50%, about 55%, about 60%, about 65%, about 70%, about 75%, about 80%, about 85%, about 90%, about 95%, or more than 95% identity to the sequence set forth in one of Accession No. NM_013962.3 or EF410152.1 or a fragment thereof.

In an aspect, a disclosed murine Neuregulin 1 can comprise the sequence set forth in SEQ ID NO:34 or a fragment thereof. In an aspect, a disclosed murine Neuregulin 1 can comprise a sequence having about 50%, about 55%, about 60%, about 65%, about 70%, about 75%, about 80%, about 85%, about 90%, about 95%, or more than 95% identity to the sequence set forth in SEQ ID NO:34 or a fragment thereof.

In an aspect, a disclosed encoded murine Neuregulin 1 can comprise the sequence set forth in SEQ ID NO:35 or a fragment thereof. In an aspect, a disclosed encoded murine Neuregulin 1 can comprise a sequence having about 50%, about 55%, about 60%, about 65%, about 70%, about 75%, about 80%, about 85%, about 90%, about 95%, or more than 95% identity to the sequence set forth in SEQ ID NO:35 or a fragment thereof.

In an aspect, a disclosed encoded murine Neuregulin 1 can comprise the sequence set forth in Accession No. BC151113.1 or a fragment thereof. In an aspect, a disclosed encoded murine Neuregulin 1 can comprise a sequence having about 50%, about 55%, about 60%, about 65%, about 70%, about 75%, about 80%, about 85%, about 90%, about 95%, or more than 95% identity to the sequence set forth in Accession No. BC151113.1 or a fragment thereof.

In an aspect, a disclosed encoded polypeptide having biological activity can comprise a mammalian VEGF or VEGFA or a fragment thereof. In an aspect, a disclosed mammalian VEGF or VEGFA can comprise a murine VEGF or VEGFA or a human VEGF or VEGFA.

In an aspect, a disclosed encoded mammalian VEGF can comprise the sequence set forth in SEQ ID NO:28 or a fragment thereof. In an aspect, a disclosed encoded mammalian VEGF can comprise a sequence having about 50%, about 55%, about 60%, about 65%, about 70%, about 75%, about 80%, about 85%, about 90%, about 95%, or more than 95% identity to the sequence set forth in SEQ ID NO:28 or a fragment thereof.

In an aspect, a disclosed encoded mammalian VEGF can comprise the sequence set forth in SEQ ID NO:31 or a fragment thereof. In an aspect, a disclosed encoded mammalian VEGF can comprise a sequence having about 50%, about 55%, about 60%, about 65%, about 70%, about 75%, about 80%, about 85%, about 90%, about 95%, or more than 95% identity to the sequence set forth in SEQ ID NO:31 or a fragment thereof.

In an aspect, a disclosed encoded mammalian VEGF can comprise the sequence set forth in SEQ ID NO:36 or a fragment thereof. In an aspect, a disclosed encoded mammalian VEGF can comprise a sequence having about 50%, about 55%, about 60%, about 65%, about 70%, about 75%, about 80%, about 85%, about 90%, about 95%, or more than 95% identity to the sequence set forth in SEQ ID NO:36 or a fragment thereof.

In an aspect, a disclosed mammalian VEGF can comprise the sequence set forth in SEQ ID NO:29 or a fragment thereof. In an aspect, a disclosed mammalian VEGF can comprise a sequence having about 50%, about 55%, about 60%, about 65%, about 70%, about 75%, about 80%, about 85%, about 90%, about 95%, or more than 95% identity to the sequence set forth in SEQ ID NO:29 or a fragment thereof.

In an aspect, a disclosed mammalian VEGF can comprise the sequence set forth in SEQ ID NO:30 or a fragment thereof. In an aspect, a disclosed mammalian VEGF can comprise a sequence having about 50%, about 55%, about 60%, about 65%, about 70%, about 75%, about 80%, about 85%, about 90%, about 95%, or more than 95% identity to the sequence set forth in SEQ ID NO:30 or a fragment thereof.

In an aspect, a disclosed mammalian VEGF can comprise the sequence set forth in SEQ ID NO:37 or a fragment thereof. In an aspect, a disclosed mammalian VEGF can comprise a sequence having about 50%, about 55%, about 60%, about 65%, about 70%, about 75%, about 80%, about 85%, about 90%, about 95%, or more than 95% identity to the sequence set forth in SEQ ID NO:37 or a fragment thereof.

In an aspect, a disclosed isolated nucleic acid molecule can be packaged in an AAV capsid or AAV particle (such as, for example, an AA9 or rAAV9 capsid or AAV9 or rAAV9 particle).

In an aspect, a disclosed isolated nucleic acid molecule can packaged in an viral vector or a recombinant viral vector. In an aspect, a disclosed viral vector can be an AAV vector or a recombinant AAV vector or can be a lentiviral vector or a recombinant lentiviral vector.

In an aspect, a disclosed isolated nucleic acid molecule can comprise inverted terminal repeats such as those, for example, derived from the adeno-associated viral (AAV) genome.

Disclosed herein are plasmids that comprise a disclosed isolated nucleic acid molecule, a disclosed transgene, and/or a disclosed transgene cassette. Plasmids are known to the art and described in the Examples provided herein. For example, disclosed herein is an hsp68::lacZ report plasmid having a subcloned TREE (e.g., LEN-hsp68::lacZ, runx1EN-hsp68::lacZ, and il11aEN-hsp68::lacZ constructs).

Disclosed herein is a transgene cassette comprising a disclosed isolated nucleic acid molecule comprising a nucleic acid sequence encoding a tissue regeneration enhancer element (TREE), a minimal promoter with little or no basal activity, a reporter transgene, and a 3′ UTR noncoding region. Disclosed herein is a transgene cassette comprising an isolated nucleic acid molecule, comprising: a nucleic acid sequence encoding a tissue regeneration enhancer element (TREE); a minimal promoter with little or no basal activity; further comprising a reporter transgene; and a 3′ UTR noncoding region. Disclosed herein is a transgene cassette comprising an isolated nucleic acid molecule, comprising a nucleic acid sequence encoding a tissue regeneration enhancer element (TREE) and a minimal promoter with little or no basal activity. Disclosed herein is a transgene cassette comprising an isolated nucleic acid molecule, comprising a nucleic acid sequence encoding a tissue regeneration enhancer element (TREE), a minimal promoter with little or no basal activity, and a 3′ UTR noncoding region. Disclosed herein is a transgene cassette comprising an isolated nucleic acid molecule, comprising a nucleic acid sequence encoding a tissue regeneration enhancer element (TREE), a minimal promoter having little or no basal activity, and at least one polypeptide having biological activity. Disclosed herein is a transgene cassette comprising an isolated nucleic acid molecule, comprising a nucleic acid sequence encoding a tissue regeneration enhancer element (TREE), a minimal promoter having little or no basal activity, at least one polypeptide having biological activity, and a 3′ UTR noncoding region.

Disclosed herein are transgenic animals comprising a disclosed transgene cassette. In an aspect, a disclosed transgenic animal can be used for identification and/or validation on a putative TREE. In an aspect, a disclosed transgenic animal can comprise a mouse or a zebrafish.

### 2. Vectors

Disclosed herein is a vector comprising a disclosed isolated nucleic molecule.

Disclosed herein is a vector comprising an isolated nucleic acid molecule, comprising a nucleic acid sequence encoding a tissue regeneration enhancer element (TREE) and a minimal promoter with little or no basal activity.

Disclosed herein is a vector comprising an isolated nucleic acid molecule, comprising a nucleic acid sequence encoding a tissue regeneration enhancer element (TREE), a minimal promoter with little or no basal activity, and a 3′ UTR noncoding region.

Disclosed herein is a vector comprising an isolated nucleic acid molecule, comprising a nucleic acid sequence encoding a tissue regeneration enhancer element (TREE), a minimal promoter having little or no basal activity, and at least one polypeptide having biological activity.

Disclosed herein is a vector comprising an isolated nucleic acid molecule, comprising a nucleic acid sequence encoding a tissue regeneration enhancer element (TREE), a minimal promoter having little or no basal activity, at least one polypeptide having biological activity, and a 3′ UTR noncoding region.

In an aspect, a disclosed vector can be a viral vector or a non-viral vector. In an aspect, a disclosed non-viral vector can be a polymer-based vector, a peptide based vector, a lipid nanoparticle, a solid lipid nanoparticle, or a cationic lipid based vector.

In an aspect, a disclosed viral vector can be an adenovirus vector, an adeno-associated virus (AAV) vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed viral vector can be an AAV vector or a recombinant AAV vector. For example, in an aspect, a disclosed AAV vector can comprise AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, or AAVcy.7. In an aspect, a disclosed AAV vector can comprise AAV9 or recombinant AAV9.

In an aspect, a disclosed AAV vector can comprise bovine AAV, caprine AAV, canine AAV, equine AAV, can ovine AAV, avian AAV, primate AAV, or non-primate AAV. In an aspect, a disclosed AAV vector can comprise AAV-DJ, AAV-HAE1, AAV-HAE2, AAVM41, AAV-1829, AAV2 Y/F, AAV2 TN, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

In an aspect, a disclosed vector can comprise a tissue-specific promoter operably linked to encoded polypeptide. Tissue-specific promoters are described herein and are known to the art. In an aspect, a disclosed vector can comprise a nucleic acid sequence having a coding sequence that is less than about 4.5 kilobases. In an aspect, a disclosed viral vector can be a lentiviral vector or a recombinant lentiviral vector.

In an aspect, a disclosed viral vector can comprise one or more CRISPR-based epigenome editing tools such as, for example, one or more gRNAs and/or dCas9 and dCas9 alternatives (e.g., VRER). In an aspect, a disclosed viral vector can comprise the sequence for one or more gRNAs. In an aspect, a disclosed gRNA can target an endogenous gene in the subject's stressed, damaged, and/or injured cells or tissues.

In an aspect, a disclosed viral vector can comprise a promoter operably linked to one or more disclosed gRNAs. In an aspect, a disclosed promoter can comprise a ubiquitous promoter, a constitutive promoter, or a tissue specific promoter. In an aspect, a disclosed promoter can comprise a U6 promoter, an H1 promoter, or a 7SK promoter.

Disclosed herein is a lentiviral vector or a recombinant lentiviral vector comprising a disclosed isolated nucleic molecule.

Disclosed herein is a lentiviral vector or a recombinant lentiviral vector comprising an isolated nucleic acid molecule, comprising a nucleic acid sequence encoding a tissue regeneration enhancer element (TREE) and a minimal promoter with little or no basal activity.

Disclosed herein is a lentiviral vector or a recombinant lentiviral vector comprising an isolated nucleic acid molecule, comprising a nucleic acid sequence encoding a tissue regeneration enhancer element (TREE), a minimal promoter with little or no basal activity, and a 3′ UTR noncoding region.

Disclosed herein is a lentiviral vector or a recombinant lentiviral vector comprising an isolated nucleic acid molecule, comprising a nucleic acid sequence encoding a tissue regeneration enhancer element (TREE), a minimal promoter having little or no basal activity, and at least one polypeptide having biological activity.

Disclosed herein is a lentiviral vector or a recombinant lentiviral vector comprising an isolated nucleic acid molecule, comprising a nucleic acid sequence encoding a tissue regeneration enhancer element (TREE), a minimal promoter having little or no basal activity, at least one polypeptide having biological activity, and a 3′ UTR noncoding region.

Disclosed herein is an AAV or an rAAV vector comprising a disclosed isolated nucleic molecule.

Disclosed herein is an AAV or an rAAV vector comprising an isolated nucleic acid molecule, comprising a nucleic acid sequence encoding a tissue regeneration enhancer element (TREE) and a minimal promoter with little or no basal activity.

Disclosed herein is an AAV or an rAAV vector comprising an isolated nucleic acid molecule, comprising a nucleic acid sequence encoding a tissue regeneration enhancer element (TREE), a minimal promoter with little or no basal activity, and a 3′ UTR noncoding region.

Disclosed herein is an AAV or an rAAV vector comprising an isolated nucleic acid molecule, comprising a nucleic acid sequence encoding a tissue regeneration enhancer element (TREE), a minimal promoter having little or no basal activity, and at least one polypeptide having biological activity.

Disclosed herein is an AAV or an rAAV vector comprising an isolated nucleic acid molecule, comprising a nucleic acid sequence encoding a tissue regeneration enhancer element (TREE), a minimal promoter having little or no basal activity, at least one polypeptide having biological activity, and a 3′ UTR noncoding region.

### 3. Pharmaceutical Formulations

Disclosed herein is a pharmaceutical formulation comprising a disclosed isolated nucleic acid molecule and a pharmaceutically acceptable carrier.

Disclosed herein is a pharmaceutical formulation comprising an isolated nucleic acid molecule, comprising a nucleic acid sequence encoding a tissue regeneration enhancer element (TREE) and a minimal promoter with little or no basal activity, and a pharmaceutically acceptable carrier.

Disclosed herein is a pharmaceutical formulation comprising an isolated nucleic acid molecule, comprising a nucleic acid sequence encoding a tissue regeneration enhancer element (TREE), a minimal promoter with little or no basal activity, and a 3′ UTR noncoding region, and a pharmaceutically acceptable carrier.

Disclosed herein is a pharmaceutical formulation comprising an isolated nucleic acid molecule, comprising a nucleic acid sequence encoding a tissue regeneration enhancer element (TREE), a minimal promoter having little or no basal activity, and at least one polypeptide having biological activity, and a pharmaceutically acceptable carrier.

Disclosed herein is a pharmaceutical formulation comprising an isolated nucleic acid molecule, comprising a nucleic acid sequence encoding a tissue regeneration enhancer element (TREE), a minimal promoter having little or no basal activity, at least one polypeptide having biological activity, and a 3′ UTR noncoding region, and a pharmaceutically acceptable carrier.

Disclosed herein is a pharmaceutical formulation comprising a disclosed vector and a pharmaceutically acceptable carrier.

Disclosed herein is a pharmaceutical formulation comprising a lentiviral vector comprising an isolated nucleic acid molecule, comprising a nucleic acid sequence encoding a tissue regeneration enhancer element (TREE) and a minimal promoter with little or no basal activity, and a pharmaceutically acceptable carrier.

Disclosed herein is a pharmaceutical formulation comprising a lentiviral vector comprising an isolated nucleic acid molecule, comprising a nucleic acid sequence encoding a tissue regeneration enhancer element (TREE), a minimal promoter with little or no basal activity, and a 3′ UTR noncoding region, and a pharmaceutically acceptable carrier.

Disclosed herein is a pharmaceutical formulation comprising a lentiviral vector comprising an isolated nucleic acid molecule, comprising a nucleic acid sequence encoding a tissue regeneration enhancer element (TREE), a minimal promoter having little or no basal activity, and at least one polypeptide having biological activity, and a pharmaceutically acceptable carrier.

Disclosed herein is a pharmaceutical formulation comprising a lentiviral vector comprising an isolated nucleic acid molecule, comprising a nucleic acid sequence encoding a tissue regeneration enhancer element (TREE), a minimal promoter having little or no basal activity, at least one polypeptide having biological activity, and a 3′ UTR noncoding region, and a pharmaceutically acceptable carrier.

Disclosed herein is a pharmaceutical formulation comprising an AAV or an rAAV vector comprising a disclosed isolated nucleic molecule and a pharmaceutically acceptable carrier.

Disclosed herein is a pharmaceutical formulation comprising an AAV or an rAAV vector comprising an isolated nucleic acid molecule, comprising a nucleic acid sequence encoding a tissue regeneration enhancer element (TREE) and a minimal promoter with little or no basal activity, and a pharmaceutically acceptable carrier.

Disclosed herein is a pharmaceutical formulation comprising an AAV or an rAAV vector comprising an isolated nucleic acid molecule, comprising a nucleic acid sequence encoding a tissue regeneration enhancer element (TREE), a minimal promoter with little or no basal activity, and a 3′ UTR noncoding region, and a pharmaceutically acceptable carrier.

Disclosed herein is a pharmaceutical formulation comprising an AAV or an rAAV vector comprising an isolated nucleic acid molecule, comprising a nucleic acid sequence encoding a tissue regeneration enhancer element (TREE), a minimal promoter having little or no basal activity, and at least one polypeptide having biological activity, and a pharmaceutically acceptable carrier.

Disclosed herein is a pharmaceutical formulation comprising an AAV or an rAAV vector comprising an isolated nucleic acid molecule, comprising a nucleic acid sequence encoding a tissue regeneration enhancer element (TREE), a minimal promoter having little or no basal activity, at least one polypeptide having biological activity, and a 3′ UTR noncoding region, and a pharmaceutically acceptable carrier.

A disclosed pharmaceutically acceptable carrier can refer to a sterile aqueous or nonaqueous solution, a dispersion, a suspension, an emulsion, or any combination thereof, as well as a sterile powder for reconstitution into a sterile injectable solution, dispersion, suspension, emulsion, or any combination thereof just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

### 4. Kits

Disclosed herein is a kit comprising one or more components and/or reagents for use in a disclosed method of treating stressed, damaged, and/or injured tissues. Disclosed herein is a kit comprising one or more components and/or reagents for use in a disclosed method of stimulating cell proliferation in stressed, damaged, and/or injured tissues. Disclosed herein is a kit comprising one or more components and/or reagents for use in a disclosed method of improving cell survival in stressed, damaged, and/or injured tissues. Disclosed herein is a kit comprising one or more components and/or reagents for use in a disclosed method of enhancing cellular regeneration in stressed, damaged, and/or injured tissues. Disclosed herein is a kit comprising one or more components and/or reagents for use in a disclosed methods of generating a disclosed viral vector such as, for example, a disclosed lentiviral vector or a disclosed AAV vector. Disclosed herein is a kit comprising one or more components and/or reagents for use in a disclosed method of identifying one or more putative TREEs. In an aspect, a disclosed kit can comprise the components and/or reagents necessary to perform one or more steps of a disclosed methods, such as, for example, obtaining a first population of cells, isolating the nuclei from the first population of cells, analyzing chromatin structure and function of the isolated nuclei to obtain a chromatin profile for the first population of cells, obtaining a second population of cells, isolating the nuclei from the second population of cells, analyzing chromatin structure and function of the isolated nuclei to obtain a chromatin profile for the second population of cells and comparing the chromatin profiles between the two populations of cells to identify one or more putative TREEs.

In an aspect, a disclosed kit can comprise one or more disclosed isolated nucleic acid molecules, disclosed vectors, disclosed pharmaceutical formulations, disclosed expression cassettes, disclosed plasmids, or any combination thereof.

In an aspect, a disclosed kit can comprise one or more restriction enzymes, fixative agents, digestion agents, primers, polymerases, ligases, or any combination thereof. In an aspect, a disclosed kit can comprise at least two components and/or reagents constituting the kit. Together, the components and/or reagents constitute a functional unit for a given purpose (such as, for example, a method of treating stressed, damaged, and/or injured tissues). Individual member components may be physically packaged together or separately. For example, a kit comprising an instruction for using the kit may or may not physically include the instruction with other individual member components and/or reagents. Instead, the instruction can be supplied as a separate member component and/or reagent, either in a paper form or an electronic form which may be supplied on computer readable memory device or downloaded from an interne website, or as recorded presentation. In an aspect, a kit for use in a disclosed method can comprise one or more containers holding a disclosed component and/or reagent and a label or package insert with instructions for use. In an aspect, suitable containers include, for example, bottles, vials, syringes, blister pack, etc. The containers can be formed from a variety of materials such as glass or plastic. The container can hold, for example, a disclosed component and/or reagent and can have a sterile access port (for example the container may be an intravenous solution bag or a vial having a stopper pierceable by a hypodermic injection needle). The label or package insert can indicate that a disclosed component and/or reagent can be used in a disclosed method. In an aspect, a disclosed kit can comprise additional components and/or reagents necessary for administration such as, for example, other buffers, polymerases, primers, chemical reagents, diluents, filters, needles, and syringes.

### C. Methods

### 1. Methods of Treating Stressed, Damaged, and/or Injured Tissues

Disclosed herein is a method of treating stressed, damaged, and/or injured tissues, the method comprising administering to a subject in need thereof a disclosed isolated nucleic acid molecule, a disclosed vector, a disclosed pharmaceutical formulation, or any combination thereof.

Disclosed herein is a method of treating stressed, damaged, and/or injured tissues, the method comprising reducing inflammation in stressed, damaged, and/or injured tissues in a subject in need thereof by administering to the subject a disclosed isolated nucleic acid molecule, a disclosed vector, a disclosed pharmaceutical formulation, or any combination thereof.

Disclosed herein is a method of treating stressed, damaged, and/or injured tissues, the method comprising improving cell survival in stressed, damaged, and/or injured tissues in a subject in need thereof by administering to the subject in need thereof a disclosed isolated nucleic acid molecule, a disclosed vector, a disclosed pharmaceutical formulation, or any combination thereof.

Disclosed herein is a method of treating stressed, damaged, and/or injured tissues, the method comprising stimulating cell proliferation in stressed, damaged, and/or injured tissues in a subject in need thereof by administering to the subject in need thereof a disclosed isolated nucleic acid molecule, a disclosed vector, a disclosed pharmaceutical formulation, or any combination thereof.

Disclosed herein is a method of treating stressed, damaged, and/or injured tissues, the method comprising stimulating cell division in stressed, damaged, and/or injured tissues in a subject in need thereof by administering to the subject in need thereof a disclosed isolated nucleic acid molecule, a disclosed vector, a disclosed pharmaceutical formulation, or any combination thereof.

Disclosed herein is a method of treating stressed, damaged, and/or injured tissues, the method comprising inhibiting cell death mechanism in stressed, damaged, and/or injured tissues in a subject in need thereof by administering to the subject in need thereof a disclosed isolated nucleic acid molecule, a disclosed vector, a disclosed pharmaceutical formulation, or any combination thereof.

In an aspect of a disclosed method, a disclosed isolated nucleic acid molecule, a disclosed vector, a disclosed pharmaceutical formulation, or any combination thereof can be systemically or directly administered to the subject. For example, in an aspect, a disclosed isolated nucleic acid molecule, a disclosed vector, a disclosed pharmaceutical formulation, or any combination thereof can be intravenously, subcutaneously, or intramuscularly administered to the subject.

In an aspect of a disclosed method, a disclosed isolated nucleic acid molecule, a disclosed vector, a disclosed pharmaceutical formulation, or any combination thereof can be directly administered to the stressed, damaged, and/or injured tissues.

In an aspect, a disclosed minimal promoter can direct the expression of an endogenous polypeptide or an encoded polypeptide in the subject's stressed, damaged, and/or injured tissues.

In an aspect, minimal promoter directed-expression can be minimal and/or can be absent in subject's non-stressed, non-damaged, and/or non-injured tissues.

In an aspect of a disclosed method, minimal promoter directed-expression of the endogenous polypeptide or an encoded polypeptide can be limited to the subject's stressed, damaged, and/or injured tissues.

In an aspect of a disclosed method, minimal promoter directed-expression of an endogenous polypeptide or an encoded polypeptide can transient or can be sustained. For example, in an aspect, minimal promoter directed-expression of an endogenous polypeptide or an encoded polypeptide can be sustained for at least at least 1 week, at least 2 weeks, at least 1 month, at least 6 weeks, at least 2 months, at least 3 months, at least 4 months, at least 6 months, at least 1 year, at least 18 months, at least 2 years, at least 2.5 years, or at least 3 years. In an aspect, minimal promoter directed-expression of an endogenous polypeptide or an encoded polypeptide can be sustained for at least 3 weeks to at least 6 weeks.

In an aspect of a disclosed method, the subject's stressed, damaged, and/or injured tissue can comprise cardiac tissue. In an aspect, the minimal promoter directed-expression of the endogenous polypeptide or an encoded polypeptide can improve a subject's cardiac function. In an aspect of a disclosed method, a subject's improved cardiac function can be transient or can be sustained. For example, in an aspect, a subject's improved cardiac function can be sustained for at least 2 weeks, at least 1 month, at least 2 months, at least 3 months, at least 4 months, at least 6 months, at least 1 year, at least 18 months, at least 2 years, at least 2.5 years, or at least 3 years. Cardiac function is known to the art and can be measured and/or assessed in any known method. For example, cardiac function can be measured and/or assessed using noninvasive or minimally invasive methods including but not limited to echocardiography and magnetic resonance imaging. In an aspect, long term improvements in a subject's cardiac function can be achieved through transient minimal promoter directed-expression of an endogenous polypeptide or an encoded polypeptide (e.g., at least 3 weeks to at least 6 weeks).

In an aspect of a disclosed method, the minimal promoter directed-expression of the endogenous polypeptide or an encoded polypeptide can reduce the amount of cardiac scar tissue. In an aspect of a disclosed method, the minimal promoter directed-expression of the endogenous polypeptide or an encoded polypeptide can improve cardiac cell survival such as, for example, cardiac smooth muscle cells. In an aspect of a disclosed method, the minimal promoter directed-expression of the endogenous polypeptide or an encoded polypeptide can stimulate proliferation of cardiac cells. In an aspect of a disclosed method, the minimal promoter directed-expression of the endogenous polypeptide or an encoded polypeptide can inhibit cardiac cell death mechanisms in the stressed, damaged, and/or injured cardiac tissue such as, for example, apoptotic cell death mechanisms and/or necrotic cell death mechanisms. In an aspect of a disclosed method, the minimal promoter directed-expression of the endogenous polypeptide or an encoded polypeptide can increase vascular supply in the stressed, damaged, and/or injured cardiac tissue. In an aspect, cardiac vascular supply can be increased in stressed, damaged, and/or injured tissues. In an aspect of a disclosed method, the minimal promoter directed-expression of the endogenous polypeptide or an encoded polypeptide can reduce inflammation in the stressed, damaged, and/or injured cardiac tissue. In an aspect of a disclosed method, the minimal promoter directed-expression of the endogenous polypeptide or an encoded polypeptide can reduce the subject's pain due to the stressed, damaged, and/or injured cardiac tissue.

In an aspect of a disclosed method, the stressed, damaged, and/or injured tissues can comprise brain tissue and/or spinal cord tissue. In an aspect of a disclosed method, the minimal promoter directed-expression of the endogenous polypeptide or an encoded polypeptide can improve the subject's brain and/or spinal cord function. Brain and/or spinal cord function is known to the art and can be measured and/or assessed in any known method. For example, brain and/or spinal cord function can be measured and/or assessed using noninvasive or minimally invasive methods. In an aspect of a disclosed method, the minimal promoter directed-expression of the endogenous polypeptide or an encoded polypeptide can reduce the amount of scar tissue in the stressed, damaged, and/or injured brain and/or spinal cord. In an aspect, the minimal promoter directed-expression of the endogenous polypeptide or an encoded polypeptide can improve cell survival of, for example, brain cells and/or spinal cord axons. In an aspect, the minimal promoter directed-expression of the endogenous polypeptide or an encoded polypeptide can stimulate proliferation of brain cells and/or growth of spinal cord axons. In an aspect of a disclosed method, the minimal promoter directed-expression of the endogenous polypeptide or an encoded polypeptide can stimulate brain cell growth and/or spinal cord axonal growth. an aspect of a disclosed method, the minimal promoter directed-expression of the endogenous polypeptide or an encoded polypeptide can increase vascular supply in the stressed, damaged, and/or injured brain and/or spinal cord. In an aspect, the minimal promoter directed-expression of the endogenous polypeptide or an encoded polypeptide can inhibit cell death mechanisms in the stressed, damaged, and/or injured brain tissue and/or spinal cord tissue such as, for example, apoptotic cell death mechanisms and/or necrotic cell death mechanisms. In an aspect of a disclosed method, the minimal promoter directed-expression of the endogenous polypeptide or an encoded polypeptide can reduce inflammation in the stressed, damaged, and/or injured brain and/or spinal cord. In an aspect, the minimal promoter directed-expression of the endogenous polypeptide or an encoded polypeptide reduces in the subject's pain associated with the stressed, damaged, and/or injured brain and/or spinal cord.

In an aspect, the stressed, damaged, and/or injured tissue can comprise cartilage and/or bone. In an aspect of a disclosed method, the minimal promoter directed-expression of the endogenous polypeptide or an encoded polypeptide can improve the cartilage and/or bone function. Cartilage and/or bone function is known to the art and can be measured and/or assessed in any known method. For example, cartilage and/or bone function can be measured and/or assessed using noninvasive or minimally invasive methods. In an aspect, cartilage can comprise hyaline cartilage, fibrous cartilage, and/or elastic cartilage. In an aspect, cartilage can comprise perichrondrium. In an aspect, the minimal promoter directed-expression of the endogenous polypeptide or an encoded polypeptide can improve cartilage and/or bone structural integrity. In an aspect, the minimal promoter directed-expression of the endogenous polypeptide or an encoded polypeptide can improve cartilage and/or bone function. In an aspect, the minimal promoter directed-expression of the endogenous polypeptide or an encoded polypeptide can reduce the amount of scar tissue in stressed, damaged, and/or injured cartilage and/or bone. In an aspect, the minimal promoter directed-expression of the endogenous polypeptide or an encoded polypeptide reduces the subject's pain in the stressed, damaged, and/or injured cartilage and/or bone. In an aspect, the minimal promoter directed-expression of the endogenous polypeptide or an encoded polypeptide can improve cell survival such as, for example, the survival of chondrocytes and/or chondroblasts. In an aspect, the minimal promoter directed-expression of the endogenous polypeptide or an encoded polypeptide can inhibit cell death mechanisms in stressed, damaged, and/or injured cartilage and/or bone such as, for example, apoptotic cell death mechanisms and/or necrotic cell death mechanisms. In an aspect, the minimal promoter directed-expression of the endogenous polypeptide or an encoded polypeptide can increase vascular supply in the stressed, damaged, and/or injured cartilage and/or bone.

In an aspect, a disclosed method can further comprise repeating the administering step.

In an aspect, a disclosed method can further comprise one or more therapeutic agents. Therapeutic agents are known to the art. In an aspect, a therapeutic agent can comprise a statin, aspirin, warfarin, clopidogrel, a beta blocker, an ACE inhibitor, or any combination thereof. In an aspect, a therapeutic agent can comprise any agent that is capable of providing a local or systemic biological, physiological, or therapeutic effect in the biological system to which it is applied. For example, the a therapeutic agent can act to control infection or inflammation, enhance cell growth and tissue regeneration, control tumor growth, act as an analgesic, promote anti-cell attachment, and enhance bone growth, among other functions. Therapeutic agents as well as the specifics of the administration of therapeutic agents (i.e., dosing amount and schedule, administration route, etc.) are known the art. As used herein, the recitation of a biologically active agent inherently encompasses the pharmaceutically acceptable salts thereof.

In an aspect, a disclosed method can further comprise monitoring the subject for adverse effects. In an aspect, in the absence of adverse effects, the method can further comprise continuing to treat the subject. In an aspect, in the presence of adverse effects, the method can further comprise modifying the treating step.

In an aspect of a disclosed method, methods and techniques to monitor a subject can comprise qualitative (or subjective) means as well as quantitative (or objective) means. In an aspect, qualitative means (or subjective means) can comprise a subject's own perspective. For example, a subject can report how he/she is feeling, whether he/she has experienced improvements and/or setbacks, whether he/she has experienced an amelioration or an intensification of one or more symptoms, or a combination thereof. In an aspect, quantitative means (or objective means) can comprise methods and techniques that include, but are not limited to, the following: (i) fluid analysis (e.g., tests of a subject's fluids including but not limited to aqueous humor and vitreous humor, bile, blood, blood serum, breast milk, cerebrospinal fluid, cerumen (earwax), digestive fluids, endolymph and perilymph, female ejaculate, gastric juice, mucus (including nasal drainage and phlegm), peritoneal fluid, pleural fluid, saliva, sebum (skin oil), semen, sweat, synovial fluid, tears, vaginal secretion, vomit, and urine), (ii) imaging (e.g., ordinary x-rays, ultrasonography, radioisotope (nuclear) scanning, computed tomography (CT), magnetic resonance imaging (MRI), positron emission tomography (PET), and angiography), (iii) endoscopy (e.g., laryngoscopy, bronchoscopy, esophagoscopy, gastroscopy, GI endoscopy, coloscopy, cystoscopy, hysteroscopy, arthroscopy, laparoscopy, mediastinoscopy, and thoracoscopy), (iv) analysis of organ activity (e.g., electrocardiography (ECG), electroencephalography (EEG), and pulse oximetry), (v) biopsy (e.g., removal of tissue samples for microscopic evaluation), and (vi) genetic testing.

In an aspect, a disclosed method can further comprise generating the viral or non-viral vector. In an aspect, generating the viral vector can comprise generating a lentiviral vector. In an aspect, generating the viral vector can comprise generating an AAV vector (such as, for example, an AAV9 vector).

In an aspect, a disclosed method of treating stressed, damaged, and/or injured tissues can comprise spatiotemporally targeted tissue regeneration.

In an aspect, a disclosed method of treating stressed, damaged, and/or injured tissues can be used in a platform for spatiotemporally targeted tissue regeneration.

### 2. Methods of Stimulating Cell Proliferation

Disclosed herein is a method of stimulating cell proliferation in stressed, damaged, and/or injured tissues in a subject in need thereof, the method comprising administering to the subject in need thereof a disclosed isolated nucleic acid molecule, a disclosed vector, a disclosed pharmaceutical formulation, or any combination thereof.

In an aspect of a disclosed method, a disclosed isolated nucleic acid molecule, a disclosed vector, a disclosed pharmaceutical formulation, or any combination thereof can be systemically or directly administered to the subject. For example, in an aspect, a disclosed isolated nucleic acid molecule, a disclosed vector, a disclosed pharmaceutical formulation, or any combination thereof can be intravenously, subcutaneously, or intramuscularly administered to the subject.

In an aspect of a disclosed method, a disclosed isolated nucleic acid molecule, a disclosed vector, a disclosed pharmaceutical formulation, or any combination thereof can be directly administered to the stressed, damaged, and/or injured tissues.

In an aspect, a disclosed minimal promoter can direct the expression of an endogenous polypeptide or an encoded polypeptide in the subject's stressed, damaged, and/or injured tissues.

In an aspect, minimal promoter directed-expression can be minimal and/or can be absent in subject's non-stressed, non-damaged, and/or non-injured tissues, and/or can be limited to the subject's stressed, damaged, and/or injured tissues.

In an aspect of a disclosed method, minimal promoter directed-expression of an endogenous polypeptide or an encoded polypeptide can transient or can be sustained. For example, in an aspect, minimal promoter directed-expression of an endogenous polypeptide or an encoded polypeptide can be sustained for at least at least 1 week, at least 2 weeks, at least 1 month, at least 6 weeks, at least 2 months, at least 3 months, at least 4 months, at least 6 months, at least 1 year, at least 18 months, at least 2 years, at least 2.5 years, or at least 3 years. In an aspect, minimal promoter directed-expression of an endogenous polypeptide or an encoded polypeptide can be sustained for at least 3 weeks to at least 6 weeks.

In an aspect of a disclosed method, the subject's stressed, damaged, and/or injured tissue can comprise cardiac tissue, brain and/or spinal cord tissue, cartilage and/or bone, or any combination thereof.

In an aspect, the stressed, damaged, and/or injured tissue can comprise cardiac tissue. In an aspect, the minimal promoter directed-expression of the endogenous polypeptide or an encoded polypeptide can improve the subject's cardiac function. Cardiac function is known to the art and can be measured and/or assessed in any known method including those discussed supra. In an aspect of a disclosed method, the subject's improved cardiac function can be transient or can be sustained (discussed supra).

In an aspect of a disclosed method, the minimal promoter directed-expression of the endogenous polypeptide or an encoded polypeptide can reduce the amount of scar tissue, can improve cardiac cell survival such as, for example, cardiac smooth muscle cells, can stimulate proliferation of cardiac cells, can inhibit cell death mechanisms in the stressed, damaged, and/or injured cardiac tissue such as, for example, apoptotic cell death mechanisms and/or necrotic cell death mechanisms, can increase vascular supply in the stressed, damaged, and/or injured cardiac tissue, can reduce inflammation in the stressed, damaged, and/or injured cardiac tissue, can reduce the subject's pain due to the stressed, damaged, and/or injured cardiac tissue, or any combination thereof.

In an aspect of a disclosed method, the stressed, damaged, and/or injured tissues can comprise brain tissue and/or spinal cord tissue. In an aspect of a disclosed method, the minimal promoter directed-expression of the endogenous polypeptide or an encoded polypeptide can improve the subject's brain and/or spinal cord function. Brain and/or spinal cord function is known to the art and can be measured and/or assessed in any known method. In an aspect of a disclosed method, the subject's improved Brain and/or spinal cord function can be transient or can be sustained (discussed supra).

In an aspect of a disclosed method, the minimal promoter directed-expression of the endogenous polypeptide or an encoded polypeptide can reduce the amount of scar tissue, can improve cell survival of, for example, brain cells and/or spinal cord axons, can stimulate proliferation of brain cells and/or growth of spinal cord axons, can stimulate brain cell growth and/or spinal cord axonal growth, can inhibit cell death mechanisms in the stressed, damaged, and/or injured brain tissue and/or spinal cord tissue such as, for example, apoptotic cell death mechanisms and/or necrotic cell death mechanisms., can reduce inflammation in the stressed, damaged, and/or injured brain tissue and/or spinal cord tissue, can reduce in the subject's pain in the stressed, damaged, and/or injured brain tissue and/or spinal cord tissue, or any combination thereof.

In an aspect, the stressed, damaged, and/or injured tissue can comprise cartilage and/or bone. In an aspect, cartilage can comprise hyaline cartilage, fibrous cartilage, and/or elastic cartilage. In an aspect, cartilage can comprise perichrondrium. In an aspect, the minimal promoter directed-expression of the endogenous polypeptide or an encoded polypeptide can improve cell survival such as, for example, the survival of chondrocytes and/or chondroblasts, can inhibit cell death mechanisms in stressed, damaged, and/or injured cartilage and/or bone such as, for example, apoptotic cell death mechanisms and/or necrotic cell death mechanisms, can increase vascular supply in the stressed, damaged, and/or injured cartilage and/or bone, can improve cartilage and/or bone structural integrity, can improve cartilage and/or bone function, can reduce the amount of scar tissue in stressed, damaged, and/or injured cartilage and/or bone, can reduce the subject's pain in the stressed, damaged, and/or injured cartilage and/or bone, or any combination thereof.

In an aspect, a disclosed method can further comprise repeating the administering step.

In an aspect, a disclosed method can further comprise one or more therapeutic agents. Therapeutic agents are known to the art and are discussed supra.

In an aspect, a disclosed method can further comprise monitoring the subject for adverse effects. In an aspect, in the absence of adverse effects, the method can further comprise continuing to treat the subject. In an aspect, in the presence of adverse effects, the method can further comprise modifying the treating step.

In an aspect of a disclosed method of stimulating cell proliferation, methods and techniques to monitor a subject can comprise qualitative (or subjective) means as well as quantitative (or objective) means. These methods and techniques are known to the art and are discussed supra.

In an aspect, the disclosed method of stimulating cell proliferation can further comprise treating the stressed, damaged, and/or injured tissues. In an aspect, treating the stressed, damaged, and/or injured tissues can comprise improving cell survival in the tissues, enhancing cellular regeneration in the tissues, stimulating cell division in the tissues, reducing inflammation in the tissues, inhibiting cell death mechanisms in the tissues, or any combination thereof. In an aspect, the disclosed method of stimulating cell proliferation can comprise improving cell survival in the tissues, enhancing cellular regeneration in the tissues, stimulating cell division in the tissues, reducing inflammation in the tissues, inhibiting cell death mechanisms in the tissues, or any combination thereof.

In an aspect, a disclosed method can further comprise generating the viral or non-viral vector. In an aspect, generating the viral vector can comprise generating a lentiviral vector. In an aspect, generating the viral vector can comprise generating an AAV vector (such as, for example, an AAV9 vector).

In an aspect, a disclosed method of stimulating cell proliferation can comprise spatiotemporally targeted tissue regeneration.

In an aspect, a disclosed method of stimulating cell proliferation can be used in a platform for spatiotemporally targeted tissue regeneration.

### 3. Methods of Improving Cell Survival

Disclosed herein is a method of improving cell survival in stressed, damaged, and/or injured tissues in a subject in need thereof, the method comprising administering to the subject in need thereof a disclosed isolated nucleic acid molecule, a disclosed vector, a disclosed pharmaceutical formulation, or any combination thereof.

In an aspect of a disclosed method, a disclosed isolated nucleic acid molecule, a disclosed vector, a disclosed pharmaceutical formulation, or any combination thereof can be systemically or directly administered to the subject. For example, in an aspect, a disclosed isolated nucleic acid molecule, a disclosed vector, a disclosed pharmaceutical formulation, or any combination thereof can be intravenously, subcutaneously, or intramuscularly administered to the subject.

In an aspect of a disclosed method, a disclosed isolated nucleic acid molecule, a disclosed vector, a disclosed pharmaceutical formulation, or any combination thereof can be directly administered to the stressed, damaged, and/or injured tissues.

In an aspect, a disclosed minimal promoter can direct the expression of an endogenous polypeptide or an encoded polypeptide in the subject's stressed, damaged, and/or injured tissues.

In an aspect, minimal promoter directed-expression can be minimal and/or can be absent in subject's non-stressed, non-damaged, and/or non-injured tissues, and/or can be limited to the subject's stressed, damaged, and/or injured tissues.

In an aspect of a disclosed method, minimal promoter directed-expression of an endogenous polypeptide or an encoded polypeptide can transient or can be sustained. For example, in an aspect, minimal promoter directed-expression of an endogenous polypeptide or an encoded polypeptide can be sustained for at least at least 1 week, at least 2 weeks, at least 1 month, at least 6 weeks, at least 2 months, at least 3 months, at least 4 months, at least 6 months, at least 1 year, at least 18 months, at least 2 years, at least 2.5 years, or at least 3 years. In an aspect, minimal promoter directed-expression of an endogenous polypeptide or an encoded polypeptide can be sustained for at least 3 weeks to at least 6 weeks.

In an aspect of a disclosed method, the subject's stressed, damaged, and/or injured tissue can comprise cardiac tissue, brain and/or spinal cord tissue, cartilage and/or bone, or any combination thereof.

In an aspect, the stressed, damaged, and/or injured tissue can comprise cardiac tissue. In an aspect, the minimal promoter directed-expression of the endogenous polypeptide or an encoded polypeptide can improve the subject's cardiac function. Cardiac function is known to the art and can be measured and/or assessed in any known method including those discussed supra. In an aspect of a disclosed method, the subject's improved cardiac function can be transient or can be sustained (discussed supra).

In an aspect of a disclosed method, the minimal promoter directed-expression of the endogenous polypeptide or an encoded polypeptide can reduce the amount of scar tissue, can improve cardiac cell survival such as, for example, cardiac smooth muscle cells, can stimulate proliferation of cardiac cells, can inhibit cell death mechanisms in the stressed, damaged, and/or injured cardiac tissue such as, for example, apoptotic cell death mechanisms and/or necrotic cell death mechanisms, can increase vascular supply in the stressed, damaged, and/or injured cardiac tissue, can reduce inflammation in the stressed, damaged, and/or injured cardiac tissue, can reduce the subject's pain due to the stressed, damaged, and/or injured cardiac tissue, or any combination thereof.

In an aspect of a disclosed method, the stressed, damaged, and/or injured tissues can comprise brain tissue and/or spinal cord tissue. In an aspect of a disclosed method, the minimal promoter directed-expression of the endogenous polypeptide or an encoded polypeptide can improve the subject's brain and/or spinal cord function. Brain and/or spinal cord function is known to the art and can be measured and/or assessed in any known method. In an aspect of a disclosed method, the subject's improved Brain and/or spinal cord function can be transient or can be sustained (discussed supra).

In an aspect of a disclosed method, the minimal promoter directed-expression of the endogenous polypeptide or an encoded polypeptide can reduce the amount of scar tissue, can improve cell survival of, for example, brain cells and/or spinal cord axons, can stimulate proliferation of brain cells and/or growth of spinal cord axons, can stimulate brain cell growth and/or spinal cord axonal growth, can inhibit cell death mechanisms in the stressed, damaged, and/or injured brain tissue and/or spinal cord tissue such as, for example, apoptotic cell death mechanisms and/or necrotic cell death mechanisms., can reduce inflammation in the stressed, damaged, and/or injured brain tissue and/or spinal cord tissue, can reduce in the subject's pain in the stressed, damaged, and/or injured brain tissue and/or spinal cord tissue, or any combination thereof.

In an aspect, the stressed, damaged, and/or injured tissue can comprise cartilage and/or bone. In an aspect, cartilage can comprise hyaline cartilage, fibrous cartilage, and/or elastic cartilage. In an aspect, cartilage can comprise perichrondrium. In an aspect, the minimal promoter directed-expression of the endogenous polypeptide or an encoded polypeptide can improve cell survival such as, for example, the survival of chondrocytes and/or chondroblasts, can inhibit cell death mechanisms in stressed, damaged, and/or injured cartilage and/or bone such as, for example, apoptotic cell death mechanisms and/or necrotic cell death mechanisms, can increase vascular supply in the stressed, damaged, and/or injured cartilage and/or bone, can improve cartilage and/or bone structural integrity, can improve cartilage and/or bone function, can reduce the amount of scar tissue in stressed, damaged, and/or injured cartilage and/or bone, can reduce the subject's pain in the stressed, damaged, and/or injured cartilage and/or bone, or any combination thereof.

In an aspect, a disclosed method can further comprise repeating the administering step.

In an aspect, a disclosed method can further comprise one or more therapeutic agents. Therapeutic agents are known to the art and are discussed supra.

In an aspect, a disclosed method can further comprise monitoring the subject for adverse effects. In an aspect, in the absence of adverse effects, the method can further comprise continuing to treat the subject. In an aspect, in the presence of adverse effects, the method can further comprise modifying the treating step.

In an aspect of a disclosed method of stimulation cell proliferation, methods and techniques to monitor a subject can comprise qualitative (or subjective) means as well as quantitative (or objective) means. These methods and techniques are known to the art and are discussed supra.

In an aspect, the disclosed method of improving cell survival can further comprise treating the stressed, damaged, and/or injured tissues. In an aspect, treating the stressed, damaged, and/or injured tissues can comprise stimulating cell proliferation in the tissues, enhancing cellular regeneration, stimulating cell division in the tissues, reducing inflammation in the tissues, inhibiting cell death mechanisms in the tissues, or any combination thereof. In an aspect, the disclosed method of improving cell survival can comprise stimulating cell proliferation in the tissues, stimulating cell division in the tissues, reducing inflammation in the tissues, inhibiting cell death mechanisms in the tissues, or any combination thereof.

In an aspect, a disclosed method can further comprise generating the viral or non-viral vector. In an aspect, generating the viral vector can comprise generating a lentiviral vector. In an aspect, generating the viral vector can comprise generating an AAV vector (such as, for example, an AAV9 vector).

In an aspect, a disclosed method of improving cell survival can comprise spatiotemporally targeted tissue regeneration.

In an aspect, a disclosed method of improving cell survival can be used in a platform for spatiotemporally targeted tissue regeneration.

### 4. Methods of Enhancing Cellular Regeneration

Disclosed herein is a method of enhancing cellular regeneration in stressed, damaged, and/or injured tissues in a subject in need thereof, the method comprising administering to the subject in need thereof a disclosed isolated nucleic acid molecule, a disclosed vector, a disclosed pharmaceutical formulation, or any combination thereof.

In an aspect of a disclosed method, a disclosed isolated nucleic acid molecule, a disclosed vector, a disclosed pharmaceutical formulation, or any combination thereof can be systemically or directly administered to the subject. For example, in an aspect, a disclosed isolated nucleic acid molecule, a disclosed vector, a disclosed pharmaceutical formulation, or any combination thereof can be intravenously, subcutaneously, or intramuscularly administered to the subject.

In an aspect of a disclosed method, a disclosed isolated nucleic acid molecule, a disclosed vector, a disclosed pharmaceutical formulation, or any combination thereof can be directly administered to the stressed, damaged, and/or injured tissues.

In an aspect, a disclosed minimal promoter can direct the expression of an endogenous polypeptide or an encoded polypeptide in the subject's stressed, damaged, and/or injured tissues.

In an aspect, minimal promoter directed-expression can be minimal and/or can be absent in subject's non-stressed, non-damaged, and/or non-injured tissues, and/or can be limited to the subject's stressed, damaged, and/or injured tissues.

In an aspect of a disclosed method, minimal promoter directed-expression of an endogenous polypeptide or an encoded polypeptide can transient or can be sustained. For example, in an aspect, minimal promoter directed-expression of an endogenous polypeptide or an encoded polypeptide can be sustained for at least at least 1 week, at least 2 weeks, at least 1 month, at least 6 weeks, at least 2 months, at least 3 months, at least 4 months, at least 6 months, at least 1 year, at least 18 months, at least 2 years, at least 2.5 years, or at least 3 years. In an aspect, minimal promoter directed-expression of an endogenous polypeptide or an encoded polypeptide can be sustained for at least 3 weeks to at least 6 weeks.

In an aspect of a disclosed method, the subject's stressed, damaged, and/or injured tissue can comprise cardiac tissue, brain and/or spinal cord tissue, cartilage and/or bone, or any combination thereof.

In an aspect, the stressed, damaged, and/or injured tissue can comprise cardiac tissue. In an aspect, the minimal promoter directed-expression of the endogenous polypeptide or an encoded polypeptide can improve the subject's cardiac function. Cardiac function is known to the art and can be measured and/or assessed in any known method including those discussed supra. In an aspect of a disclosed method, the subject's improved cardiac function can be transient or can be sustained (discussed supra).

In an aspect of a disclosed method, the minimal promoter directed-expression of the endogenous polypeptide or an encoded polypeptide can reduce the amount of scar tissue, can improve cardiac cell survival such as, for example, cardiac smooth muscle cells, can stimulate proliferation of cardiac cells, can inhibit cell death mechanisms in the stressed, damaged, and/or injured cardiac tissue such as, for example, apoptotic cell death mechanisms and/or necrotic cell death mechanisms, can increase vascular supply in the stressed, damaged, and/or injured cardiac tissue, can reduce inflammation in the stressed, damaged, and/or injured cardiac tissue, can reduce the subject's pain due to the stressed, damaged, and/or injured cardiac tissue, or any combination thereof, thereby enhancing cellular regeneration in the tissue.

In an aspect of a disclosed method, the stressed, damaged, and/or injured tissues can comprise brain tissue and/or spinal cord tissue. In an aspect of a disclosed method, the minimal promoter directed-expression of the endogenous polypeptide or an encoded polypeptide can improve the subject's brain and/or spinal cord function. Brain and/or spinal cord function is known to the art and can be measured and/or assessed in any known method. In an aspect of a disclosed method, the subject's improved brain and/or spinal cord function can be transient or can be sustained (discussed supra).

In an aspect of a disclosed method, the minimal promoter directed-expression of the endogenous polypeptide or an encoded polypeptide can reduce the amount of scar tissue, can improve cell survival of, for example, brain cells and/or spinal cord axons, can stimulate proliferation of brain cells and/or growth of spinal cord axons, can stimulate brain cell growth and/or spinal cord axonal growth, can inhibit cell death mechanisms in the stressed, damaged, and/or injured brain tissue and/or spinal cord tissue such as, for example, apoptotic cell death mechanisms and/or necrotic cell death mechanisms., can reduce inflammation in the stressed, damaged, and/or injured brain tissue and/or spinal cord tissue, can reduce in the subject's pain in the stressed, damaged, and/or injured brain tissue and/or spinal cord tissue, or any combination thereof, thereby enhancing cellular regeneration in the tissue.

In an aspect, the stressed, damaged, and/or injured tissue can comprise cartilage and/or bone. In an aspect, cartilage can comprise hyaline cartilage, fibrous cartilage, and/or elastic cartilage. In an aspect, cartilage can comprise perichrondrium. In an aspect, the minimal promoter directed-expression of the endogenous polypeptide or an encoded polypeptide can improve cell survival such as, for example, the survival of chondrocytes and/or chondroblasts, can improve cellular proliferation, can inhibit cell death mechanisms in stressed, damaged, and/or injured cartilage and/or bone such as, for example, apoptotic cell death mechanisms and/or necrotic cell death mechanisms, can increase vascular supply in the stressed, damaged, and/or injured cartilage and/or bone, can improve cartilage and/or bone structural integrity, can improve cartilage and/or bone function, can reduce the amount of scar tissue in stressed, damaged, and/or injured cartilage and/or bone, can reduce the subject's pain in the stressed, damaged, and/or injured cartilage and/or bone, or any combination thereof, thereby enhancing cellular regeneration in the tissue.

In an aspect, a disclosed method can further comprise repeating the administering step.

In an aspect, a disclosed method can further comprise one or more therapeutic agents. Therapeutic agents are known to the art and are discussed supra.

In an aspect, a disclosed method can further comprise monitoring the subject for adverse effects. In an aspect, in the absence of adverse effects, the method can further comprise continuing to treat the subject. In an aspect, in the presence of adverse effects, the method can further comprise modifying the treating step.

In an aspect of a disclosed method of enhancing cellular regeneration, methods and techniques to monitor a subject can comprise qualitative (or subjective) means as well as quantitative (or objective) means. These methods and techniques are discussed supra.

In an aspect, the disclosed method of enhancing cellular regeneration can further comprise treating the stressed, damaged, and/or injured tissues. In an aspect, treating the stressed, damaged, and/or injured tissues can comprise stimulating cell proliferation in the tissues, improving cell survival in the tissues, stimulating cell division in the tissues, reducing inflammation in the tissues, inhibiting cell death mechanisms in the tissues, or any combination thereof.

In an aspect, the disclosed method of enhancing cellular regeneration can comprise stimulating cell proliferation in the tissues, improving cell survival in the tissues, stimulating cell division in the tissues, reducing inflammation in the tissues, inhibiting cell death mechanisms in the tissues, or any combination thereof.

In an aspect, a disclosed method can further comprise generating the viral or non-viral vector. In an aspect, generating the viral vector can comprise generating a lentiviral vector. In an aspect, generating the viral vector can comprise generating an AAV vector (such as, for example, as an AAV9 vector).

In an aspect, a disclosed method of enhancing cellular regeneration can comprise spatiotemporally targeted tissue regeneration.

In an aspect, a disclosed method of enhancing cellular generation can be used in a platform for spatiotemporally targeted tissue regeneration.

### 5. Methods of Generating Viral Vectors

Disclosed herein is a method of generating a disclosed viral vector.

Disclosed herein is method of generating a disclosed lentiviral vector comprising seeding cells onto a gelatin coated dish, co-transfecting a DNA expression plasmid with psPAX2 and VSVg, collecting and concentrating the supernatant, and centrifuging and harvesting the concentrated lentiviral particles.

Disclosed herein is a method of generating a disclosed lentiviral vector comprising co-transfecting into cells a recombinant construct containing a pAd-DELTA F6 helper plasmid, a serotype-specific plasmid AAV2/9, and an AAV plasmid containing the cassette of choice, harvesting the transfected cells, and purifying the concentrated AAV particles.

Methods of generating viral vectors are known to the art and are disclosed in the Examples provided herein.

### 6. Methods of Identifying Putative TREEs

Disclosed herein is a method of identifying one or more putative TREEs comprising isolating the nuclei from a first population of cells and a second population of cells, analyzing chromatin structure and function of the isolated nuclei to obtain a chromatin profile for the first population of cells and for the second population of cells, and comparing the chromatin profiles of the two populations of cells to identify one or more putative TREEs.

In an aspect, a disclosed first population of cells can have sustained an injury and/or an insult, and a disclosed second population of cells can have not sustained an injury and/or an insult, or vice versa.

In an aspect, a disclosed first population of cells can have sustained an injury and/or an insult and is regenerating, and a disclosed second population of cells can have not sustained an injury and/or an insult, or vice versa.

In an aspect, a disclosed first population of cells can have sustained an injury and/or an insult and is regenerating, and a disclosed second population of cells can have sustained an injury and/or an insult and is not regenerating, or vice versa.

In an aspect, a disclosed first population of cells can be obtained from a subject having sustained an injury and/or insult, and a disclosed second population of cells can be obtained from a subject not having sustained an injury and/or insult, or vice versa.

In an aspect, a disclosed first population of cells can be obtained from a subject having sustained an injury and/or insult and is regenerating, and a disclosed second population of cells can be obtained from a subject not having sustained an injury and/or insult, or vice versa.

In an aspect, a disclosed first population of cells can be obtained from a subject having sustained an injury and/or insult and is regenerating, and a disclosed second population of cells can be obtained from a subject having sustained an injury and/or insult and is not regenerating, or vice versa.

In an aspect of a disclosed method, a disclosed first population of cells can comprise a compilation and/or aggregate of cells. In an aspect of a disclosed method, a disclosed second population of cells can comprise a compilation and/or aggregate of cells. In an aspect, a disclosed compilation and/or aggregate of cells can comprise cells from one or more tissues and/or organs. In an aspect, a disclosed compilation and/or aggregate of cells can comprise cultured cells. In an aspect, a disclosed compilation and/or aggregate of cells can comprise cultured cells having sustained stress, damage, and/or injury.

In an aspect of a disclosed method, disclosed cells can comprise mammalian cells. Mammalian cells can comprise, for example, human cells, porcine cells, or mouse cells. In an aspect of a disclosed method, disclosed cells can comprise zebrafish cells. In an aspect of a disclosed method, one or both subjects can comprise a mammal. In an aspect of a disclosed method, one or both subjects can comprise a human, a pig, or a mouse. In an aspect of a disclosed method, one or both subjects can comprise a zebrafish.

In an aspect of a disclosed method, the injury and/or insult can comprise a cardiac injury and/or insult, a brain and/or spinal cord injury and/or insult, a bone and/or cartilage injury and/or insult, or any combination thereof.

In an aspect of a disclosed method, the injury and/or insult can comprise a disease. In an aspect, a disclosed disease can be cardiovascular disease, a CNS disease, a PNS disease, a degenerative disease, diabetes, a chronic disease, or any combination thereof. In an aspect, a disclosed disease can be arthritis, osteoarthritis, or rheumatoid arthritis. In an aspect, a disclosed CNS or a disclosed PNS can comprise Alzheimer's disease, Parkinson's disease, Huntington's disease, Pick's disease, Kuf's disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dytonia syndrome, Leigh syndrome, Leber hereditary optic neuropathy (LHON), parkinsonism, dystonia, motor neuron disease, neuropathy-ataxia and retinitis pimentosa (NARP), maternal inherited Leigh syndrome (MILS), Friedreich ataxia, hereditary spastic paraplegia, Mohr-Tranebjaerg syndrome, Wilson disease, sporatic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, korsakoff's psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity, or movement disorders.

In an aspect of a disclosed method, one or more putative TREEs can be incorporated into a vector (such as, for example, an AAV9 vector) or an isolated nucleic acid molecule.

In an aspect, a disclosed method can further comprise validating one or more putative TREEs. In an aspect, validating one or more putative TREEs can comprise generating a transgenic zebrafish and assessing the ability of the one or more putative TREEs to drive expression of a reporter gene in stressed, damaged, and/or injured tissues, in regenerating tissue, in non-regenerating tissue, and or any combination thereof. In an aspect, validating one or more putative TREEs can comprise generating an AAV vector comprising a putative TREE and assessing the ability of the AAV vector comprising the putative TREE to drive expression of a gene (e.g., a reporter gene) in mice having stressed, damaged, and/or injured tissues, in regenerating tissue, in non-regenerating tissue, and or any combination thereof.

Methods and techniques for analyzing chromatin are well-known to the art and include, but are not limited to, ATAC-seq, chromatin immunoprecipitation (ChIP), ChIP-Seq, ChIP-exo, ChIA-PET (Chromatin Interaction Analysis by Paired-End Tag Sequencing), Chromatin Conformation Capture (3C), Circularized Chromosome Conformation Capture (4C), Carbon Copy Chromosome Conformation Capture (5C), ChIP-Loop, Hi-C, and Capture-C.

As known to the art, in an aspect, ATAC-seq can assess the accessibility to transposase and can be an indicator of open chromatin that is actively involved in gene regulation. In an aspect, during the analysis of chromatin, the skilled person can identify those sequences with chromatin that is more open and accessible. The skilled person can also identify those sequences having greater enrichment of active enhancer marks like H3K27Ac in injured and/or regenerating cells and/or tissue (as compared to the non-injured and/or non-regenerating cells and/or tissues). In an aspect, identifying these changes can enable the identification of a putative TREEs, which is preferentially or specifically able to direct gene expression in injured and/or regenerating cells and/or tissues, but not in non-injured and/or non-regenerating cells and/or tissues.

Disclosed herein is a method of identifying one or more TREEs comprising obtaining a first population of cells, isolating the nuclei from the first population of cells, analyzing chromatin structure and function of the isolated nuclei to obtain a chromatin profile for the first population of cells, obtaining a second population of cells, isolating the nuclei from the second population of cells, analyzing chromatin structure and function of the isolated nuclei to obtain a chromatin profile for the second population of cells and comparing the chromatin profiles between the two populations of cells to identify one or more putative TREEs.

In an aspect, a disclosed first population of cells can have sustained an injury and/or an insult, and a disclosed second population of cells can have not sustained an injury and/or an insult, or vice versa.

In an aspect, a disclosed first population of cells can have sustained an injury and/or an insult and is regenerating, and a disclosed second population of cells can have not sustained an injury and/or an insult, or vice versa.

In an aspect, a disclosed first population of cells can have sustained an injury and/or an insult and is regenerating, and a disclosed second population of cells can have sustained an injury and/or an insult and is not regenerating, or vice versa.

In an aspect, a disclosed first population of cells can be obtained from a subject having sustained an injury and/or insult, and a disclosed second population of cells can be obtained from a subject not having sustained an injury and/or insult, or vice versa.

In an aspect, a disclosed first population of cells can be obtained from a subject having sustained an injury and/or insult and is regenerating, and a disclosed second population of cells can be obtained from a subject not having sustained an injury and/or insult, or vice versa.

In an aspect, a disclosed first population of cells can be obtained from a subject having sustained an injury and/or insult and is regenerating, and a disclosed second population of cells can be obtained from a subject having sustained an injury and/or insult and is not regenerating, or vice versa.

In an aspect of a disclosed method, a disclosed first population of cells can comprise a compilation and/or aggregate of cells. In an aspect of a disclosed method, a disclosed second population of cells can comprise a compilation and/or aggregate of cells. In an aspect, a disclosed compilation and/or aggregate of cells can comprise cells from one or more tissues and/or organs. In an aspect, a disclosed compilation and/or aggregate of cells can comprise cultured cells. In an aspect, a disclosed compilation and/or aggregate of cells can comprise cultured cells having sustained stress, damage, and/or injury.

In an aspect of a disclosed method, disclosed cells can comprise mammalian cells. Mammalian cells can comprise, for example, human cells, porcine cells, or mouse cells. In an aspect of a disclosed method, disclosed cells can comprise zebrafish cells. In an aspect of a disclosed method, one or both subjects can comprise a mammal. In an aspect of a disclosed method, one or both subjects can comprise a human, a pig, or a mouse. In an aspect of a disclosed method, one or both subjects can comprise a zebrafish.

In an aspect of a disclosed method, the injury and/or insult can comprise a cardiac injury and/or insult, a brain and/or spinal cord injury and/or insult, a bone and/or cartilage injury and/or insult, or any combination thereof. In an aspect of a disclosed method, the injury and/or insult can comprise a disease. In an aspect, a disclosed disease can be cardiovascular disease, a CNS disease, a PNS disease, a degenerative disease, diabetes, a chronic disease, or any combination thereof. In an aspect, a disclosed disease can be arthritis, osteoarthritis, or rheumatoid arthritis. In an aspect, a disclosed CNS or a disclosed PNS can comprise Alzheimer's disease, Parkinson's disease, Huntington's disease, Pick's disease, Kuf's disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dytonia syndrome, Leigh syndrome, Leber hereditary optic neuropathy (LHON), parkinsonism, dystonia, motor neuron disease, neuropathy-ataxia and retinitis pimentosa (NARP), maternal inherited Leigh syndrome (MILS), Friedreich ataxia, hereditary spastic paraplegia, Mohr-Tranebjaerg syndrome, Wilson disease, sporatic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, korsakoff's psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity, or movement disorders.

In an aspect of a disclosed method, one or more putative TREEs can be incorporated into a vector or an isolated nucleic acid molecule.

In an aspect, a disclosed method can further comprise validating one or more putative TREEs. In an aspect, validating one or more putative TREEs can comprise generating a transgenic zebrafish and assessing the ability of the one or more putative TREEs to drive expression of a reporter gene in zebrafish having stressed, damaged, and/or injured tissues, in regenerating tissue, in non-regenerating tissue, and or any combination thereof. In an aspect, validating one or more putative TREEs can comprise generating an AAV vector comprising a putative TREE and assessing the ability of the AAV vector comprising the putative TREE to drive expression of a gene (e.g., a reporter gene) in mice having stressed, damaged, and/or injured tissues, in regenerating tissue, in non-regenerating tissue, and or any combination thereof.

In an aspect, a disclosed method of identifying putative TREEs can be used in a platform for spatiotemporally targeted tissue regeneration.

### EXAMPLES

Mammalian hearts display limited regeneration of new muscle after injury. Recent findings indicate that forced expression of mitogenic or reprogramming factors can enhance heart regeneration in mice, yet these strategies require manipulations that impact gene programs throughout the organ. Studies in species like zebrafish with elevated regenerative capacity have identified tissue regeneration enhancer elements, or TREEs, that can activate expression of their target genes in an injury site, maintain expression during regeneration, and alleviate expression as regeneration concludes. Here, TREEs identified from the zebrafish genome directed injury-associated gene expression in contexts of ischemic cardiac damage in small and large mammalian species. When employed in combination with CRISPR-based epigenome editing tools in mice, zebrafish TREEs stimulated or repressed the expression of endogenous genes in infarcted hearts. Viral vectors engineered with a TREE to direct a mutated Yap transcription factor gene and introduced to mice by systemic perfusion boost cardiomyocyte proliferation and dedifferentiation selectively in ischemic injury sites. These findings demonstrate cross-species application of regeneration enhancer elements as a potential therapeutic platform for spatiotemporally targeted tissue regeneration.

### A. Materials and Methods

### 1. Zebrafish

Wild-type or transgenic zebrafish of the EK/AB strain were used for all experiments. β-actin2:1oxp-mCherry-STOP-loxp-DTA^{pd36} (Wang J, et al. (2011) Development. 138:3421-3430), cmlc2:CreER^{pd10} (Kikuchi K, et al. (2010) Nature. 464:601-605) and ankrd1aEN-cfos:EGFP (Goldman J A, et al. (2017) Dev Cell. 40:392-404) transgenic mutant or fish have been previously described. To induce ablation of CMs, adult ZCAT animals were treated for 24 hours with 0.5 μM-1.0 μM tamoxifen titrated to ablate ˜50% of CMs (Wang J, et al. (2011)). Procedures involving zebrafish were approved by the Institutional Animal Care and Use Committee at Duke University.

### 2. Transgenic Mice

All mice were maintained in Division of Laboratory Animal Resources (mouse facility. All mouse experiments were performed in accordance with federal and institutional guidelines and were reviewed and approved by the Duke, Cincinnati, UCSF, and Weizmann Institute of Science IACUC committees.

Wild-type male and female mice of C57BL/6 were purchased from The Jackson Laboratory with adults ranging in age from 8 to 12 weeks. Male and female animals were used for experiments with the exception of assays for CM proliferation and cardiac function, in which only adult male mice were used for ligation of LAD injury model.

Rosa26:LSL-dCas9^{p300} (dCas9^{p300}) and Rosa26:LSL-dCas9^{KRAB} (dCas9^{KRAB}) transgenic mice were previously described (Gemberling M, et al. (2021) Nature Methods. 18:965-974). Briefly, these mice carry a CAG promoter, a loxP flanked triple polyadenylation signal (pA) stop cassette (lox-stop-lox; LSL), and a codon optimized dCas9-p300 (with a 1X-FLAG) or dCas9-KRAB (3X-FLAG) cassette. Mice were genotyped using the following primers.

| Description | Sequence | Identifier |
|---|---|---|
| Forward Primer | GCAGCCTCTGTTC | SEQ ID NO: 01 |
| | CACATACAC | |
| Reverse Primer | TAAGCCTGCCCAG | SEQ ID NO: 02 |
| | AAGACTC | |
| Second Forward | AAAGTCGCTCTGA | SEQ ID NO: 03 |
| Primer | GTTGTTAT | |

Expected product sizes were as follows: WT band (235 bp); Knock-In (162 bp). To ensure working with the correct transgenic line, an additional PCR specific for dCas9^{KRAB} mice was performed with primers recognizing the KRAB cassette.

| Description | Sequence | Identifier |
|---|---|---|
| KRAB Forward | GGCGCGCCTGC | SEQ ID NO: 04 |
| Primer | AGCCTTCAAG | |
| KRAB Reverse | GAATCAGGATG | SEQ ID NO: 05 |
| Primer | GGTCTCTTGG | |

Product size was 446 bp. Genotyping was performed using KAPA polymerase and the following PCR conditions: 95° C. for 1 min, 35 cycles of: 95° C. for 15 seconds, 57° C. for 15 seconds, 72° C. for 10 minutes, and 4° C. Hold. All mice used in the experiments were homozygous for dCas9^{p300} or dCas9^{KRAB}.

### 3. Generation of TREE-hsp68::LacZ Transgenic Mice

TREE-hsp68:: lacZ transgenic mice were generated by oocyte microinjection as described previously (Dodou E, et al. (2003) Mech Dev. 120:1021-1032). Zebrafish LEN, runx1EN, and il11aEN were subloned into the hsp68::lacZ reporter plasmid, and LEN-hsp68::lacZ, runx1EN-hsp68::lacZ, and il11aEN-hsp68::lacZ constructs were injected into fertilized CD-1 strain embryos to generate stable transgenic mouse lines. These lines were then backcrossed to C57BL/6 to expand the colony. Mice were genotyped using the following primers.

| Description | Sequence | Identifier |
|---|---|---|
| lacZ Forward Primer | TTTAACGCCGT | SEQ ID NO: 06 |
| | GCGCTGTTCG | |
| lacZ Reverse Primer | ATCCAGCGATA | SEQ ID NO: 07 |
| | CAGCGCGTCG | |

Expected product size was 275 bp.

### 4. Ligation of the Left Anterior Descending Coronary Artery (LAD)

MI injuries were performed with the assistance of the Duke Cardiovascular Physiology Core (Curcio A, et al. (2006) Am J Physiol Heart Circ Physiol 291:H1754-1760). Mice (8-12 weeks) were anesthetized with ketamine/xylazine or isoflurane, intubated, and placed on a rodent ventilator. Then, the chest cavity was entered in the third intercostal space at the left lateral border. The left atrium was gently deflected out of the field to expose the left anterior descending artery. Coronary ligation was performed by tying a suture ligature around the LAD artery. Following coronary ligation, the chest was closed, the pneumothorax evacuated, and the mice were extubated and allowed to recover from anesthesia.

### 5. Skeletal Muscle Injury

The procedure for skeletal muscle injury in adult mice was performed as described previously (An Y, et al. (2017) Dev. Cell. 41:382-391). Briefly, mice (8-12 weeks) were anesthetized with isoflurane. The skin surface of tibialis anterior (TA) muscle was shaved, scrubbed, and injected with 1.2% BaCl₂ to induce acute muscle injury.

### 6. Tibial Fracture

Tibial fracture surgery was performed as described previously (Baht G S, et al. (2014) J. Orthop. Res. 32:581-586). Briefly, mice (4 months) were anesthetized with isoflurane. An incision was made proximal to the knee to expose tibial plateau. A fracture was induced at the mid-shaft with aid of an insect pin inserted into the medullary cavity. The incision site was then closed, and mice were monitored until recovery.

### 7. Digit Amputation

Adult mice were anesthetized using isoflurane. A sterile scissor was used to amputate 2^{nd} and 4^{th} digits. After amputation, gentle pressure was applied on the wound with gauze to stop bleeding.

### 8. AAV Plasmid Construction and Virus Production

Zebrafish regulatory sequences were subcloned upstream of an AAV construct containing a murine minimal hsp68 promoter and either an EGFP reporter, or a mCherry reporter, or human Yap5SA. Human Yap5SA was provided from B. Varelas (Boston University). CMV::GFP plasmid was provided by A. Asokan (Duke University).

gRNAs were designed using http://crispr-era.stanford.edu/index.jsp (Liu H, et al. (2021) Methods Mol. Biol. 2189:65-69). Four to six gRNAs were tested per gene. Sequences yielding maximum efficiency and used for all experiments were as follows:

| Description | Sequence | Identifier |
|---|---|---|
| agrn gRNA | GACTGCGGCGCCCGCCGAGC | SEQ ID NO: 08 |
| sav1 gRNA | AGTTTACCGGACGTAGGCGG | SEQ ID NO: 09 |

An AAV backbone containing a gRNA cloning site under the control of a U6 promoter and a CMV::Cre cassette was used to clone gRNAs using the Gibson assembly method. Prior to AAV production, ITRs were verified by Smal digest. After selecting the most efficient gRNAs, the CMV promoter was excised and replaced with either a LEN-hsp68 or a 2ankd1aEN-hsp68 fragment using Gibson assembly. ITRs were verified by Smal digest and the resulting plasmids were used to package AAVs.

AAV particles were produced and purified as described previously (Uezu A, et al. (2016) Science. 353:1123-1129). Briefly, recombinant constructs containing a pAd-DELTA F6 helper plasmid, a serotype-specific plasmid AAV2/9, and an AAV plasmid containing the cassette of choice were transfected into HEK293T cells. 72 hrs after transfection, cells were harvested, purified with iodixanol gradient, and concentrated with 100 kDa filter. pAd-DELTA F6, serotype plasmids AAV2/9 and AAV plasmid vectors were provided from S. Soderling (Duke University). Some AAVs were generated by the University of Pennsylvania Vector Core or Duke University Viral Vector Core. Adult mice were systemically injected with 1×10¹¹ virus particles by tail vein.

### 9. Histology and Imaging in Zebrafish

Staining for immunofluorescence was performed as described previously (Kikuchi K, et al. (2011) Dev. Cell. 20:397-404). Antibodies used in this study were anti-Myosin heavy chain (mouse, F59, Developmental Studies Hybridoma Bank), anti-EGFP (rabbit; Abcam), Alexa Fluor 488 (rabbit; Life Technologies), and Alexa Fluor 594 (mouse and rabbit; Life Technologies). Confocal imaging was performed using a Zeiss LSM 700.

### 10. Cardiac Function Assessed by Echocardiography

Echocardiography was performed as described previously (Bassat E, et al. (2021) Methods Mol. Biol. 2158:3-21). Briefly, mice were anesthetized with isoflurane, cardiac function was assessed by transthoracic echocardiography using Vevo3100 VisualSonics device and were analyzed by Vevo Lab software (VisualSonics). Ejection fraction measurement was analyzed from long axis B mode images, and other measurements were analyzed from short axis M mode images.

### 11. Histology and Imaging in Mice

For cardiomyocyte proliferation assays, hearts were extracted, immediately placed into ice-cold 30% sucrose, flash frozen using TFM (VWR), and sectioned at 5 μm. Immunofluorescence staining was performed as described previously (Hirose K, et al. (2019) Science. 364:184-188. Briefly, cryosections were fixed in 3.7% formaldehyde, permeabilized with PBS containing 0.2% Triton X-100, and blocked with PBST containing 5% normal donkey serum for 1 hour. antibody was incubated overnight at 4° C. Sections were then washed and incubated with specific secondary antibodies and DAPI.

For other experiments, mice were perfused, tissues were fixed with 4% PFA, and cryo-embedded (fixation buffer containing 20% formaldehyde and 2% glutaraldehyde was used for X-gal staining). Fixed heart and liver samples were sectioned at 10 μm, at 8 μm for Masson's trichrome-stained samples, and 12 μm for samples stained to detect Cas9. Slides were washed in PBS, permeabilized with PBS containing 0.2% Triton X-100 for 20 minutes and blocked with 5% Goat serum+0.1% Tween in PBS (3% BSA+0.1% Tween in PBS was used as blocking buffer for the Cas9 stained samples) for 1 hour. Primary antibody was incubated overnight at 4° C. in blocking reagent. Sections were then washed and incubated with specific secondary antibodies and DAPI. Confocal images were acquired with Zeiss LSM 700 or Zeiss LSM 880 microscope. Antibodies used were anti-EGFP (rabbit, A11122, Life Technologies), anti-EGFP (chicken, GFP-1020, Ayes Labs), anti-Ds-Red (rabbit, 632496, Clontech), anti-Ki67 (rat, 41569880, ThermoFisher Scientific), anti-Ki67 (rat, 14569882, ThermoFisher Scientific), WGA (W21404, Invitrogen), anti-HA (rabbit, ab9110, Abcam), anti-HA (rabbit, 3724S, Cell Signaling Technology), anti-αSMA (rabbit, A7248, ABclonal), anti-troponin T (mouse, Developmental Studies Hybridoma Bank), anti-troponin T (mouse, MS-295-PABX, ThermoFisher Scientific), Cas9 (rabbit, RPCA-CAS9-Sp, EnCor Biotechnology), Alexa Fluor 488 (chicken, mouse, rabbit, and rat; Life Technologies), Alexa Fluor 546 (mouse and rabbit; Life Technologies), Alexa Fluor 633 (mouse, rabbit, and rat; Life Technologies). X-gal staining was performed using β-galactosidase reporter gene staining kit (Sigma). In situ hybridization was performed on 4% PFA-fixed cryosections with assistance of an InSituPro robot (Intavis).

For CM proliferation and dedifferentiation assays, three sections showing the largest infarcted area were selected from each heart. Quantification analyses at the border zone (<400 μm around infarcted area) (Bassat E, et al. (2021) Methods Mol Biol. 2158:3-21) were performed with 15 fields per heart imaged for Ki67 or EdU staining, and 9 fields per heart imaged for αSMA staining. Quantification analyses at distal areas were performed with 9 fields per heart imaged for Ki67, EdU or αSMA staining. The percentages of Ki67⁺DAPI⁺WGA⁺/DAPI⁺WGA⁺, EdU⁺DAPI⁺WGA⁺/DAPI⁺WGA⁺, or αSMA⁺Tnnt⁺/Tnnt⁺ cells from the three selected sections were averaged to determine a proliferation or dedifferentiation index for each heart.

### 12. Scar Analyses

Assessment of scar area was performed as previously descried (Takagawa J, et al. (1985) J Appl Physiol. 102:2104-2111). Briefly, Masson's trichrome staining was performed on serial sections (from ligation plane to apex) from each heart. Scar size for each heart was calculated by dividing sum of midline infarct lengths from serial sections by the sum of the midline circumferences from serial sections.

### 13. RNA Isolation and QRT-PCR

Hearts were homogenized in Trizol using a Tissue Lyser II (QIAGEN). RNA was extracted using the standard Trizol protocol and genomic DNA removed using RNA clean and Concentrator Kit (Zymo Research/Cat#R1013). cDNA synthesis was performed using Transcriptor First Strand cDNA Synthesis Kit (Roche/Cat #04897030001) and qPCR run was performed with LightCycler 480 SYBR Green I Master (Roche/Cat #04707516001). All gene expression values were normalized to Gapdh in the same well to control for sample handling. Primers for qPCR were as follows:

| Description | Sequence | Identifier |
|---|---|---|
| agrn Forward | TTCGATGGTCCTTGTGACCC | SEQ ID NO: 10 |
| agrn Reverse | AGATAGGTGTGTGTTGGGCG | SEQ ID NO: 11 |
| sal1 Forward | GGGAGGCACACTTCAGGTAT | SEQ ID NO: 12 |
| sal1 Reverse | CAGCATTCCCTGGTACGTGT | SEQ ID NO: 13 |
| β-Actin | AAGGCCAACCGTGAAAAGAT | SEQ ID NO: 14 |
| Forward | | |
| β-Actin | GTGGTACGACCAGAGGGATA | SEQ ID NO: 15 |
| Reverse | C | |

### 14. Protein Isolation and Western Blotting

For protein extraction, hearts were homogenized in RIPA buffer containing Proteinase and Phosphatase inhibitor (Thermo Fisher/Cat #78442). Samples were denatured at 95° C. for 5 min and tissue lysates were run on Mini-Protein tetra cell (Bio-Rad) using sodium dodecyl sulfate polyacrylamide gel electrophoresis (SDS-PAGE) in Tris/glycine/SDS buffer. Proteins were transferred to a PVDF membrane using Mini-Protein tetra cell in Tris/glycine buffer (v/v). Membranes were blocked for non-specific binding 1 hr at room temperature with 3% BSA in Tris- buffered saline and Tween-20 (TBS-T) or 3% Milk in TBS-T, according to the antibody used. Membranes were then incubated with primary antibodies in the following manner: Anti-Cas9 (mouse, EnCor Biotechnology/Cat #MCA-3F9) 1:1000 over-night (ON) at 4° C. in 3% milk TBS-T; Anti-Gapdh (mouse, Proteintech/Cat #60004-1-IG) 1:500 in 3% milk TBS-T or 3% BSA TBS-T ON at 4° C.; Anti-Agrn (mouse, Santa Cruz Biotechnology/Cat #sc-374117) 1:100 in 3% BSA TBS-T ON at 4° C.; Anti-Salvador (mouse, Santa Cruz Biotechnology/Cat #sc-101205) 1:100 in 3% milk TBS-T ON at 4° C. After washing, membranes were incubated with Anti-Mouse HRP secondary antibody (Thermo Fisher Scientific/Cat #31430) in TBS-T containing 3% BSA or milk at 1:50000, for 1 hr RT. Proteins were visualized using Thermo Scientific SuperSignal West Dura Chemiluminescent Substrate (Fisher Scientific/Cat #34075). ImageJ were used for western blots quantifications (Davarinej ad H. (2017)).

### 15. Preparation of Lentivirus

Lentiviral vectors were prepared as previously described (Juhas M, et al. (2018) Nat. Biomed. Eng. 2:942-954); Rao L, et al. (2018) Nat. Commun. 9:126). Briefly, twenty-four hours prior to transfection, 6×10⁶ 293FT cells (Life Technologies, R700-07) were seeded onto a gelatin-coated 10-cm dish. Following the manufacturer instructions, 10 μg DNA expression lentiviral plasmid (runx1EN-hsp68::EGFP, LEN-hsp68::EGFP or hsp68::EGFP) was co-transfected with 5 μg psPAX2 and 2 μg VSVg by Lipofectamine2000 (Thermo). The supernatant was collected 4 hr-72 hr post-transfection and concentrated with Lenti-X concentrator (Clontech) at a 3:1 ratio (supernatant: concentrator) in 4° C. overnight. Concentrated lentiviral particles were harvested following 45 min centrifugation (1500×g, 4° C.), resuspended in 1:100 of the original volume in PBS, and stored at ˜80° C. for further use.

### 16. Neonatal Rat Cardiomyocyte Culture

Neonatal rat ventricular myocytes (NRVMs) were isolated from hearts of 2-day-old Sprague-Dawley rats using previously described methods (Jackman C P, et al. (2018) Biomaterials. 159:48-58; Li Y, et al. (2020) Biomaterials. 236:119824). Isolated NRVMs were plated on 4-well chamber slides at a density of 2.0×10⁵ cells/well, cultured in maintenance media (DMEM F12+2% FBS+0.2% Pen-strep) for 24 hr, and subsequently utilized for lentiviral transduction experiments.

### 17. hiPSC-CM Cultures

Human iPSC-derived cardiomyocytes (hiPSC-CMs) were differentiated as previously described (Shadrin I Y, et al. (2017) Nat. Commun. 8:1825; Jackman C P, et al. (2016) Biomaterials. 111:66-79). Briefly, differentiation was carried out using small molecules CHIR99021 and IWP-2 to upregulate and inhibit Wnt signaling, respectively (Lian, X. et al. (2013) Nature Protocols. 8:162-175). At differentiation days 10-12, hiPSC-CMs were purified by exposure to a glucose-free, chemically defined medium (CDM₃) (Tohyama S, et al. (2013) Cell Stem Cell. 12:127-137; Burridge P W, et al. (2014) Nat. Meth. 11(8):855-860). After 12-22 days of differentiation, purified hiPSC-CMs were seeded on 4-well chamber slides at a density of 2.0×10⁵ cells/well, cultured in maintenance media (RPMI+2% B-27 supplement) for 24 hr, and then utilized for lentiviral transduction experiments.

### 18. Lentiviral Transduction of Cells and In Vitro Injury

NRVMs or hiPSC-CMs were transduced with lentiviruses (runx1EN-hsp68-EGFP, LEN-hsp68-EGFP, or hsp68-EGFP) for 17 hrs. Twenty-for hours after viral transduction, cells were either heat-shocked or injured by an ischemia/reperfusion (I/R) protocol. Heat-shock of cultured cells was performed as previously described (Zhang M, et al. (2001) J. Mol. Cell. Cardiol. 33:907-921). Briefly, dishes with cultured NRVMs or hiPSC-CMs were placed in a 42° C. water bath for 1 hr and then returned to 37° C. incubator for 24 hrs before the endpoint analysis. I/R injury of cultured NRVMs or hiPSC-CMs was performed based on a previously described protocol (Chen T, et al. (2019) Tissue Eng. Part A. 25:711-724). Briefly, cells on 4-chamber slides were incubated in 200 μL/well of an ischemic medium (119 mM NaCl, 12 mM KCl, 1.2 mM NaH₂PO₄, 1.3 mM MgSO₄, 0.5 mM MgCl₂, 0.9 mM CaCl₂, 20 mM sodium lactate, and 5 mM HEPES, pH=6.4), placed into a hypoxic chamber gassed with a mixture of 95% N₂ and 5% CO₂, and cultured in these conditions at 37° C. for 1 hr. The ischemic medium was then replaced with the maintenance medium and cells were returned to a standard 37° C., 5% CO₂ incubator for 24 hrs before the endpoint analysis.

Twenty-four hours after heat-shock or I/R injury, cells were washed in PBS and fixed with 4% paraformaldehyde in PBS for 15 mins at room temperature, then immunostained as previously described (Shadrin I, et al. (2017) Nat. Commun. 8:1825). Briefly, following fixation, samples were washed with PBS and blocked in 5% donkey serum and 0.5% Triton-X 100 solution. Primary antibody for sarcomeric α-actinin (Sigma, a7811, 1:200) was applied at 4° C. overnight, followed by incubation with DAPI and secondary antibody Alexa-594 (1:400) for 1.5 hrs at room temperature. Images were acquired using a confocal fluorescent microscope (ZEISS LSM 880).

### 19. Porcine Injuries and Tissue Harvest

TREEs were tested in a series of large mammal studies performed in Yorkshire swine (30-40 kg) using a closed-chest, ischemia-reperfusion model of myocardial infarction. Pig study protocols were reviewed and approved by the Duke University IACUC. All procedures in swine were performed under deep anesthesia using 4 mg/kg ketamine (IM) and 0.5 mg/kg midazolam (IM) for induction and maintained using 0.5-3.0% isoflurane inhalation via endotrachaeal intubation.

Carotid artery cutdown was used for arterial access, heparin 200-300 units administered intravenously, and a 6-French sheath inserted. Animals were administered a 3 mg/kg bolus of lidocaine followed by a continuous infusion of 2 mg/min. Using angioplasty techniques, balloon occlusion with a 2.50-2.75 mm×12 mm device (Emerge Monorail PTCA Dilation Catheter, Boston Scientific, Marlborough, MA, USA) of the mid-LAD for 90 minutes was performed followed by full reperfusion to induce an anterior wall MI of the apex. ST segment elevation and frequent PVCs were observed on electrocardiogram monitoring to confirm transmural myocardial ischemia

Delivery of TREE vector to the heart was via direct intramyocardial injection (IM) or intracoronary infusion (IC), at 1×10¹⁴ total viral particles per animal. For the IC delivery, a right Judkins Right diagnostic catheter was guided from a carotid artery sheath to the left main coronary artery under fluoroscopy and the TREE vector slowly infused over 60-90 seconds. The catheter was flushed with 5 cc of normal saline to ensure infusion of all vector from the delivery system. Completion coronary angiography confirmed patency of the LAD and circumflex artery. Animals were recovered from anesthesia and survived for few days to few weeks. For direct IM delivery, a right mini-anterior thoracotomy was performed to expose the apex of the heart. A ⅝″ needle was used to inject 0.5 mL aliquots of viral vector into the infarct border zone and a separate remote area of the RV.

Hearts were harvested via sternotomy after systemic administration of a high potassium chloride solution to arrest the heart in diastole. Fresh biopsies were obtained from the infarct, border zone, and remote regions of the heart and samples also preserved in 4% PFA.

### 20. Data Collection and Statistics

No animal or sample was excluded from the analysis unless viral infection, MI, or IF staining was not performed successfully. For echocardiography measurements, only mice with ejection fraction changes from the baseline range of 10% to 60% were included in the experiment. Quantification of CM proliferation and dedifferentiation, echocardiography measurement, and scar analysis were assessed by a person blinded to the treatments.

Other experiments were not blinded during experiments and outcome assessment. Sample sizes, statistical tests, and P values are indicated in the figures or the legends. All statistical values are displayed as Mean±Standard Deviation or Mean±Standard Error of the Mean, as indicated in the figure legends. Statistical tests were calculated using two-tailed Student's t tests or Mann-Whitney U tests with aid of Prism software. The ANOVA followed by Dunnett's post hoc test or Mann-Whitney U tests was applied in echocardiography measurement.

### B. Specific Examples

### Example 1

### Zebrafish Trees Were Injury-Responsive in Adult Mouse Tissues

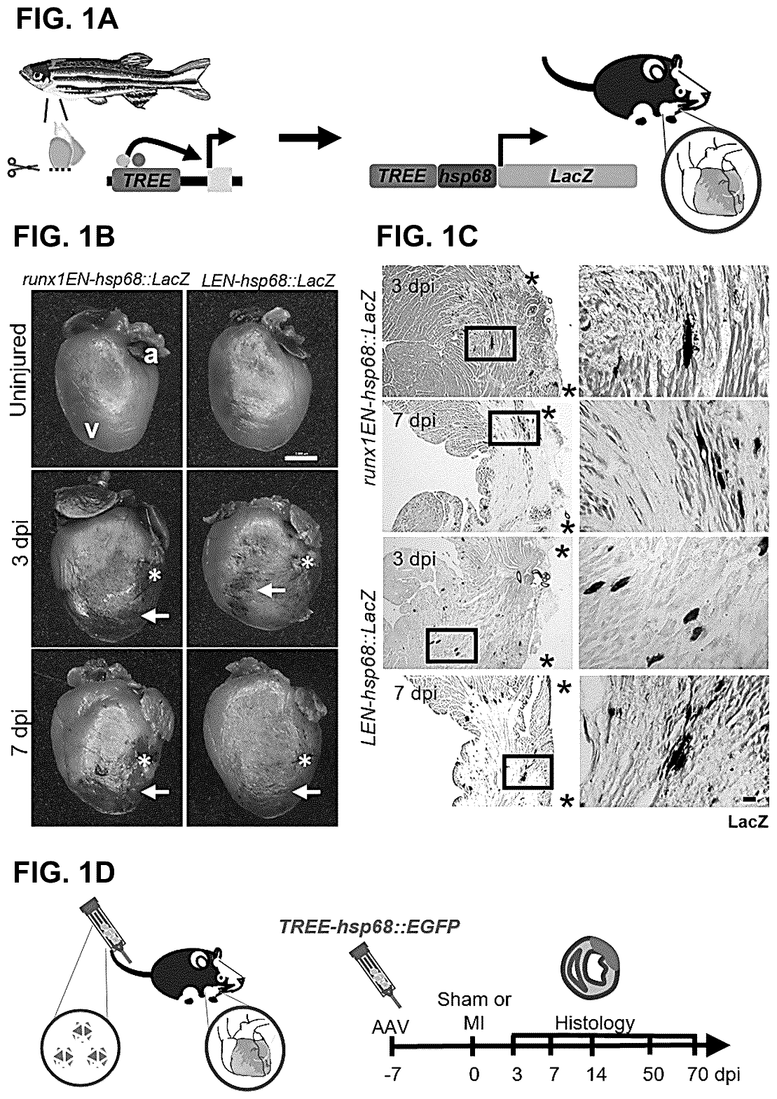

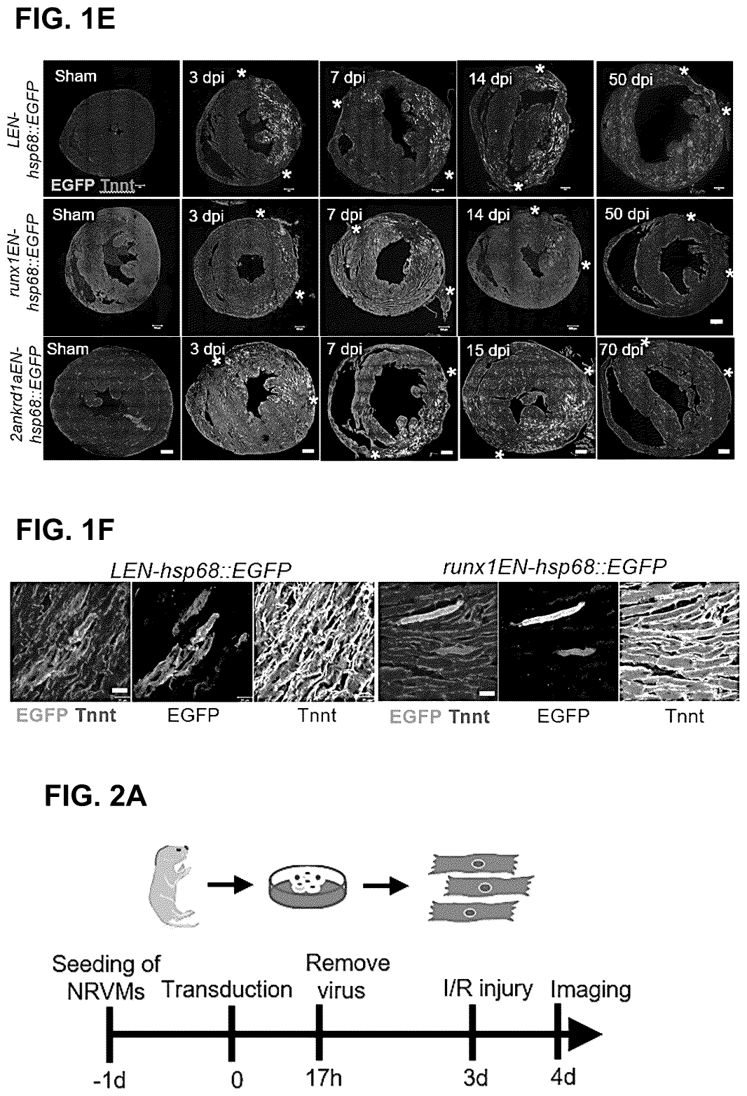

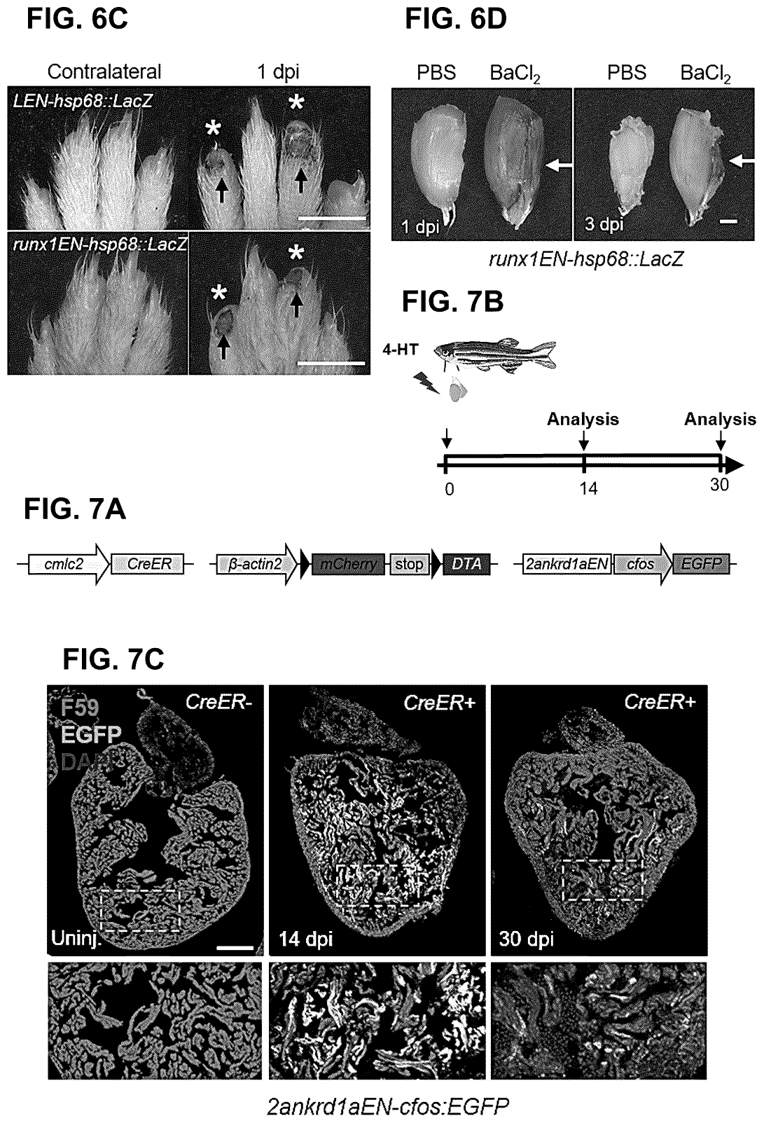

To assess if zebrafish TREEs direct gene expression to cardiac injuries in adult mice, previously described transgenic mice or new transgenic reporter mice generated with constructs containing a zebrafish TREE, minimal hsp68 promoter, and lacZ gene cassette were examined (FIG. 1A). The zebrafish TREEs lepb-linked enhancer (LEN), runx1linked enhancer (runx1EN), and il11a-linked enhancer (il11aE1V) ((Liu H, et al. (2021); Jackman C P, et al. (2018)) each directed robust lacZ reporter gene expression to border zone tissue following ligation of the left anterior descending coronary artery (LAD), whereas little or no expression was detectable in uninjured animals (FIG. 1B, FIG. 6A). Histology indicated increased reporter gene expression selectively at injury sites at 3 and 7 days post injury (dpi) (FIG. 1C). These findings indicate that zebrafish enhancers can target gene expression to injury sites in adult mammalian heart.

To assess whether zebrafish enhancers direct injury-induced gene expression in other adult tissues, tibial bone fractures were imparted, digits were amputated, and muscle injuries were generated in transgenic reporter mice by BaCl₁ injection. Injury-restricted LacZ expression was observed in tibial fracture sites at 21 dpi and at the amputation planes of digits at 1 dpi in LEN-hsp68::LacZ transgenic animals (FIG. 6B and FIG. 6C). runx1EN directed LacZ expression at digit amputation plane and injured skeletal muscle (FIG. 6C and FIG. 6D). These findings indicate that zebrafish enhancers direct injury-induced expression in various adult mouse tissues.

### Example 2

### Zebrafish Trees Target Gene Expression to Injured Myocardial Tissue when Delivered by Viral Vectors

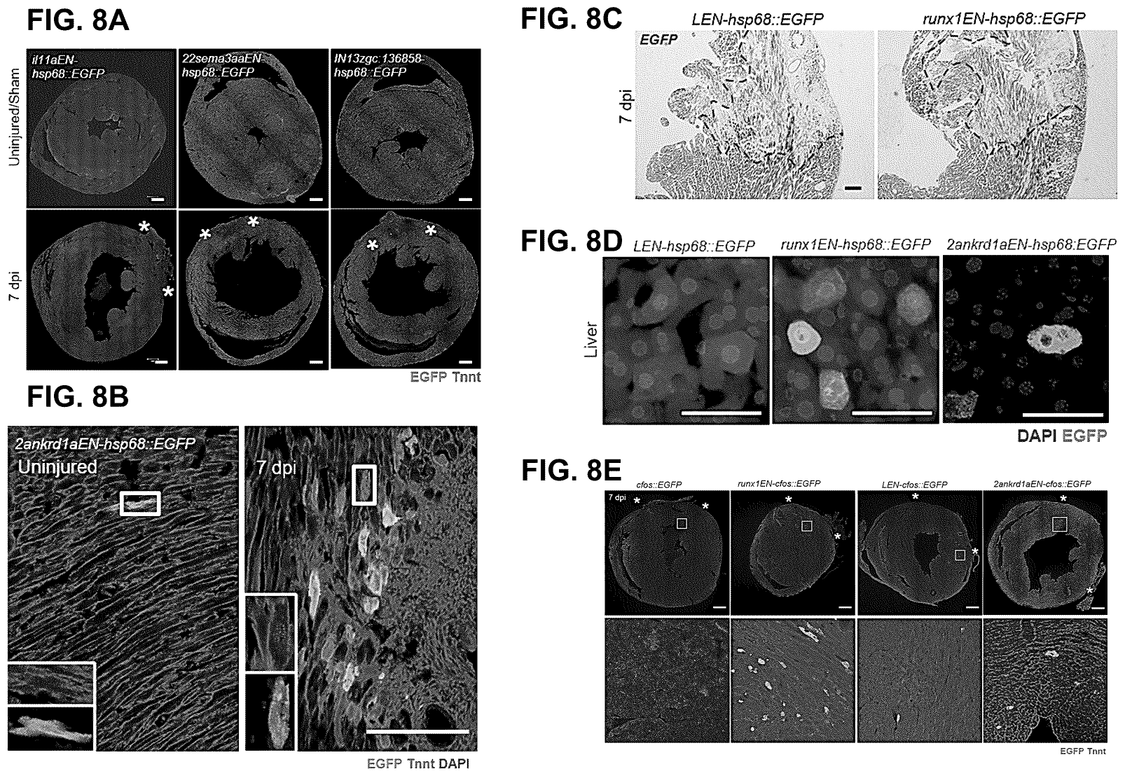

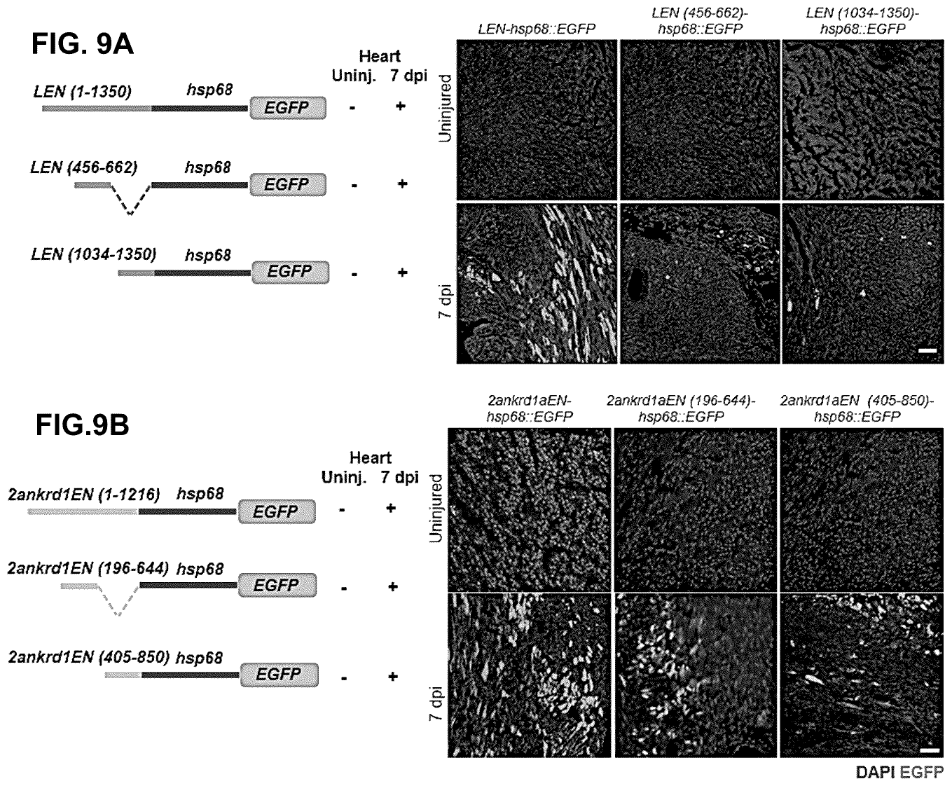

To explore the activity of zebrafish enhancers in a therapeutic context, adeno-associated viruses (AAVs) were employed to supply TREE-based expression modules. AAV9 vectors with zebrafish enhancer sequences (runx1EN, LEN or 2ankdr1aEN) fused to a murine minimal hsp68 promoter and an enhanced green fluorescent protein (EGFP) reporter cassette were generated, and administered them systemically to mice one week before MI (FIG. 1D, FIG. 7A-FIG. 7C). At different time points after MI, hearts were assessed for EGFP expression by immunofluorescence and in situ hybridization (ISH). Three of the six zebrafish TREEs that were tested induced reporter expression in injured mice with kinetics similar to those observed in zebrafish (Kang J, et al. (2016) Nature. 532:201-206; Goldman J A, et al. (2017) Developmental Cell. 40:392-404) (FIG. 7A-FIG. 7C) and stable mouse transgenic lines (FIG. 1B), but EGFP expression was negligible in sham-injured or uninjured hearts (FIG. 1E, FIG. 8A, FIG. 8C). TREE-directed expression domains were heavily biased to injury sites, largely in cells also expressing the cardiomyocyte marker Troponin T (FIG. 1F, FIG. 8B). This is consistent with the tropism of AAV9 for heart muscle cells in cardiac tissue (Eulalio A, et al. (2012) Nature. 492:376-381). Expression was detectable as early as 3 dpi, maintained for at least 2 weeks, and undetectable by 50 to 70 dpi. The TREEs LEN, runx1EN, and 2ankdr1aEN also directed EGFP expression when coupled with a murine minimal c-fos promoter, although with lower efficiency than with the hsp68 promoter (FIG. 8E). The ability of TREE fragments to direct gene expression following cardiac injury was assessed, finding the tested fragments were less consistent or robust than full-length TREEs (FIG. 9A-FIG. 9B). To assess off-target expression, the livers of mice given MI injuries were examined, given the expected heavy viral load in this tissue. While histology revealed weak or occasional runx1EN- and 2ankdr1aEN-based EGFP expression in hepatocytes, little or no hepatic LEN-based EGFP expression was observed (FIG. 8D). These findings indicate that zebrafish TREEs can be employed in AAV-based gene therapy vectors to rapidly direct transient gene expression to injury sites.

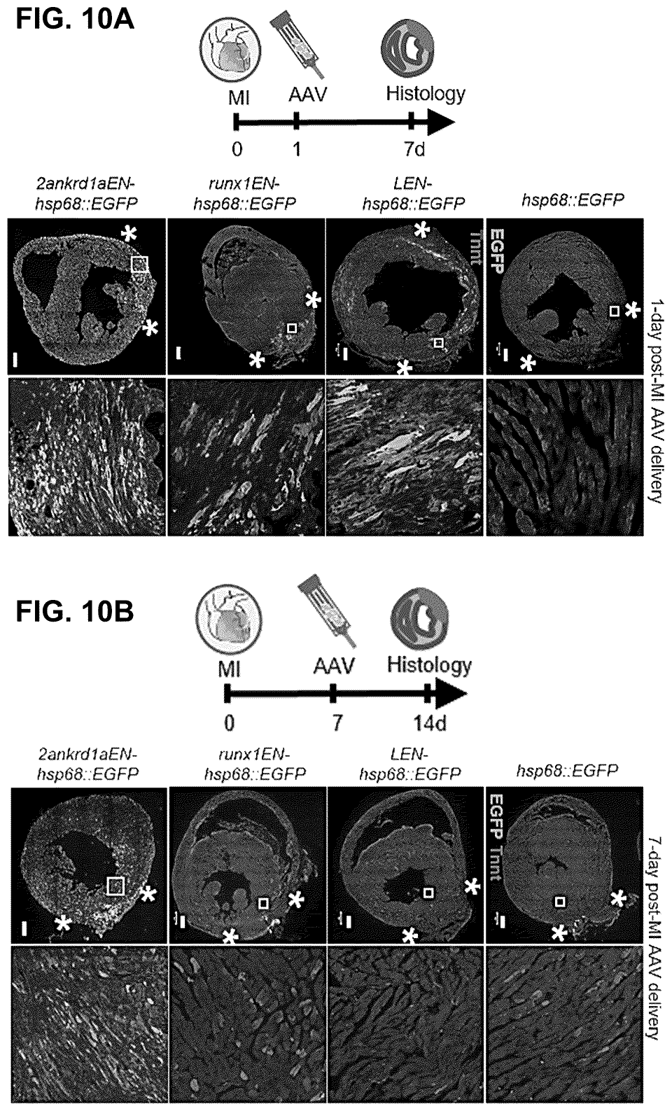

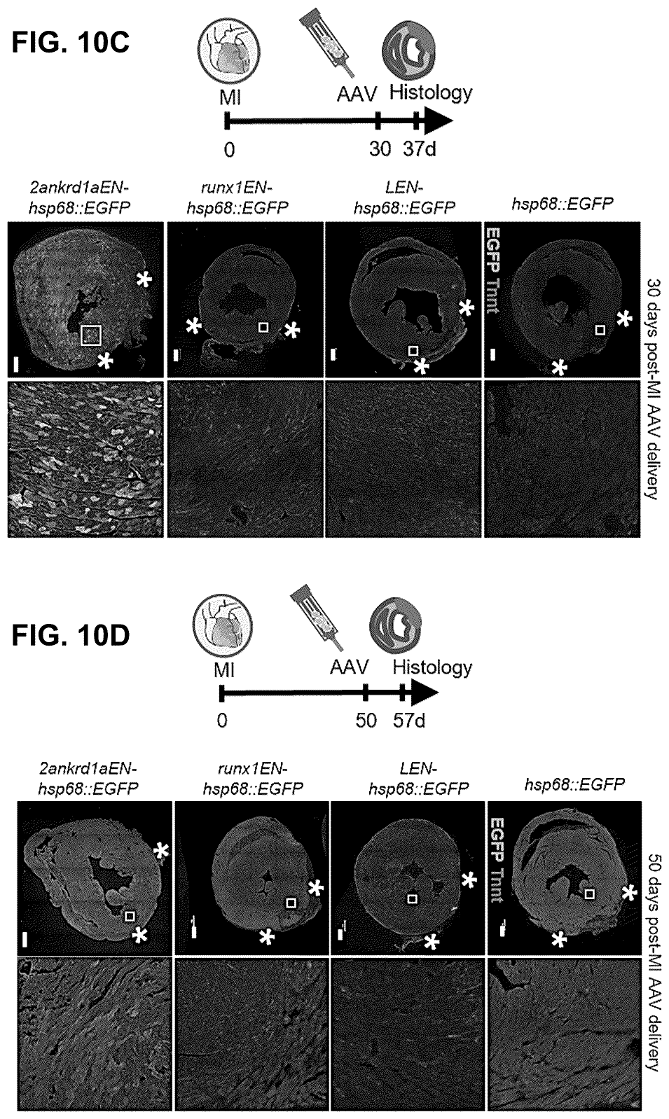

To test whether zebrafish TREEs could direct gene expression if delivered by AAV after the MI event, adult mice were injured and AAV9 carrying TREE constructs were delivered at different time points after injury (FIG. 10A-FIG. 10D). Notably, LEN, runx1EN, and 2ankrd1aEN directed EGFP expression in myocardium from AAVs when delivered 1 day after MI (FIG. 10A). Whereas LEN and runx1EN directed little to no EGFP expression when AAVs were delivered 7, 30, or 50 days post-MI, 2ankrd1aEN could still direct strong EGFP expression when delivered at 7 and 30 days post MI (FIG. 10B-FIG. 10D). Thus, while different zebrafish TREEs have different expression capabilities, they are able to target gene expression to MI border zones whether delivered before or after the injury. This is relevant to their potential use in therapeutic regimens.

### Example 3

### Rat, Porcine, and Human Transcriptional Machineries Obey Instructions from Zebrafish TREEs

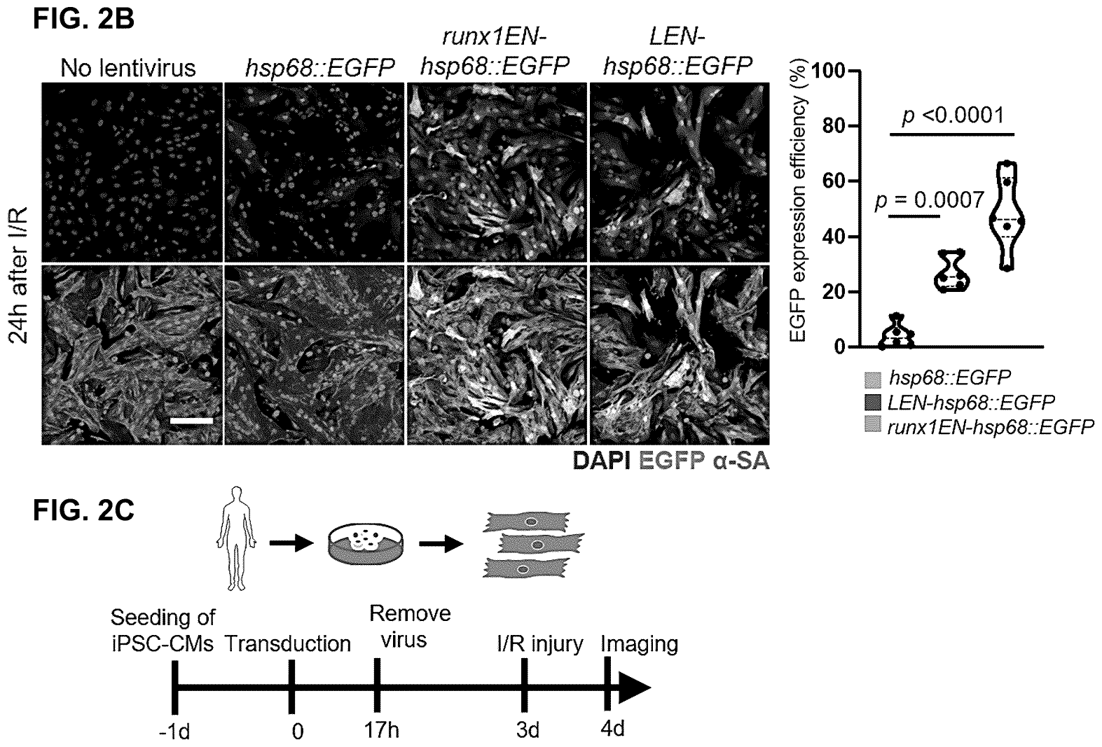

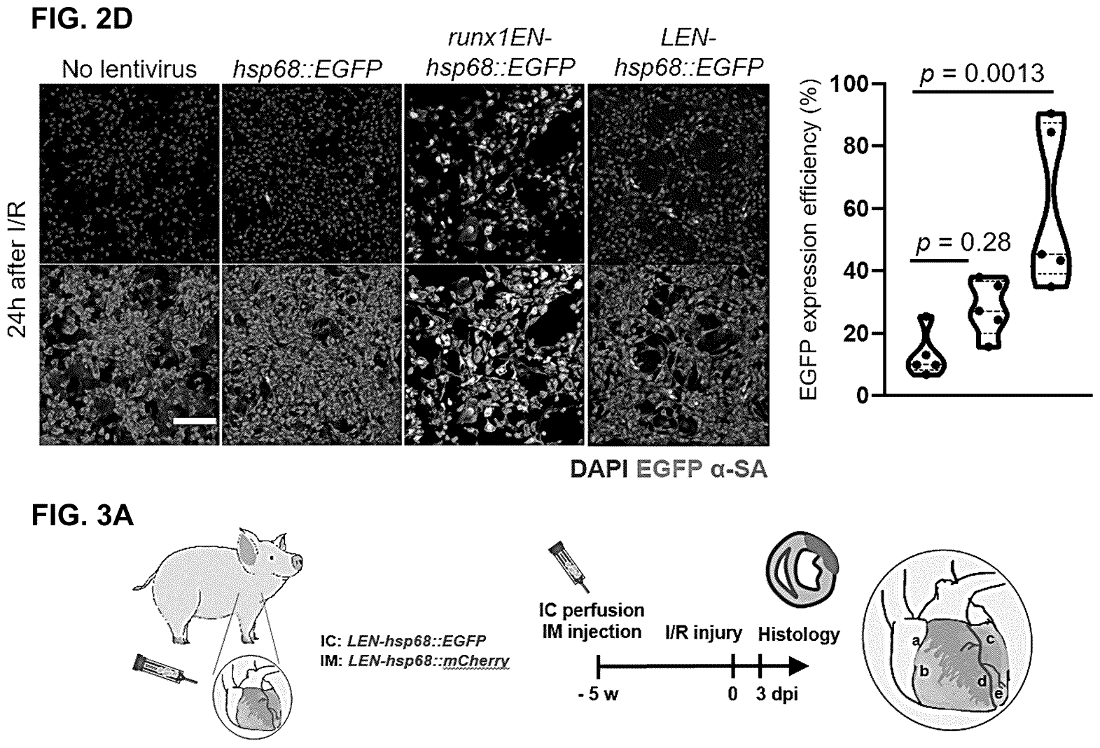

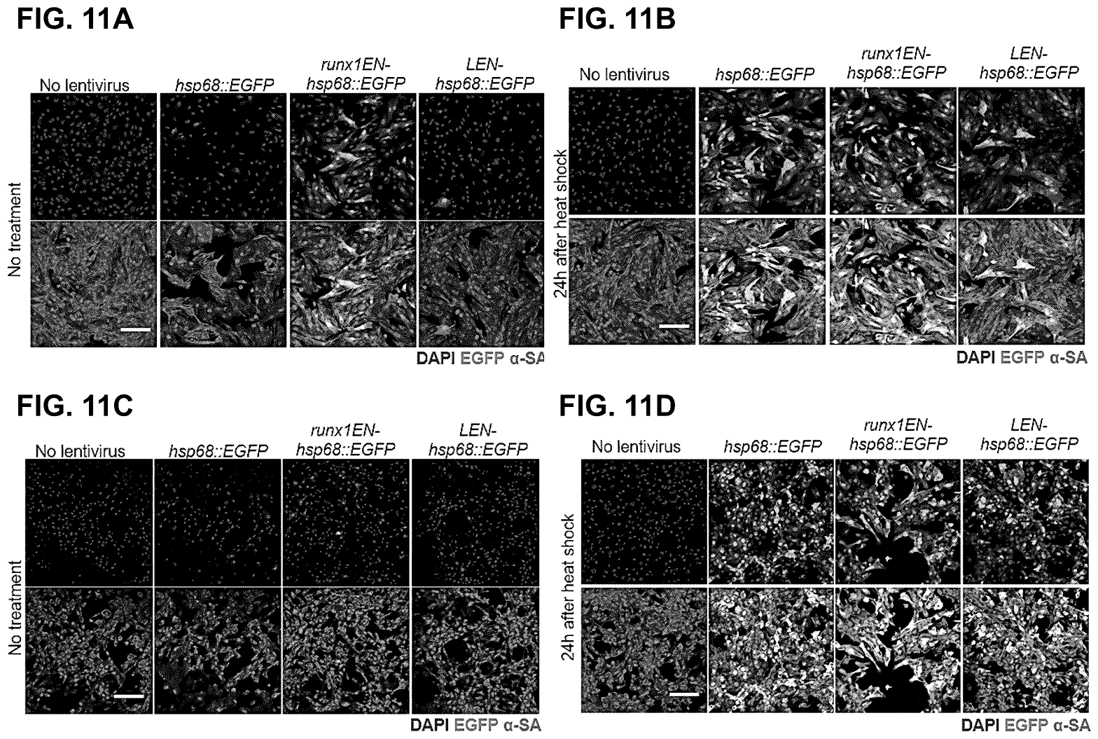

To further probe cross-species properties of TREEs, lentiviruses harboring TREE-hsp68::EGFP or control hsp68::EGFP sequences were generated and introduced them into cultured neonatal rat ventricular myocytes (NRVMs) or human iPSC-derived cardiomyocytes (iPSC-CMs) (FIG. 2A, FIG. 2C). Little or no basal EGFP fluorescence was detected in NRVMs or iPSC-CMs transduced with LEN-hsp68::EGFP lentivirus. iPSC-CMs transduced with runx1EN-hsp68::EGFP lentivirus (FIG. 11A-FIG. 11B) also displayed little or no EGFP; however, moderate EGFP expression was detected in NRVMs transduced with runx1EN-hsp68::EGFP lentivirus (FIG. 11A). Because Runx1 is a known marker of immature CMs (Kubin T, et al. (2011) Cell Stem Cell. 9:420-432), whether the developmental stage of the cells impacted runx1EN activity (FIG. 11B, FIG. 11D) was examined. Hypoxic injury conditions were introduced to mimic ischemia/reperfusion injury (I/R), finding that EGFP was sharply induced in runx1EN-hsp68::EGFP NRVM or iPSC-CM cultures 24 hrs after exposure, with hsp68::EGFP CMs displaying little or no EGFP signal (FIG. 2B, FIG. 2D). LEN directed UR-associated EGFP expression in NRVMs but not iPSC-CMs (FIG. 2B, FIG. 2D).

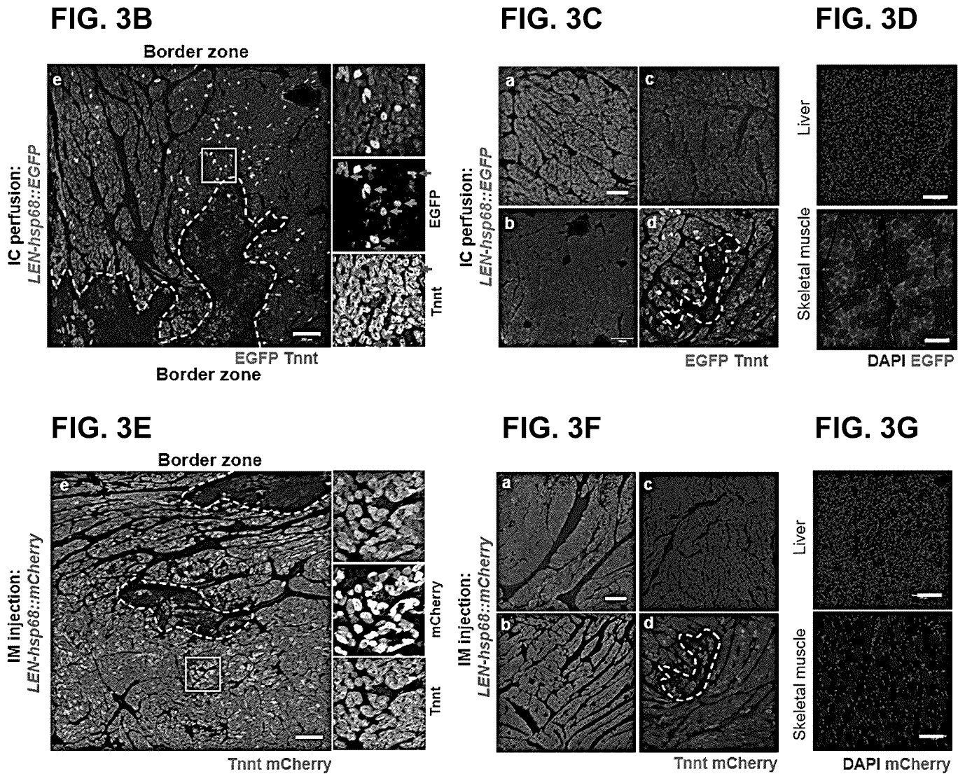

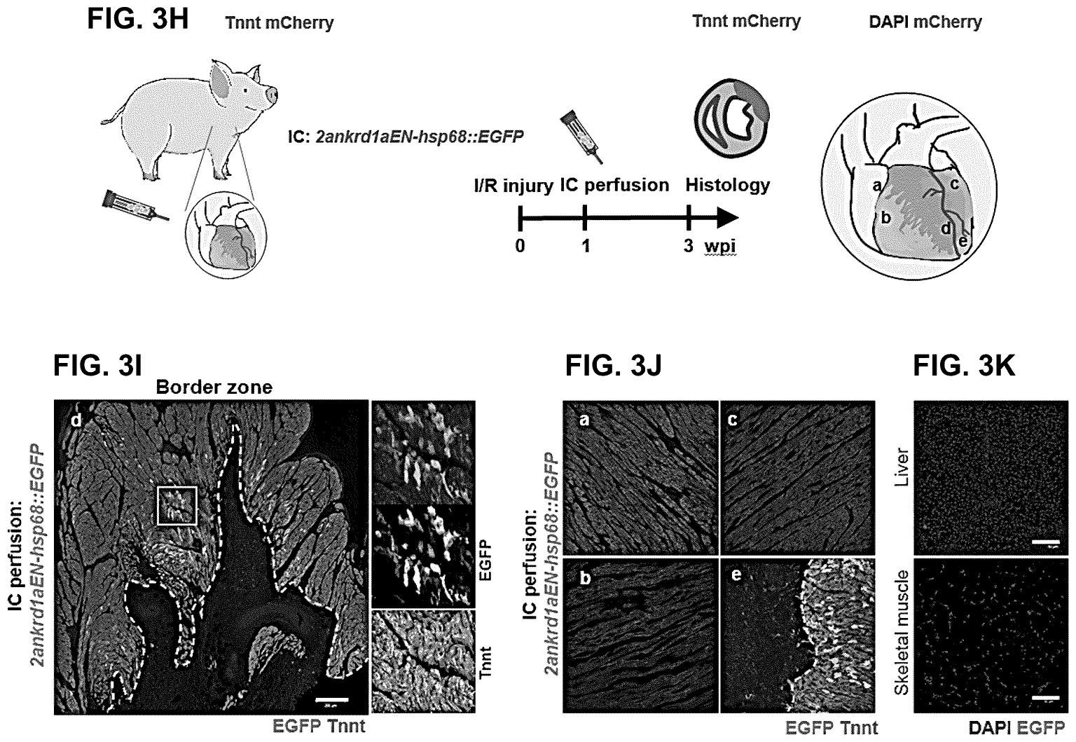

To test in vivo recognition in large mammals, LEN-hsp68::mCherry AAV9 was introduced by many intramuscular (IM) injections, as well as LEN-hsp68::EGFP AAV9 by intracoronary (IC) perfusion, to young Yorkshire breed swine 5 weeks before an UR injury (FIG. 3A). Many regions of cardiac tissue within and distal to the injury were collected for expression analysis at 3 dpi. The expression of both LEN-based reporter genes were detected preferentially in border zone tissue, indicating efficacy whether delivered directly to muscle or through the vasculature (FIG. 3B, FIG. 3C, FIG. 3E, FIG. 3F). EGFP or mCherry expression was negligible in liver and skeletal muscle of these animals, indicating effective tissue-targeting (FIG. 3D, FIG. 3G). To examine the ability to direct expression when delivered after injury, pigs were given I/R injuries and 2ankrd1aEN-hsp68::EGFP AAV9 was introduced by IC injection at one week post injury (FIG. 3H). Under this regimen, 2ankrd1aEN-directed EGFP reporter gene expression selectively in the border zone (FIG. 3I, FIG. 3J), with negligible expression in distant myocardium, liver, or skeletal muscle (FIG. 3K). These findings reveal that zebrafish TREEs are capable of targeting gene expression to cardiac injury sites in large mammals.

### Example 4

### CRISPR-TREE Systems Modulate Endogenous Gene Expression in Myocardial Injury Sites of Mice

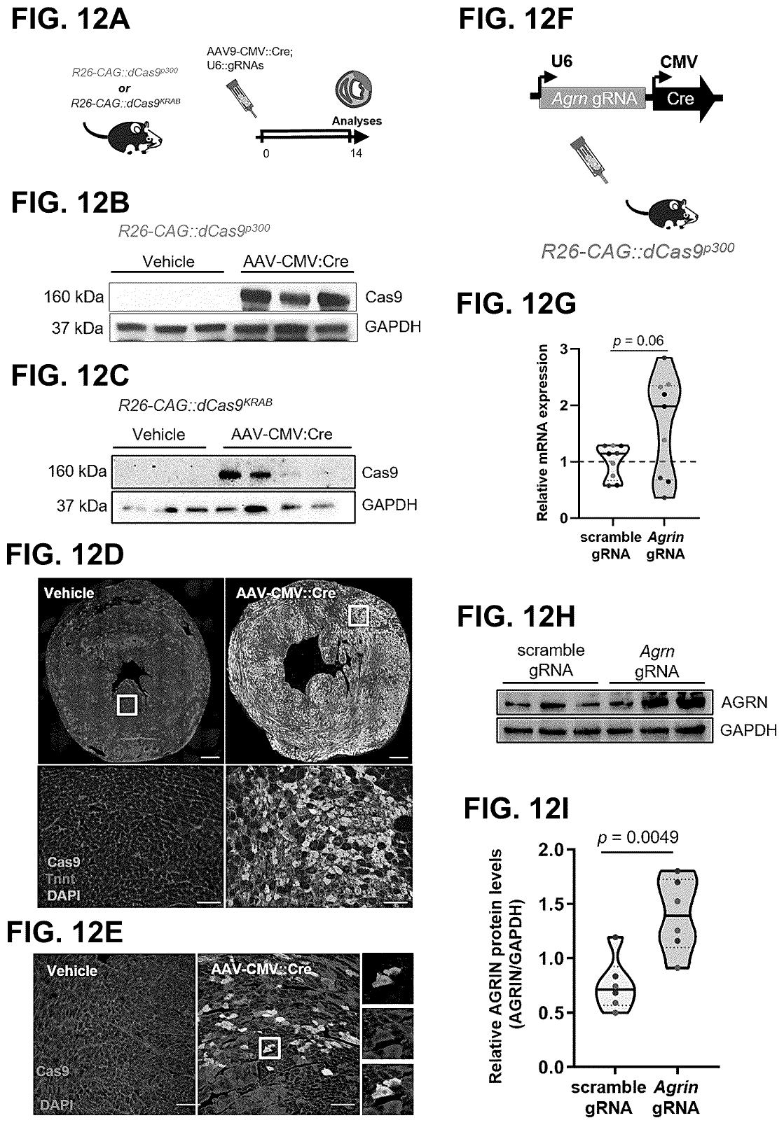

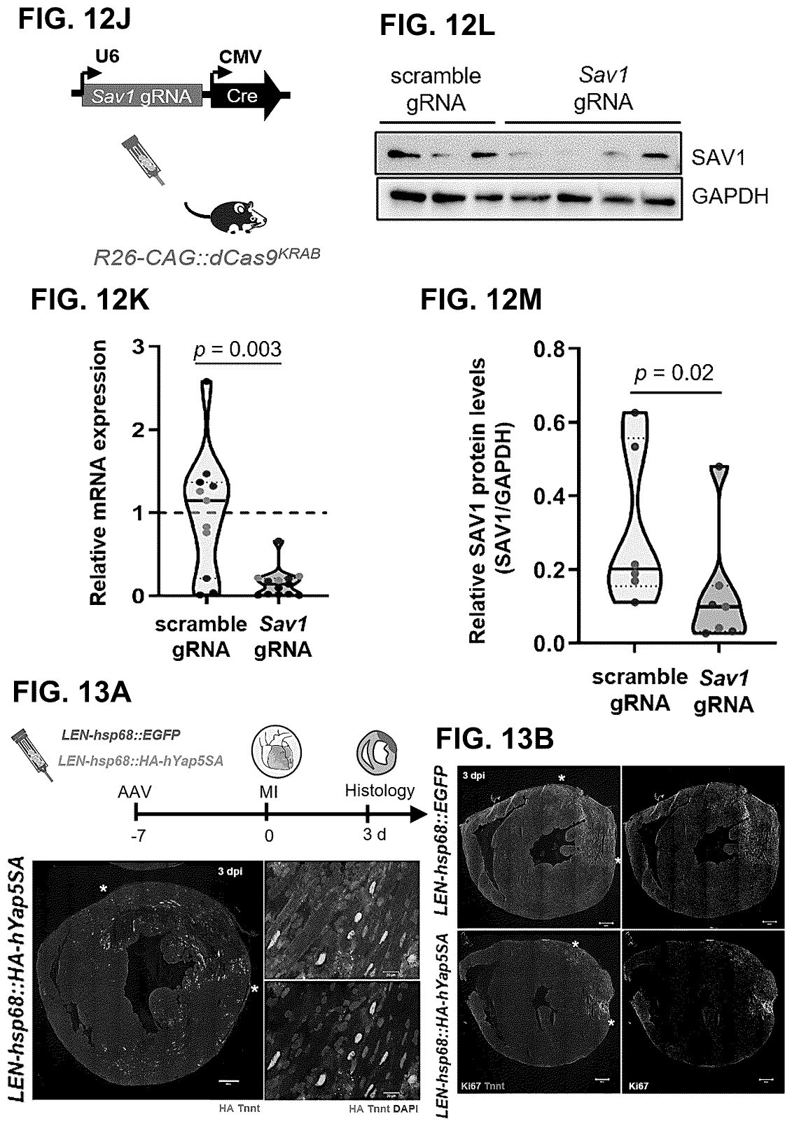

The ability to perturb endogenous gene expression in vivo is a powerful tool with potential applications to regeneration. To determine whether zebrafish TREEs could promote injury-associated epigenome editing in mouse heart, previously characterized mouse lines carrying a CAG promoter upstream of a loxP-stop-loxP (LSL) cassette were employed, followed by dCas9 fused either to the p300 or KRAB protein coding sequences (Rosa26:LSL-dCas9^{p300} and Rosa26:LSL-dCas9^{KRAB}) (FIG. 12A) (Gemberling M P, et al. (2021) Nat. Methods. 18:965-974). These constructs act by activating or repressing gene expression, respectively. To evaluate the efficacy of the dCas9^{p300} and dCas9^{KRAB} lines in adult mouse heart, mice were injected with AAV9 expressing Cre recombinase under the control of a CMV promoter by tail vein injection. Injected mice displayed induced dCas9^{p300} and dCas9^{KRAB} expression in the ventricle 2 weeks after injection, as assessed by western blot and immunofluorescence (FIG. 12B-FIG. 12E). To test whether endogenous gene expression could be manipulated, this system was tested with two genes implicated in cardiomyocyte biology, Agrin (Agrn) and Salvador (Sav1). Four gRNAs targeting the promoter region of Agrn and 8 gRNAs targeting the promoter region of Sav1 were designed, incorporated them individually downstream of a U6 promoter into AAV9 constructs together with a CMV::Cre cassette, and infected dCas9^{p300} or dCas9^{KRAB} mice. Two weeks after injection of Agrn gRNAs into dCas9^{p300} mice, a ˜60% increase in Agrn mRNA levels was observed and a ˜54% increase in AGRN protein levels using one of the 4 gRNAs tested (FIG. 12F, FIG. 12I). In dCas9^{KRAB} mice injected with Sav1 gRNAs, a ˜85% reduction of Sav1 mRNA levels was observed and a ˜56% decrease in SAV1 protein levels was observed with one of the 8 gRNAs tested at 2 weeks after AAV injection (FIG. 12J, FIG. 12M).

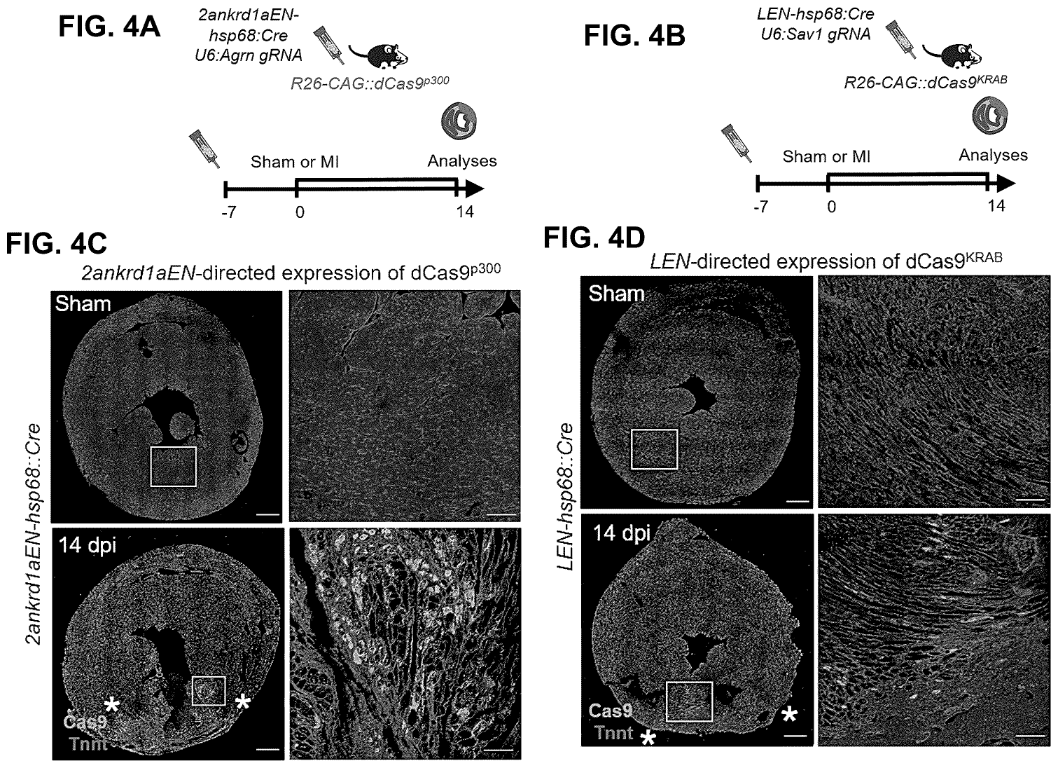

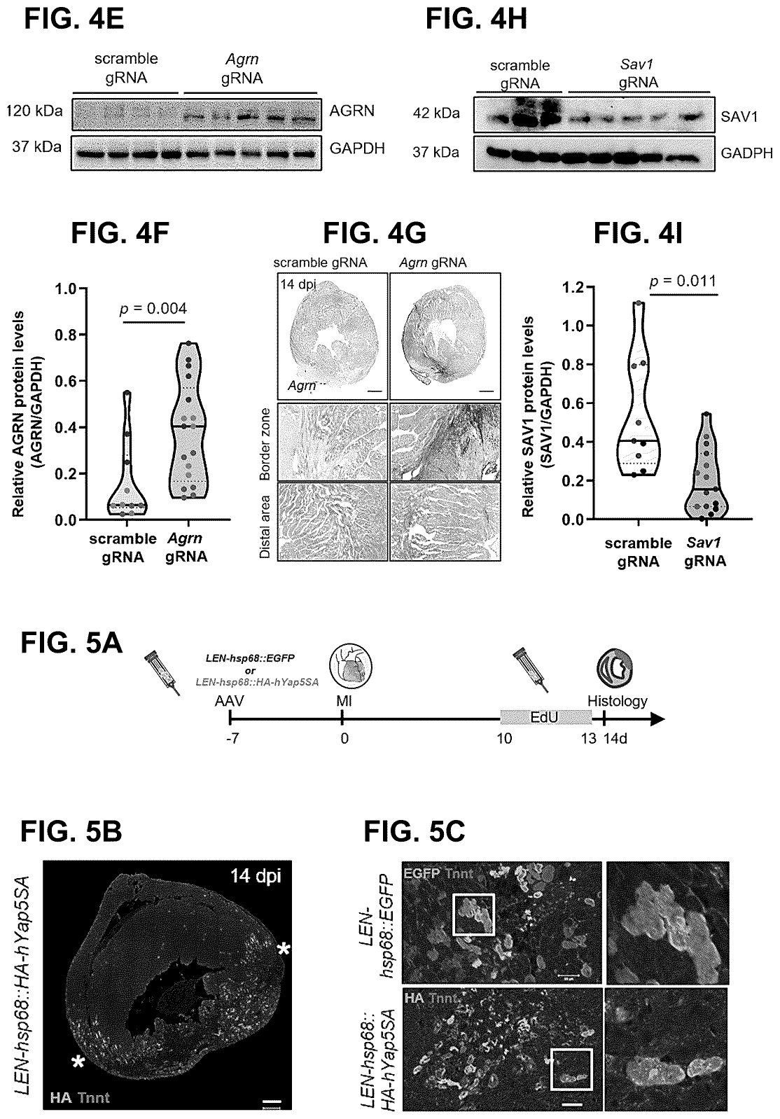

To assess whether zebrafish TREEs could target dCas9^{p300} and dCas9^{KRAB} activities to injury sites, AAV9 constructs harboring LEN or 2ankd1aEN upstream of a hsp68::Cre cassette were generated. dCas9^{p300} or dCas9^{KRAB} mice were injected with AAVs, MI injuries were performed, and hearts were analyzed by immunofluorescence at 14 dpi (FIG. 4A-FIG. 4B). In these experiments, dCas9^{p300} or dCas9^{KRAB} expression was selectively induced near and within the injured myocardium (FIG. 4C-FIG. 4D). Next, AAVs carrying 2ankd1aEN-hsp68::Cre; U6::Agrn gRNA or LEN-hsp68::Cre; U6:: Sav1 gRNA were injected into dCas9^{p300} and dCas9^{KRAB} mice, respectively, performed MI, and harvested hearts at 14 dpi. AGRN protein levels were ˜41% higher in dCas9^{p300} ventricles injected with 2ankd1aEN-hsp68::Cre; U6::Agrn gRNA AAV compared to mice treated with scramble gRNA (FIG. 4E-FIG. 4F). These changes largely occurred in the border zone, as assessed by ISH (FIG. 4G). Conversely, SAV1 protein levels were ˜62% lower in hearts of dCas9^{KRAB} mice infected with LEN-hsp68::Cre; U6::Sav1 gRNA AAV compared to controls (FIG. 4H-FIG. 4I). These experiments provided evidence that TREEs can be incorporated into viral vectors to increase or reduce the expression of endogenous genes at injury sites using CRISPR-based epigenome editing.

### Example 5

### TREE-Based Mitogen Delivery Elevates Cardiomyocyte Proliferation in Murine MI Injuries

Proliferation of pre-existing CMs is the primary cellular source of new muscle during cardiac regeneration in zebrafish and neonatal mice. Evidence indicated that suppression of the Hippo pathway or overexpression of the active form of Yap (Yap5SA, mutating its five inhibitory phosphorylation sites) elevates CM division in developing or adult mice. Notably, transgenic mice with organ-wide Yap5SA expression die of heart failure within days of induced expression, an event ostensibly caused by associated occlusion of ventricular chambers (Monroe T O, et al. (2019) Dev. Cell. 48:765-779). Using TREEs to spatiotemporally control such a potent gene cassette was believed to reduce deleterious effects.

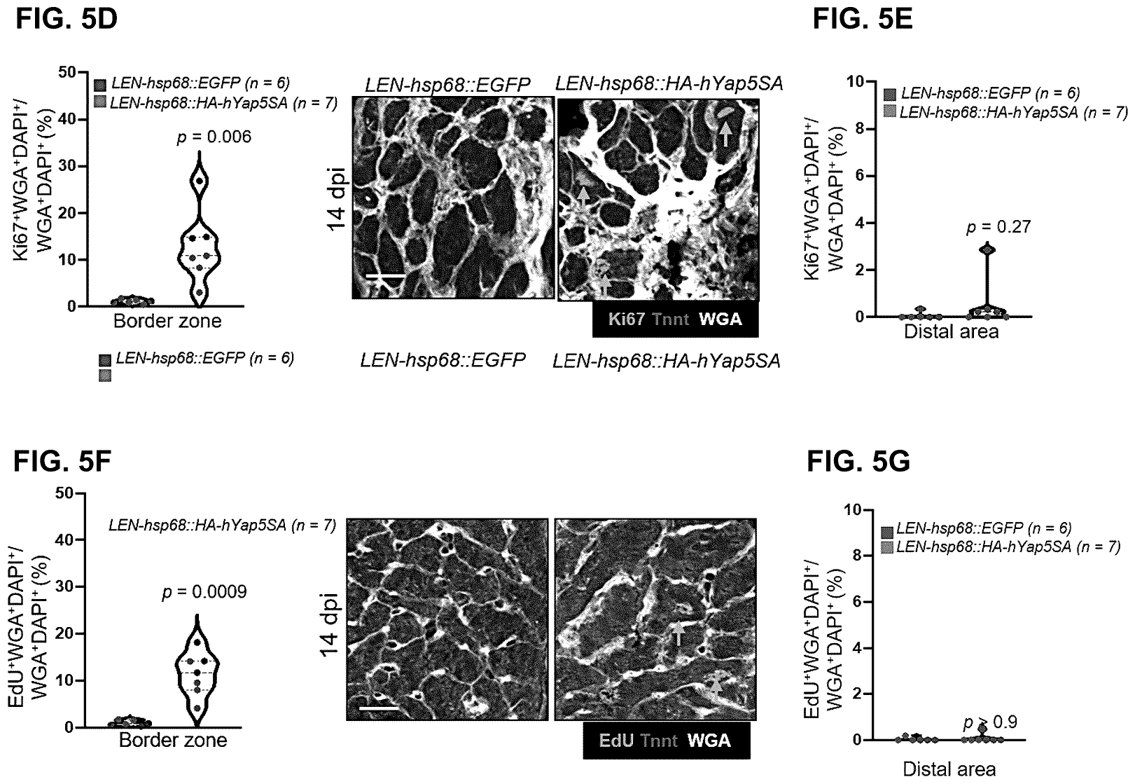

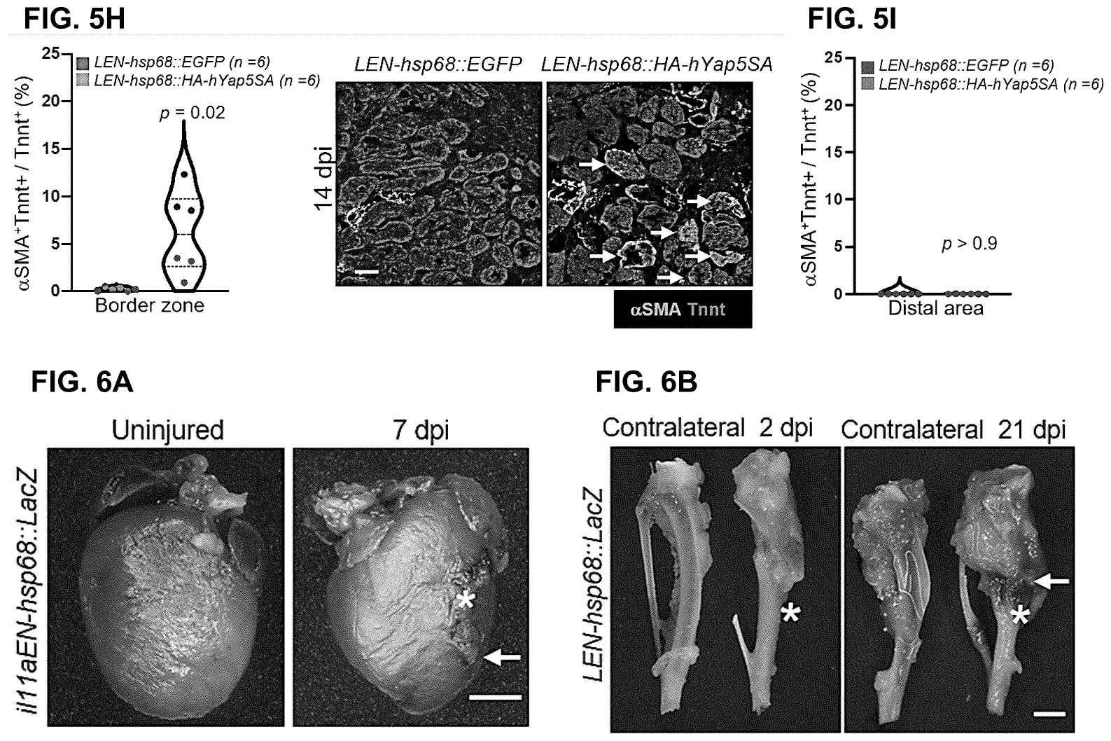

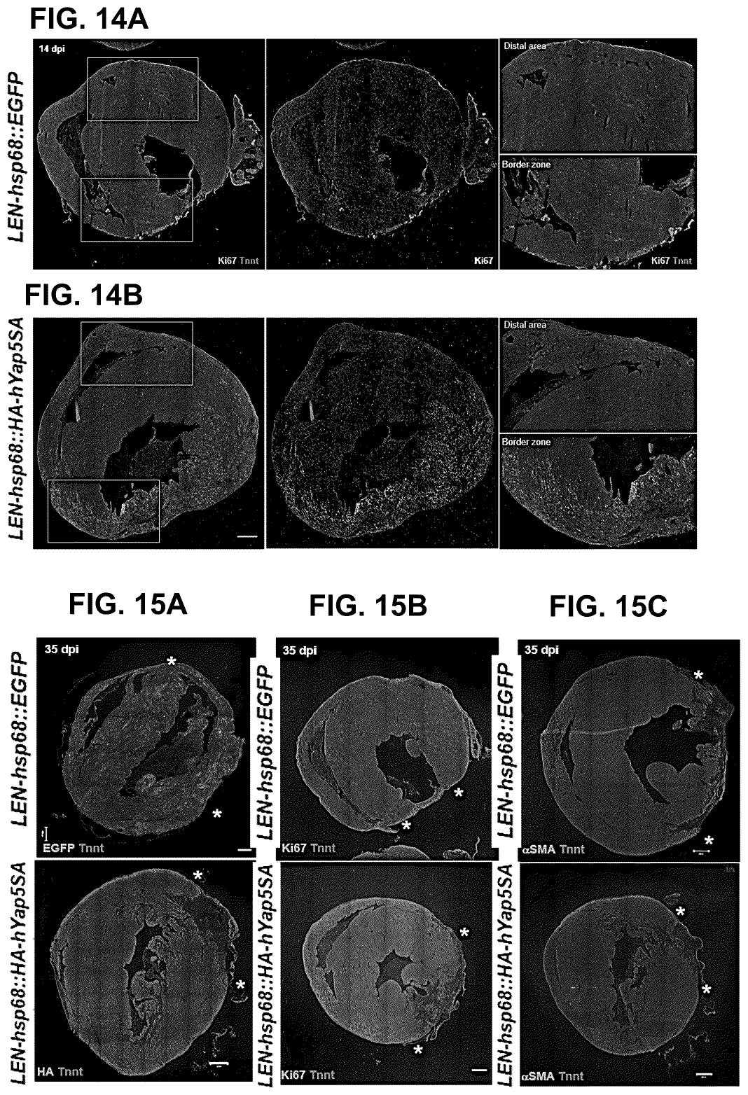

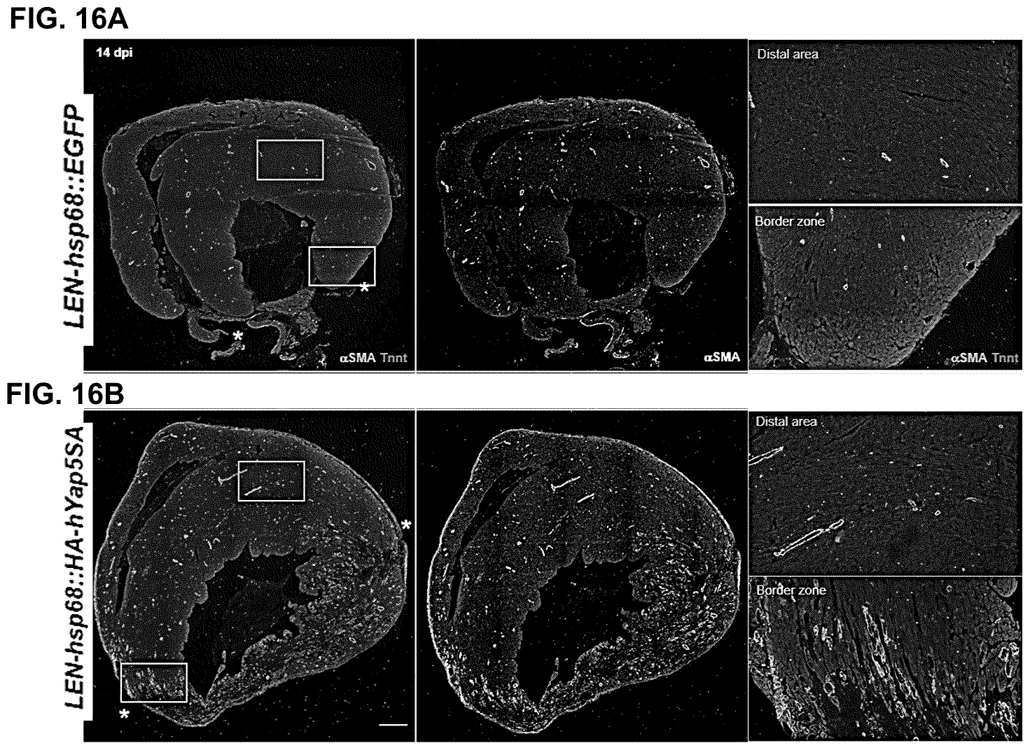

To determine the effects of a TREE-delivered pro-regenerative factor in adult mice, either LEN-hsp68::HA-hYap5SA or control LEN-hsp68::EGFP AAVs was introduced by tail vein injection. One week later, the LAD was ligated, and then hearts were collected at 3, 14, or 35 dpi (FIG. 5A). As expected, LEN-based Yap5SA expression was observed selectively at the border zone at 3 and 14 dpi (FIG. 5B, FIG. 13A) with immunofluorescence revealing nuclear accumulation of Yap (FIG. 5C, FIG. 13A). Strikingly, LEN-hsp68::HA-hYap5SA displayed a visible and selective increase in the number of cells positive for Ki67, a marker of cycling cells, in the border zone at 14 dpi, compared to control LEN-hsp68::EGFP hearts (FIG. 14A-FIG. 14B), with no obvious difference was observed at 3 and 35 dpi (FIG. 13B, FIG. 15B). Quantification of CM cycling indices in border zones of LEN-hsp68::HA-hYap5SA-infected animals compared with LEN-hsp68::EGFP controls revealed increases of 12.71%+/−2.80 vs. 1.11%+/−0.19 in Ki67 indices, and increases of 11.42%+/−1.75 vs. 0.97%+/−0.24 in EdU incorporation indices (FIG. 5D, FIG. 5F). By contrast, there were no significant differences in CMs distant from injuries (FIG. 5E, FIG. 5G). In addition to these indicators of cell cycling, a similar increase in the percentage of CMs positive for α-smooth muscle actin (αSMA), a marker of CM dedifferentiation, was observed selectively in 14 dpi border zones of LEN-hsp68::HA-hYap5SA-infected animals (6.23%+/−1.77 vs. 0.22%+/−0.08; FIG. 5H-FIG. 5I, FIG. 16A-FIG. 16B). This difference was not evident at 35 dpi (FIG. 15C). These findings indicated that zebrafish TREEs delivered systemically on gene therapy vectors guided expression of pro-regenerative factors to injury sites, selectively boosting CM proliferation and dedifferentiation.

### Summary of Experimental Results

A prevailing view is that mammals have all gene products required to regenerate an injured heart, crushed spinal cord connections, or amputated limb. Indeed, neonatal mammals can regenerate after major cardiac or spinal cord injury (Porrello E R, et al. (2011) Science. 331:1078-1080; Li Y, et al. (2020) Nature. 587:613-618), and even humans can regenerate digit tips. (Lee L P, et al. (1995) J. Hand Surg. Br. 20:63-71; Vidal P, et al. (1993) J. Hand Surg. Br. 18:230-233; Illingworth C M. (1974) J. Pediatr. Surg. 9:853-858). Instead, mammals lack or gradually lose the competency to deliver and/or receive instructions that strategically modulate those gene products for regeneration. One element of this competency exists in the form of gene regulatory elements that remain or become accessible to execute instructions in contexts of elevated regenerative capacity like that displayed by zebrafish. Here, even in situations of limited regenerative capacity, the transcriptional machinery of small and large adult mammals recognized and bound to TREE sequences from zebrafish, present as transgenes or on viral vectors. Injury-responsive sequences within TREEs were thus interpreted by mammalian transcription factors, rather than regeneration-responsive sequences per se. This recognition is founded on functional conservation of cis-regulatory elements rather than sequence conservation, which could possibly represent a remnant of elevated regeneration potential in a common ancestor.

The holy grail of regenerative medicine is molecular; that is, compounds or gene vectors that boost the latent regenerative capacity of injured human tissues without the addition of cells, parts, or pieces. The major challenges to molecular therapies are not only identifying effectors and agents, but exquisitely targeting interventions for specificity and safety. Implanted devices can conceivably deliver a pro-regenerative compound to a defined area, using machine and human monitoring to control dosing (Lansky A J, et al. (2021) Circulation. 143:2143-2154). Here, regulatory elements contained within short sequences represent a natural solution to monitor injury and dynamically control amounts, range, and duration of dosing. Incorporation of TREEs into non-integrating AAV vectors can form and shape concepts of precision regenerative medicine.

The experiments described here represent avenues for gene therapies to spatiotemporally target and control tissue regeneration and achieve long-lasting functional improvement. The use of TREEs originating from zebrafish should not be essential to these concepts, and panels of regulatory elements from diverse species and tissues can be identified and tested for efficacy. TREEs should be examined in the most truncated form possible to conserve viral load, potentially enabling tests of combinatorial effects. Payloads need not be mitogens, but could include agents that impact vascularization, innervation, cell survival, inflammation, or scarring. These could be provided combinatorically in multiple vectors controlled by different regulatory sequences, Finally, off-target gene expression might be difficult to entirely eliminate, and even low levels in remote organs may be detrimental. Continued evolution of AAVs to target specific tissues and cell types can mitigate this and provide a yet additional level of specificity.

1. An isolated nucleic acid molecule, comprising:
a nucleic acid sequence encoding
a tissue regeneration enhancer element (TREE);
a minimal promoter with little or no basal activity; and
a 3′ UTR noncoding region.

2. The isolated nucleic acid molecule of claim 1, further comprising a reporter transgene.

3. The isolated nucleic acid molecule of claim 1, further comprising inverted terminal repeats derived from an adeno-associated viral (AAV) genome.

4. The isolated nucleic acid molecule of claim 1, wherein the TREE comprises a noncoding sequence that is sufficient to direct expression of an encoded polypeptide or endogenous gene when introduced into stressed, damaged, and/or injured tissues.

5. The isolated nucleic acid molecule of claim 1, wherein the TREE comprises LEN, runx1EN, or 2ankdr1aEN.

6. The isolated nucleic acid molecule of claim 1, further comprising an encoded polypeptide having biological activity.

7. The isolated nucleic acid molecule of claim 6, wherein the TREE controls the ability of the minimal promoter to direct expression of the encoded polypeptide in stressed, damaged, and/or injured tissues.

8. The isolated nucleic acid molecule of claims 7, wherein the TREE activates expression of the encoded polypeptide in the stressed, damaged, and/or injured tissues, maintains expression of the encoded polypeptide during regeneration in the stressed, damaged, and/or injured tissues, and/or alleviates expression of the encoded polypeptide after regeneration concludes in the stressed, damaged, and/or injured tissues.

9. The isolated nucleic acid molecule of claim 1, wherein the TREE controls the ability of the minimal promoter to direct expression of an endogenous gene in stressed, damaged, and/or injured tissues.

10. (canceled)

11. The isolated nucleic acid molecule of claim 1, wherein the minimal promoter comprises a Hsp68 minimal promoter, a cfos minimal promoter, an e1b minimal promoter, or a fragment thereof.

12. The isolated nucleic acid molecule of claim 6, wherein the at least one encoded polypeptide having biological activity comprises a mammalian YAP1, a mammalian Neuregulin 1, a mammalian VegfA, or a fragment thereof.

13. (canceled)

14. A vector, comprising: the isolated nucleic molecule of claim 1.

15. The vector of claim 14, wherein the viral vector is a lentiviral vector or a recombinant lentiviral vector.

16. The vector of claim 14, wherein the viral vector is a AAV vector or a recombinant AAV vector.

17. (canceled)

18. A method of treating stressed, damaged, and/or injured tissues in a subject, the method comprising:
administering to a subject in need thereof the vector of claim 13.

19. (canceled)

20. The method of claim 18, wherein the vector is directly administered to the stressed, damaged, and/or injured tissue.

21. The method of claim 20, wherein the minimal promoter directs the expression of an endogenous polypeptide or an encoded polypeptide in the subject's stressed, damaged, and/or injured tissue.

22. (canceled)

23. The method of claim 20, wherein the subject's stressed, damaged, and/or injured tissue comprises cardiac tissue, brain tissue and/or spinal cord tissue, or cartilage and/or bone.

24.-29. (canceled)

30. The method of claim 18, further comprising repeating the administering step.

31. The method of claim 18, further comprising administering one or more therapeutic agents.

32.-56. (canceled)
